SEQUENCE LISTING (190 sequences)

SEQ ID NO: 1 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 2 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 3 - protein, 107 aa, synthetic construct
DIQLTQSPSSLSASVGDRVSITCKASQDVSIAVAWYQQKPGKAPKLLIYSASDRYTGVPDRFSGSGSGTDFTLTISSLQPEDFAVYYCQQHYITPLTFGAGTKVEIK

SEQ ID NO: 4 - protein, 5 aa, synthetic construct
NYGMN

SEQ ID NO: 5 - protein, 17 aa, synthetic construct
WINTYTGEPTYTDDFKG

SEQ ID NO: 6 - protein, 12 aa, synthetic construct
GGFGSSYWYFDV

SEQ ID NO: 7 - protein, 11 aa, synthetic construct
KASQDVSIAVA

SEQ ID NO: 8 - protein, 7 aa, synthetic construct
SASYRYT

SEQ ID NO: 9 - protein, 9 aa, synthetic construct
QQHYITPLT

SEQ ID NO: 10 - protein, 7 aa, synthetic construct
SASDRYT

SEQ ID NO: 11 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 214 aa, synthetic construct
DIQLTQSPSSLSASVGDRVSITCKASQDVSIAVAWYQQKPGKAPKLLIYSASDRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 121 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSS

SEQ ID NO: 15 - protein, 107 aa, synthetic construct
DIQLTQSPSSLSASVGDRVTITCKASQDVSIAVAWYQQKPGKAPKLLIYSASYRYTGVPSRFSGSGSGTDFTLTISSLQPEDFATYYCQQHYITPLTFGPGTKVEIK

SEQ ID NO: 16 - protein, 107 aa, synthetic construct
DIQLTQSPSSLSASVGDRVTITCKASQDVSIAVAWYQQKPGKAPKLLIYSASDRYTGVPSRFSGSGSGTDFTLTISSLQPEDFATYYCQQHYITPLTFGPGTKVEIK

SEQ ID NO: 17 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 20 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 21 - protein, 214 aa, synthetic construct
DIQLTQSPSSLSASVGDRVTITCKASQDVSIAVAWYQQKPGKAPKLLIYSASYRYTGVPSRFSGSGSGTDFTLTISSLQPEDFATYYCQQHYITPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 214 aa, synthetic construct
DIQLTQSPSSLSASVGDRVTITCKASQDVSIAVAWYQQKPGKAPKLLIYSASDRYTGVPSRFSGSGSGTDFTLTISSLQPEDFATYYCQQHYITPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 30 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 31 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 37 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 38 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 44 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 451 aa, synthetic construct
QVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, synthetic construct
DIQLTQSPSSLSASVGDRVSITCKASQDVSIAVAWYQQKPGKAPKLLIYSASDRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTCQDSKDSTYSLSSTLTLSKADYEKHKVYACEVTHQGLSSPVTKSFNRGEC

SEQ ID NO: 51 - protein, 214 aa, synthetic construct
DIQLTQSPSSLSASVGDRVSITCKASQDVSIAVAWYQQKPGKAPKLLIYSASDRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDCKDSTYSLSSTLTLSKADYEKHKVYACEVTHQGLSSPVTKSFNRGEC

SEQ ID NO: 52 - protein, 214 aa, synthetic construct
DIQLTQSPSSLSASVGDRVTITCKASQDVSIAVAWYQQKPGKAPKLLIYSASYRYTGVPSRFSGSGSGTDFTLTISSLQPEDFATYYCQQHYITPLTFGPGTKVEIKRTVAAPSVFIFPPSDEQLKSGTASVVCLLNNFYPREAKVQWKVDNALQSGNSQESVTCQDSKDSTYSLSSTLTLSKADYEKHKVYACEVTHQGLSSPVTKSFNRGEC

SEQ ID NO: 53 - protein, 214 aa, synthetic construct
DIQLTQSPSSLSASVGDRVTITCKASQDVSIAVAWYQQKPGKAPKLLIYSASYRYTGVPSRFSGSGSGTDFTLTISSLQPEDFATYYCQQHYITPLTFGPGTKVEIKRTVAAPSVFIFPPSDEQLKSGTASVVCLLNNFYPREAKVQWKVDNALQSGNSQESVTEQDCKDSTYSLSSTLTLSKADYEKHKVYACEVTHQGLSSPVTKSFNRGEC

SEQ ID NO: 54 - protein, 214 aa, synthetic construct
DIQLTQSPSSLSASVGDRVTITCKASQDVSIAVAWYQQKPGKAPKLLIYSASDRYTGVPSRFSGSGSGTDFTLTISSLQPEDFATYYCQQHYITPLTFGPGTKVEIKRTVAAPSVFIFPPSDEQLKSGTASVVCLLNNFYPREAKVQWKVDNALQSGNSQESVTCQDSKDSTYSLSSTLTLSKADYEKHKVYACEVTHQGLSSPVTKSFNRGEC

SEQ ID NO: 55 - protein, 214 aa, synthetic construct
DIQLTQSPSSLSASVGDRVTITCKASQDVSIAVAWYQQKPGKAPKLLIYSASDRYTGVPSRFSGSGSGTDFTLTISSLQPEDFATYYCQQHYITPLTFGPGTKVEIKRTVAAPSVFIFPPSDEQLKSGTASVVCLLNNFYPREAKVQWKVDNALQSGNSQESVTEQDCKDSTYSLSSTLTLSKADYEKHKVYACEVTHQGLSSPVTKSFNRGEC

SEQ ID NO: 56 - protein, 20 aa, synthetic construct
MSVPTQVLGLLLLWLTDARC

SEQ ID NO: 57 - protein, 19 aa, synthetic construct
MEWSWVFLFFLSVTTGVHS

SEQ ID NO: 58 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 59 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 60 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 61 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 62 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 63 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 64 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 65 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 66 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 67 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 68 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 69 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 70 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 71 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 72 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 73 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 74 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 75 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 76 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 77 - protein, 451 aa, synthetic construct
  VARIANT 1: note = Q, E, or pyroglutamate
  VARIANT 450..451: note = This region may encompass G, GK, or may be absent
XVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 78 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 79 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 80 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 81 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 82 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 83 - protein, 451 aa, synthetic construct; identical to SEQ ID NO: 61
  VARIANT 1: note = Q, E, or pyroglutamate
  VARIANT 450..451: note = This region may encompass G, GK, or may be absent

SEQ ID NO: 84 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 85 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 86 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPECVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 87 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 88 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 89 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 90 - protein, 451 aa, synthetic construct; identical to SEQ ID NO: 62
  VARIANT 1: note = Q, E, or pyroglutamate
  VARIANT 450..451: note = This region may encompass G, GK, or may be absent

SEQ ID NO: 91 - protein, 451 aa, synthetic construct
  VARIANT 1: note = Q, E, or pyroglutamate
  VARIANT 450..451: note = This region may encompass G, GK, or may be absent
XVQLLQSGSELKKPGASVKVSCKASGYTFTNYGMNWVRQAPGQGLKWMGWINTYTGEPTYTDDFKGRFSFSLDTSVSQAYLQISSLKAEDTAVYYCARGGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPXX

SEQ ID NO: 92 - protein, 330 aa, Homo sapiens [sequence omitted]

SEQ ID NO: 93 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 330 aa, synthetic construct
  VARIANT 180: note = Any amino acid other than N
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 107 aa, synthetic construct [sequence omitted]

SEQ ID NO: 103 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 105 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 106 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 107 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 108 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 109 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 110 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 111 - protein, 330 aa, synthetic construct
  VARIANT 180: note = Any amino acid other than N
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 112 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 113 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYIFPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYGTPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 214 aa, synthetic construct
DIQLTQSPSSLSASVGDRVSITCKASQDRSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYQDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 117 - protein, 214 aa, synthetic construct
DIQLTQSPSSLSASVGDRVSITCKASQHVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 451 aa, synthetic construct
QVQLQQSGSELKKPGASVKVSCKASGYTGTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 119 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYSDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 120 - protein, 214 aa, synthetic construct
DIQLTQSPSSLSASVGDRVSITCKASQDVSIAVAWYQQKPGKAPKLLIYSASVRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWNFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 122 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGTGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 123 - protein, 214 aa, synthetic construct
DIQLTQSPSSLSASVGDRVSITCKASQDVSIAVAWYQQKPGKAPKLLIYSASSRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 451 aa, synthetic construct
QVQLQQSGSELKKPGASVKVSCKASGYTFTPYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 125 - protein, 451 aa, synthetic construct
QVQLQQSGSELKKPGASVKVSCKASGYTFTNEGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 126 - protein, 451 aa, synthetic construct
QVQLQQSGSELKKPGASVKVSCKASGYTFTNYIMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 127 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSDYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 128 - protein, 214 aa, synthetic construct
DIQLTQSPSSLSASVGDRVSITCKASQDVSIAVAWYQQKPGKAPKLLIYSASRRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 214 aa, synthetic construct
DIQLTQSPSSLSASVGDRVSITCKASQDVSIAVAWYQQKPGKAPKLLIYSASNRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 214 aa, synthetic construct
DIQLTQSPSSLSASVGDRVSITCKASQDVSIAVAWYQQKPGKAPKLLIYTASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCVQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGEGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 133 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWIFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 134 - protein, 214 aa, synthetic construct
DIQLTQSPSSLSASVGDRVSITCKASQDVRIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQKYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHEITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 451 aa, synthetic construct
QVQLQQSGSELKKPGASVKVSCKASGYTFTNYSMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 138 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYLYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 139 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYHYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 140 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSSWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 141 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHNITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 214 aa, synthetic construct
DIQLTQSPSSLSASVGDRVSITCKASQDVSIPVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYAYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 144 - protein, 214 aa, synthetic construct
DIQLTQSPSSLSASVGDRVSITCKASQDVSIQVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQYYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYTDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 147 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYEYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 148 - protein, 451 aa, synthetic construct
QVQLQQSGSELKKPGASVKVSCKASGYTFTNYPMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 149 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGTSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 150 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHAITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 214 aa, synthetic construct
DIQLTQSPSSLSASVGDRVSITCKASQDVSIAVAWYQQKPGKAPKLLIYLASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 214 aa, synthetic construct; identical to SEQ ID NO: 150

SEQ ID NO: 153 - protein, 214 aa, synthetic construct; identical to SEQ ID NO: 142

SEQ ID NO: 154 - protein, 214 aa, synthetic construct; identical to SEQ ID NO: 151

SEQ ID NO: 155 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSDYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYEYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 451 aa, synthetic construct
QVQLQQSGSELKKPGASVKVSCKASGYTFTNYSMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSSWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYAYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYLYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGTSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYTDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 451 aa, synthetic construct
QVQLQQSGSELKKPGASVKVSCKASGYTFTNYIMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYFYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 451 aa, synthetic construct
QVQLQQSGSELKKPGASVKVSCKASGYTGTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 451 aa, synthetic construct
QVQLQQSGSELKKPGASVKVSCKASGYTFTPYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYSDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGTGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWNFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYQDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWIFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 451 aa, synthetic construct
QVQLQQSGSELKKPGASVKVSCKASGYTFTNYPMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 451 aa, synthetic construct
QVQLQQSGSELKKPGASVKVSCKASGYTFTNEGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGEGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 177 - protein, 107 aa, synthetic construct [sequence omitted]

SEQ ID NO: 178 - protein, 330 aa, synthetic construct [sequence omitted]

SEQ ID NO: 179 - protein, 330 aa, synthetic construct; identical to SEQ ID NO: 93

SEQ ID NO: 180 - protein, 330 aa, synthetic construct; identical to SEQ ID NO: 103

SEQ ID NO: 181 - protein, 330 aa, synthetic construct; identical to SEQ ID NO: 104

SEQ ID NO: 182 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 184 - protein, 451 aa, synthetic construct; identical to SEQ ID NO: 29

SEQ ID NO: 185 - protein, 4 aa, synthetic construct
  SITE 1: note = Maleimide-caproyl modified residue
GGFG

SEQ ID NO: 186 - protein, 4 aa, synthetic construct
  SITE 1: note = Maleimide-propanyl modified residue
GGGG

SEQ ID NO: 187 - protein, 5 aa, synthetic construct
GGGGS

SEQ ID NO: 188 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 189 - protein, 15 aa, synthetic construct
GGGGSGGGGSGGGGS

SEQ ID NO: 190 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

(19) United States
(12) Patent Application Publication
(10) Pub. No.: US 2024/0358847 A1
(43) Pub. Date: Oct. 31, 2024

(54) TROP2 BINDERS AND CONJUGATES THEREOF

(71) Applicant: MERCK SHARP & DOHME LLC, RAHWAY, NJ (US)

(72) Inventors: Minilik Angagaw, Malden, MA (US); Edward Paul Bowman, Redwood City, CA (US); Ming-Tang Chen, Dover, MA (US); Laurence Fayadat-Dilman, San Carlos, CA (US); Malgorzata A. Gil, Belmont, MA (US); Marlene C. Hinton, Mansfield, MA (US); Zhong Hua, Sunnyvale, CA (US); Rebecca Elizabeth Johnson, Oakland, CA (US); Simon B. Lang, San Francisco, CA (US); W. Michael Seganish, Castro Valley, CA (US); Andrew B. Waight, Los Altos, CA (US)

(73) Assignee: MERCK SHARP & DOHME LLC, RAHWAY, NJ (US)

(21) Appl. No.: 18/642,038

(22) Filed: Apr. 22, 2024

Related U.S. Application Data
(60) Provisional application No. 63/497,994, filed on Apr. 24, 2023.

(51) Int. Cl. A61K 47/68; A61P 35/00; C07K 16/30

(52) U.S. Cl. CPC A61K 47/68031; A61K 47/6849; A61P 35/00; C07K 16/30; C07K 2317/565

(57) ABSTRACT

TROP2 binders and variants thereof are described. In a specific embodiment, the TROP2 binders that are antibodies that preferentially bind high-expressing TROP2 cells over low-expressing TROP2 cells and conjugates thereof comprising the TROP2 binders conjugated to a payload are described.

### CROSS-REFERENCE TO RELATED APPLICATIONS

This application claims the benefit of U.S. Provisional Patent Application Ser. No. 63/497,994 filed Apr. 24, 2023, the entire contents of which are incorporated by reference herein.

### REFERENCE TO SEQUENCE LISTING SUBMITTED ELECTRONICALLY

The contents of the electronic sequence listing (25665 US NP_SL.xml; Size: 253 Kb; and Date of Creation: Feb. 27, 2024) are herein incorporated by reference in their entirety.

### BACKGROUND OF THE INVENTION

### (1) Field of the Invention

The present invention relates to TROP2 binders and variants thereof. In a specific embodiment, the present invention relates to TROP2 binders that are antibodies that preferentially bind high-expressing TROP2 cells over low-expressing TROP2 cells and conjugates thereof comprising said TROP2 binders conjugated to a payload.

### (2) Description of Related Art

Proteins that have roles in breast cancer growth, differentiation, invasion and/or metastasis can influence the biological progress of tumors and can thus provide important prognostic information. One such candidate is TROP2 (GA733-1, EGP-1), a 45 kDa monomeric trans-membrane glycoprotein that belongs to the TACSTD gene family, specifically TACSTD2, which is expressed in human epithelial cells at diverse stages of differentiation. Over-expression of TROP2 has been demonstrated to be necessary and sufficient to stimulate tumor growth and has been linked to an overall poor prognosis. Expression of TROP2 is associated with poor prognosis of several human cancers, including oral, pancreatic, gastric, ovarian, colorectal, breast and lung tumors. For example, TROP2 overexpression was observed in 55% of pancreatic cancer patients studied, with a positive correlation with metastasis, tumor grade, and poor progression-free survival of patients who underwent surgery with curative intent. Likewise, in gastric cancer, 56% of patients may exhibit TROP2 overexpression on their tumors, which again correlated with shorter disease-free survival and a poorer prognosis in those patients with lymph node involvement of TROP2-positive tumor cells.

Given these characteristics and the fact that TROP2 is linked to so many intractable cancers, TROP2 is an attractive target for therapeutic intervention. Nevertheless, TROP2 is also expressed in some normal tissues, although usually at much lower amounts when compared to those in neoplastic tissue, and often in regions of the tissues with restricted vascular access.

Several monoclonal antibodies against TROP2 have been established. Some anti-TROP2 monoclonal antibodies such as 77220 are commercially available as reagents. Some of these established anti-TROP2 monoclonal antibodies are being investigated for treating cancers.

Patent application WO9714796 describes a monoclonal antibody, BR110, which is known to bind to TROP2 on the cell surface and internalize within the cells. Patent applications WO2003074566, US2004001825, US2007212350 and US2008131363 teach RS7 antibodies and their uses for treating or diagnosing tumors. These patent applications further relate to humanized, human and chimeric RS7 antigen binding proteins (hRS7), and the use of such binding proteins in diagnosis and therapy. Anti-TROP2 monoclonal antibody AR47A6.4.2 is disclosed in WO2007095748 and AR52A301.5 is disclosed in WO2007095749, both of which are antibodies that specifically binds the TROP2-expressing cancer cell.

Patent application WO2008/144891 teaches a humanized version of AR47A6.4.2 as anti-TROP2 monoclonal antibody for the treatment of tumors. Patent application WO2011155579 teaches a monoclonal antibody or an antibody fragment thereof, which binds to the extracellular region of human TROP2 with high affinity and exhibits high ADCC activity and high antitumor activity. Patent application WO2013077458 teaches anti-human TROP2 antibodies with antitumor activity, in particular humanized antibodies including Huk5-70-2, especially having anti-tumor activity in vivo. Patent application WO 2013068946 teaches antibodies that specifically bind to TROP2.

A promising application of antibodies for the targeted treatment of tumors entails the conjugation of a multitude (2 to 8) of highly toxic payloads to the antibody, thereby generating an antibody-drug conjugate (ADC). ADCs are well known in the art, as for example described by Chari et al. (Angew. Chem. Int. Ed. 53:3796 (2014)) and Beck et al. (Nat. Rev. Drug Discov. 16:315-37 (2017)). Mechanistically, the antibody is designed to bind with high specificity to a tumor-associated receptor that is overexpressed versus healthy tissue. The ADC is thought to internalize into the tumor cell after binding to the receptor, then to release the toxic payload upon degradation of the antibody and/or the linker in the lysosome.

ADCs targeting TROP2 are known in the art and are at various stages of clinical development. DS-1062a is an ADC derived from humanized antibody hTINA conjugated to the campthothecin analogue exatecan through a protease-sensitive cleavable linker disclosed in patent application WO2015098099, and is under clinical evaluation for the treatment of solid tumors. PF-06664178 is an ADC derived from monoclonal antibody RN926 that is site-specifically conjugated to auristatin analogue PF-06380101 under the action of microbial transglutaminase. PF-06664178 had been evaluated in a phase I clinical study in patients with advanced or metastatic solid tumors, however the ADC showed toxicity at high dose levels with only modest antitumor activity, so development was discontinued.

Sacituzumab govitecan-hziy (TRODELVY, Immunomedics, Inc.) (SG) was approved in April 2020 for patients with metastatic triple-negative breast cancer (TNBC), who had received at least two prior therapies for metastatic disease (Bardia et al., N. Engl. J. Med. 380:741-51 (2019)). SG is an antibody-drug conjugate (ADC) consisting of a humanized anti-TROP2 monoclonal antibody (mAb), hRS7, linked to about 8 molecules of SN-38—the active metabolite of irinotecan and a potent inhibitor of topoisomerase 1 (Thomas et al., Clin. Cancer Res. 25:6581-9 (2019)). Notably, until SG no topoisomerase I inhibitors had been used in metastatic triple negative breast cancer (TNBC) and SG effectively constitutes a new cytotoxic drug for treating a disease that is still heavily depended on chemotherapy. However, the efficacy of SG has been hampered by its toxicity.

The SG-targeted epitope in TROP2 may further limit its efficacy. The hRS7 mAb was shown to bind the same epitope as T16, 162-46.2 (Alberti et al., Hybridoma; 11:539-45 (1992); Ikeda et al., Biochem Biophys. Res. Commun. 458:877-82 (2015)) and E1 mAb (Trerotola et al., Neoplasia 23:415-28 (2021)). Hence, RS7 adds to the list that includes most anti-TROP2 antibodies, among them MOv16 (Alberti et al., Hybridoma; 11:539-45 (1992)), cAR47A6.4.2 (Truong et al., Mol. Cancer Ther. 6:3334 (2007)), 77220, MM0588, and YY-01 (Ikeda et al., Biochem Biophys. Res. Commun. 458:877-82 (2015)) which were shown to bind an immunodominant epitope (Alberti et al., Hybridoma; 11:539-45 (1992); Ikeda et al., Biochem Biophys. Res. Commun. 458:877-82 (2015)) located in the N-terminal region of the stem domain of TROP2 (D146-T274) (Ikeda et al., Biochem Biophys. Res. Commun. 458:877-82 (2015)). This epitope was shown to be equally accessible in cancer cells and in normal tissues (Trerotola et al., Oncogene 32:222-33 (2013); Alberti et al., Hybridoma 11:539-45 (1992); Stepan et al., J. Histochem. Cytochem. 59:701-10 (2011); Kaufmann et al., Arch. Dermatol. Res. 286:6-11 (1994)), thus raising issues of a lack of cancer specificity (Trerotola et al., Biochim. Biophys. Acta 1805:119-20 (2010)). The Rinat-Pfizer RN926 anti-Trop-2 mAb, which was developed in the PF-06664178/Aur0101 ADC, was also shown to bind this immunodominant region of TROP2 (domain 3, residues 152-206, and domain 4, residues 209-274; WO 2013/068946). PF-06664178 had shown early promise (Strop et al., Mol. Cancer Ther.; 15:2698-708 (2016)). A Phase I, open-label, dose-escalation study of PF-06664178 was conducted in patients with advanced solid tumors. Doses of 3.60, 4.2 and 4.8 mg/kg were found to be intolerable, due to skin rash, mucosal lesions and neutropenia. PF-06664178 showed modest antitumor activity, and was ultimately discontinued (King et al., Invest. New Drugs 36:836-47 (2018)). Hence, exposure of normal tissues to anti-TROP2 ADCs bearing high-potency payloads can lead to unmanageable toxicity.

Furthermore, SG has a short half-life in patients (Ocean et al., Cancer 123:3843-54 (2017)), and frequent dosing is required, which leads to the induction of side effects, such as neutropenia and diarrhea, which have been suggested to be due to the release of SN38 as a free drug in the circulation (Santi et al., Ann. Transl. Med. 9:1113 (2021)). In view of the above, for anti-TROP2 ADCs to reach their full potential for treating cancers associated with high expression of TROP2, improvements in cell targeting and ADC half-life are needed.

### BRIEF SUMMARY OF THE INVENTION

The present invention provides the following TROP2 binders: (i) avidity-tuned anti-TROP2 antibodies derived from antibody hRS7 (Sacituzumab) that preferentially bind high TROP2-expressing cells (TROP2^{high} cells) over low TROP2-expressing cells (TROP2^{low} cells) and display reduced hydrophobicity compared to hRS7 while retaining an anti-tumor benefit in preclinical tumor models that is comparable to that of hRS7; (ii) anti-TROP2 antibodies, which are rehumanized derivatives of antibody hRS7 that display reduced hydrophobicity compared to hRS7 and reduced immunogenicity compared to hRS7; and (iii) avidity-tuned, rehumanized anti-TROP2 antibodies that combine the benefits of the avidity-tuned anti-TROP2 antibody and the rehumanized anti-TROP2 antibodies; or antigen-binding fragments thereof.

The present invention further provides ADCs comprising a TROP2 binder of the present invention conjugated to a payload. The TROP2 binders and ADCs of the present invention are useful for treating, imaging, diagnosing, preventing the proliferation of, containing and reducing TROP2-expressing cells, and in particular TROP2^{high} cells, and TROP2-expressing tumors.

In one aspect, the present invention provides an antibody or antigen-binding fragment thereof that specifically binds to human TROP2, wherein the antibody or antigen-binding fragment thereof comprises a heavy chain variable domain comprising a CDRH1, a CDRH2, and a CDRH3, and a light chain variable domain comprising a CDRL1, a CDRL2, and a CDRL3, wherein the CDRH1, CDRH2, CDRH3, CDRL1, CDRL2, and CDRL3, respectively, comprise the amino acid sequence NYGMN (SEQ ID NO: 4), WINTYTGEPTYTDDFKG (SEQ ID NO: 5), GGFGSSYWYFDV (SEQ ID NO: 6), KASQDVSIAVA (SEQ ID NO: 7), SASDRYT (SEQ ID NO: 10), and QQHYITPLT (SEQ ID NO: 9). In a further embodiment, the antibody is a humanized antibody and the antigen binding fragment of the antibody is a Fab fragment, a Fab′ fragment, or a F(ab′)₂ fragment.

In a further embodiment, the antibody or antigen binding fragment thereof displays reduced binding to low TROP2-expressing cells compared to high TROP2-expressing cells and has reduced hydrophobicity compared to Sacituzumab as determined by hydrophobic interaction chromatography (HIC).

In a further embodiment of the antibody or antigen binding fragment thereof, the heavy chain variable domain comprises the amino acid sequence of SEQ ID NO: 1 or 14 and the light chain variable domain comprises the amino acid sequence of SEQ ID NO: 3 or 16.

In a further embodiment of the antibody or antigen binding fragment thereof, the heavy chain variable domain comprises the amino acid sequence of SEQ ID NO: 1 and the light chain variable domain comprises the amino acid sequence of SEQ ID NO: 3 or the heavy chain variable domain comprises the amino acid sequence of SEQ ID NO: 14 and the light chain variable domain comprises the amino acid sequence of SEQ ID NO: 16.

In a further embodiment of the antibody or antigen binding fragment thereof, the antibody comprises a light chain comprising the amino acid sequence of SEQ ID NO: 13 or 22 and the heavy chain comprises the amino acid sequence of SEQ ID NO: 11, 17, or 18.

In a further embodiment of the antibody or antigen binding fragment thereof, the antibody comprises (a) a light chain comprising the amino acid sequence of SEQ ID NO: 13 and the heavy chain comprises the amino acid sequence of SEQ ID NO: 11; (b) a light chain comprising the amino acid sequence of SEQ ID NO: 22 and the heavy chain comprises the amino acid sequence of SEQ ID NO: 17; or (c) a light chain comprising the amino acid sequence of SEQ ID NO: 22 and the heavy chain comprises the amino acid sequence of SEQ ID NO: 18.

In a further embodiment of the antibody or antigen binding fragment thereof, the antibody comprises a light chain comprising the amino acid sequence of SEQ ID NO: 13 or 22 and the heavy chain comprises the amino acid sequence of SEQ ID NO: 58, 59, or 60.

In a further embodiment of the antibody or antigen binding fragment thereof, the antibody comprises (a) a light chain comprising the amino acid sequence of SEQ ID NO: 13 and the heavy chain comprises the amino acid sequence of SEQ ID NO: 58; (b) a light chain comprising the amino acid sequence of SEQ ID NO: 22 and the heavy chain comprises the amino acid sequence of SEQ ID NO: 59; or (c) a light chain comprising the amino acid sequence of SEQ ID NO: 22 and the heavy chain comprises the amino acid sequence of SEQ ID NO: 60.

In a further embodiment of the antibody or antigen binding fragment thereof, the antibody further comprises a cysteine or a non-canonical amino acid amino acid substitution at one or more position(s) selected from the group consisting of: positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain according to sequential numbering of the whole light chain sequence.

In a further embodiment of the antibody or antigen binding fragment thereof, the antibody comprises a cysteine or a non-canonical amino acid amino acid substitution at position 375 of the constant domain of the heavy chain.

In a further embodiment of the antibody or antigen binding fragment thereof, the antibody comprises a heavy chain comprising the amino acid sequence of SEQ ID NO: 19, 20, 61, or 62.

In a further embodiment of the antibody or antigen binding fragment thereof, the antibody is conjugated to a payload. In a further embodiment of the antibody or antigen binding fragment thereof, the cysteine or noncanonical amino acid is conjugated to a payload. In a further embodiment of the antibody or antigen binding fragment thereof, the payload is a therapeutic moiety, a detectable label, a radionuclide, or a protecting group. In a further embodiment of the antibody or antigen binding fragment thereof, the therapeutic moiety is a cytotoxic moiety, an anti-inflammatory moiety, a peptide, a nucleic acid molecule, or a nucleic acid analog. In a further embodiment of the antibody or antigen binding fragment thereof, the cytotoxic moiety is taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoid (such as DM1, DM2, DM3, DM4), auristatins including monomethylauristatin E (MMAE), monomethylauristatin F (MMAF), monomethylauristatin D (MMAD), 7-ethyl-10-hydroxy-camptothecin (SN-38), anthracycline, alkylcycline, and derivatives thereof.

In a further embodiment of the antibody or antigen binding fragment thereof, the cytotoxic moiety is an inhibitor of topoisomerase I, topoisomerase II, or microtubule polymerization.

In a further aspect, the present invention provides for uses of the aforementioned antibodies.

In one aspect, the present invention provides a method for treating a cancer in an individual in need thereof comprising administering to the individual a therapeutically effective amount of an antibody or antigen-binding fragment thereof that specifically binds to human TROP2, wherein the antibody or antigen-binding fragment thereof comprises a heavy chain variable domain comprising a CDRH1, a CDRH2, and a CDRH3, and light chain variable domain comprising a CDRL1, a CDRL2, and a CDRL3, wherein each CDRH1, CDRH2, CDRH3, CDRL1, CDRL2, and CDRL3, respectively, comprise the amino acid sequence of SEQ ID NO: 4, SEQ ID NO: 5, SEQ ID NO: 6, SEQ ID NO: 7, SEQ ID NO: 10, and SEQ ID NO: 9 to treat the cancer, wherein the cancer is a cancer that overexpresses TROP2.

In a further embodiment of the method, the cancer is selected from the group consisting of breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides an antibody or antigen-binding fragment thereof that specifically binds to human TROP2, wherein the antibody or antigen-binding fragment thereof comprises a heavy chain variable domain comprising a CDRH1, a CDRH2, and a CDRH3, and a light chain variable domain comprising a CDRL1, a CDRL2, and a CDRL3, wherein the CDRH1, CDRH2, CDRH3, CDRL1, CDRL2, and CDRL3, respectively, comprise the amino acid sequence of SEQ ID NO: 4, SEQ ID NO: 5, SEQ ID NO: 6, SEQ ID NO: 7, SEQ ID NO: 10, and SEQ ID NO: 9 for use in the manufacture of a medicament for the treatment of a cancer that overexpresses TROP2.

In a further embodiment of the use, the cancer is selected from the group consisting of breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides an antibody or antigen-binding fragment thereof that specifically binds to human TROP2, wherein the antibody or antigen-binding fragment thereof comprises a heavy chain variable domain comprising a CDRH1, a CDRH2, and a CDRH3, and a light chain variable domain comprising a CDRL1, a CDRL2, and a CDRL3, wherein the CDRH1, CDRH2, CDRH3, CDRL1, CDRL2, and CDRL3, respectively, comprise the amino acid sequence of SEQ ID NO: 4, SEQ ID NO: 5, SEQ ID NO: 6, SEQ ID NO: 7, SEQ ID NO: 10, and SEQ ID NO: 9 for the treatment of a cancer that overexpresses TROP2.

In a further embodiment, the cancer is selected from the group consisting of breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides a combination therapy for treating cancer comprising an antibody or antigen-binding fragment thereof that specifically binds to human TROP2, wherein the antibody or antigen-binding fragment thereof comprises a heavy chain variable domain comprising a CDRH1, a CDRH2, and a CDRH3, and light chain variable domain comprising a CDRL1, a CDRL2, and a CDRL3, wherein the CDRH1, CDRH2, CDRH3, CDRL1, CDRL2, and CDRL3, respectively, comprise the amino acid sequence of SEQ ID NO: 4, SEQ ID NO: 5, SEQ ID NO: 6, SEQ ID NO: 7, SEQ ID NO: 10, and SEQ ID NO: 9 and a therapeutic agent, wherein the cancer is a cancer that overexpresses TROP2.

In a further embodiment of the combination therapy, the therapeutic agent is a chemotherapy agent or a therapeutic antibody. In a further embodiment, the therapeutic antibody is a checkpoint inhibitor. In a further embodiment, the therapeutic antibody is an anti-PD1 antibody or an anti-PD-L1 antibody.

In a further embodiment of the combination therapy, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

In another aspect, the present invention provides an antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15.

In a further embodiment, the antibody or antigen-binding fragment thereof displays reduced hydrophobicity compared to Sacituzumab as determined by hydrophobic interaction chromatography (HIC).

In a further embodiment, the antigen binding fragment of the antibody is a Fab fragment, a Fab′ fragment, or a F(ab′)₂ fragment.

In a further embodiment, the light chain comprises the amino acid sequence of SEQ ID NO: 21 and the heavy chain comprises the amino acid sequence of SEQ ID NO: 17 or 18.

In a further embodiment, the antibody comprises a light chain comprising the amino acid sequence of SEQ ID NO: 21 and a heavy chain comprising the amino acid sequence of SEQ ID NO: 59 or 60.

In a further embodiment, the antibody further comprises a cysteine or a non-canonical amino acid amino acid substitution at one or more position(s) selected from the group consisting of positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

In a further embodiment, the antibody comprises a cysteine or a non-canonical amino acid amino acid substitution at position 375 of the constant domain of the heavy chain.

In a further embodiment, the antibody comprises a heavy chain comprising the amino acid sequence of SEQ ID NO: 19, 20, 61, or 62.

In a further embodiment, the antibody is conjugated to a payload. In a further embodiment, the cysteine or noncanonical amino acid is conjugated to a payload. In a further embodiment, the payload is a therapeutic moiety, a detectable label, a radionuclide, or a protecting group. In a further embodiment, the therapeutic moiety is a cytotoxic moiety, an anti-inflammatory moiety, a peptide, a nucleic acid molecule, or a nucleic acid analog. In a further embodiment, the cytotoxic moiety is taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoid (such as DM1, DM2, DM3, DM4), auristatins including monomethylauristatin E (MMAE), monomethylauristatin F (MMAF), monomethylauristatin D (MMAD), 7-ethyl-10-hydroxy-camptothecin (SN-38), anthracycline, alkylcycline, and derivatives thereof.

In a further embodiment, the cytotoxic moiety is an inhibitor of topoisomerase I, topoisomerase II, or microtubule polymerization.

The present invention further provides a method for treating a cancer in an individual in need thereof comprising administering to the individual a therapeutically effective amount of an antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15 to treat the cancer, wherein the cancer is a cancer that overexpresses TROP2.

In a further embodiment of the method, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides for the use of an antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15 for the manufacture of a medicament for the treatment of a cancer that overexpresses TROP2.

In a further embodiment of the use, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides an antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15 for the treatment of a cancer that overexpresses TROP2.

In a further embodiment, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides a combination therapy for treating cancer comprising an antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15 and a therapeutic agent, wherein the cancer is a cancer that overexpresses TROP2.

In a further embodiment of the combination therapy, the therapeutic agent is a chemotherapy agent or a therapeutic antibody.

In a further embodiment of the combination therapy, the therapeutic antibody is a checkpoint inhibitor.

In a further embodiment of the combination therapy, the therapeutic antibody is an anti-PD1 antibody or an anti-PD-L1 antibody.

In a further embodiment of the combination therapy, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

In a further aspect, the present invention provides an ADC comprising an antibody that specifically binds to human TROP2 conjugated to a linker-monomethylauristatin E (linker-MMAE) payload (MMAE-conjugated ADC), wherein the antibody comprises two heavy chains, each heavy chain comprising a variable domain and a constant domain, the variable domain comprising a complementarity determining region (CDR) H1, a CDRH2, and a CDRH3, and two light chains, each light chain comprising a variable domain and a constant domain, the variable domain comprising a CDRL1, a CDRL2, and a CDRL3, wherein the CDRH1, CDRH2, CDRH3, CDRL1, CDRL2, and CDRL3, respectively, comprise the amino acid sequence of NYGMN (SEQ ID NO: 4), WINTYTGEPTYTDDFKG (SEQ ID NO: 5), GGFGSSYWYFDV (SEQ ID NO: 6), KASQDVSIAVA (SEQ ID NO: 7), SASDRYT (SEQ ID NO: 10), and QQHYITPLT (SEQ ID NO: 9).

In a further embodiment of the MMAE-conjugated ADC, the antibody or antigen-binding fragment thereof displays reduced binding to low TROP2-expressing cells compared to high TROP2-expressing cells and has reduced hydrophobicity compared to Sacituzumab as determined by hydrophobic interaction chromatography (HIC).

In a further embodiment of the MMAE-conjugated ADC, the antibody or antigen-binding fragment thereof is humanized.

In a further embodiment of the MMAE-conjugated ADC, the antigen binding fragment of the antibody is a Fab fragment, a Fab′ fragment, or a F(ab′)2 fragment.

In a further embodiment of the MMAE-conjugated ADC, the heavy chain variable domain comprises the amino acid sequence of SEQ ID NO: 1 or 14 and the light chain variable domain comprises the amino acid sequence of SEQ ID NO: 3 or 16.

In a further embodiment of the MMAE-conjugated ADC, the heavy chain variable domain comprises the amino acid sequence of SEQ ID NO: 1 and the light chain variable domain comprises the amino acid sequence of SEQ ID NO: 3 or the heavy chain variable domain comprises the amino acid sequence of SEQ ID NO: 14 and the light chain variable domain comprises the amino acid sequence of SEQ ID NO: 16.

In a further embodiment of the MMAE-conjugated ADC, the antibody comprises a light chain comprising the amino acid sequence of SEQ ID NO: 13 or 22 and the heavy chain comprises the amino acid sequence of SEQ ID NO: 11, 17, or 18.

In a further embodiment of the MMAE-conjugated ADC, the antibody comprises (a) a light chain comprising the amino acid sequence of SEQ ID NO: 13 and the heavy chain comprises the amino acid sequence of SEQ ID NO: 11; (b) a light chain comprising the amino acid sequence of SEQ ID NO: 22 and the heavy chain comprises the amino acid sequence of SEQ ID NO: 17; or (c) a light chain comprising the amino acid sequence of SEQ ID NO: 22 and the heavy chain comprises the amino acid sequence of SEQ ID NO: 18.

In a further embodiment of the MMAE-conjugated ADC, the antibody comprises a light chain comprising the amino acid sequence of SEQ ID NO: 13 or 22 and the heavy chain comprises the amino acid sequence of SEQ ID NO: 58, 59, or 60.

In a further embodiment of the MMAE-conjugated ADC, the antibody comprises (a) a light chain comprising the amino acid sequence of SEQ ID NO: 13 and the heavy chain comprises the amino acid sequence of SEQ ID NO: 58; (b) a light chain comprising the amino acid sequence of SEQ ID NO: 22 and the heavy chain comprises the amino acid sequence of SEQ ID NO: 59; or (c) a light chain comprising the amino acid sequence of SEQ ID NO: 22 and the heavy chain comprises the amino acid sequence of SEQ ID NO: 60.

In a further embodiment of the MMAE-conjugated ADC, the antibody further comprises a cysteine or a non-canonical amino acid amino acid substitution at one or more position(s) selected from the group consisting of: positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

In a further embodiment of the MMAE-conjugated ADC, the antibody comprises a cysteine or a non-canonical amino acid amino acid substitution at position 375 of the constant domain of the heavy chain.

In a further embodiment of the MMAE-conjugated ADC, the antibody comprises a heavy chain comprising the amino acid sequence of SEQ ID NO: 19, 20, 61, or 62.

In a further embodiment of the MMAE-conjugated ADC, the linker-MMAE payload is conjugated to the cysteine or noncanonical amino acid.

In a further embodiment of the MMAE-conjugated ADC, the antibody comprises a cysteine residue in which the SH group thereof is conjugated to a linker-MMAE payload comprising the formula:

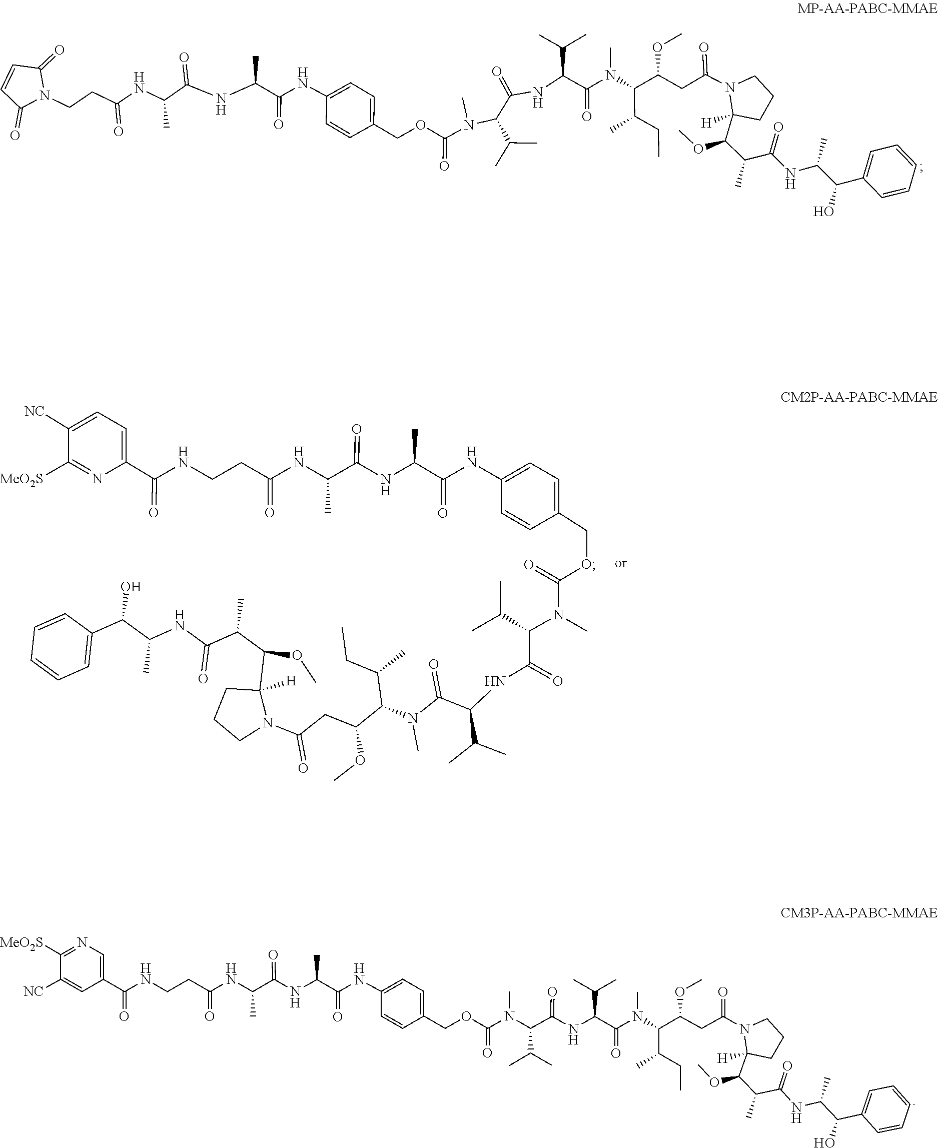

In a further embodiment of the MMAE-conjugated ADC, the ADC comprises the formula:

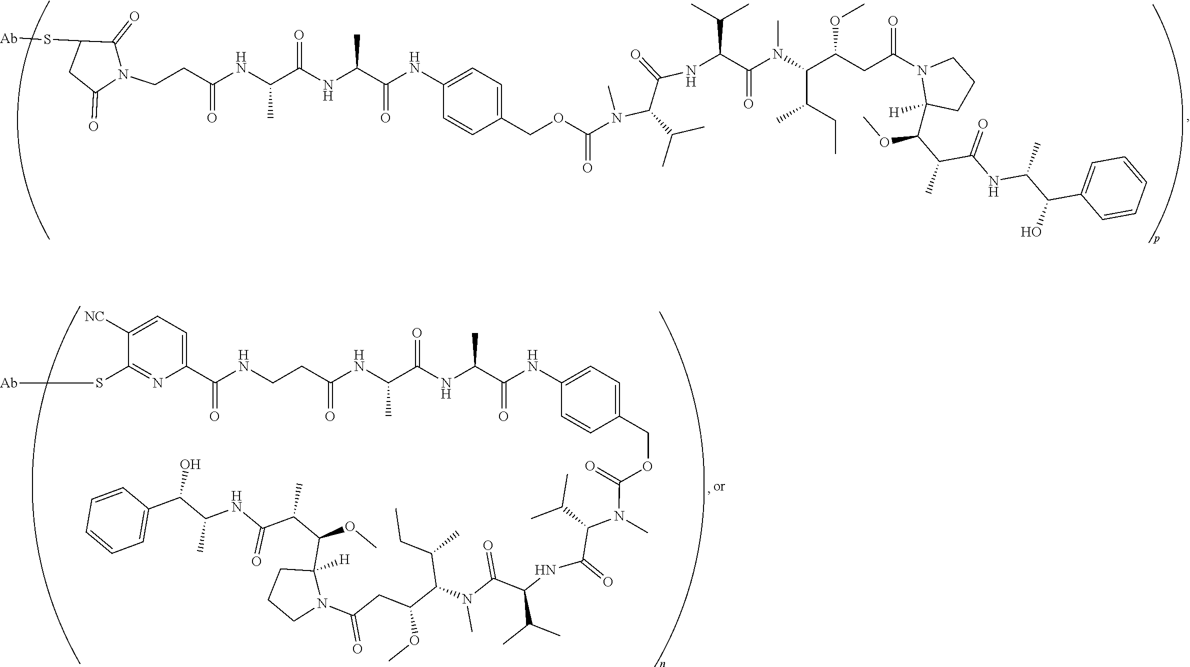

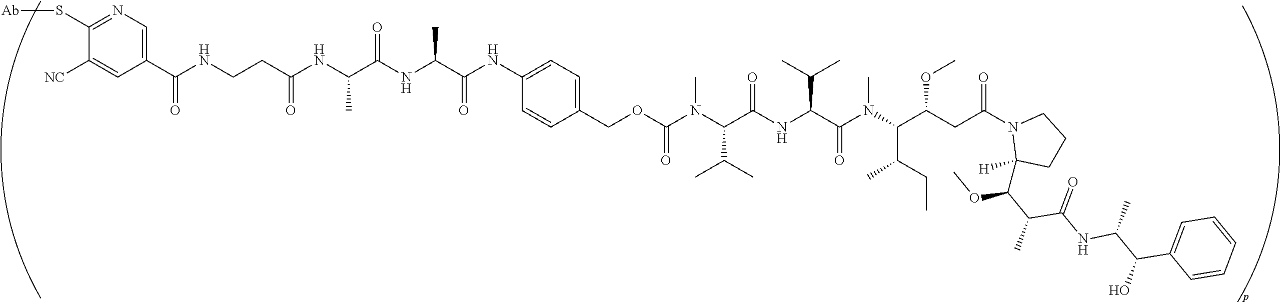

wherein Ab is an anti-TROP2 antibody; and p, is an integer from 1 to 8, wherein S is from the side chain of a cysteine residue of the antibody.

In a further embodiment of the MMAE-conjugated ADC, the ADC comprises the formula:

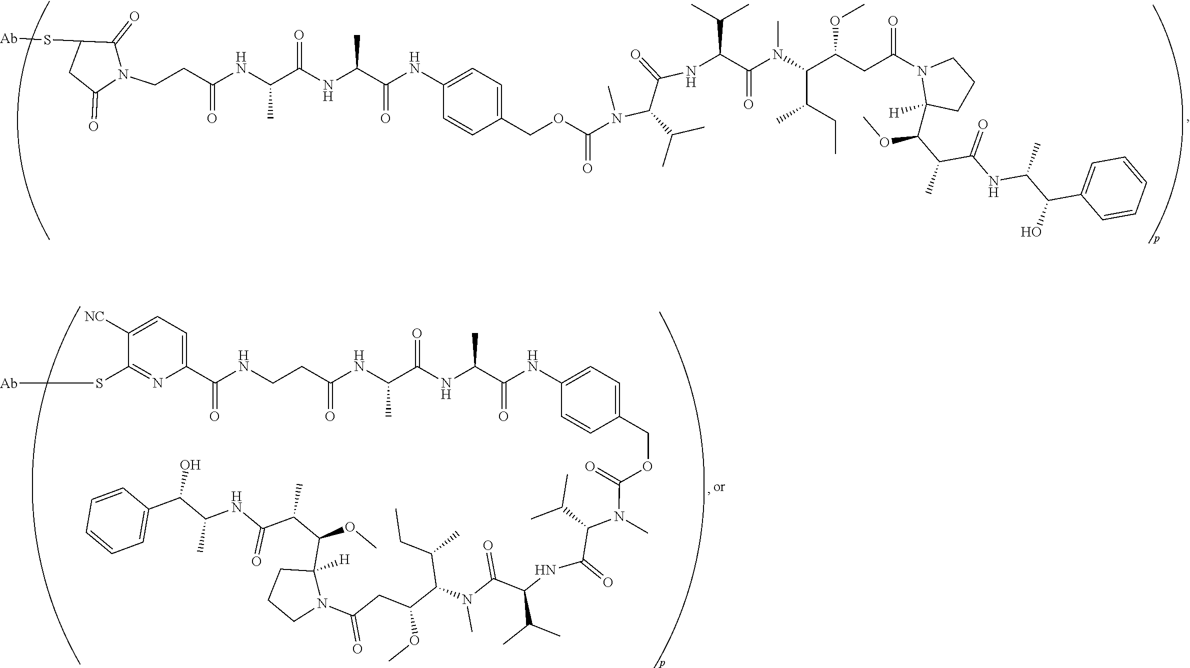

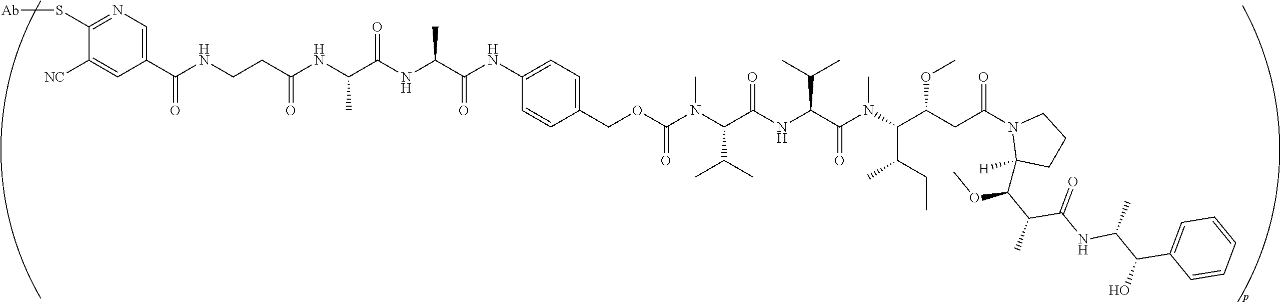

wherein Ab is an anti-TROP2 antibody comprising heavy chain engineered cysteine residues or light chain engineered cysteine residues, wherein the anti-TROP2 antibody comprising the engineered cysteine residues is (A) selected from the group consisting of:
(a) αTROP2 (HC:Sac-E152C) (LC:Sac-Y53D) comprising two heavy chains having the amino acid sequence set forth in SEQ ID No: 64 and two light chains having the amino acid sequence set forth in SEQ ID NO: 13;
(b) αTROP2 (HC:Sac-E153C) (LC:Sac-Y53D) two heavy chains having the amino acid sequence set forth in SEQ ID No: 65 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 66 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 67 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 68 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 69 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 71 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 72 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 73 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 74 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 75 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 76 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 58 and two light chains having the amino acid sequence set forth in SEQ ID NO: 50;
(n) αTROP2 (HC:Sac) (LC:Sac-Y53D-E168C) comprising two heavy chains having the amino acid sequence set forth in SEQ ID No: 58 and two light chains having the amino acid sequence set forth in SEQ ID NO: 51;
(o) αTROP2 (HC:Sac-YTE) (LC:Sac-Y53D-E165C) comprising two heavy chains having the amino acid sequence set forth in SEQ ID No: 63 and two light chains having the amino acid sequence set forth in SEQ ID NO: 50;
(p) αTROP2 (HC:Sac-YTE) (LC:Sac-Y53D-E168C) comprising two heavy chains having the amino acid sequence set forth in SEQ ID No: 63 and two light chains having the amino acid sequence set forth in SEQ ID NO: 51;
(q) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 21;
(r) αTROP2 (HC:BSM-E152C) (LC:BSM-Y53D) comprising two heavy chains having the amino acid sequence set forth in SEQ ID No: 78 and two light chains having the amino acid sequence set forth in SEQ ID NO: 22;
(s) αTROP2 (HC:BSM-E153C) (LC:BSM-Y53D) comprising two heavy chains having the amino acid sequence set forth in SEQ ID No: 79 and two light chains having the amino acid sequence set forth in SEQ ID NO: 22;
(t) αTROP2 (HC:BSM-E171C) (LC:BSM-Y53D) comprising two heavy chains having the amino acid sequence set forth in SEQ ID No: 80 and two light chains having the amino acid sequence set forth in SEQ ID NO: 22;
(u) αTROP2 (HC:BSM-E172C) (LC:BSM-Y53D) comprising two heavy chains having the amino acid sequence set forth in SEQ ID No: 81 and two light chains having the amino acid sequence set forth in SEQ ID NO: 22;
(v) αTROP2 (HC:BSM-E173C) (LC:BSM-Y53D) comprising two heavy chains having the amino acid sequence set forth in SEQ ID No: 82 and two light chains having the amino acid sequence set forth in SEQ ID NO: 22;
(w) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 22;
(x) αTROP2 (HC:BSM) (LC:BSM-Y53D-E165C) comprising two heavy chains having the amino acid sequence set forth in SEQ ID No: 59 and two light chains having the amino acid sequence set forth in SEQ ID NO: 54;
(y) αTROP2 (HC:BSM) (LC:BSM-Y53D-E168C) comprising two heavy chains having the amino acid sequence set forth in SEQ ID No: 59 and two light chains having the amino acid sequence set forth in SEQ ID NO: 55;
(z) αTROP2 (HC:BSM-YTE) (LC:BSM-Y53D-E165C) comprising two heavy chains having the amino acid sequence set forth in SEQ ID No: 60 and two light chains having the amino acid sequence set forth in SEQ ID NO: 54;
(aa) αTROP2 (HC:BSM-YTE) (LC:BSM-Y53D-E168C) comprising two heavy chains having the amino acid sequence set forth in SEQ ID No: 58 and two light chains having the amino acid sequence set forth in SEQ ID NO: 57;
(bb) αTROP2 (HC:BSM-YTE-E152C) (LC:BSM-Y53D) comprising two heavy chains having the amino acid sequence set forth in SEQ ID No: 85 and two light chains having the amino acid sequence set forth in SEQ ID NO: 22;
(cc) αTROP2 (HC:BSM-YTE-E153C) (LC:BSM-Y53D) comprising two heavy chains having the amino acid sequence set forth in SEQ ID No: 86 and two light chains having the amino acid sequence set forth in SEQ ID NO: 22;
(dd) αTROP2 (HC:BSM-YTE-E171C) (LC:BSM-Y53D) comprising two heavy chains having the amino acid sequence set forth in SEQ ID No: 87 and two light chains having the amino acid sequence set forth in SEQ ID NO: 22;
(ee) αTROP2 (HC:BSM-YTE-E172C) (LC:BSM-Y53D) comprising two heavy chains having the amino acid sequence set forth in SEQ ID No: 88 and two light chains having the amino acid sequence set forth in SEQ ID NO: 22;
(ff) αTROP2 (HC:BSM-YTE-E173C) (LC:BSM-Y53D) comprising two heavy chains having the amino acid sequence set forth in SEQ ID No: 89 and two light chains having the amino acid sequence set forth in SEQ ID NO: 22; and
(gg) αTROP2 (HC:BSM-YTE-S375C) (LC:BSM-Y53D) comprising two heavy chains having the amino acid sequence set forth in SEQ ID No: 90 and two light chains having the amino acid sequence set forth in SEQ ID NO: 22;

wherein S is from the side chain of the engineered cysteine residue; and, wherein p is an integer selected from 1 or 2; or (B) selected from the group consisting of:
(a) αTROP2 (HC:Sac-E152C-S375C) (LC:Sac-Y53D) comprising two heavy chains having the amino acid sequence set forth in SEQ ID No: 70 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 77 and two light chains having the amino acid sequence set forth in SEQ ID NO: 13;
(c) αTROP2 (HC:BSM-E152C-S375C) (LC:BSM-Y53D) comprising two heavy chains having the amino acid sequence set forth in SEQ ID No: 84 and two light chains having the amino acid sequence set forth in SEQ ID NO: 22; and
(d) αTROP2 (HC:BSM-YTE-E152C-S375C) (LC:BSM-Y53D) comprising two heavy chains having the amino acid sequence set forth in SEQ ID No: 91 and two light chains having the amino acid sequence set forth in SEQ ID NO: 22;
wherein S is from the side chain of the engineered cysteine residue; and, wherein p is an integer selected from 1, 2, 3, or 4.

In a further embodiment of the MMAE-conjugated ADC, the ADC comprises the

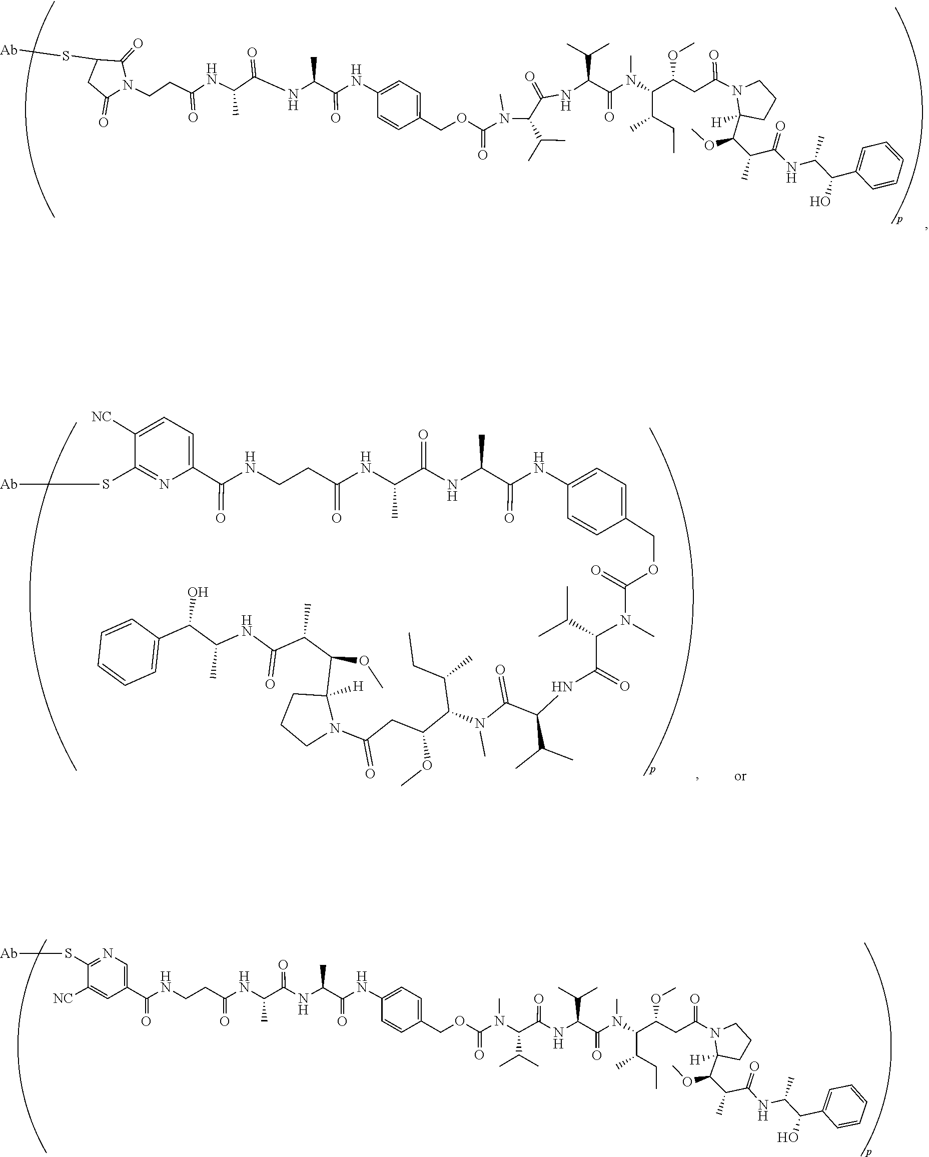

wherein Ab is an anti-TROP2 antibody comprising heavy chain engineered cysteine residues or light chain engineered cysteine residues, wherein the anti-TROP2 antibody comprising the engineered cysteine residues is (A) selected from the group consisting of:
(a) αTROP2 (HC:Sac-E152C) (LC:Sac-Y53D) comprising two heavy chains having the amino acid sequence set forth in SEQ ID No: 24 and two light chains having the amino acid sequence set forth in SEQ ID NO: 13;
(b) αTROP2 (HC:Sac-E153C) (LC:Sac-Y53D) comprising two heavy chains having the amino acid sequence set forth in SEQ ID No: 25 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 26 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 27 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 28 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 29 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 31 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 32 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 33 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 34 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 35 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 36 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 11 and two light chains having the amino acid sequence set forth in SEQ ID NO: 52;
(n) αTROP2 (HC:Sac) (LC:Sac-Y53D-E168C) comprising two heavy chains having the amino acid sequence set forth in SEQ ID No: 11 and two light chains having the amino acid sequence set forth in SEQ ID NO: 53;
(o) αTROP2 (HC:Sac-YTE) (LC:Sac-Y53D-E165C) comprising two heavy chains having the amino acid sequence set forth in SEQ ID No: 23 and two light chains having the amino acid sequence set forth in SEQ ID NO: 52;
(p) αTROP2 (HC:Sac-YTE) (LC:Sac-Y53D-E168C) comprising two heavy chains having the amino acid sequence set forth in SEQ ID No: 23 and two light chains having the amino acid sequence set forth in SEQ ID NO: 53;
(q) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 21;
(r) αTROP2 (HC:BSM-E152C) (LC:BSM-Y53D) comprising two heavy chains having the amino acid sequence set forth in SEQ ID No: 38 and two light chains having the amino acid sequence set forth in SEQ ID NO: 22;
(s) αTROP2 (HC:BSM-E153C) (LC:BSM-Y53D) comprising two heavy chains having the amino acid sequence set forth in SEQ ID No: 39 and two light chains having the amino acid sequence set forth in SEQ ID NO: 22;
(t) αTROP2 (HC:BSM-E171C) (LC:BSM-Y53D) comprising two heavy chains having the amino acid sequence set forth in SEQ ID No: 40 and two light chains having the amino acid sequence set forth in SEQ ID NO: 22;
(u) αTROP2 (HC:BSM-E172C) (LC:BSM-Y53D) comprising two heavy chains having the amino acid sequence set forth in SEQ ID No: 41 and two light chains having the amino acid sequence set forth in SEQ ID NO: 22;
(v) αTROP2 (HC:BSM-E173C) (LC:BSM-Y53D) comprising two heavy chains having the amino acid sequence set forth in SEQ ID No: 42 and two light chains having the amino acid sequence set forth in SEQ ID NO: 22;
(w) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 22;
(x) αTROP2 (HC:BSM) (LC:BSM-Y53D-E168C) comprising two heavy chains having the amino acid sequence set forth in SEQ ID No: 17 and two light chains having the amino acid sequence set forth in SEQ ID NO: 55;
(y) αTROP2 (HC:BSM-YTE) (LC:BSM-Y53D-E165C) comprising two heavy chains having the amino acid sequence set forth in SEQ ID No: 18 and two light chains having the amino acid sequence set forth in SEQ ID NO: 54;
(z) αTROP2 (HC:BSM-YTE) (LC:BSM-Y53D-E168C) comprising two heavy chains having the amino acid sequence set forth in SEQ ID No: 18 and two light chains having the amino acid sequence set forth in SEQ ID NO: 55;
(aa) αTROP2 (HC:BSM-YTE-E152C) (LC:BSM-Y53D) comprising two heavy chains having the amino acid sequence set forth in SEQ ID No: 44 and two light chains having the amino acid sequence set forth in SEQ ID NO: 22;
(bb) αTROP2 (HC:BSM-YTE-E153C) (LC:BSM-Y53D) comprising two heavy chains having the amino acid sequence set forth in SEQ ID No: 45 and two light chains having the amino acid sequence set forth in SEQ ID NO: 22;
(cc) αTROP2 (HC:BSM-YTE-E171C) (LC:BSM-Y53D) comprising two heavy chains having the amino acid sequence set forth in SEQ ID No: 46 and two light chains having the amino acid sequence set forth in SEQ ID NO: 22;
(dd) αTROP2 (HC:BSM-YTE-E172C) (LC:BSM-Y53D) comprising two heavy chains having the amino acid sequence set forth in SEQ ID No: 47 and two light chains having the amino acid sequence set forth in SEQ ID NO: 22;
(ee) αTROP2 (HC:BSM-YTE-E173C) (LC:BSM-Y53D) comprising two heavy chains having the amino acid sequence set forth in SEQ ID No: 48 and two light chains having the amino acid sequence set forth in SEQ ID NO: 22; and
(ff) αTROP2 (HC:BSM-YTE-S375C) (LC:BSM-Y53D) comprising two heavy chains having the amino acid sequence set forth in SEQ ID No: 20 and two light chains having the amino acid sequence set forth in SEQ ID NO: 22;

wherein S is from the side chain of the engineered cysteine residue; and, wherein p is an integer selected from 1 or 2; or (B) selected from the group consisting of:
(a) αTROP2 (HC:Sac-E152C-S375C) (LC:Sac-Y53D) comprising two heavy chains having the amino acid sequence set forth in SEQ ID No: 30 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 37 and two light chains having the amino acid sequence set forth in SEQ ID NO: 13;
(c) αTROP2 (HC:BSM-E152C-S375C) (LC:BSM-Y53D) comprising two heavy chains having the amino acid sequence set forth in SEQ ID No: 43 and two light chains having the amino acid sequence set forth in SEQ ID NO: 22; and
(d) αTROP2 (HC:BSM-YTE-E152C-S375C) (LC:BSM-Y53D) comprising two heavy chains having the amino acid sequence set forth in SEQ ID No: 49 and two light chains having the amino acid sequence set forth in SEQ ID NO: 22;

wherein S is from the side chain of the engineered cysteine residue; and, wherein p is an integer selected from 1, 2, 3, or 4.

In a further embodiment of the MMAE-conjugated ADC, the ADC comprises the

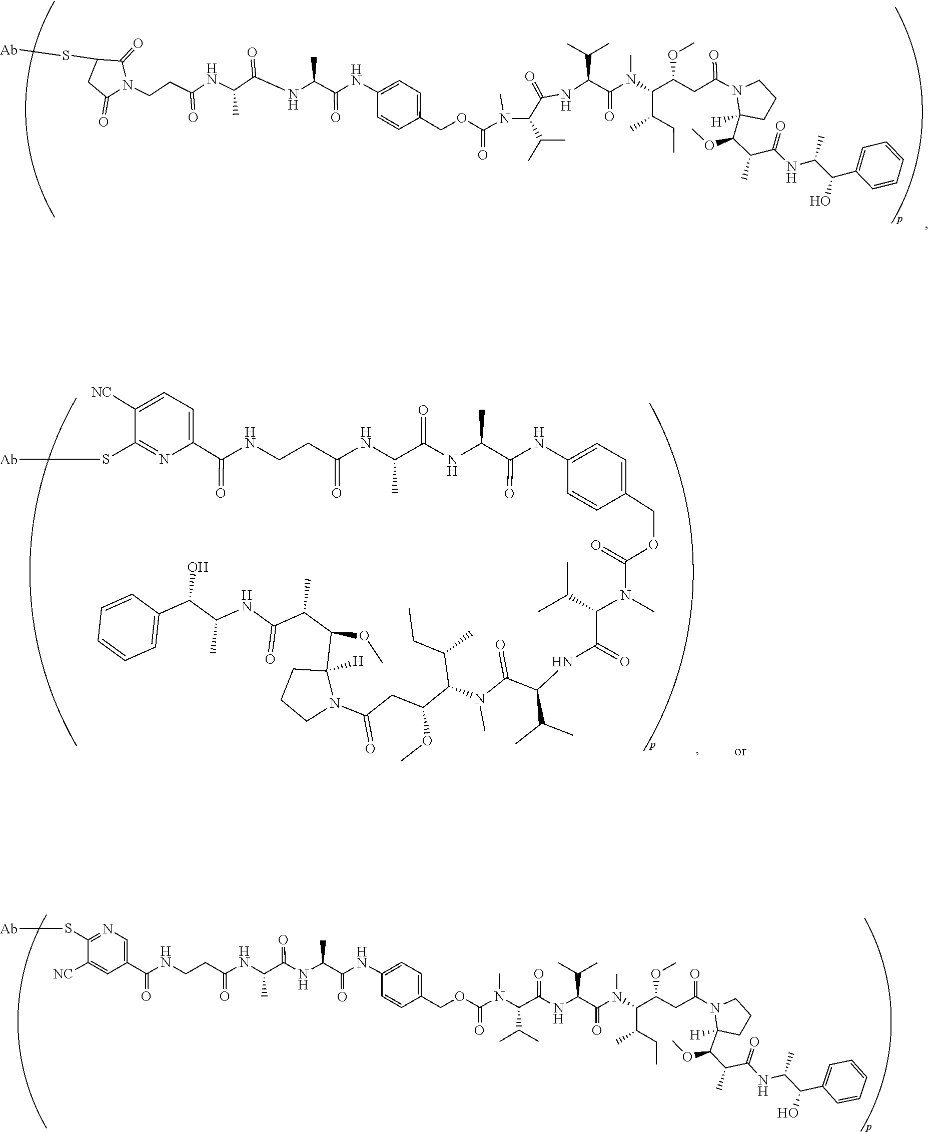

wherein Ab is an anti-Trop2 antibody comprising two heavy chains having the amino acid sequence set forth in SEQ ID NO: 20 and two light chains having the amino acid sequence set forth in SEQ ID NO: 22; wherein p is 1 or 2; and wherein S is from the side chain of a cysteine residue at position 375 of the constant domain of the heavy chain as defined according to Eu numbering.

The present invention further provides an ADC comprising the formula:

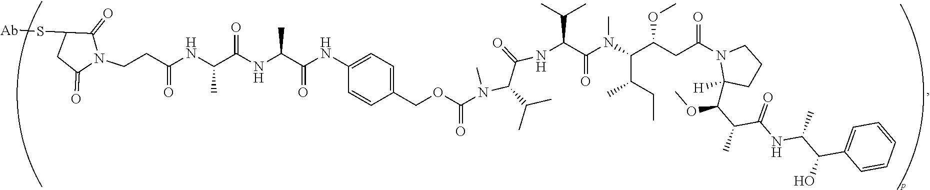

wherein Ab is an anti-Trop2 antibody comprising two heavy chains having the amino acid sequence set forth in SEQ ID NO: 20 and two light chains having the amino acid sequence set forth in SEQ ID NO: 22; wherein p is 1 or 2; and wherein S is from the side chain of a cysteine residue at position 375 of the constant domain of the heavy chain as defined according to Eu numbering.

In a further embodiment of the MMAE-conjugated ADC, the ADC comprises the formula

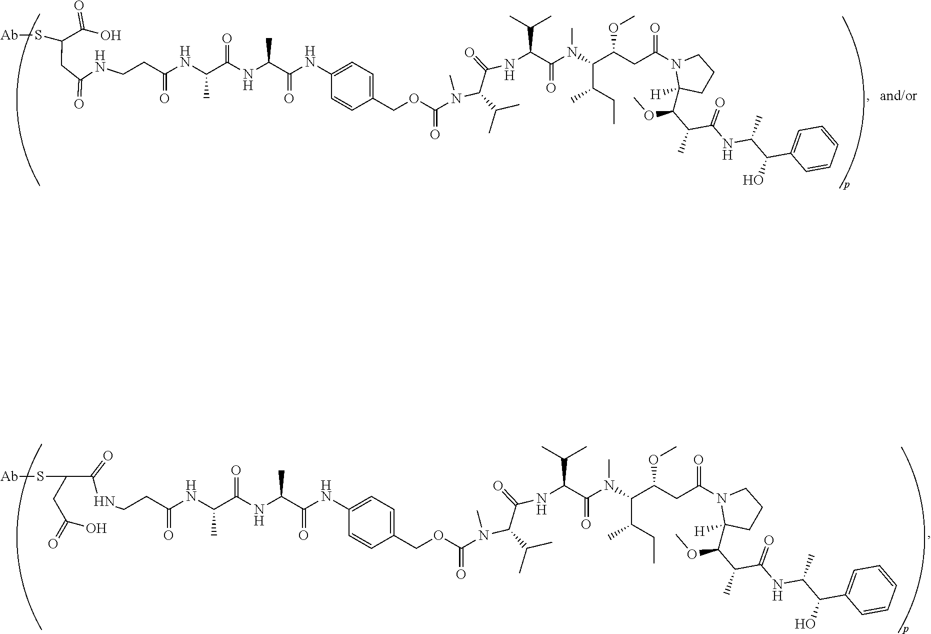

wherein Ab is an anti-TROP2 antibody; wherein p is 1 or 2; and wherein S is from the side chain of a cysteine residue at position 375 of the constant domain of the heavy chain as defined according to Eu numbering.

The present invention further provides a first composition comprising one or more of the aforementioned MMAE-conjugated ADC and a pharmaceutically acceptable carrier. In a further embodiment, the predominant ADC species in the composition comprises (i) antibodies in which the heavy chain C-terminus lacks a lysine residue; (ii) antibodies in which the heavy chain N-terminus is glutamine, glutamic acid, or pyroglutamate; or, (iii) antibodies in which the heavy chain C-terminus lacks a lysine residue and the heavy chain N-terminus is glutamine, glutamic acid, or pyroglutamate.

The present invention further provides a method for treating a cancer in an individual in need thereof comprising administering to the individual a therapeutically effective amount of any one of the aforementioned MMAE-conjugated ADCs or the aforementioned first composition to treat the cancer, wherein the cancer is a cancer that overexpresses TROP2. In a further embodiment of the method, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides use of any one of the aforementioned MMAE-conjugated ADCs or the aforementioned first composition for the manufacture of a medicament for treatment of a cancer that overexpresses TROP2. In a further embodiment of the use, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides any one of the aforementioned MMAE-conjugated ADCs or the aforementioned first composition for the treatment of a cancer that overexpresses TROP2. In a further embodiment, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides a combination therapy for treating cancer comprising any one of the aforementioned MMAE-conjugated ADCs or the aforementioned first composition and a therapeutic agent, wherein the cancer is a cancer that overexpresses TROP2. In a further embodiment of the combination therapy, the therapeutic agent is a chemotherapy agent or a therapeutic antibody. In a further embodiment of the combination therapy, the therapeutic antibody is a checkpoint inhibitor. In a further embodiment of the combination therapy, the therapeutic antibody is an anti-PD1 antibody or an anti-PD-L1 antibody. In a further embodiment of the combination therapy, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

In a further aspect, the present invention provides an ADC comprising an antibody that specifically binds to human TROP2 conjugated to a linker-MMAE payload (second MMAE-conjugated ADC), wherein the antibody comprises two heavy chains, each heavy chain comprising a variable domain and a constant domain, the variable domain comprising the amino acid sequence of SEQ ID NO: 14, and two light chains, each light chain comprising a variable domain comprise the amino acid sequence of SEQ ID NO: 15.

In a further embodiment of the second MMAE-conjugated ADC, the antibody displays reduced hydrophobicity compared to Sacituzumab as determined by hydrophobic interaction chromatography (HIC).

In a further embodiment of the second MMAE-conjugated ADC, the antibody further comprises a cysteine or a non-canonical amino acid amino acid substitution at one or more position(s) selected from the group consisting of: positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

In a further embodiment of the second MMAE-conjugated ADC, the antibody comprises a cysteine or a non-canonical amino acid amino acid substitution at position 375 of the constant domain of the heavy chain.

In a further embodiment of the second MMAE-conjugated ADC, the antibody comprises a heavy chain comprising the amino acid sequence of SEQ ID NO: 17, 18, 19, 59, 60, or 61 and a LC comprising the amino acid sequence of SEQ ID NO: 21.

In a further embodiment of the second MMAE-conjugated ADC, the linker-MMAE payload is conjugated to the cysteine or noncanonical amino acid.

In a further embodiment of the second MMAE-conjugated ADC, the antibody comprises a cysteine residue in which the SH group thereof is conjugated to a linker-MMAE payload comprising the formula:

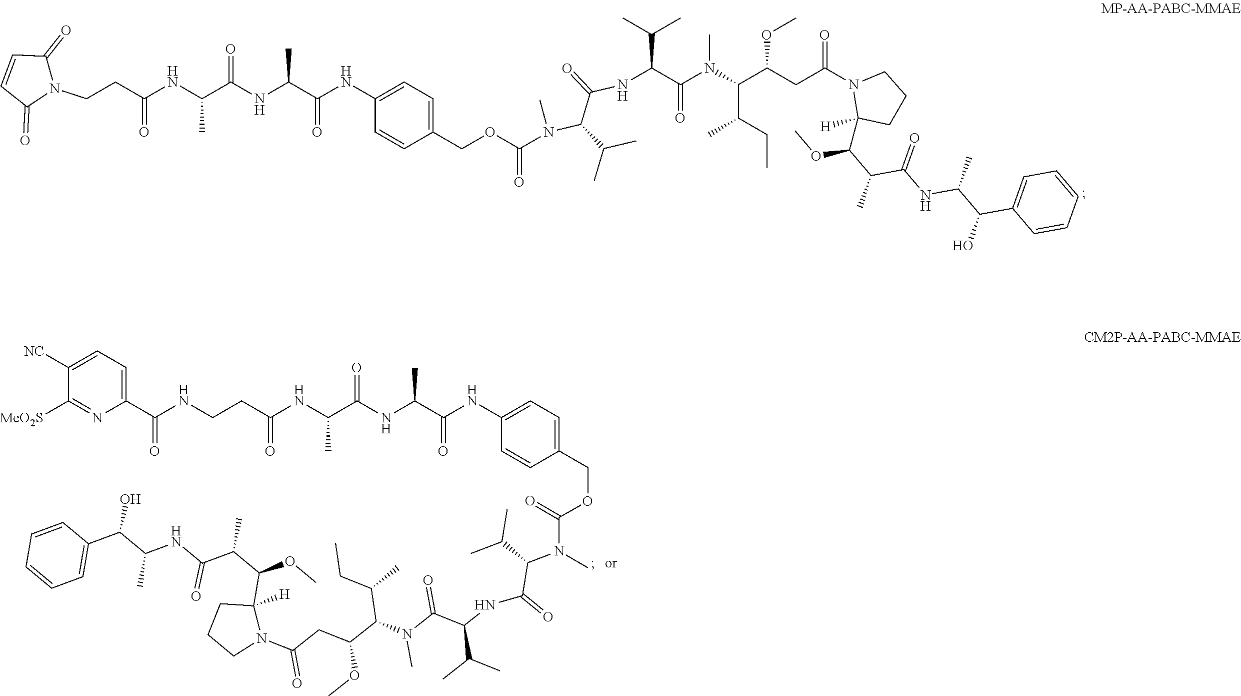 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2ccc(C#N)c(S(C)(=O)=O)n2)cc1)C(C)C]

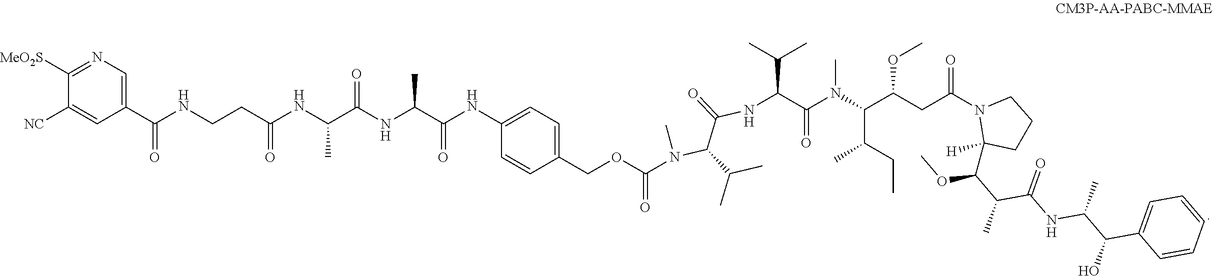 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2cnc(S(C)(=O)=O)c(C#N)c2)cc1)C(C)C]

In a further embodiment of the second MMAE-conjugated ADC, the ADC comprises the formula:

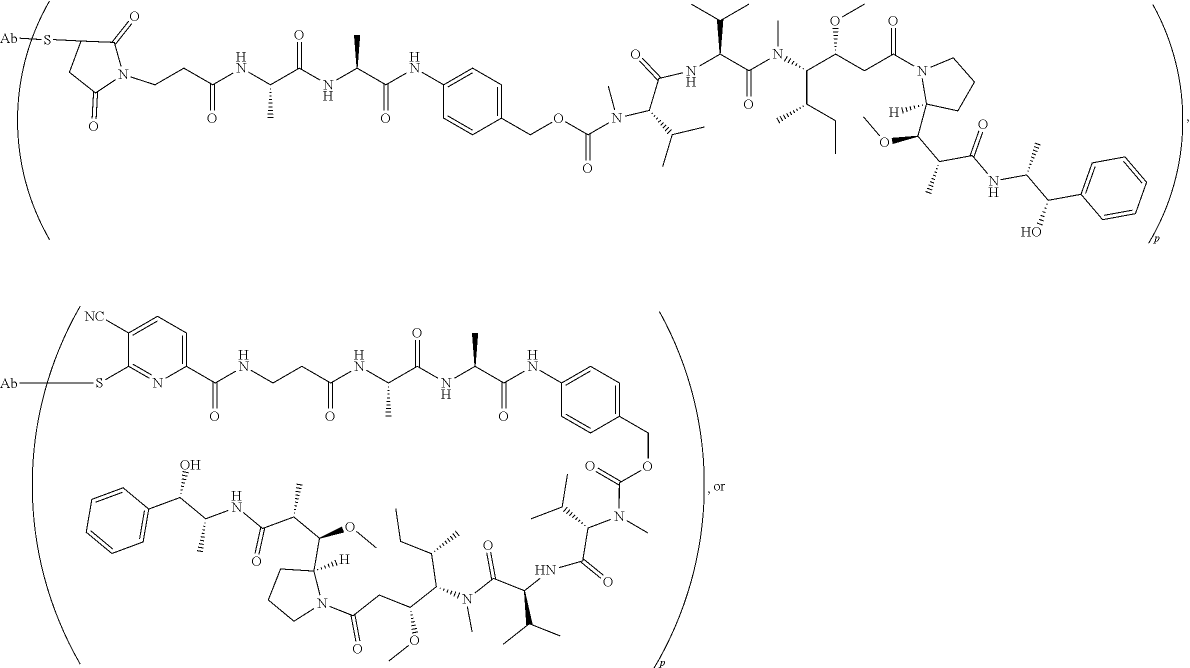

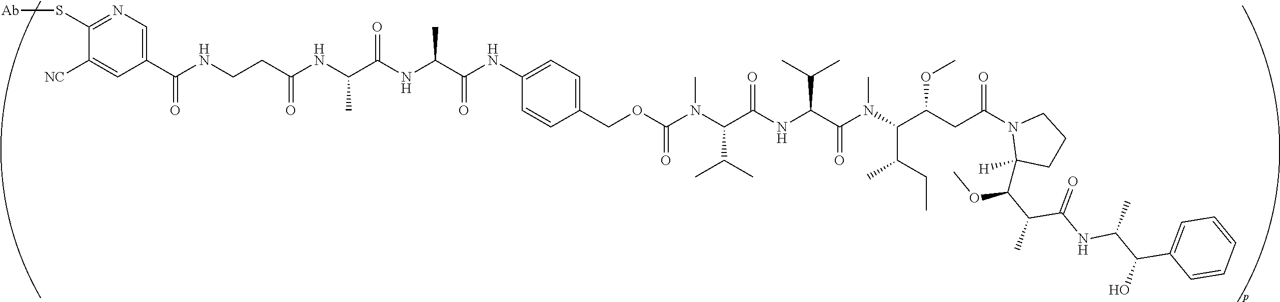

wherein Ab is the antibody of the second MMAE-conjugated ADC that specifically binds to human TROP2; and p, is an integer from 1 to 8, wherein S is from the side chain of a cysteine residue of the antibody.

In a further embodiment of the second MMAE-conjugated ADC, the ADC comprises the formula:

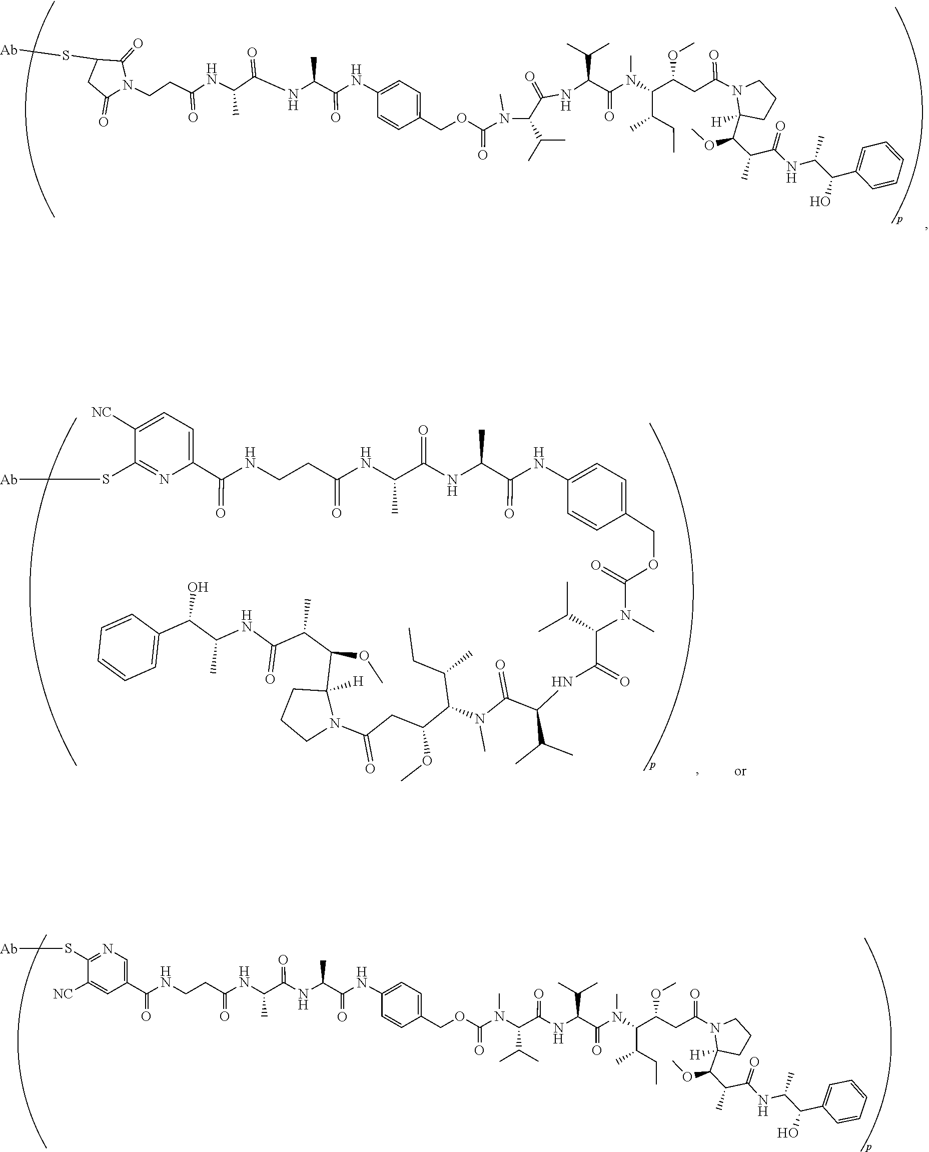

wherein Ab is the antibody of the second MMAE-conjugated ADC that specifically binds to human TROP2, wherein the antibody comprises heavy chain engineered cysteine residues or light chain engineered cysteine residues, wherein antibody comprising the engineered cysteine residues is (A) selected from the group consisting of
(a) αTROP2 (HC:BSM-E152C) (LC:BSM) comprising two heavy chains having the amino acid sequence set forth in SEQ ID No: 78 and two light chains having the amino acid sequence set forth in SEQ ID NO: 21;
(b) αTROP2 (HC:BSM-E153C) (LC:BSM) comprising two heavy chains having the amino acid sequence set forth in SEQ ID No: 79 and two light chains having the amino acid sequence set forth in SEQ ID NO: 21;
(c) αTROP2 (HC:BSM-E171C) (LC:BSM) comprising two heavy chains having the amino acid sequence set forth in SEQ ID No: 80 and two light chains having the amino acid sequence set forth in SEQ ID NO: 21;
(d) αTROP2 (HC:BSM-E172C) (LC:BSM) comprising two heavy chains having the amino acid sequence set forth in SEQ ID No: 81 and two light chains having the amino acid sequence set forth in SEQ ID NO: 21;
(e) αTROP2 (HC:BSM-E173C) (LC:BSM) comprising two heavy chains having the amino acid sequence set forth in SEQ ID No: 82 and two light chains having the amino acid sequence set forth in SEQ ID NO: 21;
(f) αTROP2 (HC:BSM) (LC:BSM-E165C) comprising two heavy chains having the amino acid sequence set forth in SEQ ID No: 59 and two light chains having the amino acid sequence set forth in SEQ ID NO: 52;
(g) αTROP2 (HC:BSM) (LC:BSM-E168C) comprising two heavy chains having the amino acid sequence set forth in SEQ ID No: 59 and two light chains having the amino acid sequence set forth in SEQ ID NO: 53;
(h) αTROP2 (HC:BSM-YTE) (LC:BSM-E165C) comprising two heavy chains having the amino acid sequence set forth in SEQ ID No: 60 and two light chains having the amino acid sequence set forth in SEQ ID NO: 52;
(i) αTROP2 (HC:BSM-YTE) (LC:BSM-E168C) comprising two heavy chains having the amino acid sequence set forth in SEQ ID No: 60 and two light chains having the amino acid sequence set forth in SEQ ID NO: 53;
(j) αTROP2 (HC:BSM-YTE-E152C) (LC:BSM) comprising two heavy chains having the amino acid sequence set forth in SEQ ID No: 85 and two light chains having the amino acid sequence set forth in SEQ ID NO: 21;
(k) αTROP2 (HC:BSM-YTE-E153C) (LC:BSM) comprising two heavy chains having the amino acid sequence set forth in SEQ ID No: 86 and two light chains having the amino acid sequence set forth in SEQ ID NO: 21;
(l) αTROP2 (HC:BSM-YTE-E171C) (LC:BSM) comprising two heavy chains having the amino acid sequence set forth in SEQ ID No: 87 and two light chains having the amino acid sequence set forth in SEQ ID NO: 21;
(m) αTROP2 (HC:BSM-YTE-E172C) (LC:BSM) comprising two heavy chains having the amino acid sequence set forth in SEQ ID No: 88 and two light chains having the amino acid sequence set forth in SEQ ID NO: 21;
(n) αTROP2 (HC:BSM-YTE-E173C) (LC:BSM) comprising two heavy chains having the amino acid sequence set forth in SEQ ID No: 89 and two light chains having the amino acid sequence set forth in SEQ ID NO: 21; and
(o) αTROP2 (HC:BSM-YTE-S375C) (LC:BSM) comprising two heavy chains having the amino acid sequence set forth in SEQ ID No: 90 and two light chains having the amino acid sequence set forth in SEQ ID NO: 21;

wherein S is from the side chain of the engineered cysteine residue; and, wherein p is an integer selected from 1, 2, 3, or 4; or (B) selected from the group consisting of:
(a) αTROP2 (HC:BSM-E152C-S375C) (LC:BSM) comprising two heavy chains having the amino acid sequence set forth in SEQ ID No: 84 and two light chains having the amino acid sequence set forth in SEQ ID NO: 21; and
(b) αTROP2 (HC:BSM-YTE-E152C-S375C) (LC:BSM) comprising two heavy chains having the amino acid sequence set forth in SEQ ID No: 91 and two light chains having the amino acid sequence set forth in SEQ ID NO: 21; and

wherein S is from the side chain of the engineered cysteine residue; and, wherein p is an integer selected from 1, 2, 3, or 4.

In a further embodiment of the second MMAE-conjugated ADC, the ADC comprises the formula:

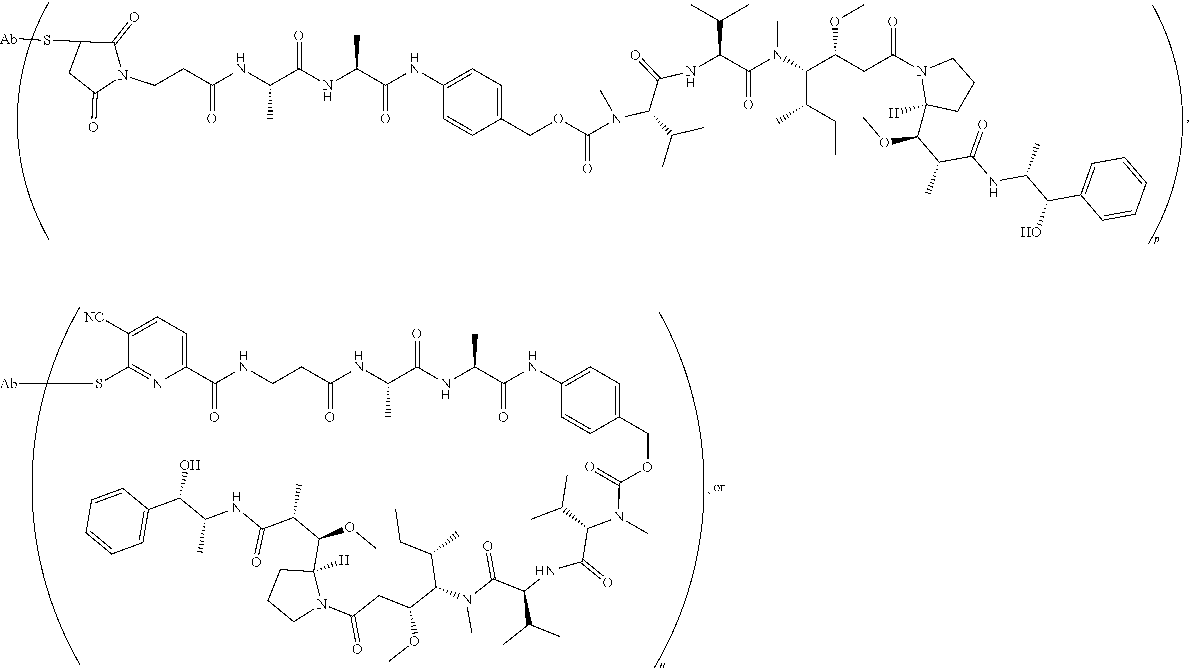

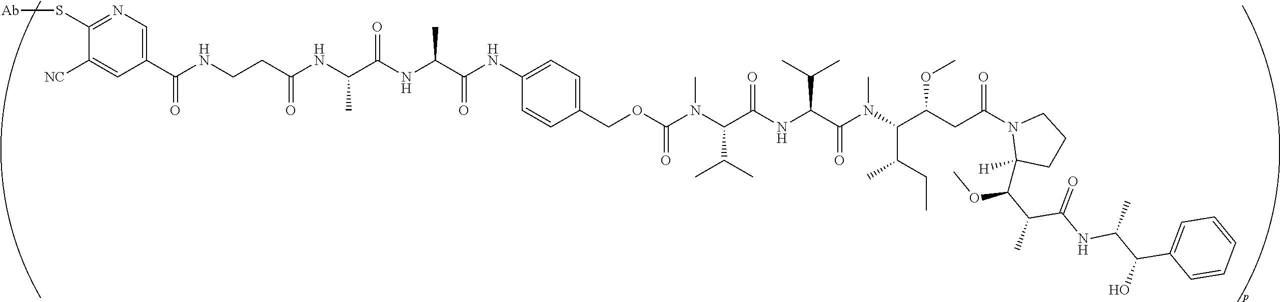

wherein Ab is the antibody of the second MMAE-conjugated ADC that specifically binds to human TROP2, wherein the antibody comprises comprising heavy chain engineered cysteine residues or light chain engineered cysteine residues, wherein the antibody comprising the engineered cysteine residues is (A) selected from the group consisting of:
(a) αTROP2 (HC:BSM-E152C) (LC:BSM) comprising two heavy chains having the amino acid sequence set forth in SEQ ID No: 38 and two light chains having the amino acid sequence set forth in SEQ ID NO: 21;
(b) αTROP2 (HC:BSM-E153C) (LC:BSM) comprising two heavy chains having the amino acid sequence set forth in SEQ ID No: 39 and two light chains having the amino acid sequence set forth in SEQ ID NO: 21;
(c) αTROP2 (HC:BSM-E171C) (LC:BSM) comprising two heavy chains having the amino acid sequence set forth in SEQ ID No: 40 and two light chains having the amino acid sequence set forth in SEQ ID NO: 21;
(d) αTROP2 (HC:BSM-E172C) (LC:BSM) comprising two heavy chains having the amino acid sequence set forth in SEQ ID No: 41 and two light chains having the amino acid sequence set forth in SEQ ID NO: 21;
(e) αTROP2 (HC:BSM-E173C) (LC:BSM) comprising two heavy chains having the amino acid sequence set forth in SEQ ID No: 42 and two light chains having the amino acid sequence set forth in SEQ ID NO: 21;
(f) αTROP2 (HC:BSM) (LC:BSM-E165C) comprising two heavy chains having the amino acid sequence set forth in SEQ ID No: 17 and two light chains having the amino acid sequence set forth in SEQ ID NO: 52;
(g) αTROP2 (HC:BSM) (LC:BSM-E168C) comprising two heavy chains having the amino acid sequence set forth in SEQ ID No: 17 and two light chains having the amino acid sequence set forth in SEQ ID NO: 53;
(h) αTROP2 (HC:BSM-YTE) (LC:BSM-E165C) comprising two heavy chains having the amino acid sequence set forth in SEQ ID No: 18 and two light chains having the amino acid sequence set forth in SEQ ID NO: 52;
(i) αTROP2 (HC:BSM-YTE) (LC:BSM-E168C) comprising two heavy chains having the amino acid sequence set forth in SEQ ID No: 18 and two light chains having the amino acid sequence set forth in SEQ ID NO: 53;
(j) αTROP2 (HC:BSM-YTE-E152C) (LC:BSM) comprising two heavy chains having the amino acid sequence set forth in SEQ ID No: 44 and two light chains having the amino acid sequence set forth in SEQ ID NO: 21;
(k) αTROP2 (HC:BSM-YTE-E153C) (LC:BSM) comprising two heavy chains having the amino acid sequence set forth in SEQ ID No: 45 and two light chains having the amino acid sequence set forth in SEQ ID NO: 21;
(l) αTROP2 (HC:BSM-YTE-E171C) (LC:BSM) comprising two heavy chains having the amino acid sequence set forth in SEQ ID No: 46 and two light chains having the amino acid sequence set forth in SEQ ID NO: 21;
(m) αTROP2 (HC:BSM-YTE-E172C) (LC:BSM) comprising two heavy chains having the amino acid sequence set forth in SEQ ID No: 47 and two light chains having the amino acid sequence set forth in SEQ ID NO: 21;
(n) αTROP2 (HC:BSM-YTE-E173C) (LC:BSM) comprising two heavy chains having the amino acid sequence set forth in SEQ ID No: 48 and two light chains having the amino acid sequence set forth in SEQ ID NO: 21; and
(o) αTROP2 (HC:BSM-YTE-S375C) (LC:BSM) comprising two heavy chains having the amino acid sequence set forth in SEQ ID No: 20 and two light chains having the amino acid sequence set forth in SEQ ID NO: 21;

wherein S is from the side chain of the engineered cysteine residue; and, wherein p is an integer selected from 1 or 2; or (B) selected from the group consisting of:
(a) αTROP2 (HC:BSM-E152C-S375C) (LC:BSM) comprising two heavy chains having the amino acid sequence set forth in SEQ ID No: 43 and two light chains having the amino acid sequence set forth in SEQ ID NO: 21; and
(b) αTROP2 (HC:BSM-YTE-E152C-S375C) (LC:BSM) comprising two heavy chains having the amino acid sequence set forth in SEQ ID No: 49 and two light chains having the amino acid sequence set forth in SEQ ID NO: 21;

wherein S is from the side chain of the engineered cysteine residue; and, wherein p is an integer selected from 1, 2, 3, or 4.

In a further embodiment of the second MMAE-conjugated ADC, the ADC comprises the formula:

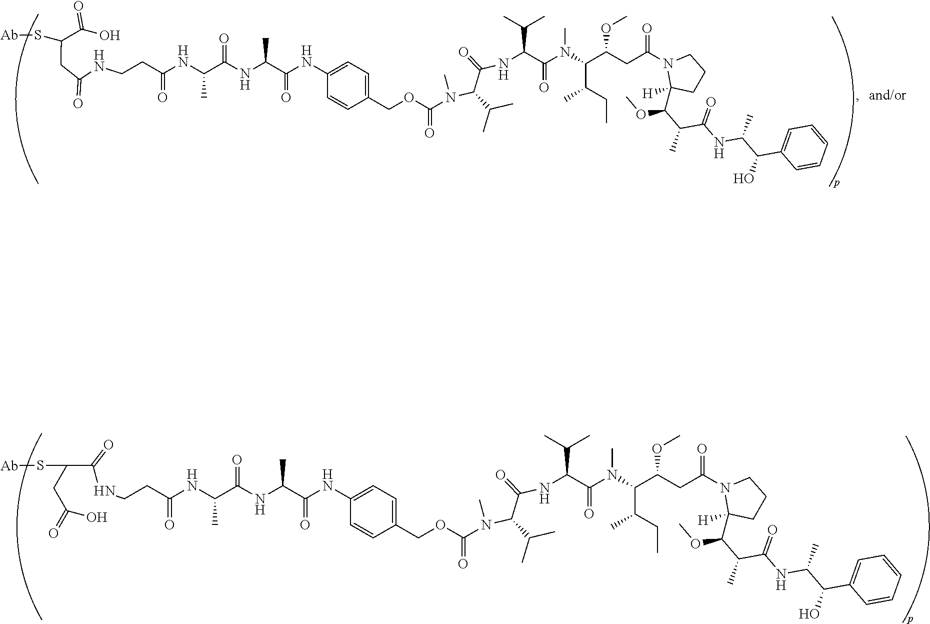

wherein Ab is an the antibody of the second MMAE-conjugated ADC that specifically binds to human TROP2; wherein p is 1 or 2; and wherein S is from the side chain of a cysteine residue of the antibody.

The present invention further provides a second composition comprising one or more of the aforementioned second MMAE-conjugated ADCs and a pharmaceutically acceptable carrier. In a further embodiment of the composition, the predominant ADC species in the composition comprises (i) antibodies in which the heavy chain C-terminus lacks a lysine residue; (ii) antibodies in which the heavy chain N-terminus is glutamine, glutamic acid, or pyroglutamate; or, (iii) antibodies in which the heavy chain C-terminus lacks a lysine residue and the heavy chain N-terminus is glutamine, glutamic acid, or pyroglutamate.

The present invention further provides a method for treating a cancer in an individual in need thereof comprising administering to the individual a therapeutically effective amount of any one of the aforementioned second MMAE-conjugated ADCs or the aforementioned second composition to treat the cancer, wherein the cancer is a cancer that overexpresses TROP2. In a further embodiment of the method, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides for use of any one of the aforementioned second MMAE-conjugated ADCs or the aforementioned second composition for the manufacture of a medicament for treatment of a cancer that overexpresses TROP2. In a further embodiment of the use, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides any one of the aforementioned second MMAE-conjugated ADCs or the aforementioned second composition for treatment of a cancer that overexpresses TROP2. In a further embodiment, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The present invention further provides a combination therapy for treating cancer comprising any one of the aforementioned second MMAE-conjugated ADCs or the aforementioned second composition and a therapeutic agent, wherein the cancer is a cancer that overexpresses TROP2. In a further embodiment of the combination therapy, the therapeutic agent is a chemotherapy agent or a therapeutic antibody. In a further embodiment of the combination therapy, the therapeutic antibody is a checkpoint inhibitor. In a further embodiment of the combination therapy, the therapeutic antibody is an anti-PD1 antibody or an anti-PD-L1 antibody.

In a further embodiment of the combination therapy, the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer. The present invention further provides pharmaceutically acceptable salts or solvates of any one of the aforementioned ADCs.

### BRIEF DESCRIPTION OF THE DRAWINGS

FIG. 1 shows an ELISA that demonstrates that the Y53D substitution of CDR2 of the Sacituzumab light chain produces an anti-TROP2 antibody that preferentially binds cells that express high levels of TROP2 on the cell surface over cells that express low levels of TROP2 on the cell surface. BxPC3 cells express high levels of TROP2 on the cell surface and MDA-MB-231 express low levels of TROP2 on the cell surface.

FIGS. 2 and 2-1 shows a comparison of the performance of Sacituzumab (hRS7 clinical Sequence) to Sacituzumab BSM (hRS7 BSM Sequence) on reverse phase high performance liquid chromatography (RP-HPLC) at 70° C. AU on Y-axis refers to absorbance units.

FIG. 3 shows a comparison of the hydrophobicity of the αTROP2 (HC:BSM-YTE-S375C) (LC:BSM-Y53D) antibody to the αTROP2 (HC:BSM-S375C) (LC:BSM-Y53D) antibody and the αTROP2 (HC:BSM-YTE-S375C) (LC:BSM-Y105S) antibody to the αTROP2 (HC:BSM-S375C) (LC:BSM-Y105S) antibody.

FIG. 4 shows a comparison of the hydrophobicity of the αTROP2 (HC:BSM-YTE-S375C) (LC:BSM-Y53D) antibody to the αTROP2 (HC:BSM-S375C) (LC:BSM) antibody.

FIG. 5 shows Rhesus monkey pharmacokinetics (PK) of several framework antibody variants following a 3 milligrams per kilograms (mpk) intravenous (IV) bolus administration. The antibodies were Sacituzumab (αTROP2), αTROP2 (HC:Sac-S375C) (LC:Sac), and αTROP2 (HC:BSM-S375C) (LC:BSM).

FIG. 6 shows Rhesus monkey PK of light chain Y53D and heavy chain Y105S antibody affinity variants after 1 mpk IV bolus administration. The antibodies were Sacituzumab (αTROP2), αTROP2 (HC:BSM-S375C) (LC:BSM-Y53D), and αTROP2 (HC:BSM-S375C-Y105S) (LC:BSM).

FIG. 7 shows Rhesus monkey PK of antibody affinity variants after a single 20 mpk IV administration. The antibodies were αTROP2 (HC:BSM-YTE-S375C) (LC:BSM) and αTROP2 (HC:BSM-YTE-S375C) (LC:BSM-Y53D).

FIG. 8 shows Rhesus monkey PK of antibody affinity variants after a single 20 mpk IV administration. The antibodies were αTROP2 (HC:BSM-S375C) (LC:BSM-Y53D) and αTROP2 (HC:BSM-YTE-S375C) (LC:BSM-Y53D).

FIG. 9 shows the immunogenicity risk profiles for the amino acid sequences of the heavy and light chains of αTROP2 (HC:BSM-S375C) (LC:BSM-Y53D) compared to that of the heavy and light chains of Sacituzumab.

FIG. 10 shows a comparison of RP-HPLC performance of αTROP2 (HC:BSM-YTE-S375C-MMAE) (LC:BSM) ADC to the αTROP2 (HC:Sac-S375C-MMAE) (LC:Sac) ADC.

FIG. 11 shows a mass spectroscopy (MS) profile of a composition comprising an αTROP2-MP-AA-PABC-MMAE ADC with the positions for DAR0, DAR1, DAR2, and DAR3 indicated. The αTROP2 antibody comprises HC:BSM-YTE-S375C-MMAE and LC:BSM-Y53D.

FIG. 12 shows a hydrophobic chromatography (HIC) profile of a composition comprising an αTROP2-MP-AA-PABC-MMAE ADC with the positions for DAR0, DAR1, DAR2, and DAR3 indicated. The αTROP2 antibody comprises HC:BSM-YTE-S375C-MMAE and LC:BSM-Y53D. The control antibody profile is the unconjugated antibody.

FIG. 13 shows a comparison of the anti-tumor effect of several avidity-tuned Sacituzumab variants conjugated to maleimide-C2-Ala-Ala-PABC-MMAE in the BxPC3 mouse model. The graph displays the change in tumor volume over time following a single dose at day 0.

FIG. 14 shows a comparison of the anti-tumor effect of αTROP2 (HC:BSM-YTE-S375C-MMAE) (LC:BSM-Y53D) ADC at four doses in the BxPC3 mouse model. The graph displays the change in tumor volume over 35 days following a single dose at day 0.

FIG. 15 shows the Rat PK for αTROP2 (HC:BSM-YTE-S375C-MMAE) (LC:BSM-Y53D).

FIG. 16 shows the V_{H} and V_{L} of αTROP2 (HC:BSM) (LC:BSM-Y53D). The CDRs as defined by Kabat are underlined and the BSM amino acid substitutions are shown in bold-faced type. The numbering of the V_{H} according to Kabat and sequentially numbering are shown. The sequential numbering of the V_{L} is also shown.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the term “TROP2” refers to trophoblast cell-surface antigen 2, also known as tumor-associated calcium signal transducer 2 (TACSTD2) or epithelial glycoprotein-1 antigen (EGP-1). TROP2 is a protein that in humans is encoded by the TACSTD2 gene. This intron-less gene encodes a carcinoma-associated antigen defined by the monoclonal antibody GA733. This antigen is a member of a family including at least two type I membrane proteins. It transduces an intracellular calcium signal and acts as a cell surface receptor. TROP2 expression was originally described in trophoblasts (placenta) and fetal tissues (e.g., lung). Later, its expression was also described in the normal stratified squamous epithelium of the skin, uterine cervix, esophagus, and tonsillar crypts. TROP2 plays a role in tumor progression by actively interacting with several key molecular signaling pathways traditionally associated with cancer development and progression. Aberrant overexpression of TROP2 has been described in several solid cancers, such as colorectal, renal, lung, bladder, and breast cancers. TROP2 expression has also been described in some rare and aggressive malignancies, e.g., salivary duct, anaplastic thyroid, uterine/ovarian, and neuroendocrine prostate cancers.

As used herein, the term “affinity”, represented by the equilibrium constant for the dissociation of an antigen with an antigen binding polypeptide (K_{D}), is a measure of the binding strength between an antigenic determinant and an antigen-binding site on the antibody (or fragment thereof): the lesser the value of the K_{D}, the stronger the binding strength between an antigenic determinant and the antigen-binding polypeptide. Alternatively, the affinity can also be expressed as the affinity constant (K_{A}), which is 1/K_{D}. Affinity can be determined by known methods, depending on the specific antigen of interest. For example, K_{D} may be determined by surface plasmon resonance (SPR; Biacore™). Any K_{D} value less than 10-6 is considered to indicate binding. Specific binding of an antibody, or fragment thereof, to an antigen or antigenic determinant can be determined in any suitable known manner, including, for example, Scatchard analysis and/or competitive binding assays, such as radioimmunoassays (RIA), enzyme immunoassays (EIA) and sandwich competition assays, equilibrium dialysis, equilibrium binding, gel filtration, enzyme-linked immunosorbent assay (ELISA), SPR, or spectroscopy (e.g., using a fluorescence assay) and the different variants thereof known in the art.

As used herein, the term “avidity” is the measure of the strength of binding between an antibody, or fragment thereof, and the pertinent antigen. Avidity is related to both the affinity between an antigenic determinant and its antigen binding site on the antibody and the number of pertinent binding sites present on the antibody. Avidity affects both the association and dissociation step of a binding reaction. The association rate increases as the antibody can bind to several sites, which simply increase the association rate-constant by the multiplicity of the reaction. For example, a typical IgG antibody is bivalent for a particular target, each arm of the antibody comprises a Fab moiety that can independently bind the target. Following the initial association, the other Fab moiety can bind an adjacent copy of the target in an intra-molecular reaction called ring-closing. Ring-closing occurs intra-molecularly and is thus independent of the concentration. Instead, it depends on the structure of the antibody and antigen, which together define an effective concentration (Mack et al., J. Am. Chem. Soc. 133:11701-11715 (2011); Mack et al., J. Am. Chem. Soc. 134:333-345 (2012)). Dissociation from two targets target requires simultaneous release of both Fab moieties, and thus depends on the ring-closing equilibrium and the effective concentration. In principle, the avidity of a bivalent interaction could be predicted from the effective concentration of ring-closing (Bobrovnik, J. Mol. Recognit. 20:253-262 (2007)). Effective concentrations and avidity has previously been studied using either model systems (Mack et al., ibid.) or theoretical models (Diestler & Knapp, Phys. Rev. Lett. 100:178101 (2008); Diestler & Knapp, J. Phys. Chem. C 114 (12), 5287-5304 (2010); Numata et al., J. Phys. Chem. B 116:2595-2604 (2012)).

As used herein, the term “administration” and “treatment”, as it applies to an animal, human, experimental subject, cell, tissue, organ, or biological fluid, refers to contact of an exogenous pharmaceutical, therapeutic, diagnostic agent, or composition comprising a human TROP2 binder or ADC as disclosed herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subject” refers to a human.

As used herein, the term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following Table 1.

TABLE 1

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “antibody” or “immunoglobulin” as used herein refers to a glycoprotein comprising at least two heavy chains (HCs) and two light chains (LCs) inter-connected by disulfide bonds. Each HC is comprised of a heavy chain variable region or domain (V_{H}) and a heavy chain constant region or domain. Each light chain is comprised of an LC variable region or domain (V_{L}) and a LC constant domain. In certain naturally occurring IgG, IgD, IgE, IgM, and IgA antibodies, the heavy chain constant region is comprised of three domains, CH1, CH2 and CH3. In general, the basic antibody structural unit for antibodies is a Y-shaped tetramer comprising two HC/LC pairs (2H). Each tetramer includes two identical pairs of polypeptide chains, each pair having one LC (about 25 kDa) and HC chain (about 50-70 kDa) (H+L). Each HC:LC pair comprises one V_{H}:one V_{L} pair. The one V_{H}:one V_{L} pair may be referred to by the term “Fab”. Thus, each antibody tetramer comprises two Fabs, one per each arm of the Y-shaped antibody.

The LC constant domain is comprised of one domain, CL. The human V_{H} includes seven family members: V_{H}1, V_{H}2, V_{H}3, V_{H}4, V_{H}5, V_{H}6, and V_{H}7; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} can be further subdivided into regions of hypervariability, termed complementarity determining region (CDR) areas, interspersed with regions that are more conserved, termed framework regions (FR). Each V_{H} and V_{L} is composed of three CDR regions and four FR regions, arranged from amino-terminus to carboxy-terminus in the following order: FR1, CDR 1, FR2, CDR 2, FR3, CDR 3, FR4. Numbering of the amino acids in a V_{H} may be determined using the Kabat numbering scheme. See Béranger, et al., Ed. Ginetoux, Correspondence between the IMGT unique numbering for C-DOMAIN, the IMGT exon numbering, the Eu and Kabat numberings: Human IGHG, created: 17/05/2001, Version: Aug. 6, 2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63:78-85 (1969), and is shown for the IgG1, IgG2, IgG3, and IgG4 constant domains in Béranger et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following: Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence); Chothia numbering scheme is based on the location of the structural loop region (See Chothia & Lesk, J. Mol. Biol. 196:901-917 (1987); Al-Lazikani et al., J. Mol. Biol. 273:927-948 (1997)); AbM numbering scheme is a compromise between the two used by Oxford Molecular's AbM antibody modelling software (see Karu et al., ILAR Journal 37:132-141 (1995); Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008)); IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germline V sequence alignment (see Giudicelli et al., Nucleic Acids Res. 25:206-11 (1997); Lefranc, Immunol Today 18: 509 (1997); Lefranc et al., Dev Comp Immunol. 27:55-77 (2003)).

The following general rules disclosed in www.bioinf.org.uk: Prof. Andrew C. R. Martin's Group and reproduced in Table 2 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 2

| Loop | Kabat | AbM | Chothia¹ | Contact² | IMGT |
|---|---|---|---|---|---|
| L1 | L24 - - - L34 | L24 - - - L34 | L24 - - - L34 | L30 - - - L36 | L27 - - - L32 |
| L2 | L50 - - - L56 | L50 - - - L56 | L50 - - - L56 | L46 - - - L55 | L50 - - - L52 |
| L3 | L89 - - - L97 | L89 - - - L97 | L89 - - - L97 | L89 - - - L96 | L89 - - - L97 |
| H1 | H31 - - - H35B | H26 - - - | H26 - - - | H30 - - - H35B | H26 - - - H35B |
| | (Kabat | H35B | H32 . . . 34 | | |
| | Numbering)³ | | | | |
| H1 | H31 - - - H35 | H26 - - - H35 | H26 - - - H32 | H30 - - - H35 | H26 - - - H33 |
| | (Chothia | | | | |
| | Numbering) | | | | |
| H2 | H50 - - - H65 | H50 - - - H58 | H52 - - - H56 | H47 - - - H58 | H51 - - - H56 |
| H3 | H95 - - - H102 | H95 - - - H102 | H95 - - - H102 | H93 - - - H101 | H93 - - - H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

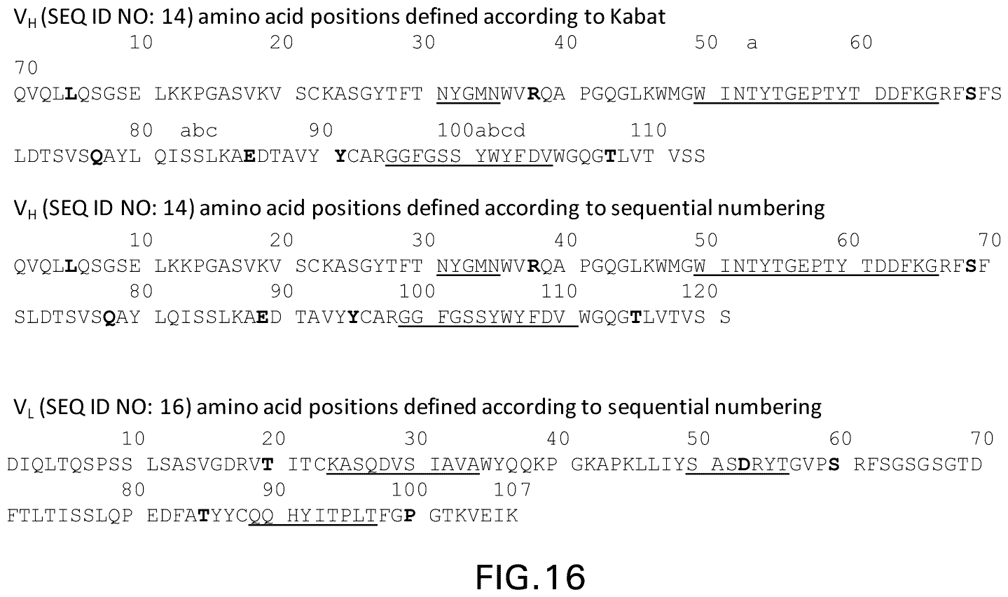

The entire amino acid sequence of the V_{H} is commonly numbered according to Kabat while the three CDRs within the variable region may be defined according to any one of the aforementioned numbering schemes. In particular embodiments, the numbering of the amino acid positions in the V_{H} may be sequential beginning with amino acid position 1 and continuing sequentially to the end of the sequence or according to Kabat. FIG. 16 shows the V_{H} of αTROP2 BSM numbered sequentially and according to Kabat. Unless specified otherwise, the amino acid positions in the V_{H} and V_{L} herein are defined according to sequential numbering.

The numbering of the amino acid positions in the heavy chain constant domain may be sequential beginning with amino acid position 1 and continuing sequentially to the end of the sequence or according to Eu numbering. The IgG1 heavy chain constant domain amino acid sequence has 330 amino acids sequentially numbered 1 to 330. The corresponding sequence numbered according to Eu begins with position number 118 and ends with position number 447. Unless specified otherwise, the amino acid positions in the heavy and light chains herein are defined according to sequential numbering.

As used herein, the term “Fc domain”, or “Fc” as used herein is the crystallizable fragment domain or region obtained from an antibody that comprises the CH2 and CH3 domains of an antibody. In an antibody, the two Fc domains are held together by two or more disulfide bonds and by hydrophobic interactions of the CH3 domains. The Fc domain may be obtained by digesting an antibody with the protease papain. Typically, amino acids in the Fc domain are numbered according to the Eu numbering convention (See Edelmann et al., Biochem. 63:78-85 (1969)).

As used herein, the term “antigen” as used herein refers to any foreign substance which induces an immune response in the body.

As used herein, the term “antigen binding fragment” refers to a polypeptide or polypeptides comprising a fragment of a full-length antibody, which retains the ability to specifically bind to the antigen bound by the full-length antibody, and/or to compete with the full-length antibody for specific binding to the antigen. Examples of antigen binding fragments include but are not limited to Fab fragment, Fab′ fragment, F(ab′)2 fragment, Fv region, and scFv.

As used herein, “specifically binds” refers, with respect to a target antigen, the preferential association of a binder, in whole or part, with the target antigen and not to other molecules, particularly molecules found in human blood or serum. Binders as shown herein typically bind specifically to the target antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, a binder that “specifically binds” or “binds specifically” to a target antigen refers to a binder that binds to the target antigen with high affinity, which means having a K_{D} of 10⁻⁷ M or less, in particular embodiments a K_{D} of 10⁻⁸ M or less, or 5×10⁻⁹ M or less, or between 10⁻⁸ M and 10⁻¹¹ M or less, but does not bind with measurable binding to a non-target antigen as determined in a cell ELISA or Surface Plasmon Resonance assay (SPR) using 10 μg/mL antibody. The term does not exclude antibodies that bind a homologue of the target. For example, an antibody that specifically binds the human TROP2 may also bind homologues of the human TROP2 such as the Rhesus monkey TROP2 and or rat TROP2 as long as the binding is specific to the TROP2 homologue.

As used herein, the term “Fab fragment” refers to an antigen binder comprising one antibody light chain and the CH1 and V_{H} of one antibody heavy chain. The heavy chain of a Fab molecule cannot form a disulfide bond with another heavy chain molecule. A “Fab fragment” can be the product of papain cleavage of an antibody.

As used herein, the term “Fab′ fragment” refers to an antigen binder comprising one antibody light chain and a portion or fragment of one antibody heavy chain that contains the V_{H} and the CH1 domain up to a region between the CH1 and CH2 domains, such that an interchain disulfide bond can be formed between the two heavy chains of two Fab′ fragments to form a F(ab′)₂ molecule.

As used herein, the term “F(ab′)₂ fragment” refers to an antigen binder comprising two antibody light chains and two heavy chains containing the V_{H} and the CH1 domain up to a region between the CH1 and CH2 domains, such that an interchain disulfide bond is formed between the two heavy chains. An F(ab′)₂ fragment thus is composed of two Fab′ fragments that are held together by a disulfide bond between the two heavy chains. An “F(ab′)₂ fragment” can be the product of pepsin cleavage of an antibody.

As used herein, the term “Fv region” refers to an antigen binder comprising the variable regions from both the heavy and light chains of an antibody but lacks the constant regions.

As used herein, the term “ScFv” or “single-chain variable fragment” refers to a fusion protein comprising a V_{H} and V_{L} fused or linked together by a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker.

As used herein, the term “diabody” refers to an antigen binder comprising a small antibody fragment with two antigen-binding regions, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementarity domains of another chain and create two antigen-binding regions. Diabodies are described more fully in, e.g., EP 404,097; WO 93/11161; and Holliger et al. (1993) Proc. Natl. Acad. Sci. USA 90:6444-6448. For a review of engineered antibody variants generally see Holliger and Hudson (2005) Nat. Biotechnol. 23:1126-1136.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, the term “binder” refers to an antibody or antigen-binding fragment thereof.

The term “antibody-drug conjugate”, or “ADC” is an antibody or binder that is conjugated to one or more (typically 1 to 8) payloads, each through a linker to a specific site on the antibody or binder. The antibody is typically a monoclonal antibody specific to a cancer antigen and is capable of delivering the payload into a cell expressing the cancer antigen on the extracellular surface of the cell.

The term “DAR” or “Drug Antibody Ratio,” as used herein, refers to the average number of linker/payload moieties attached to the antibodies present in a composition. For a composition comprising an antibody-drug conjugate of the present disclosure, the DAR for the composition is the average of the DARs (linker-payload moieties of all of the individual antibody-drug conjugate molecules present in said composition), and this average is expressed as a decimal. As such, in some embodiments for a composition comprising an antibody-drug conjugate of the present disclosure, the DAR of the composition is a decimal from 0 to 24, from 0 to 8, from 0 to 7, from 0 to 6, from 0 to 5, from 0 to 4, from 0 to 3, from 0 to 2, and from 0 to 1. In additional embodiments, for a composition comprising an antibody-drug conjugate of the present disclosure, the DAR of the composition is a decimal from 1 to 4, 2 to 5, 3 to 6, 4 to 7, 5 to 8, and 6 to 8. In other embodiments, for a composition comprising an antibody-drug conjugate of the present disclosure, the DAR of the composition is a decimal from 1 to 3, 2 to 4, 3 to 5, 4 to 6, 5 to 7, and 6 to 8. In further embodiments, for a composition comprising an antibody-drug conjugate of the present disclosure, the DAR of the composition is a decimal from 1 to 2, 2 to 3, 3 to 4, 4 to 5, 5 to 6, 6 to 7, and 7 to 8. In particular embodiments, the DAR of the composition is 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0. The term “composition” as used above, is understood to encompass pharmaceutical compositions. Average DAR can be determined by various conventional means such as UV spectroscopy, mass spectroscopy, ELISA assay, radiometric methods, hydrophobic interaction chromatography (HIC), electrophoresis and HPLC.

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “extracellular antigen binding domain,” “extracellular domain,” or “extracellular ligand binding domain” when used in reference to a CAR refers to the part of a CAR that is located outside of the cell membrane and is capable of binding to an antigen, target or ligand.

As used herein, the term “hinge region” when used in reference to a CAR refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane.

As used herein, the term “intracellular T cell receptor-activating signaling domain”, “cytoplasmic signaling domain,” or “intracellular signaling domain” refers to the part of a CAR that is located inside of the cell membrane and is capable of transducing an effector signal.

As used herein, the term “isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature 256:495 (1975) or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991), and Marks et al., J. Mol. Biol. 222:581-597 (1991), for example. See also Presta, J. Allergy Clin. Immunol. 116:731 (2005).

As used herein, the term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters. Genes include both naturally occurring nucleotide sequences encoding a molecule of interest and synthetically derived nucleotide sequences encoding a molecule of interest, for example, complementary DNA (cDNA) obtained from a messenger RNA (mRNA) nucleotide sequence.

As used herein, the term “polynucleotides” discussed herein form part of the present invention. A “polynucleotide”, “nucleic acid” or “nucleic acid molecule” include DNA and RNA, single- or double-stranded. Polynucleotides e.g., encoding an immunoglobulin chain or component of the antibody display system of the present invention, may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Polynucleotides e.g., encoding an immunoglobulin chain or component of the antibodies or ADCs of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Villa-Komaroff et al., Proc. Natl. Acad. Sci. USA 75:3727-3731 (1978)), or the tac promoter (DeBoer et al., Proc. Natl. Acad. Sci. USA 80:21-25 (1983)); see also “Useful proteins from recombinant bacteria” in Scientific American 242:74-94 (1980); and promoter elements from yeast or other fungi such as the Gal 4 promoter, the ADC (alcohol dehydrogenase) promoter, PGK (phosphoglycerol kinase) promoter or the alkaline phosphatase promoter.

As used herein, the terms “vector”, “cloning vector” and “expression vector” include a vehicle (e.g., a plasmid) by which a DNA or RNA sequence can be introduced into a host cell so as to transform the host and, optionally, promote expression and/or replication of the introduced sequence. Polynucleotides encoding an immunoglobulin chain or component of the antibodies or ADCs of the present invention may, in an embodiment of the invention, be in a vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny of a parent cell will have precisely identical DNA content, due to deliberate or inadvertent mutations. Mutant progeny having the same function or biological activity as screened for in the originally transformed cell are included. Where distinct designations are intended, it will be clear from the context.

As used herein, the term “control sequences” or “regulatory sequences” refers to DNA sequences necessary for the expression of an operably linked coding sequence in a particular host organism. The control sequences that are suitable for expression in eukaryotes, for example, include a promoter, operator or enhancer sequences, transcription termination sequences, and polyadenylation sequences for expression of a messenger RNA encoding a protein and a ribosome binding site for facilitating translation of the messenger RNA.

As used herein, a nucleic acid is “operably linked” when it is placed into a functional relationship with another nucleic acid sequence, e.g., a regulatory sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, the term “TROP2 binder” refers to the anti-TROP2 antibodies of the present invention and antigen-binding fragments thereof. The term specifically excludes other anti-TROP2 antibodies such as Sacituzumab.

As used herein, the term “treat” or “treating” means to administer a therapeutic moiety, such as a composition containing any of the anti-TROP2 binders or ADCs of the present invention, topically, subcutaneously, intramuscular, intradermally, intravenously, or systemically to an individual in need. The amount of a therapeutic moiety that is effective to treat cancer or proliferative disease in the individual may vary according to factors such as the injury or disease state, age, and/or weight of the individual, and the ability of the therapeutic agent to elicit a desired response in the individual. Whether the therapeutic objective has been achieved can be assessed by the individual and/or any clinical measurement typically used by physicians or other skilled healthcare providers to assess the severity or progression status of the treatment. Thus, the terms denote that a beneficial result has been or will be conferred on a human or animal individual in need. Treating may be therapeutic or prophylactic.

As used herein, the term “treatment,” as it applies to a human or veterinary individual, refers to therapeutic treatment or prophylactic treatment, as well as diagnostic applications. “Treatment” as it applies to a human or veterinary individual, encompasses contact of the TOP2 binders or ADCs of the present invention to a human or animal subject.

As used herein, the term “therapeutically effective amount” refers to a quantity of a specific substance sufficient to achieve a desired effect in an individual being treated. For instance, this may be the amount necessary to inhibit or reduce the severity of a disease or disorder in an individual.

As used herein, the term “combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

As used herein, the term “Solvate” means a physical association of an ADC disclosed herein with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more ADCs disclosed herein may optionally be converted to a solvate. Preparation of solvates is generally known. Thus, for example, M. Caira et al., J. Pharmaceutical Sci., 93 (3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTechours., 5 (1), article 12 (2004); and A. L. Bingham et al., Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

As used herein, the term “pharmaceutically acceptable salt” includes acid addition salts and basic salts.

Exemplary acid addition salts include acetates, ammonium, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (also known as mesylates), naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. 2^{nd} Revised Ed. (2011) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto. In one embodiment, an acid salt is an ammonium salt or a di-ammonium salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the present disclosure and all acid and base salts are considered equivalent to the free forms of the corresponding compounds for purposes of the present disclosure.

### INTRODUCTION

The present invention provides TROP2 binders that preferentially bind high TROP2-expressing cells (TROP2^{high} cells) over low TROP2-expressing cells (TROP2^{low} cells) and conjugates comprising said TROP2 binders conjugated to a payload. In particular embodiments, the TROP2 binder is an anti-TROP2 antibody of the present invention that is conjugated to a payload to provide an anti-TROP2 antibody-drug conjugate (ADC) of the present invention. A shown in the examples, anti-TROP2 ADCs are stable and efficacious in mouse and non-human primate (NHP) models. The ADCs of the present invention are useful for treating, imaging, diagnosing, preventing the proliferation of, containing and reducing TROP2-expressing cells, in particular TROP2-expressing tumors.

The ADCs of the present invention may be used to treat a disorder that comprises cells that overexpress TROP2 on the cell surface. Examples of such disorders include but are not limited to breast cancer, triple negative breast cancer (TNBC), ovary cancer, non-small cell lung cancer (NSCLC), squamous cell cancer of head and neck (SCCHN), colon cancer, neuroendocrine cancer, prostate cancer, sarcoma, stomach cancer, esophageal cancer, and cervical cancer.

### TROP2 Binders

The present invention provides TROP2 binders (anti-TROP2 antibodies and antigen-binding fragments thereof) that preferentially bind TROP2^{high} cells over TROP2^{low} cells as may be determined by a cell-based enzyme-linked immunosorbent assay (ELISA) and display reduced hydrophobicity compared to Sacituzumab as may be determined by hydrophobicity interaction chromatography (HIC). Sacituzumab is a humanized anti-TROP2 antibody that comprises a heavy chain having the amino acid sequence of SEQ ID NO: 11 and a light chain having the amino acid sequence of SEQ ID NO:12. Sacituzumab has been disclosed in U.S. Pat. No. 9,745,380 (See antibody hRS7 comprising SEQ ID NO: 14 (V_{H}) and 13 (V_{L}) therein). The antibody-drug conjugate (ADC) Sacituzumab govitecan-hziy is currently being marketed under the tradename TRODELVY.

In general, TROP2 is overexpressed in various carcinomas, such as colorectal, pancreatic, gastric, oral squamous cell carcinoma, ovarian, bladder, and breast cancers, compared with expression in the corresponding normal tissue and various other tissues (Ohmachi et al., Clin Cancer Res. 12:3057-63 (2006): Fong et al., Br J Cancer. 99:1290-5 (2008); Lin et al., Exp Mol Pathol. 94:73-8 (2013); Bignotti et al., Eur J Cancer. 46:944-53 (2010); Mühlmann et al., J Clin Pathol. 62:152-8 (2009); Fong et al., Mod Pathol. 21:186-91 (2008)). In these studies, carcinomas with high TROP2 expression showed poor prognosis. The preferential binding of the TROP2 binders of the present invention for TROP2^{high} cells reduces the risk of off-target binding, thus limiting unwanted adverse events (AE) during therapy regimes. The selectivity is particularly advantageous for use in cancer treatment regimens targeting cancers that overexpress TROP2. Thus, ADCs comprising the anti-TROP2 antibodies of the present invention conjugated to a therapeutic moiety, e.g., a cytotoxin such as an inhibitor of topoisomerase I or II or an inhibitor of microtubule assembly, are particularly useful for treatment regimens targeting cancers that overexpress TROP2.

The TROP2 binders of the present invention incorporate the discovery that introducing a tyrosine to aspartic acid amino acid substitution at position 53 (Y53D amino acid substitution) of the light chain of Sacituzumab provides a modified Sacituzumab (αTROP2 (HC:Sac) (LC:Sac-Y53D) antibody) having reduced avidity to TROP2 and thus, having preferential or selective binding for TROP2^{high} cells over TROP2^{low} cells. In addition, it was unexpectedly found that the Y53D amino acid substitution reduced the hydrophobicity of the αTROP2 (HC:Sac) (LC:Sac-Y53D) antibody compared to Sacituzumab as may be determined by hydrophobic interaction chromatography (HIC). Reduced hydrophobicity may provide an TROP2 binder that has reduced propensity to aggregate and allows preparation of concentrated aqueous solutions of the antibody having reduced viscosity.

In an exemplary embodiment, the present invention provides an avidity tuned TROP2 binder comprising a heavy chain variable domain (V_{H}) having the amino acid sequence set forth in SEQ ID NO: 1 and a light chain variable domain (V_{L}) comprising the amino acid sequence set forth in SEQ ID NO: 3. This exemplary anti-TROP2 antibody comprises the amino acid sequence of the V_{H} of Sacituzumab and the amino acid sequence of the V_{L} of Sacituzumab having a Y53D amino acid substitution. In a further exemplary embodiment, the present invention provides a TROP2 binder, which is an antibody, comprising a heavy chain having the amino acid sequence set forth in SEQ ID NO: 11 and a light chain comprising the amino acid sequence set forth in SEQ ID NO: 13. This exemplary anti-TROP2 antibody comprises the amino acid sequence of the heavy chain of Sacituzumab and the amino acid sequence of the light chain of Sacituzumab having Y53D amino acid substitution.

Thus, the avidity-tuned TROP2 binders of the present invention comprise (i) a heavy chain variable domain (V_{H}) comprising the heavy chain complementarity determining regions (HC-CDRs) 1, 2, 3 as set forth in the amino acid sequence of SEQ ID NO: 1 in which the CDRs are defined according to Kabat, ABM, IMGT, Chothia, or Contact; and (ii) a light chain variable domain (V_{L}) comprising the light chain complementarity determining regions (LC-CDRs) 1, 2, 3 as set forth in the amino acid sequence of SEQ ID NO: 3 in which the CDRs are defined according to Kabat, ABM, IMGT, Chothia, or Contact.

In a further embodiment, the avidity-tuned TROP2 binders of the present invention comprise (i) a V_{H} comprising HC-CDR 1 comprising the amino acid sequence set NYGMN as set forth in SEQ ID NO: 4, HC-CDR 2 comprising the amino acid sequence WINTYTGEPTYTDDFKG as set forth in SEQ ID NO: 5, and HC-CDR 3 comprising the amino acid sequence GGFGSSYWYFDV as set forth in SEQ ID NO: 6, wherein the CDRs are defined according to Kabat; and (ii) a V_{L} comprising LC-CDR 1 comprising the amino acid sequence KASQDVSIAVA as set forth in SEQ ID NO: 7, LC-CDR 2 comprising the amino acid sequence SASDRYT as set forth in SEQ ID NO: 10, and LC-CDR 3 comprising the amino acid sequence QQHYITPLT as set forth in SEQ ID NO: 9, wherein the CDRs are defined according to Kabat.

In a further embodiment, the avidity-tuned TROP2 binders of the present invention comprise (i) a V_{H} comprising the amino acid sequence set forth in SEQ ID NO: 1; and (ii) a V_{L} comprising the amino acid sequence set forth in SEQ ID NO: 3. In particular embodiments, the V_{H} is linked to a heavy chain constant domain of the IgG1, IgG2, IgG3, or IgG4 isotype and V_{L} is linked to a light chain constant domain of the human kappa or human lambda isotype. In further embodiments, the V_{H} is linked to the heavy chain constant domain of the IgG1 or IgG4 isotype and the V_{L} linked to a light chain constant domain of the human kappa or human lambda isotype. In further embodiments, the IgG1 or IgG4 may include 1, 2, 3, 4, 5, 6, 7, 8, 9, or 10 amino acid substitutions and/or deletions compared to the native human IgG1 or IgG4 isotype. In particular embodiments, the heavy chain constant domain is of the IgG1 isotype and may comprise 1, 2, 3, 4, 5, 6, 7, 8, 9, or 10 amino acid substitutions, additions, deletions, or combinations thereof compared to the amino acid sequence of the native IgG1 isotype.

In particular embodiments, the V_{H} is linked to the constant domain of a human IgG1 comprising the amino acid sequence set forth in SEQ ID NO: 92 or variant thereof comprising an S375C substitution and having the amino acid sequence shown in SEQ ID NO: 103.

In further embodiments, the constant domain of the human IgG1 comprises a substitution of the amino acids at positions 252, 254, and 256 of the constant domain of the heavy chain with amino acids Tyr (Y), Thr (T), and Glu (E), respectively (M252Y, S254T, T256E substitution) wherein the numbering is according to Eu (The positions according to sequential number are 256, 258, and 260, respectively), to provide a heavy chain constant domain comprising a “YTE” substitution and having the amino acid sequence set forth in SEQ ID NO: 93 or variant thereof comprising an S375C substitution and having the amino acid sequence shown in SEQ ID NO: 104.

In further embodiments, the human IgG1 heavy chain constant domain comprises E233A and L235A amino acid substitutions wherein the numbering is according to Eu, to provide a heavy chain constant domain having the amino acid sequence set forth in SEQ ID NO: 94 or variant thereof comprising an S375C substitution and having the amino acid sequence shown in SEQ ID NO: 105.

In further embodiments, the human IgG1 heavy chain constant domain comprises L234A L235A D265S substitutions wherein the numbering is according to Eu, to provide a heavy chain constant domain having the amino acid sequence set forth in SEQ ID NO: 95 or variant thereof comprising an S375C substitution and having the amino acid sequence shown in SEQ ID NO: 106.

In further embodiments, the human IgG1 heavy chain constant domain comprises L234A L235A P329G substitutions wherein the numbering is according to Eu, to provide a heavy chain constant domain having the amino acid sequence set forth in SEQ ID NO: 96 or variant thereof comprising an S375C substitution and having the amino acid sequence shown in SEQ ID NO: 107.

In further embodiments, the human IgG1 heavy chain constant domain comprises L235E substitutions wherein the numbering is according to Eu, to provide a heavy chain constant domain having the amino acid sequence set forth in SEQ ID NO: 97 or variant thereof comprising an S375C substitution and having the amino acid sequence shown in SEQ ID NO: 108.

In further embodiments, the human IgG1 heavy chain constant domain comprises D265A substitution wherein the numbering is according to Eu, to provide a heavy chain constant domain having the amino acid sequence set forth in SEQ ID NO: 98 or variant thereof comprising an S375C substitution and having the amino acid sequence shown in SEQ ID NO: 109.

In further embodiments, the human IgG1 heavy chain constant domain comprises D265A N297G substitutions wherein the numbering is according to Eu, to provide a heavy chain constant domain having the amino acid sequence set forth in SEQ ID NO: 99 or variant thereof comprising an S375C substitution and having the amino acid sequence shown in SEQ ID NO: 110.

In further embodiments, the human IgG1 heavy chain constant domain comprises N297X, wherein X is any amino acid other than N substitution wherein the numbering is according to Eu, to provide a heavy chain constant domain having the amino acid sequence set forth in SEQ ID NO: 100 or variant thereof comprising an S375C substitution and having the amino acid sequence shown in SEQ ID NO: 111.

In further embodiments, the human IgG1 heavy chain constant domain comprises N297A/D356E/L358M substitutions wherein the numbering is according to Eu, to provide a heavy chain constant domain having the amino acid sequence set forth in SEQ ID NO: 101 or variant thereof comprising an S375C substitution and having the amino acid sequence shown in SEQ ID NO: 112.

In particular embodiments of the invention, the IgG1 or IgG4 heavy chain constant domains as disclosed herein may comprise a C-terminal lysine or lack either a C-terminal lysine or a C-terminal glycine-lysine dipeptide. In some embodiments, the N-terminal amino acid of the antibody variable domains may undergo cyclization to pyroglutamate. Thus, in a composition comprising a particular antibody disclosed herein, the composition may comprise a population of antibody species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

The present invention further provides TROP2 binders that are rehumanized variants of Sacituzumab in which the rehumanization process unexpectedly produced an antibody with reduced hydrophobicity compared to the hydrophobicity of Sacituzumab as may be determined by hydrophobic interaction chromatography (HIC) and have a more human-like sequence than Sacituzumab, e.g., comprising less predicted epitope content compared to Sacituzumab as may be determined in silico using an immunogenicity predictive program.

These rehumanized TROP2 binders comprise the amino acid sequence of Sacituzumab in which the amino acid sequence thereof has been modified to comprise (i) a light chain having amino acid substitutions S20T, D60S, V85T, and A100P compared to the amino acid sequence of the light chain of Sacituzumab as set forth in SEQ ID NO: 12 wherein the positions are defined by sequential numbering and (ii) a heavy chain having amino acid substitutions Q5L, K38R, A69S, T78Q, D89E, F95Y, S115T, R218K, E360D, and M362L wherein the positions are defined by sequential numbering (the same positions in the V_{H} defined by Kabat numbering are Q5L, K38R, A68S, T77Q, D85E, F91Y, and S107T; and, in the heavy chain constant domain by Eu numbering are R214K, E256D, and M359L) compared to the amino acid sequence of the heavy chain of Sacituzumab having the amino acid sequence set forth in SEQ ID NO: 11. These mutations when present together on the heavy chain or light chain are referred to herein as “BSM” (Best Single Mutations). In an exemplary embodiment, the rehumanized Sacituzumab is αTROP2 (HC:BSM) (LC BSM), which comprises a heavy chain comprising the amino acid sequence of SEQ ID NO: 17 or 61 and a light chain comprising the amino acid sequence of SEQ ID NO: 21. The rehumanized Sacituzumab αTROP2 (HC:BSM) (LC:BSM) displays a higher degree of humanness compared to Sacituzumab and displays lower hydrophobicity than Sacituzumab as determined by hydrophobic interaction chromatography (HIC).

In further embodiments, the heavy chain comprising a TROP2 binder disclosed herein comprises a YTE substitution in the constant domain. The YTE substitution provides a TROP2 binder with improved PK in humans and non-human primates even though in rodents it appears to decrease PK compared to that of Sacituzumab. The YTE substitution promotes FcRn-mediated recycling to minimize ADC catabolism in non-tumor normal tissue. An exemplary TROP2 binder is αTROP2 (HC:BSM-YTE) (LC:BSM), which comprises a light chain having the amino acid sequence set forth in SEQ ID NO: 21 and a heavy chain having the amino acid sequence set forth in SEQ ID NO: 18 or 62, displays lower hydrophobicity than Sacituzumab as determined by HIC, and has a serum half-life that is longer than the serum half-life of Sacituzumab.

In a further embodiment, the αTROP2 (HC:BSM-YTE) (LC:BSM) binder comprises a light chain further comprising a Y53D amino acid substitution to provide TROP2 binder αTROP2 (HC:BSM-YTE) (LC:BSM-Y53D). These TROP2 binders display (i) preferential binding to cells that express high amounts of TROP2 as may be found in TROP2-expressing cancer cells over cells that express low amounts of TROP2 as may be found in non-cancer cells, (ii) reduced hydrophobicity compared to Sacituzumab, and (iii) reduced potential immunogenicity compared to Sacituzumab.

### Antibody Drug Conjugates

The present invention further provides antibody-drug conjugates (ADCs) comprising an anti-TROP2 antibody of the present invention conjugated to one or more payload molecules through a linker.

The payload used in the present invention is not particularly limited. Payloads for use in the present invention include cytotoxic moieties, particularly those which are used for cancer therapy. Such cytotoxic moieties include, but are not limited to, DNA damaging agents, DNA binding agents, anti-metabolites, enzyme inhibitors such as thymidylate synthase inhibitors and topoisomerase inhibitors, tubulin inhibitors, and toxins (for example, toxins of a bacterial, fungal, plant or animal origin).

Specific examples of cytotoxic moieties include, but are not limited to, taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoids (including DM1, DM2, DM3, DM4), auristatins including monomethylauristatin E (MMAE), monomethylauristatin F (MMAF), monomethylauristatin D (MMAD), 7-ethyl-10-hydroxy-camptothecin (SN-38), anthracycline, alkylcycline, or derivative thereof. The therapeutic moiety may be linked to the linker via any suitable methods known in the art.

The payload used in the present invention can be bound to an anti-TROP2 antibody via a linker. Various linkers for ADCs are known in the art. Linkers useful in the present invention are not particularly limited, as long as it includes a moiety capable reacting with a thiol group on an antibody and thereby linking to the antibody. In particular embodiments, the linker is an maleimido or haloactyl functionalized linker. Examples of linkers include, but are not limited to linkers having the following structures
i. -MC-vc-PABC- (“MC”: Maleimide-caproyl (Maleimide-C6); “-vc-”: the dipeptide of -Val-Cit-; “PABC”: para-aminobenzylcarbamate),
ii. -MC-GGFG- (“-GGFG-”: tetrapeptide-Gly-Gly-Phe-Gly-), GGFG is disclosed as SEQ ID NO: 185.
iii. -MC-vc-,
iv. -MP- (“MP”: Maleimide-propanyl (Maleimide-C3)),
v. -MP-GGGG- (“-GGGG-”: tetrapeptide-Gly-Gly-Gly-Gly-), GGGG is disclosed as SEQ ID NO: 186.
vi. -MP-GGG- (“-GGG-”: tripeptide-GGG-),
vii. -MP-GGG-PABC-,
viii. -MC-,
ix. -SMCC- (succinimidyl-4-(N-maleimidomethyl) cyclohexane-1-carboxylate),
x. -MP-AA-PABC- (“Ala-Ala” dipeptide),
xi. -CM2P- (5-cyano-6-methylsulfonyl 2-Pyridinecarboxamide),
xii. -CM3P- (5-cyano-6-methylsulfonyl 3-Pyridinecarboxamide),
xiii. CM2P-AA-PABC-, and
xiv. CM3P-AA-PABC-
wherein the proximal end of the linker comprising the reactive group MP, MC, CM2P, CM3P, or SMCC is capable of being conjugated to the reactive group (a thiol group) on an antibody and the distal end of the linker is capable of being linked to a payload.

In further embodiments, the payload is provided for conjugation in the form of a linker-payload compound intermediate having one of the following structures:
I. MC-vc-PABC-Payload,
II. MC-GGFG-Payload,
III. MC-vc-Payload,
IV. MP-Payload,
V. MP-GGG-Payload,
VI. MP-GGGG-Payload,
VII. MP-GGG-PABC-Payload,
VIII. MC-Payload,
IX. SMCC-Payload,
X. MP-AA-PABC-Payload,
XI. -CM2P-payload,
XII. -CM3P-payload,
XIII. CM2P-AA-PABC-payload, and
XIV. CM3P-AA-PABC-payload
wherein the proximal end of the linker comprising the reactive group MP, MC, CM2P, CM3P, or SMCC is capable of conjugating to a reactive group (thiol group) on the antibody.

In particular embodiments of the aforementioned linker payloads, the payload is a taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, DM1, DM2, DM3, DM4, MMAE, MMAF, MMAD, SN-38, anthracycline, alkylcycline, or derivative thereof. The aforementioned payloads further include pharmaceutically acceptable salts and solvates thereof. Examples of exemplary linker-payloads include but are not limited to MC-vc-PABC-MMAE, MP-AA-PABC-MMAE, CM2P-AA-PABC-MMAE, and CM3P-AA-PABC-MMAE.

The linker-payloads can form salts or solvates, which are also within the scope of the present disclosure. Exemplary linker payloads further include compounds of Formula I, or a pharmaceutically acceptable salt, solvate, or stereoisomer thereof:

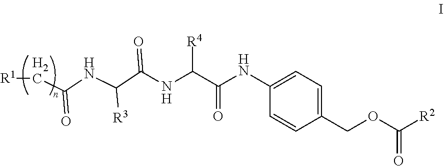

wherein:
R¹ is selected from:

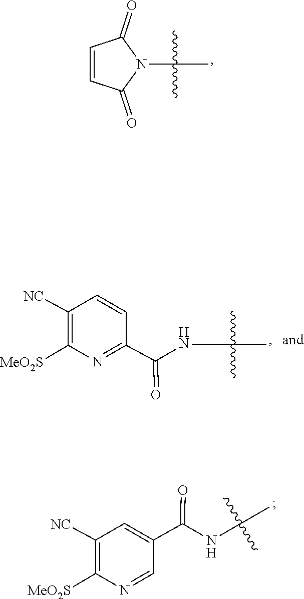 [SMILES: *N1C(=O)C=CC1=O ; *NC(=O)c1ccc(C#N)c(S(C)(=O)=O)n1 ; *NC(=O)c1cnc(S(C)(=O)=O)c(C#N)c1]

[symbol]

on wavy line indicates the site of covalent attachment; R² is a cytotoxic moiety; R³ and R⁴ independently represent C1-3 alkyl or a naturally occurring or unnatural amino acid side chain; and n is an integer from 1 to 4.

In particular embodiments, R² is selected from an anthracycline, an auristatin, a camptothecin, a duocarmycin, an etoposide, a maytansinoid, a pyrrolobenzodiazepine dimer, a DNA minor groove binder, a taxane, ean nediyne, an anti-tubulin, and a vinca alkaloid. In further embodiments, R² is selected from auristatin T, auristatin E, auristatin F phenylenediamine, benzolyl-auristatin E ester, 5-benzoylvaleric acid-AE ester, monomethyl auristatin F, lipophilic MMAF, MMAE, lexitropsins, duocarmycins, paclitaxel and docetaxel, T67 (Tularik), vincristine, vinblastine, vindesine, vinorelbine, nicotinamide phosphoribosyltranferase inhibitor (NAMPTi), tubulysin M, alkylcycline, melphalan, methotrexate, mitomycin C, etoposide, CC-1065 analogue, calicheamicin, maytansine, an analog of dolastatin 10, rhizoxin, palytoxin, baccatin derivatives, taxane analogs (e.g., epothilone A and B), nocodazole, colchicine and colcimid, estramustine, cryptophysins, cemadotin, maytansinoids, combretastatins, discodermoide, tesirine, and eleuthrobin.

In particular embodiments, R³ and R⁴ are independently selected from C₁₋₃ alkyl or are both CH₃. An exemplary linker payload may comprise a structure of Formula II:

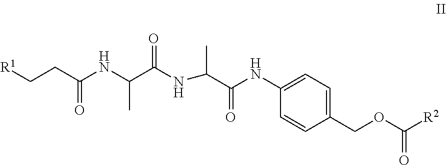

wherein R¹ and R² are as described herein. In particular embodiments, R² is an auristatin selected from auristatin E, auristatin F phenylenediamine, benzolyl-auristatin E ester, 5-benzoylvaleric acid-AE ester, MMAF, MMAE, or a pyrrolobenzodiazepine dimer.

Exemplary linker payloads comprising an MMAE derivative include but are not limited to the following compounds:

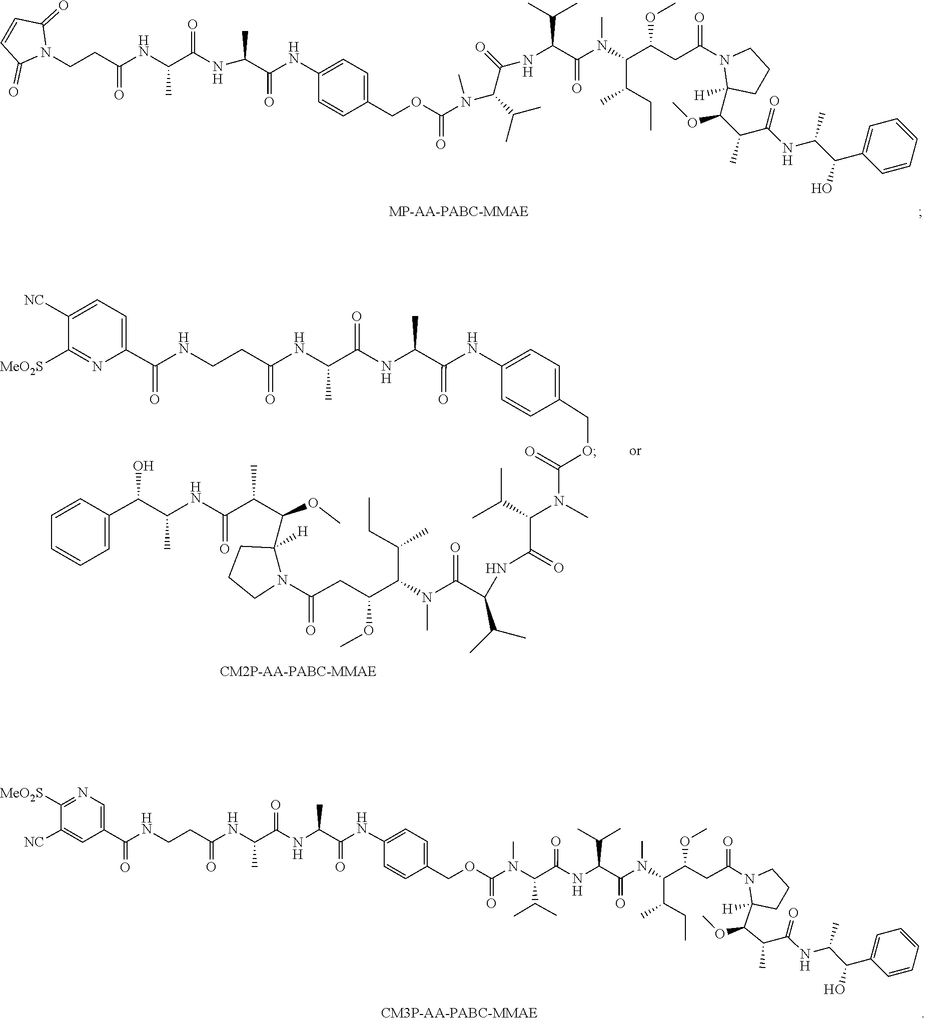

Exemplary linker payloads further include pharmaceutically acceptable salts and solvates of the following compounds:

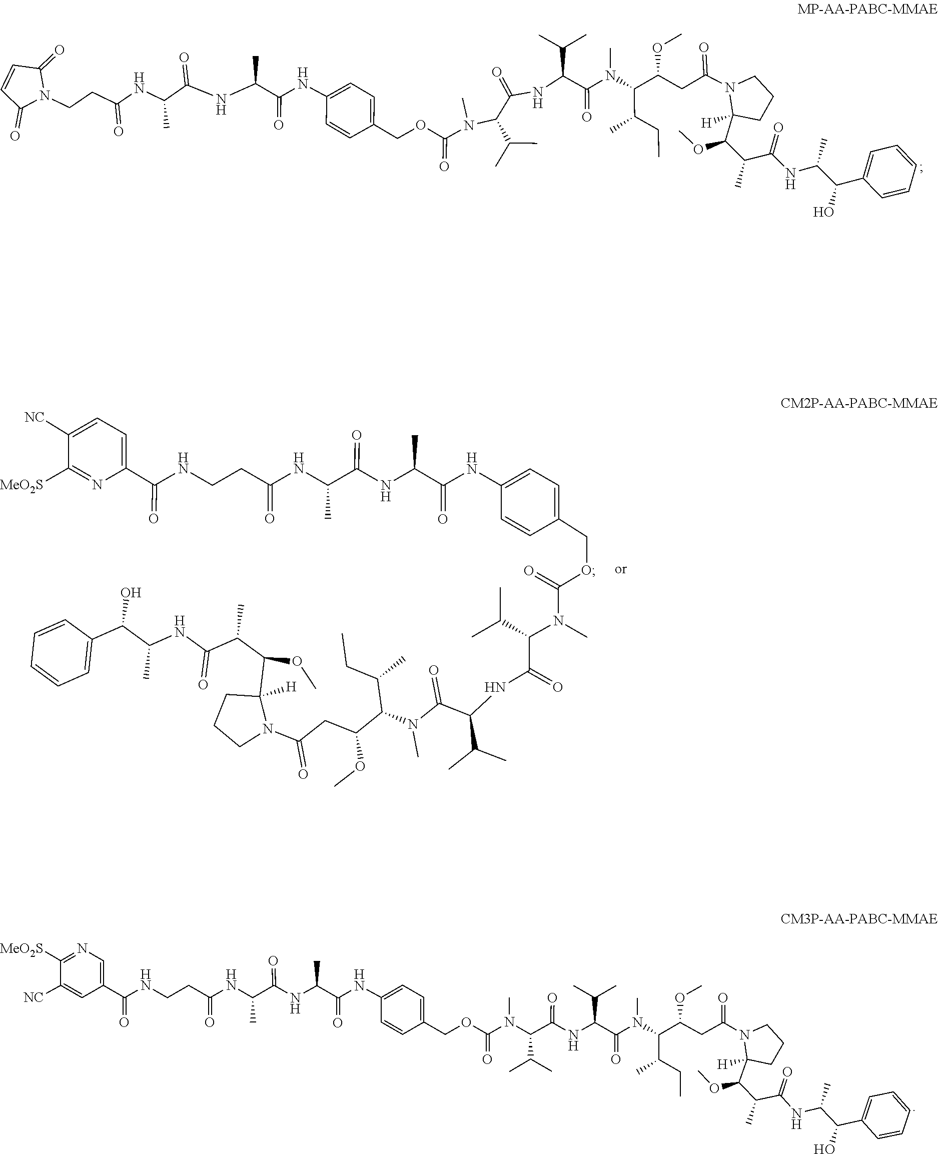

In some embodiments, the aforementioned exemplary linker-payloads are connected to an anti-TROP2 antibody of the present invention via the cysteine residues provided by selected inter-chain disulfide bonds opened by reduction of the anti-TROP2 antibody to provide the ADC of the present invention. In some embodiments, an ADC may comprise 1, 2, 3, 4, 5, 6, 7, or 8 payloads conjugated thereto. For a composition or mixture of ADCs, the mixture or composition may have a ratio of drug to antibody (DAR) ranging from about 2 to about 8. In particular embodiments, the DAR may be from about 2 to about 6, and in certain embodiments, the DAR may be about 2 or between 1 and 2. The ratio may refer to an average ratio in a population, such as an average of DAR 2 for a population of ADCs. In particular embodiments, the conjugate comprises payload mostly attached at Fab domains, and in some cases, comprises all the four payloads attached at Fab domains.

In a further embodiment, provided herein is a composition or mixture comprising or consisting of ADCs of the present invention, wherein at least about 80%, at least about 85%, at least about 90%, or at least about 95% of the ADCs in the mixture or composition has a DAR of about 1, 2, 3, 4, 5, 6, 7, or 8. In some cases, the mixture of ADCs has a DAR of about 4, in which a majority of the ADCs in the mixture has four payload molecules linked thereto. In other words, wherein the predominant species of ADC in the mixture or composition comprises four payload molecules.

In some embodiments, the anti-TROP2 antibody of the present invention is genetically engineered to comprise one or more cysteine or non-canonical amino acid substitutions of amino acids at defined locations within the anti-TROP2 antibody. These cysteine residues or non-canonical amino acid residues may then be conjugated to linker-payloads via the sulfhydryl group of the cysteine residues or the reactive group of the non-canonical amino acid.

Thus, the anti-TROP2 antibodies of the present invention may further comprise one or more substitutions of an amino acid in the heavy chain or light chain thereof with a cysteine residue or non-canonical amino acid residue, which may then be used for conjugating a payload thereto. In particular embodiments, the amino acid positions that may be substituted are selected from positions 152, 153, 171, 172, 173, and 375 of the heavy chain constant domain (numbering according to Eu numbering scheme) and positions 165 and 168 of the light chain constant domain (numbering beginning with amino acid 1 at N-terminus). In particular embodiments, cysteine may be substituted for the amino acid at one or more of the positions 152, 153, 171, 172, 173, and 375 of the heavy chain constant domain (numbering according to Eu numbering scheme) and positions 165 and 168 of the light chain constant domain (numbering beginning with amino acid 1 at N-terminus). In particular embodiments, the anti-TROP2 antibody comprises an S375C amino acid substitution. In particular embodiments, the antibody comprises an S375C amino acid substitution and an E152C amino acid substitution. In particular embodiments, the antibody comprises an S375C amino acid substitution and an S168C amino acid substitution. A non-exclusive list of exemplary embodiments of anti-TROP2 antibodies of the present invention comprising one or more substitutions of an amino acid therein with a cysteine that may then be conjugated to an aforementioned payload are shown in Tables 3-8.

TABLE 3

| | Heavy | Light |
|---|---|---|
| | Chain | Chain |
| | (HC) | (LC) |
| | SEQ | SEQ |
| Anti-TROP2 Antibody | ID NO: | ID NO: |
| αTROP2 (HC: Sac-E152C) (LC: Sac-Y53D) | 24 or 64 | 13 |
| αTROP2 (HC: Sac-P153C) (LC: Sac-Y53D) | 25 or 65 | 13 |
| αTROP2 (HC: Sac-P171C) (LC: Sac-Y53D) | 26 or 66 | 13 |
| αTROP2 (HC: Sac-A172C) (LC: Sac-Y53D) | 27 or 67 | 13 |
| αTROP2 (HC: Sac-V173C) (LC: Sac-Y53D) | 28 or 68 | 13 |
| αTROP2 (HC: Sac-S375C) (LC: Sac-Y53D) | 29 or 69 | 13 |
| αTROP2 (HC: Sac) (LC: Sac-Y53D-E165C) | 11 or 58 | 50 |
| αTROP2 (HC: Sac) (LC: Sac-Y53D-S168C) | 11 or 58 | 51 |
| αTROP2 (HC: Sac-E152C-S375C) | 30 or 70 | 13 |
| (LC: Sac-Y53D) | | |

Sac = Sacituzumab
HC positions are in the constant domain and defined according to Eu numbering
LC positions are defined according to sequential numbering
Each anti-TROP2 antibody comprises two heavy chains and two light chains

TABLE 4

| | Heavy | Light |
|---|---|---|
| | Chain | Chain |
| | (HC) | (LC) |
| | SEQ | SEQ |
| Anti-TROP2 Antibody | ID NO: | ID NO: |
| αTROP2 (HC: Sac-YTE-E152C) (LC: Sac-Y53D) | 31 or 71 | 13 |
| αTROP2 (HC: Sac-YTE-P153C) (LC: Sac-Y53D) | 32 or 72 | 13 |
| αTROP2 (HC: Sac-YTE-P171C) (LC: Sac-Y53D) | 33 or 73 | 13 |
| αTROP2 (HC: Sac-YTE-A172C) (LC: Sac-Y53D) | 34 or 74 | 13 |
| αTROP2 (HC: Sac-YTE-V173C) (LC: Sac-Y53D) | 35 or 75 | 13 |
| αTROP2 (HC: Sac-YTE-S375C) (LC: Sac-Y53D) | 36 or 76 | 13 |
| αTROP2 (HC: Sac-YTE) (LC: Sac-Y53D-E165C) | 23 or 63 | 50 |
| αTROP2 (HC: Sac-YTE) (LC: Sac-Y53D-S168C) | 23 or 63 | 51 |
| αTROP2 (HC: Sac-YTE-E152C-S375C) | 37 or 77 | 13 |
| (LC: Sac-Y53D) | | |

Sac = Sacituzumab
HC positions are in the constant domain and defined according to Eu numbering
LC positions are defined according to sequential numbering
Each anti-TROP2 antibody comprises two heavy chains and two light chains

TABLE 5

| | Heavy | Light |
|---|---|---|
| | Chain | Chain |
| | (HC) | (LC) |
| | SEQ | SEQ |
| Anti-TROP2 Antibody | ID NO: | ID NO: |
| αTROP2 (HC: BSM-E152C) (LC: BSM) | 38 or 78 | 21 |
| αTROP2 (HC: BSM-P153C) (LC: BSM) | 39 or 79 | 21 |
| αTROP2 (HC: BSM-P171C) (LC: BSM) | 40 or 80 | 21 |
| αTROP2 (HC: BSM-A172C) (LC: BSM) | 41 or 81 | 21 |
| αTROP2 (HC: BSM-V173C) (LC: BSM) | 42 or 82 | 21 |
| αTROP2 (HC: BSM-S375C) (LC: BSM) | 19 or 83 | 21 |
| αTROP2 (HC: BSM)(LC: BSM-E165C) | 17 or 59 | 52 |
| αTROP2 (HC: BSM)(LC: BSM-S168C) | 17 or 59 | 53 |
| αTROP2 (HC: BSM-E152C-S375C) | 43 or 84 | 21 |
| (LC: BSM) | | |

BSM = Best Single Mutations
HC positions are in the constant domain and defined according to Eu numbering
LC positions are defined according to sequential numbering
Each anti-TROP2 antibody comprises two heavy chains and two light chains

TABLE 6

| | Heavy | Light |
|---|---|---|
| | Chain | Chain |
| | (HC) | (LC) |
| | SEQ | SEQ |
| Anti-TROP2 Antibody | ID NO: | ID NO: |
| αTROP2 (HC: BSM-E152C) (LC: BSM-Y53D) | 38 or 78 | 22 |
| αTROP2 (HC: BSM-P153C) (LC: BSM-Y53D) | 39 or 79 | 22 |
| αTROP2 (HC: BSM-P171C) (LC: BSM-Y53D) | 40 or 80 | 22 |
| αTROP2 (HC: BSM-A172C) (LC: BSM-Y53D) | 41 or 81 | 22 |
| αTROP2 (HC: BSM-V173C) (LC: BSM-Y53D) | 42 or 82 | 22 |
| αTROP2 (HC: BSM-S375C) (LC: BSM-Y53D) | 19 or 83 | 22 |
| αTROP2 (HC: BSM) (LC: BSM-Y53D-E165C) | 17 or 59 | 54 |
| αTROP2 (HC: BSM) (LC: BSM-Y53D-S168C) | 17 or 59 | 55 |
| αTROP2 (HC: BSM-E152C-S375C) | 43 or 84 | 22 |
| (LC: BSM-Y53D) | | |

BSM = Best Single Mutations
HC positions are in the constant domain and defined according to Eu numbering
LC positions are defined according to sequential numbering
Each anti-TROP2 antibody comprises two heavy chains and two light chains

TABLE 7

| | Heavy | Light |
|---|---|---|
| | Chain | Chain |
| | (HC) | (LC) |
| | SEQ | SEQ |
| Anti-TROP2 Antibody | ID NO: | ID NO: |
| αTROP2 (HC: BSM-YTE-E152C) (LC: BSM) | 44 or 85 | 21 |
| αTROP2 (HC: BSM-YTE-P153C) (LC: BSM) | 45 or 86 | 21 |
| αTROP2 (HC: BSM-YTE-P171C) (LC: BSM) | 46 or 87 | 21 |
| αTROP2 (HC: BSM-YTE-A172C) (LC: BSM) | 47 or 88 | 21 |
| αTROP2 (HC: BSM-YTE-V173C) (LC: BSM) | 48 or 89 | 21 |
| αTROP2 (HC: BSM-YTE-S375C) (LC: BSM) | 20 or 90 | 21 |
| αTROP2 (HC: BSM-YTE) (LC: BSM-E165C) | 18 or 60 | 52 |
| αTROP2 (HC: BSM-YTE) (LC: BSM-S168C) | 18 or 60 | 53 |
| αTROP2 (HC: BSM-YTE-E152C-S375C) | 49 or 91 | 21 |
| (LC: BSM) | | |

BSM = Best Single Mutations
HC positions are in the constant domain and defined according to Eu numbering
LC positions are defined according to sequential numbering
Each anti-TROP2 antibody comprises two heavy chains and two light chains

TABLE 8

| | Heavy | Light |
|---|---|---|
| | Chain | Chain |
| | (HC) | (LC) |
| | SEQ | SEQ |
| Anti-TROP2 Antibody | ID NO: | ID NO: |
| αTROP2 (HC: BSM-YTE-E152C) (LC: BSM-Y53D) | 44 or 85 | 22 |
| αTROP2 (HC: BSM-YTE-P153C) (LC: BSM-Y53D) | 45 or 86 | 22 |
| αTROP2 (HC: BSM-YTE-P171C) (LC: BSM-Y53D) | 46 or 87 | 22 |
| αTROP2 (HC: BSM-YTE-A172C) (LC: BSM-Y53D) | 47 or 88 | 22 |
| αTROP2 (HC: BSM-YTE-V173C) (LC: BSM-Y53D) | 48 or 89 | 22 |
| αTROP2 (HC: BSM-YTE-S375C) (LC: BSM-Y53D) | 20 or 90 | 22 |
| αTROP2 (HC: BSM-YTE) (LC: BSM-Y53D-E165C) | 18 or 60 | 54 |
| αTROP2 (HC: BSM-YTE) (LC: BSM-Y53D-S168C) | 18 or 60 | 55 |
| αTrop2 (HC: BSM-YTE-E152C-S375C) (LC: BSM- | 49 or 91 | 22 |
| Y53D) | | |

BSM = Best Single Mutations
HC positions are in the constant domain and defined according to Eu numbering
LC positions are defined according to sequential numbering
Each anti-TROP2 antibody comprises two heavy chains and two light chains

In exemplary embodiments, the ADC comprises an anti-TROP2 antibody of the present invention conjugated to an exemplary linker-payload disclosed herein and has the structure
a) Ab-MC-vc-PABC-Payload,
b) Ab-MC-GGFG-Payload,
c) Ab-MC-vc-Payload,
d) Ab-MP-Payload,
e) Ab-MP-GGG-Payload,
f) Ab-MP-GGGG-Payload,
g) Ab-MP-GGG-BCP-Payload,
h) Ab-MP-GGG-PABC-Payload,
i) Ab-MP-GGG-EDA-Payload,
j) Ab-MC-Payload,
k) Ab-SMCC-Payload,
l) Ab-MP-AA-PABC-Payload,
m) Ab-CM2P-AA-PABC-Payload, or
n) Ab-CM3P-AA-PABC-Payload
   wherein Ab is an anti-TROP2 antibody of the present invention.

In particular embodiments of the aforementioned ADCs, the payload is a taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, DM1, DM2, DM3, DM4, MMAE, MMAF, MMAD, 7SN-38, anthracycline, alkylcycline, or derivative thereof. Examples of exemplary linker payloads include but are not limited to Ab-MC-vc-PAB-MMAE, Ab-MP-AA-PABC-MMAE, Ab-CM2P-AA-PABC-MMAE, and Ab-CM3P-AA-PABC-MMAE.

The present invention further provides exemplary ADCs comprising an anti-TROP2 antibody of the present invention conjugated to a linker payload having the formula as set forth in Formula III or a stereoisomer thereof:

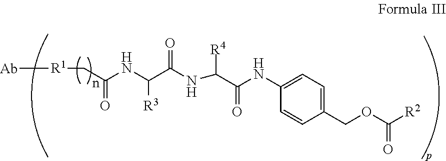

wherein

R¹ is selected from:

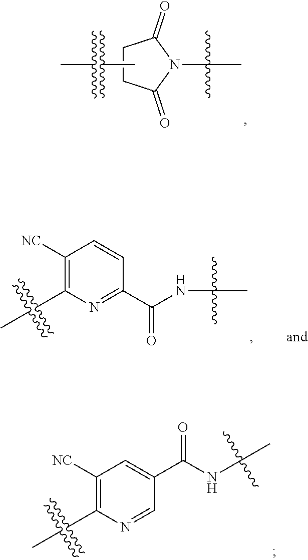

[symbol] single wavy line indicates the site of covalent attachment to —(CH₂)ₙ;
[symbol] double wavy line indicates the site of covalent attachment to a sulfur of a cysteine residue of Ab; R² is a cytotoxic moiety; R³ and R⁴ independently represent C1-3 alkyl or a naturally occurring or unnatural amino acid side chain; n is an integer from 1 to 4; Ab is an anti-TROP2 binder of the present invention; and p is a positive rational number from 1 to 24, including fractions and decimals. In further embodiments, R² is an auristatin drug selected from auristatin E, auristatin F phenylenediamine, benzolyl-auristatin E ester, 5-benzoylvaleric acid-AE ester, MMAF, MMAE, or a pyrrolobenzodiazepine dimer, and R³ and R⁴ both are CH₃.

In a further embodiment, the present invention further provides ADCs comprising an anti-TROP2 antibody of the present invention conjugated to a linker-MMAE payload, wherein the ADC comprises the formula:

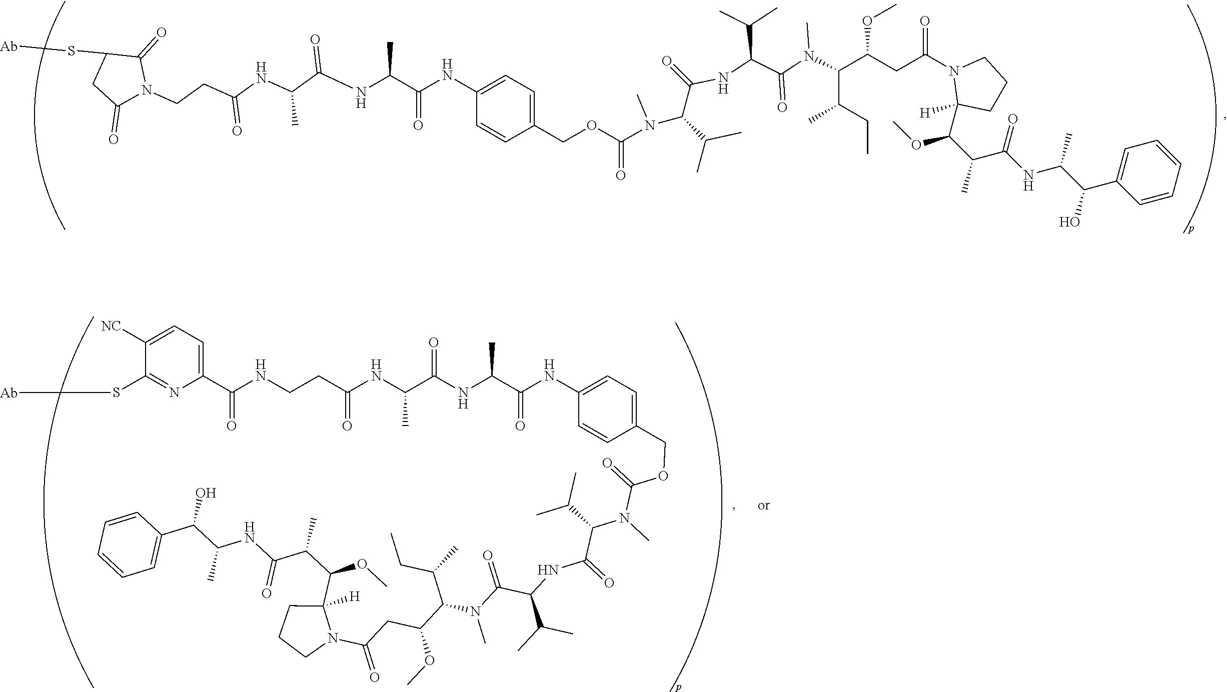

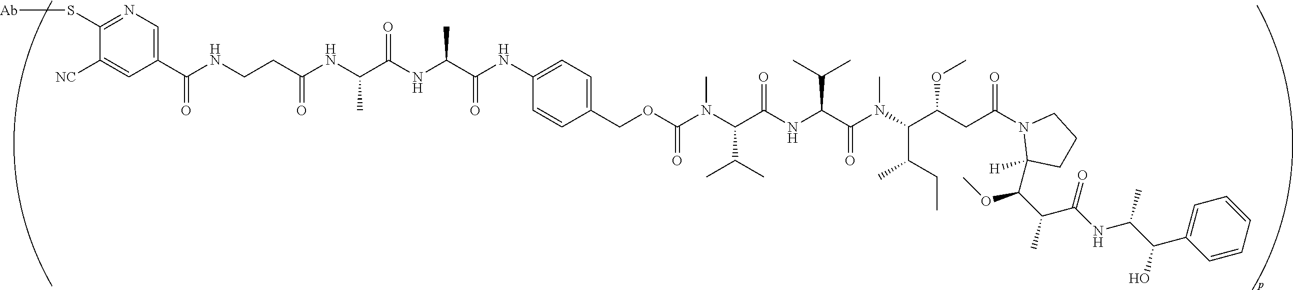

wherein Ab is an anti-Trop2 antibody of the present invention; S is the sulfur atom of a cysteine residue present in the heavy chain or light chain of Ab; and p, is an integer from 1 to 8, wherein the linker-MMAE payload is conjugated to the SH group of a cysteine residue of the Ab. In a further embodiment, the S is provided by the side chain of the cysteine residue at position 375 of the heavy chain constant domain (wherein the position is defined by Eu numbering).

In a particular embodiments, the present invention provides ADCs comprising an anti-TROP2 antibody of the present invention having an engineered cysteine residue that is conjugated to a linker-MMAE payload, wherein the ADC comprises the formula:

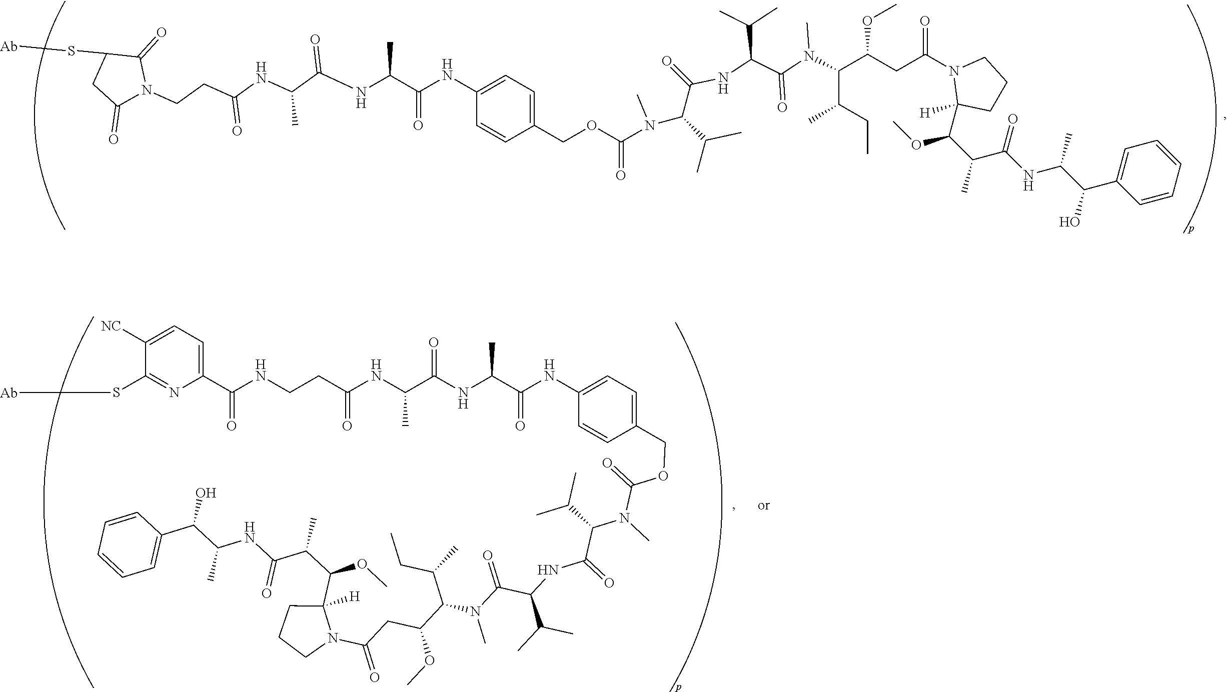

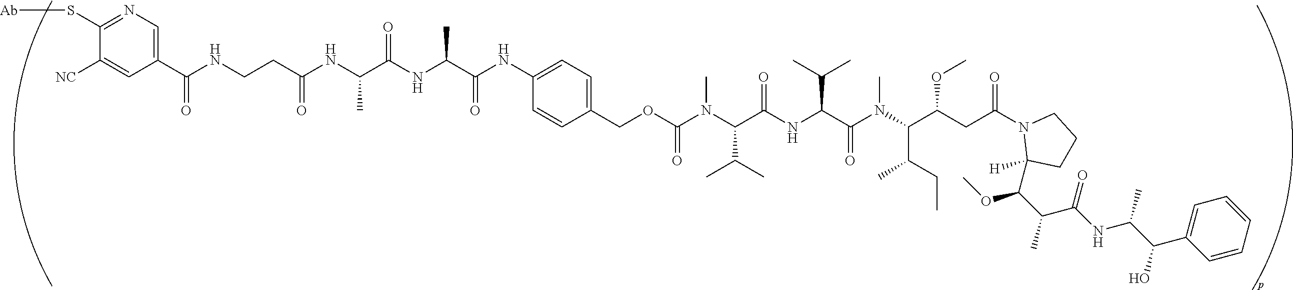

wherein Ab is an anti-Trop2 antibody of the present invention comprising two heavy chains and two light chains wherein the heavy chains or the light chains comprise engineered cysteines, wherein the heavy chains and the light chains are (A) selected from the group consisting of
(a) αTROP2 (HC:Sac-E152C) (LC:Sac-Y53D) comprising two heavy chains having the amino acid sequence set forth in SEQ ID No: 64 and two light chains having the amino acid sequence set forth in SEQ ID NO: 13;
(b) αTROP2 (HC:Sac-E153C) (LC:Sac-Y53D) two heavy chains having the amino acid sequence set forth in SEQ ID No: 65 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 66 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 67 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 68 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 69 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 71 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 72 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 73 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 74 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 75 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 76 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 58 and two light chains having the amino acid sequence set forth in SEQ ID NO: 50;
(n) αTROP2 (HC:Sac) (LC:Sac-Y53D-E168C) comprising two heavy chains having the amino acid sequence set forth in SEQ ID No: 58 and two light chains having the amino acid sequence set forth in SEQ ID NO: 51;
(o) αTROP2 (HC:Sac-YTE) (LC:Sac-Y53D-E165C) comprising two heavy chains having the amino acid sequence set forth in SEQ ID No: 63 and two light chains having the amino acid sequence set forth in SEQ ID NO: 50;
(p) αTROP2 (HC:Sac-YTE) (LC:Sac-Y53D-E168C) comprising two heavy chains having the amino acid sequence set forth in SEQ ID No: 63 and two light chains having the amino acid sequence set forth in SEQ ID NO: 51;
(q) αTROP2 (HC:BSM-E152C) (LC:BSM) comprising two heavy chains having the amino acid sequence set forth in SEQ ID No: 78 and two light chains having the amino acid sequence set forth in SEQ ID NO: 21;
(r) αTROP2 (HC:BSM-E153C) (LC:BSM) comprising two heavy chains having the amino acid sequence set forth in SEQ ID No: 79 and two light chains having the amino acid sequence set forth in SEQ ID NO: 21;
(s) αTROP2 (HC:BSM-E171C) (LC:BSM) comprising two heavy chains having the amino acid sequence set forth in SEQ ID No: 80 and two light chains having the amino acid sequence set forth in SEQ ID NO: 21;
(t) αTROP2 (HC:BSM-E172C) (LC:BSM) comprising two heavy chains having the amino acid sequence set forth in SEQ ID No: 81 and two light chains having the amino acid sequence set forth in SEQ ID NO: 21;
(u) αTROP2 (HC:BSM-E173C) (LC:BSM) comprising two heavy chains having the amino acid sequence set forth in SEQ ID No: 82 and two light chains having the amino acid sequence set forth in SEQ ID NO: 21;
(v) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 21;
(w) αTROP2 (HC:BSM-E152C) (LC:BSM-Y53D) comprising two heavy chains having the amino acid sequence set forth in SEQ ID No: 78 and two light chains having the amino acid sequence set forth in SEQ ID NO: 22;
(x) αTROP2 (HC:BSM-E153C) (LC:BSM-Y53D) comprising two heavy chains having the amino acid sequence set forth in SEQ ID No: 79 and two light chains having the amino acid sequence set forth in SEQ ID NO: 22;
(y) αTROP2 (HC:BSM-E171C) (LC:BSM-Y53D) comprising two heavy chains having the amino acid sequence set forth in SEQ ID No: 80 and two light chains having the amino acid sequence set forth in SEQ ID NO: 22;
(z) αTROP2 (HC:BSM-E172C) (LC:BSM-Y53D) comprising two heavy chains having the amino acid sequence set forth in SEQ ID No: 81 and two light chains having the amino acid sequence set forth in SEQ ID NO: 22;
(aa) αTROP2 (HC:BSM-E173C) (LC:BSM-Y53D) comprising two heavy chains having the amino acid sequence set forth in SEQ ID No: 82 and two light chains having the amino acid sequence set forth in SEQ ID NO: 22;
(bb) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 22;
(cc) αTROP2 (HC:BSM) (LC:BSM-E165C) comprising two heavy chains having the amino acid sequence set forth in SEQ ID No: 59 and two light chains having the amino acid sequence set forth in SEQ ID NO: 52;
(dd) αTROP2 (HC:BSM) (LC:BSM-E168C) comprising two heavy chains having the amino acid sequence set forth in SEQ ID No: 59 and two light chains having the amino acid sequence set forth in SEQ ID NO: 53;
(ee) αTROP2 (HC:BSM) (LC:BSM-Y53D-E165C) comprising two heavy chains having the amino acid sequence set forth in SEQ ID No: 59 and two light chains having the amino acid sequence set forth in SEQ ID NO: 54;
(ff) αTROP2 (HC:BSM) (LC:BSM-Y53D-E168C) comprising two heavy chains having the amino acid sequence set forth in SEQ ID No: 59 and two light chains having the amino acid sequence set forth in SEQ ID NO: 55;
(gg) αTROP2 (HC:BSM-YTE) (LC:BSM-E165C) comprising two heavy chains having the amino acid sequence set forth in SEQ ID No: 60 and two light chains having the amino acid sequence set forth in SEQ ID NO: 52;
(hh) αTROP2 (HC:BSM-YTE) (LC:BSM-E168C) comprising two heavy chains having the amino acid sequence set forth in SEQ ID No: 60 and two light chains having the amino acid sequence set forth in SEQ ID NO: 53;
(ii) αTROP2 (HC:BSM-YTE) (LC:BSM-Y53D-E165C) comprising two heavy chains having the amino acid sequence set forth in SEQ ID No: 60 and two light chains having the amino acid sequence set forth in SEQ ID NO: 54;
(jj) αTROP2 (HC:BSM-YTE) (LC:BSM-Y53D-E168C) comprising two heavy chains having the amino acid sequence set forth in SEQ ID No: 58 and two light chains having the amino acid sequence set forth in SEQ ID NO: 57;
(kk) αTROP2 (HC:BSM-YTE-E152C) (LC:BSM) comprising two heavy chains having the amino acid sequence set forth in SEQ ID No: 85 and two light chains having the amino acid sequence set forth in SEQ ID NO: 21;
(ll) αTROP2 (HC:BSM-YTE-E153C) (LC:BSM) comprising two heavy chains having the amino acid sequence set forth in SEQ ID No: 86 and two light chains having the amino acid sequence set forth in SEQ ID NO: 21;
(mm) αTROP2 (HC:BSM-YTE-E171C) (LC:BSM) comprising two heavy chains having the amino acid sequence set forth in SEQ ID No: 87 and two light chains having the amino acid sequence set forth in SEQ ID NO: 21;
(nn) αTROP2 (HC:BSM-YTE-E172C) (LC:BSM) comprising two heavy chains having the amino acid sequence set forth in SEQ ID No: 88 and two light chains having the amino acid sequence set forth in SEQ ID NO: 21;
(oo) αTROP2 (HC:BSM-YTE-E173C) (LC:BSM) comprising two heavy chains having the amino acid sequence set forth in SEQ ID No: 89 and two light chains having the amino acid sequence set forth in SEQ ID NO: 21;
(pp) αTROP2 (HC:BSM-YTE-S375C) (LC:BSM) comprising two heavy chains having the amino acid sequence set forth in SEQ ID No: 90 and two light chains having the amino acid sequence set forth in SEQ ID NO: 21;
(qq) αTROP2 (HC:BSM-YTE-E152C) (LC:BSM-Y53D) comprising two heavy chains having the amino acid sequence set forth in SEQ ID No: 85 and two light chains having the amino acid sequence set forth in SEQ ID NO: 22;
(rr) αTROP2 (HC:BSM-YTE-E153C) (LC:BSM-Y53D) comprising two heavy chains having the amino acid sequence set forth in SEQ ID No: 86 and two light chains having the amino acid sequence set forth in SEQ ID NO: 22;
(ss) αTROP2 (HC:BSM-YTE-E171C) (LC:BSM-Y53D) comprising two heavy chains having the amino acid sequence set forth in SEQ ID No: 87 and two light chains having the amino acid sequence set forth in SEQ ID NO: 22;
(tt) αTROP2 (HC:BSM-YTE-E172C) (LC:BSM-Y53D) comprising two heavy chains having the amino acid sequence set forth in SEQ ID No: 88 and two light chains having the amino acid sequence set forth in SEQ ID NO: 22;
(uu) αTROP2 (HC:BSM-YTE-E173C) (LC:BSM-Y53D) comprising two heavy chains having the amino acid sequence set forth in SEQ ID No: 89 and two light chains having the amino acid sequence set forth in SEQ ID NO: 22; and
(vv) αTROP2 (HC:BSM-YTE-S375C) (LC:BSM-Y53D) comprising two heavy chains having the amino acid sequence set forth in SEQ ID No: 90 and two light chains having the amino acid sequence set forth in SEQ ID NO: 22;

wherein the S is the sulfur atom of the engineered cysteine conjugated to the linker-MMAE payload; and, wherein p is an integer selected from 1 or 2; or

(B) selected from the group consisting of
(a) αTROP2 (HC:Sac-E152C-S375C) (LC:Sac-Y53D) comprising two heavy chains having the amino acid sequence set forth in SEQ ID No: 70 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 77 and two light chains having the amino acid sequence set forth in SEQ ID NO: 13;
(c) αTROP2 (HC:BSM-E152C-S375C) (LC:BSM) comprising two heavy chains having the amino acid sequence set forth in SEQ ID No: 84 and two light chains having the amino acid sequence set forth in SEQ ID NO: 21;
(d) αTROP2 (HC:BSM-E152C-S375C) (LC:BSM-Y53D) comprising two heavy chains having the amino acid sequence set forth in SEQ ID No: 84 and two light chains having the amino acid sequence set forth in SEQ ID NO: 22;
(e) αTROP2 (HC:BSM-YTE-E152C-S375C) (LC:BSM) comprising two heavy chains having the amino acid sequence set forth in SEQ ID No: 91 and two light chains having the amino acid sequence set forth in SEQ ID NO: 21; and
(f) αTROP2 (HC:BSM-YTE-E152C-S375C) (LC:BSM-Y53D) comprising two heavy chains having the amino acid sequence set forth in SEQ ID No: 91 and two light chains having the amino acid sequence set forth in SEQ ID NO: 22;

wherein the S is the sulfur atom of the engineered cysteine conjugated to the linker-MMAE payload; and, wherein p is an integer selected from 1, 2, 3, or 4.

In a particular embodiments, the present invention provides ADCs comprising an anti-TROP2 antibody of the present invention having an engineered cysteine residue that is conjugated to a linker-MMAE payload, wherein the ADC comprises the formula

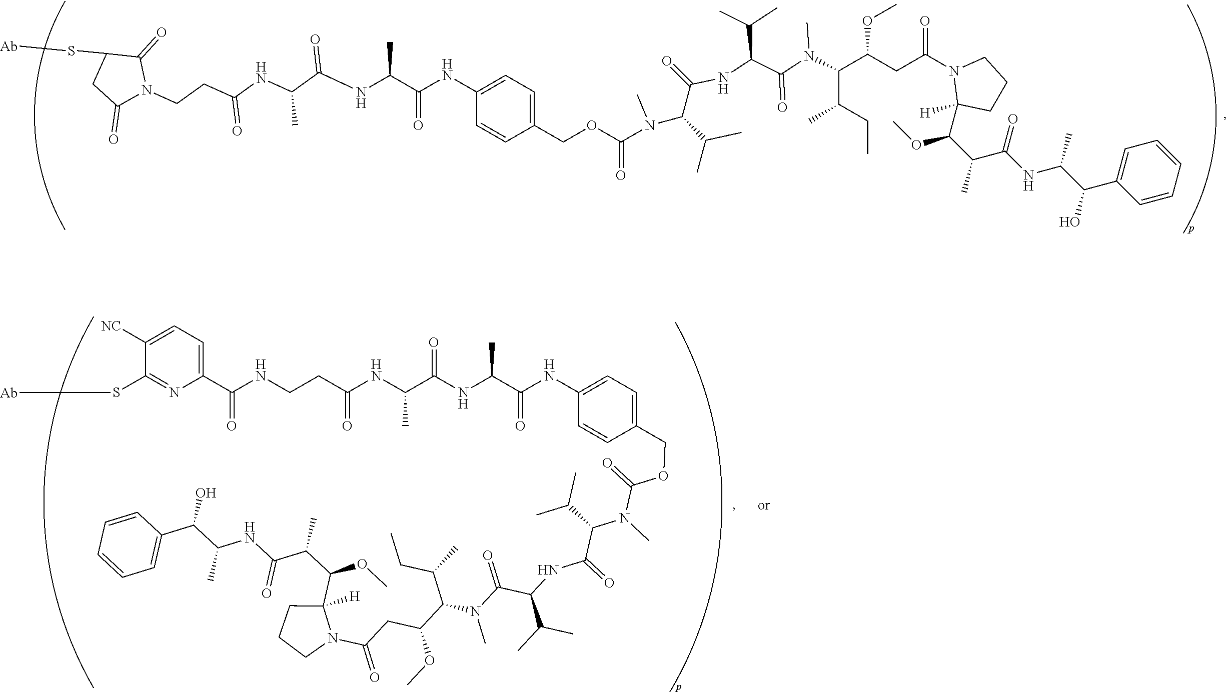

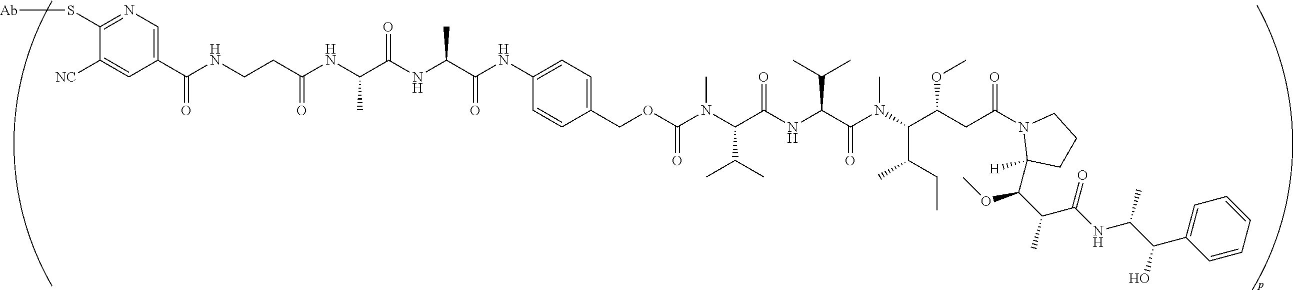

wherein Ab is an anti-Trop2 antibody of the present invention comprising two heavy chains and two light chains wherein the heavy chains or the light chains comprise engineered cysteines, wherein the heavy and light chains are (A) selected from the group consisting of
(a) αTROP2 (HC:Sac-E152C) (LC:Sac-Y53D) comprising two heavy chains having the amino acid sequence set forth in SEQ ID No: 24 and two light chains having the amino acid sequence set forth in SEQ ID NO: 13;
(b) αTROP2 (HC:Sac-E153C) (LC:Sac-Y53D) comprising two heavy chains having the amino acid sequence set forth in SEQ ID No: 25 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 26 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 27 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 28 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 29 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 31 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 32 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 33 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 34 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 35 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 36 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 11 and two light chains having the amino acid sequence set forth in SEQ ID NO: 52;
(n) αTROP2 (HC:Sac) (LC:Sac-Y53D-E168C) comprising two heavy chains having the amino acid sequence set forth in SEQ ID No: 11 and two light chains having the amino acid sequence set forth in SEQ ID NO: 53;
(o) αTROP2 (HC:Sac-YTE) (LC:Sac-Y53D-E165C) comprising two heavy chains having the amino acid sequence set forth in SEQ ID No: 23 and two light chains having the amino acid sequence set forth in SEQ ID NO: 52;
(p) αTROP2 (HC:Sac-YTE) (LC:Sac-Y53D-E168C) comprising two heavy chains having the amino acid sequence set forth in SEQ ID No: 23 and two light chains having the amino acid sequence set forth in SEQ ID NO: 53;
(q) αTROP2 (HC:BSM-E152C) (LC:BSM) comprising two heavy chains having the amino acid sequence set forth in SEQ ID No: 38 and two light chains having the amino acid sequence set forth in SEQ ID NO: 21;
(r) αTROP2 (HC:BSM-E153C) (LC:BSM) comprising two heavy chains having the amino acid sequence set forth in SEQ ID No: 39 and two light chains having the amino acid sequence set forth in SEQ ID NO: 21;
(s) αTROP2 (HC:BSM-E171C) (LC:BSM) comprising two heavy chains having the amino acid sequence set forth in SEQ ID No: 40 and two light chains having the amino acid sequence set forth in SEQ ID NO: 21;
(t) αTROP2 (HC:BSM-E172C) (LC:BSM) comprising two heavy chains having the amino acid sequence set forth in SEQ ID No: 41 and two light chains having the amino acid sequence set forth in SEQ ID NO: 21;
(u) αTROP2 (HC:BSM-E173C) (LC:BSM) comprising two heavy chains having the amino acid sequence set forth in SEQ ID No: 42 and two light chains having the amino acid sequence set forth in SEQ ID NO: 21;
(v) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 21;
(w) αTROP2 (HC:BSM-E152C) (LC:BSM-Y53D) comprising two heavy chains having the amino acid sequence set forth in SEQ ID No: 38 and two light chains having the amino acid sequence set forth in SEQ ID NO: 22;
(x) αTROP2 (HC:BSM-E153C) (LC:BSM-Y53D) comprising two heavy chains having the amino acid sequence set forth in SEQ ID No: 39 and two light chains having the amino acid sequence set forth in SEQ ID NO: 22;
(y) αTROP2 (HC:BSM-E171C) (LC:BSM-Y53D) comprising two heavy chains having the amino acid sequence set forth in SEQ ID No: 40 and two light chains having the amino acid sequence set forth in SEQ ID NO: 22;
(z) αTROP2 (HC:BSM-E172C) (LC:BSM-Y53D) comprising two heavy chains having the amino acid sequence set forth in SEQ ID No: 41 and two light chains having the amino acid sequence set forth in SEQ ID NO: 22;
(aa) αTROP2 (HC:BSM-E173C) (LC:BSM-Y53D) comprising two heavy chains having the amino acid sequence set forth in SEQ ID No: 42 and two light chains having the amino acid sequence set forth in SEQ ID NO: 22;
(bb) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 22;
(cc) αTROP2 (HC:BSM) (LC:BSM-E165C) comprising two heavy chains having the amino acid sequence set forth in SEQ ID No: 17 and two light chains having the amino acid sequence set forth in SEQ ID NO: 52;
(dd) αTROP2 (HC:BSM) (LC:BSM-E168C) comprising two heavy chains having the amino acid sequence set forth in SEQ ID No: 17 and two light chains having the amino acid sequence set forth in SEQ ID NO: 53;
(ee) αTROP2 (HC:BSM) (LC:BSM-Y53D-E165C) comprising two heavy chains having the amino acid sequence set forth in SEQ ID No: 17 and two light chains having the amino acid sequence set forth in SEQ ID NO: 54;
(ff) αTROP2 (HC:BSM) (LC:BSM-Y53D-E168C) comprising two heavy chains having the amino acid sequence set forth in SEQ ID No: 17 and two light chains having the amino acid sequence set forth in SEQ ID NO: 55;
(gg) αTROP2 (HC:BSM-YTE) (LC:BSM-E165C) comprising two heavy chains having the amino acid sequence set forth in SEQ ID No: 18 and two light chains having the amino acid sequence set forth in SEQ ID NO: 52;
(hh) αTROP2 (HC:BSM-YTE) (LC:BSM-E168C) comprising two heavy chains having the amino acid sequence set forth in SEQ ID No: 18 and two light chains having the amino acid sequence set forth in SEQ ID NO: 53;
(ii) αTROP2 (HC:BSM-YTE) (LC:BSM-Y53D-E165C) comprising two heavy chains having the amino acid sequence set forth in SEQ ID No: 18 and two light chains having the amino acid sequence set forth in SEQ ID NO: 54;
(jj) αTROP2 (HC:BSM-YTE) (LC:BSM-Y53D-E168C) comprising two heavy chains having the amino acid sequence set forth in SEQ ID No: 18 and two light chains having the amino acid sequence set forth in SEQ ID NO: 55;
(kk) αTROP2 (HC:BSM-YTE-E152C) (LC:BSM) comprising two heavy chains having the amino acid sequence set forth in SEQ ID No: 44 and two light chains having the amino acid sequence set forth in SEQ ID NO: 21;
(ll) αTROP2 (HC:BSM-YTE-E153C) (LC:BSM) comprising two heavy chains having the amino acid sequence set forth in SEQ ID No: 45 and two light chains having the amino acid sequence
(mm) αTROP2 (HC:BSM-YTE-E171C) (LC:BSM) comprising two heavy chains having the amino acid sequence set forth in SEQ ID No: 46 and two light chains having the amino acid sequence set forth in SEQ ID NO: 21;
(nn) αTROP2 (HC:BSM-YTE-E172C) (LC:BSM) comprising two heavy chains having the amino acid sequence set forth in SEQ ID No: 47 and two light chains having the amino acid sequence set forth in SEQ ID NO: 21;
(oo) αTROP2 (HC:BSM-YTE-E173C) (LC:BSM) comprising two heavy chains having the amino acid sequence set forth in SEQ ID No: 48 and two light chains having the amino acid sequence set forth in SEQ ID NO: 21;
(pp) αTROP2 (HC:BSM-YTE-S375C) (LC:BSM) comprising two heavy chains having the amino acid sequence set forth in SEQ ID No: 20 and two light chains having the amino acid sequence set forth in SEQ ID NO: 21;
(qq) αTROP2 (HC:BSM-YTE-E152C) (LC:BSM-Y53D) comprising two heavy chains having the amino acid sequence set forth in SEQ ID No: 44 and two light chains having the amino acid sequence set forth in SEQ ID NO: 22;
(rr) αTROP2 (HC:BSM-YTE-E153C) (LC:BSM-Y53D) comprising two heavy chains having the amino acid sequence set forth in SEQ ID No: 45 and two light chains having the amino acid sequence set forth in SEQ ID NO: 22;
(ss) αTROP2 (HC:BSM-YTE-E171C) (LC:BSM-Y53D) comprising two heavy chains having the amino acid sequence set forth in SEQ ID No: 46 and two light chains having the amino acid sequence set forth in SEQ ID NO: 22;
(tt) αTROP2 (HC:BSM-YTE-E172C) (LC:BSM-Y53D) comprising two heavy chains having the amino acid sequence set forth in SEQ ID No: 47 and two light chains having the amino acid sequence set forth in SEQ ID NO: 22;
(uu) αTROP2 (HC:BSM-YTE-E173C) (LC:BSM-Y53D) comprising two heavy chains having the amino acid sequence set forth in SEQ ID No: 48 and two light chains having the amino acid sequence set forth in SEQ ID NO: 22; and
(vv) αTROP2 (HC:BSM-YTE-S375C) (LC:BSM-Y53D) comprising two heavy chains having the amino acid sequence set forth in SEQ ID No: 20 and two light chains having the amino acid sequence set forth in SEQ ID NO: 22;

wherein the S is the sulfur atom of the side chain of the engineered cysteine conjugated to the linker-MMAE payload; and, wherein p is an integer selected from 1 or 2; or

(B) selected from the group consisting of
(a) αTROP2 (HC:Sac-E152C-S375C) (LC:Sac-Y53D) comprising two heavy chains having the amino acid sequence set forth in SEQ ID No: 30 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 37 and two light chains having the amino acid sequence set forth in SEQ ID NO: 13;
(c) αTROP2 (HC:BSM-E152C-S375C) (LC:BSM) comprising two heavy chains having the amino acid sequence set forth in SEQ ID No: 43 and two light chains having the amino acid sequence set forth in SEQ ID NO: 21;
(d) αTROP2 (HC:BSM-E152C-S375C) (LC:BSM-Y53D) comprising two heavy chains having the amino acid sequence set forth in SEQ ID No: 43 and two light chains having the amino acid sequence set forth in SEQ ID NO: 22;
(e) αTROP2 (HC:BSM-YTE-E152C-S375C) (LC:BSM) comprising two heavy chains having the amino acid sequence set forth in SEQ ID No: 49 and two light chains having the amino acid sequence set forth in SEQ ID NO: 21; and
(f) αTROP2 (HC:BSM-YTE-E152C-S375C) (LC:BSM-Y53D) comprising two heavy chains having the amino acid sequence set forth in SEQ ID No: 49 and two light chains having the amino acid sequence set forth in SEQ ID NO: 22;

wherein the S is the sulfur atom of the side chain of the engineered cysteine conjugated to the linker-MMAE payload; and, wherein p is an integer selected from 1, 2, 3, or 4.

In a further embodiment, the present invention further provides ADCs comprising an anti-TROP2 antibody of the present invention having an engineered cysteine residue at position 375 of the heavy chain constant domain that is conjugated to a linker payload, wherein the ADC comprises the formula:

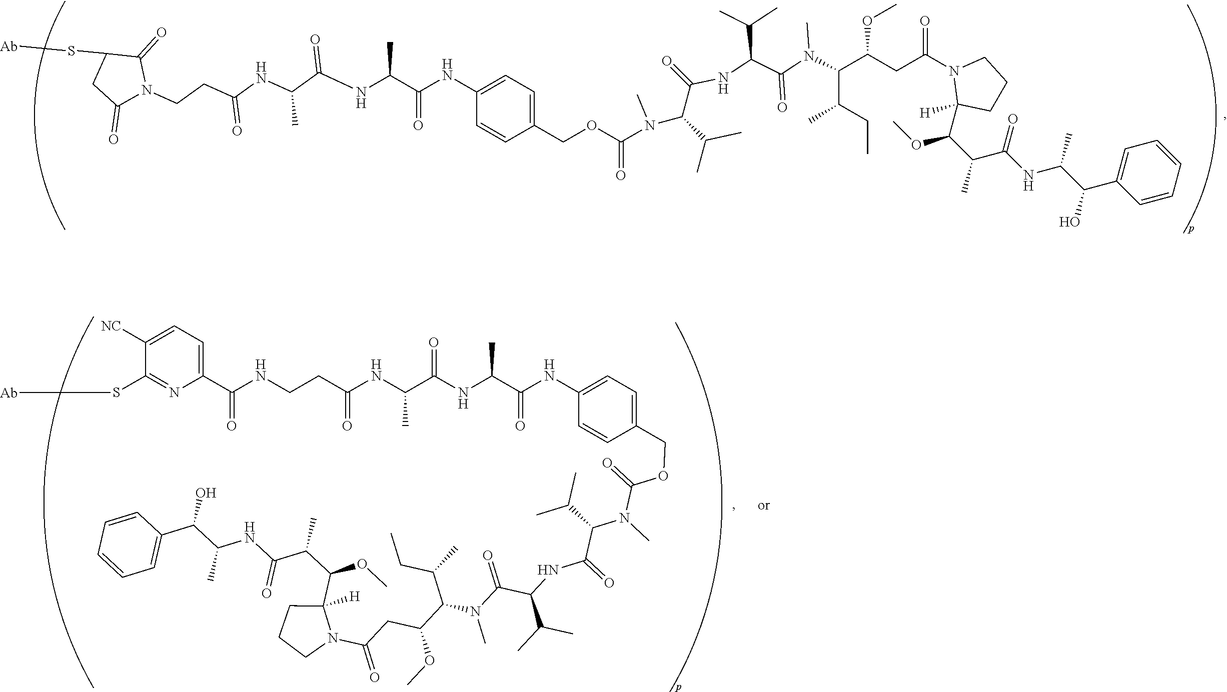

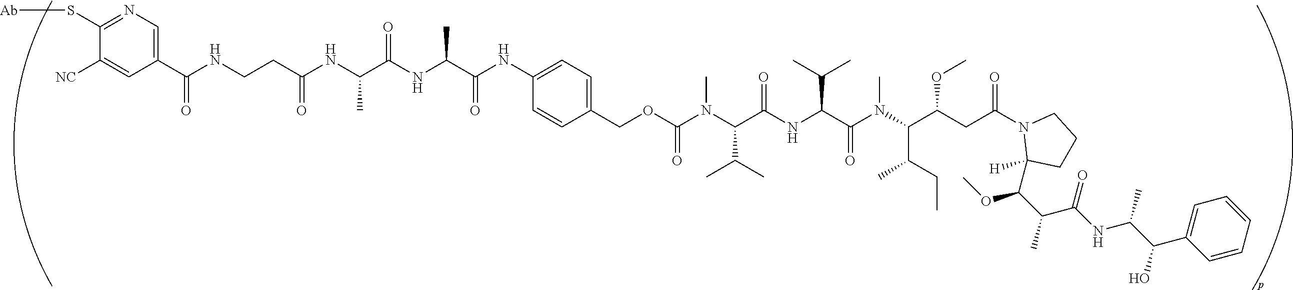

wherein Ab is the anti-TROP2 antibody of the present invention comprising two heavy chains having the amino acid sequence set forth in SEQ ID NO: 20 and two light chains having the amino acid sequence set forth in SEQ ID NO: 22; wherein p is 1 or 2; wherein S is the sulfur atom of the side chain of the cysteine residue at amino acid position 375 as defined by Eu numbering

In the ADCs of the present invention, the maleimide residue of the MP-AA-PABC-MMAE linker payload when conjugated to the cysteine residue in the antibody undergoes a ring opening reaction, which results in a more stable linkage between the antibody and the maleimide group.

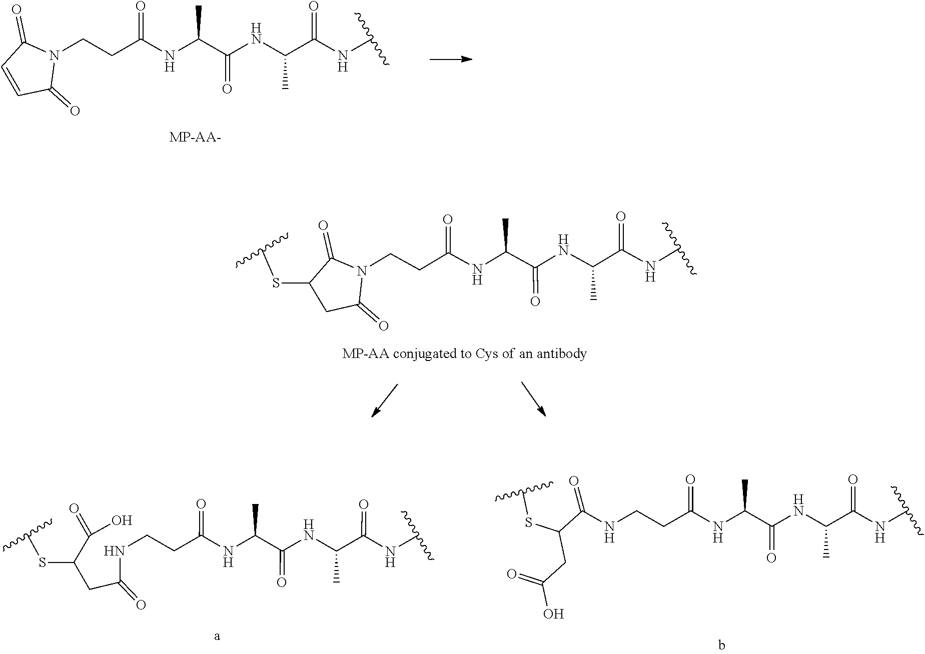

The ring opening occurs more rapidly than the ring opening of vedotin (MC-vc-PABC-MMAE) so there is less deconjugation occurring prior to ring opening for the ADCs of the present invention compared to ADCs conjugated to vedotin. Thus, disclosed herein are compositions of any one of the aforementioned ADCs of the present invention having an anti-TROP2 antibody disclosed herein conjugated to MP-AA-PABC-MMAE in which a portion of the ADCs in the composition have the structure:

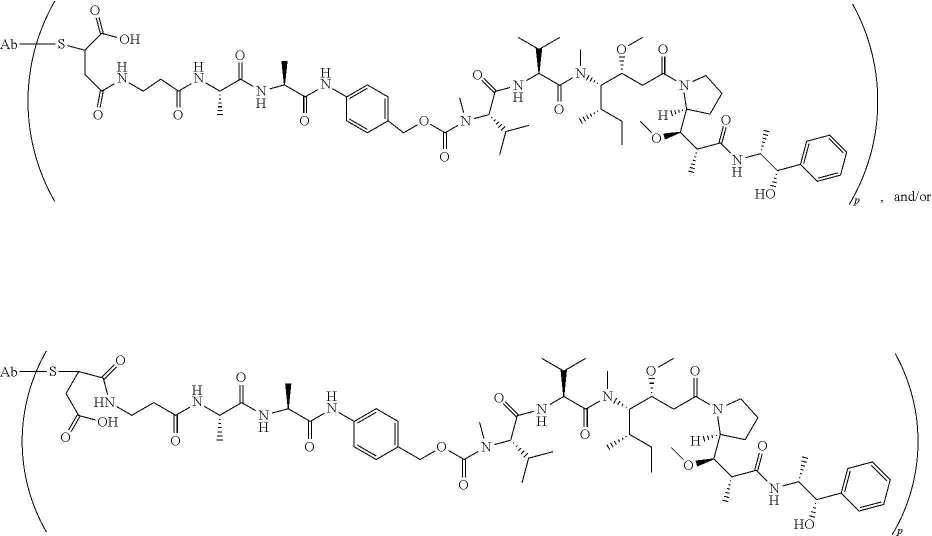

wherein p is 1 or 2; and S is the sulfur atom of a cysteine residue present in the heavy chain or light chain of Ab. In particular embodiments, the ADC comprises a cysteine residue at position 375 and the sulfur atom of the cysteine residue is conjugated to the MP-AA-PABC-MMAE linker payload, wherein the amino acid numbering of the heavy chain constant domain is according to the Eu numbering scheme.

In a further embodiment, provided herein is a pharmaceutical composition comprising any one of the aforementioned ADCs or mixtures thereof and a pharmaceutically acceptable carrier. In particular embodiments of the pharmaceutical composition, the predominant species of ADC comprises antibodies in which the heavy chain comprises a C-terminal lysine. In particular embodiments of the pharmaceutical composition, the predominant species of ADC comprises antibodies in which the heavy chain lacks a C-terminal lysine. In particular embodiments of the pharmaceutical composition, the predominant species of ADC comprises antibodies in which the heavy chain lacks a C-terminal glycine lysine dipeptide. In particular embodiments of the pharmaceutical composition, the predominant species of ADC comprises antibodies in which the heavy chain N-terminal amino acid is glutamine. In particular embodiments of the pharmaceutical composition, the predominant species of ADC comprises antibodies in which the heavy chain N-terminal amino acid is glutamic acid. In particular embodiments of the pharmaceutical composition, the predominant species of ADC comprises antibodies in which the heavy chain N-terminal amino acid is glutamine that has cyclized to pyroglutamate. In particular embodiments of the pharmaceutical composition, the predominant species of ADC comprises antibodies in which the heavy chain N-terminal amino acid is glutamic acid that has cyclized to pyroglutamate. In particular embodiments of the pharmaceutical composition, the predominant species of ADC comprises antibodies in which the heavy chain N-terminal amino acid is pyroglutamate. In particular embodiments of the pharmaceutical composition, the predominant species of ADC comprises antibodies in which the heavy chain N-terminal amino acid is pyroglutamate and the heavy chain C-terminus lacks lysine. In particular embodiments of the pharmaceutical composition, the predominant species of ADC comprises antibodies in which the heavy chain N-terminal amino acid is pyroglutamate and the heavy chain C-terminus lacks a glycine lysine dipeptide.

The present invention further provides pharmaceutically acceptable salts or solvates of any one of the aforementioned ADCs.

ScFv Fusion Proteins that Bind TROP2

In particular embodiments, the V_{H} and V_{L} disclosed herein are expressed as an ScFv fusion protein in which the V_{L} and V_{H} domains are linked together by a peptide linker. The peptide linker joins the carboxyl terminus of one variable region domain to the amino terminus of the other variable domain without compromising the fidelity of the V_{H}-V_{L} paring and antigen-binding sites. Thus, the ScFv may comprise a fusion protein in which the C-terminus of a V_{L} is linked by a peptide linker to the N-terminus of a V_{H} or a fusion protein in which the C-terminus of a V_{H} is linked by a peptide linker to the N-terminus of a V_{L}. Peptide linkers for linking the variable domains can vary from 10 to 25 amino acids in length and are typically, but not always, composed of hydrophilic amino acids such as glycine (G) and serine(S) having the structure G4S (SEQ ID NO: 187), for example, (G4S) n (SEQ ID NO: 188), wherein n is 1, 2, 3, 4, or 5. Peptide linkers of shorter lengths (0-4 amino acids) have also been used; however, ScFv bearing shorter linkers may form multimers. Generally, the (G4S)₃ peptide comprising three repeating G4S units (“(G4S)₃” disclosed as SEQ ID NO: 189) is used as an ScFv peptide linker (See for example, Leath et al., Int. J. Oncol. 24:765-771 (2004); Holliger et al., Proc. Natl. Acad. Sci. USA 90:6444-6448 (1993); Iliades et al., FEBS Lett. 409:437-441 (1997)).

Exemplary ScFv fusion proteins comprise the structure V_{L}-(G4S) n-V_{H} or V_{H}-(G4S) n-V_{L} wherein the V_{H} domain comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6; and the V_{L} domain comprises a CDR 1 comprising the amino acid sequence set forth in SEQ ID NO: 7, a CDR 2 comprising the amino acid sequence set forth in SEQ ID NO: 10, and a CDR 3 comprising the amino acid sequence set forth in SEQ ID NO: 9, wherein the CDR sequences are defined by the Kabat numbering scheme. In particular embodiment, n is 1, 2, 3, 4, or 5.

Exemplary ScFv fusion proteins comprise the structure V_{L}-(G4S) n-V_{H} or V_{H}-(G4S) n-V_{L} wherein the V_{H} comprises the amino acid sequence set forth in SEQ ID NO: 1 and the V_{L} comprises the amino acid sequence set forth in SEQ ID NO: 3; and, wherein n is 1, 2, 3, 4, or 5.

Exemplary ScFv fusion proteins comprise the structure V_{L}-(G4S) n-V_{H} or V_{H}-(G4S) n-V_{L} wherein the V_{H} comprises the amino acid sequence set forth in SEQ ID NO: 14 and V_{L} comprises the amino acid sequence set forth in SEQ ID NO: 15; and, wherein n is 1, 2, 3, 4, or 5.

Exemplary ScFv fusion proteins comprise the structure V_{L}-(G4S) n-V_{H} or V_{H}-(G4S) n-V_{L} wherein the V_{H} comprises the amino acid sequence set forth in SEQ ID NO: 14 and the V_{L} comprises the amino acid sequence set forth in SEQ ID NO: 16; and, wherein n is 1, 2, 3, 4, or 5.

The ScFvs disclosed herein may be provided in a bispecific format comprising a CD3 binder (ScFv) linked by a peptide linker to an ScFv that binds TROP2 as disclosed herein. When these molecules, called Bispecific T-cell engagers (BiTE®s), bind CD3 on T cells and TROP2 expressed on the surface of a cell, it brings the T cells to a tumor site.

ScFvs disclosed herein may also be fused to cellular toxins, radioisotopes, cytokines, and enzymes for cancer, autoimmune, and/or inflammatory therapeutic applications. In particular embodiments, the peptide linker may comprise 1 to 10 G4S peptide units (SEQ ID NO: 190).

In further embodiments, the ScFvs disclosed herein may be linked to or inserted in different locations of an intact IgG molecule to confer dual epitope binding. For example, a bispecific antibody may be provided comprising two heterodimeric heavy chain constant domains wherein the N-terminus of one heavy chain constant domain is fused to the C-terminus of an ScFv disclosed herein and the N-terminus of the other heavy chain constant domain is fused to the C-terminus of an ScFv that targets an antigen other than TROP2 or a Fab′ that targets an antigen other than TROP2.

### Nucleic Acid Molecules Encoding the TROP2 Binders of the Present Invention

The present invention further provides nucleic acid molecules that encode the TROP2 binders of the present invention. In particular embodiments, the TROP2 binder comprises a V_{H} encoded by a first nucleic acid molecule and a V_{L} encoded by a second nucleic acid molecule. In particular embodiments, the TROP2 binder is an antibody in which the heavy chain is encoded by a first nucleic acid molecule and the light chain is encoded by a second nucleic acid molecule.

In particular embodiments, the heavy chain and light chain (or V_{H} and V_{L}) are expressed as a fusion protein in which the N-terminus of the heavy chain and light chain (or V_{H} and V_{L}) are fused at the N-terminus to a leader peptide to facilitate the transport of the TROP2 binder through the secretory pathway. In particular embodiments, the N-terminus of the ScFv fusion protein is fused at the N-terminus to a leader or signal peptide to facilitate the transport of the ScFv through the secretory pathway. Examples of leader/signal peptides that may be used include those comprising the amino acid sequence set forth in SEQ ID NO: 56 or SEQ ID NO: 57. Thus, in particular embodiments, the aforementioned nucleic acid molecules may comprise a polynucleotide encoding a leader peptide linked to the 5′ end of the nucleic acid molecule encoding the anti-TROP2 binder.

The nucleic acid molecules disclosed herein may include one or more substitutions that optimize one or more of the codons for enhancing the expression of the nucleic acid molecule in a particular host cell, e.g., yeast or fungal host cell, non-human mammalian host cell, human host cell, insect host cell, or prokaryote host cell.

### Methods for Making a TROP2 Binder of the Present Invention

The present invention includes recombinant methods for making a TROP2 binder of the present invention comprising introducing into a host cell (i) an expression vector comprising nucleic acid molecule(s) that encode the V_{H} and V_{L} of the TROP2 binder or the heavy chain and light chain of an anti-TROP2 binder, or (ii) two expression vectors comprising nucleic acid molecules, one vector comprising a nucleic acid molecule encoding the V_{H} of a TROP2 binder or the heavy chain of an anti-TROP2 binder, the other vector comprising a nucleic acid molecule encoding the V_{L} of a TROP2 binder or the light chain of a TROP2 binder. The nucleic acid molecules or polynucleotides encoding the V_{H}, V_{L}, heavy chain, or light chain are operably linked to a promoter and other transcription and translation regulatory sequences. The host cell is cultured under conditions and a time period suitable for expression of the nucleic acid molecules followed by isolating the TROP2 binder from the host cell and/or medium in which the host cell is grown. See e.g., WO2004041862, WO2006122786, WO2008020079, WO2008142164 or WO2009068627. The expression vector may be a plasmid or viral vector. The invention also relates to host cells that comprise such nucleic acid molecules encoding the TROP2 binders (host cells comprising nucleic acid molecules encoding the V_{H} and V_{L} or nucleic acid molecules encoding the heavy chain and light chain) or components thereof (host cells comprising nucleic acid molecule encoding solely the V_{H} or heavy chain or solely the V_{L} or light chain).

Eukaryotic and prokaryotic host cells, including mammalian cells as hosts for expression of the TROP2 binder are well known in the art and include many immortalized cell lines available from the American Type Culture Collection (ATCC). These include, but are not limited to, Chinese hamster ovary (CHO) cells, NSO cells, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Thus, mammalian host cells include human, mouse, rat, dog, monkey, pig, goat, bovine, horse, and hamster cells. In particular cell lines are selected through determining which cell lines have high expression levels. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Saccharomyces cerevisiae, and Trichoderma reesei. The present invention further includes any host cell comprising an TROP2 binder of the present invention or comprising one or more nucleic acid molecules encoding such TROP2 binder or comprising an expression vector that comprises one or more nucleic acid molecules encoding such TROP2 binder.

Further, expression of a TROP2 binder from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0216846B1, 0256055B1, 0323997B1, and 0338841B1. Thus, in an embodiment of the invention, the mammalian host cells lack a glutamine synthetase gene and are grown in the absence of glutamine in the medium wherein, however, the nucleic acid molecule encoding the immunoglobulin chain comprises a glutamine synthetase gene which complements the lack of the gene in the host cell. Such host cells containing the TROP2 binder or nucleic acid molecule(s) or expression vector(s) as discussed herein as well as expression methods, as discussed herein, for making the TROP2 binder using such a host cell are part of the present invention.

The present invention further includes methods for purifying a TROP2 binder comprising introducing a sample (e.g., culture medium, cell lysate or cell lysate fraction, e.g., a soluble fraction of the lysate) comprising the TROP2 binder to a purification medium (e.g., cation-exchange medium, anion-exchange medium and/or hydrophobic exchange medium) and either collecting purified TROP2 binder from the flow-through fraction of said sample that does not bind to the medium; or, discarding the flow-through fraction and eluting bound TROP2 binder from the medium and collecting the eluate. In an embodiment of the invention, the medium is in a column to which the sample is applied. In an embodiment of the invention, the purification method is conducted following recombinant expression of the TROP2 binder in a host cell, e.g., wherein the host cell is first lysed and, optionally, the lysate is purified of insoluble materials prior to purification on a medium; or wherein the TROP2 binder is secreted into the culture medium by the host cell and the medium or a fraction thereof is applied to the purification medium.

In general, glycoproteins produced in a particular cell line or transgenic animal will have a glycosylation pattern that is characteristic for glycoproteins produced in the cell line or transgenic animal. Therefore, the particular glycosylation pattern of a TROP2 binder will depend on the particular cell line or transgenic animal used to produce the TROP2 binder. TROP2 binders comprising only non-fucosylated N-glycans are part of the present invention and may be advantageous, because non-fucosylated antibodies have been shown to typically exhibit more potent efficacy than their fucosylated counterparts both in vitro and in vivo (See for example, Shinkawa et al., J. Biol. Chem. 278:3466-3473 (2003); U.S. Pat. Nos. 6,946,292 and 7,214,775). These TROP2 binders with non-fucosylated N-glycans are not likely to be immunogenic because their carbohydrate structures are a normal component of the population that exists in human serum IgG.

The present invention includes TROP2 binders comprising N-linked glycans that are typically added to immunoglobulins produced in Chinese hamster ovary cells (CHO N-linked glycans) or to engineered yeast cells (engineered yeast N-linked glycans), such as, for example, Pichia pastoris. For example, in an embodiment of the invention, the TROP2 binder comprises one or more of the “engineered yeast N-linked glycans” or “CHO N-linked glycans” (e.g., G0 and/or G0-F and/or G1 and/or G1-F and/or G2-F and/or Man5). In an embodiment of the invention, the TROP2 binder comprises the engineered yeast N-linked glycans, i.e., G0 and/or G1 and/or G2, optionally, further including Man5. In an embodiment of the invention, the TROP2 binders comprise the CHO N-linked glycans, i.e., G0-F, G1-F and G2-F, optionally, further including G0 and/or G1 and/or G2 and/or Man5. In an embodiment of the invention, about 80% to about 95% (e.g., about 80-90%, about 85%, about 90% or about 95%) of all N-linked glycans on the TROP2 binders are engineered yeast N-linked glycans or CHO N-linked glycans. See Nett et al. Yeast. 28:237-252 (2011); Hamilton et al. Science. 313:1441-1443 (2006); Hamilton et al. Curr Opin Biotechnol. 18 (5): 387-392 (2007). For example, in an embodiment of the invention, an engineered yeast cell is GF15.0 or YGLY8316 or strains set forth in U.S. Pat. No. 7,795,002 or Zha et al. Methods Mol Biol. 988:31-43 (2013). See also International Patent Application Publication No. WO2013066765.

### Pharmaceutical Compositions Comprising TROP2 Binders or ADCs of the Present Invention

The TROP2 binder or ADC of the present invention disclosed herein may be provided in suitable pharmaceutical compositions comprising one or more TROP2 binders or ADCs of the present invention and a pharmaceutically acceptable carrier. The carrier may be a diluent, adjuvant, excipient, or vehicle with which the TROP2 binder or ADC of the present invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the TROP2 binders or ADCs of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in Remington: The Science and Practice of Pharmacy, 21.sup.st Edition, Troy, D. B. ed., Lipincott Williams and Wilkins, Philadelphia, Pa. 2006, Part 5, Pharmaceutical Manufacturing pp 691-1092, see especially pp. 958-989.

The mode of administration of the TROP2 binder or ADC of the present invention or pharmaceutical composition comprising the TROP2 binder or ADC of the present invention thereof may be any suitable route such as parenteral administration, e.g., intradermal, intramuscular, intraperitoneal, intravenous, subcutaneous, pulmonary, transmucosal (oral, intranasal, intravaginal, rectal) or other means appreciated by the skilled artisan, as well known in the art.

The TROP2 binder or ADC of the present invention may be administered to an individual (e.g., patient) by any suitable route, for example parentally by intravenous (i.v.) infusion or bolus injection, intramuscularly or subcutaneously, or intraperitoneally. An i.v. infusion may be given over for, example, 15, 30, 60, 90, 120, 180, or 240 minutes, or from 1, 2, 3, 4, 5, 6, 7, 8, 9, 10, 11 or 12 hours.

The administration of the TROP2 binder or ADC of the present invention or a pharmaceutical composition comprising the TROP2 binder or ADC of the present invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

The TROP2 binder or ADC of the present invention or a pharmaceutical composition comprising the TROP2 binder or ADC of the present invention may be administered by maintenance therapy, such as, e.g., once a week for a period of 6 months or more.

The anti-TROP2 binder or ADC of the present invention or a pharmaceutical composition comprising the TROP2 binder or ADC of the present invention may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The TROP2 binder or ADC of the present invention or pharmaceutical composition comprising the TROP2 binder or ADC of the present invention may be lyophilized for storage and reconstituted in a suitable carrier prior to use. This technique has been shown to be effective with conventional protein preparations and well known lyophilization and reconstitution techniques can be employed.

### Combination Therapy Treatments

Combination therapies of the present invention comprising a TROP2 binder or ADC of the present invention or pharmaceutical composition comprising the TROP2 binder or ADC of the present invention and another therapeutic agent (e.g., small molecule or antibody) may be used for the treatment any proliferative disease, in particular, the treatment of cancer. In particular embodiments, the combination therapy of the present invention may be used to treat breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

### Combination Therapy Comprising a TROP2 Binder or ADC of the Present Invention and Chemotherapy

The combination therapy of the present invention comprising a TROP2 binder or ADC of the present invention or a pharmaceutical composition comprising the TROP2 binder or ADC of the present invention may be administered to an individual having a cancer in combination with chemotherapy. The individual may undergo the chemotherapy at the same time the individual is undergoing the combination therapy of the present invention. The individual may undergo the combination therapy of the present invention after the individual has completed chemotherapy. The individual may be administered the chemotherapy after completion of the combination therapy. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of:
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracycline or alkylcycline;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the agent, the level of symptoms, the immunogenicity of the agent, and the accessibility of the target cells, tissue or organ in the individual being treated.

The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. Sec, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, on the individual's clinical history (e.g., previous therapy), the type and stage of the cancer to be treated and biomarkers of response to one or more of the therapeutic agents in the combination therapy.

Thus, the present invention contemplates embodiments of the combination therapy of the present invention that further includes a chemotherapy step comprising platinum-containing chemotherapy, e.g., pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment of any proliferative disease, in particular, the treatment of cancer. In particular embodiments, the combination therapy of the present invention may be used to treat breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

### Combination Therapy Comprising a TROP2 Binder or ADC and a Therapeutic Antibody

The TROP2 binder or ADC of the present invention or a pharmaceutical composition comprising the TROP2 binder or ADC of the present invention may be administered in combination with one or more therapeutic antibodies for the treatment of cancer or proliferative disease. The individual may undergo treatment with the therapeutic antibody at the same time the individual is undergoing the combination therapy of the present invention. The individual may undergo the combination therapy of the present invention after the individual has completed treatment with the therapeutic antibody. The individual may be administered the treatment with the therapeutic antibody after completion of the combination therapy. The combination therapy of the present invention may also be administered to an individual having recurrent or metastatic cancer with disease progression or relapse cancer and who is undergoing chemotherapy or who has completed chemotherapy. In particular embodiments, the therapeutic agent targets the programmed death 1 receptor or ligand, PD-1 and PD-L1, respectively.

Exemplary anti-PD-1 antibodies that may be used in a combination therapy with the TROP2 binders or ADCs of the present invention disclosed herein include any antibody that binds PD-1 and inhibits PD-1 from binding PD-L1 and/or PD-L2 or binds PD-L1 or PD-L2 and inhibits it from binding PD-1. In a particular embodiment, the exemplary anti-PD-1 antibody is pembrolizumab (KEYTRUDA). In a particular embodiment, the exemplary anti-PD-1 antibody is nivolumab (OPDIVO). In a particular embodiment, the exemplary anti-PD-1 antibody is cemiplimab (LIBTAYO). In a particular embodiment, the exemplary anti-PD-L1 antibody is durvalumab (IMFINZI). In a particular embodiment, the exemplary anti-PD-L1 antibody is atezolizumab (TECENTRIQ). In a particular embodiment, the exemplary anti-PD-L1 antibody is avelumab (BAVENCIO).

### Injection Device for Administering a TROP2 Binder or ADC

The present invention also provides an injection device comprising any one of the TROP2 binders or ADCs of the present invention or a pharmaceutical compositions comprising any one of the anti-TROP2 binders or ADCs of the present invention. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising any one of the TROP2 binders or ADCs of the present invention or pharmaceutical compositions comprising any one of the TROP2 binders or ADCs of the present invention), a needle for piecing skin and/or blood vessels for injection of the fluid; and a plunger for pushing the fluid out of the cylinder and through the needle bore. In an embodiment of the invention, an injection device comprising any one of the TROP2 binders or ADCs of the present invention or a pharmaceutical composition comprising any one of the TROP2 binders or ADCs of the present invention is an intravenous (IV) injection device. Such a device includes a composition comprising said TROP2 binder or ADC or a pharmaceutical composition in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the subject through the cannula or trocar/needle.

The TROP2 binders or ADCs of the present invention or a pharmaceutical compositions comprising the TROP2 binders or ADCs of the present invention may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid, which penetrates the epidermis to introduce the TROP2 binder or ADC of the present invention or a pharmaceutical composition comprising the TROP2 binder or ADC of the present invention to a patient's body. External infusion pumps are medical devices that deliver the TROP2 binder or ADC of the present invention or a pharmaceutical composition comprising the TROP2 binder or ADC of the present invention into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the clastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### Kits Comprising the TROP2 Binder or ADC or a Composition of the Present Invention or Composition Thereof

Further provided are kits comprising one or more components that include, but are not limited to, a TROP2 binder or ADC of the present invention or a pharmaceutical composition comprising the TROP2 binder or ADC of the present invention in association with one or more additional components including, but not limited to, a further therapeutic agent, as discussed herein. The TROP2 binder or ADC of the present invention or a pharmaceutical composition comprising the TROP2 binder or ADC of the present invention and/or the therapeutic agent can be formulated as a pure composition or in combination with a pharmaceutically acceptable carrier, in a pharmaceutical composition.

In one embodiment, the kit includes the TROP2 binder or ADC of the present invention or a pharmaceutical composition comprising the TROP2 binder or ADC of the present invention in one container (e.g., in a sterile glass or plastic vial) and a further therapeutic agent in another container (e.g., in a sterile glass or plastic vial).

In another embodiment, the kit comprises a combination of the invention, including TROP2 binder or ADC of the present invention or a pharmaceutical composition comprising the TROP2 binder or ADC of the present invention in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above. Thus, the present invention includes a kit comprising an injection device and the TROP2 binder or ADC of the present invention or a pharmaceutical composition comprising the TROP2 binder or ADC of the present invention, e.g., wherein the injection device includes the TROP2 binder or ADC of the present invention or a pharmaceutical composition comprising the TROP2 binder or ADC of the present invention, or wherein the TROP2 binder or ADC of the present invention or pharmaceutical composition comprising the TROP2 binder or ADC of the present invention is in a separate vessel.

The kit can include a package insert including information concerning the pharmaceutical composition and dosage form in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### Additional Specific Embodiments of the Present Invention Include the Following.

Embodiment 1. An antibody drug conjugate (ADC) comprising an antibody or antigen-binding fragment thereof that specifically binds to human TROP2 conjugated to a linker-monomethylauristatin E (linker-MMAE) payload, wherein the antibody comprises two heavy chains, each heavy chain comprising a variable domain and a constant domain, the variable domain comprising a complementarity determining region (CDR) H1, a CDRH2, and a CDRH3, and two light chains, each light chain comprising a variable domain and a constant domain, the variable domain comprising a CDRL1, a CDRL2, and a CDRL3, wherein the CDRH1, CDRH2, CDRH3, CDRL1, CDRL2, and CDRL3, respectively, comprise the amino acid sequence of NYGMN (SEQ ID NO: 4), WINTYTGEPTYTDDFKG (SEQ ID NO: 5), GGFGSSYWYFDV (SEQ ID NO: 6), KASQDVSIAVA (SEQ ID NO: 7), SASDRYT (SEQ ID NO: 10), and QQHYITPLT (SEQ ID NO: 9).

Embodiment 2. The ADC of embodiment 1, wherein the antibody or antigen-binding fragment thereof displays reduced binding to low TROP2-expressing cells compared to high TROP2-expressing cells and has reduced hydrophobicity compared to Sacituzumab as determined by hydrophobic interaction chromatography (HIC).

Embodiment 3. The ADC of embodiment 1, wherein the antibody or antigen-binding fragment thereof is humanized.

Embodiment 4. The ADC of embodiment 1, wherein the antigen binding fragment of the antibody is a Fab fragment, a Fab′ fragment, or a F(ab′)2 fragment.

Embodiment 5. The ADC of embodiment 1, wherein the heavy chain variable domain comprises the amino acid sequence of SEQ ID NO: 1 or 14 and the light chain variable domain comprises the amino acid sequence of SEQ ID NO: 3 or 16.

Embodiment 6. The ADC of embodiment 5, wherein the heavy chain variable domain comprises the amino acid sequence of SEQ ID NO: 1 and the light chain variable domain comprises the amino acid sequence of SEQ ID NO: 3 or the heavy chain variable domain comprises the amino acid sequence of SEQ ID NO: 14 and the light chain variable domain comprises the amino acid sequence of SEQ ID NO: 16.

Embodiment 7. The ADC of embodiment 1, wherein the antibody comprises a light chain comprising the amino acid sequence of SEQ ID NO: 13 or 22 and a heavy chain comprising the amino acid sequence of SEQ ID NO: 11, 17, or 18.

Embodiment 8. The ADC of embodiment 7, wherein the antibody comprises (a) a light chain comprising the amino acid sequence of SEQ ID NO: 13 and a heavy chain comprising the amino acid sequence of SEQ ID NO: 11; (b) a light chain comprising the amino acid sequence of SEQ ID NO: 22 and a heavy chain comprising the amino acid sequence of SEQ ID NO: 17; or (c) a light chain comprising the amino acid sequence of SEQ ID NO: 22 and a heavy chain comprising the amino acid sequence of SEQ ID NO: 18.

Embodiment 9. The ADC of embodiment 1, wherein the antibody comprises a light chain comprising the amino acid sequence of SEQ ID NO: 13 or 22 and a heavy chain comprising the amino acid sequence of SEQ ID NO: 58, 59, or 60.

Embodiment 10. The ADC of embodiment 9, wherein the antibody comprises (a) a light chain comprising the amino acid sequence of SEQ ID NO: 13 and a heavy chain comprising the amino acid sequence of SEQ ID NO: 58; (b) a light chain comprising the amino acid sequence of SEQ ID NO: 22 and a heavy chain comprising the amino acid sequence of SEQ ID NO: 59; or (c) a light chain comprising the amino acid sequence of SEQ ID NO: 22 and a heavy chain comprising the amino acid sequence of SEQ ID NO: 60.

Embodiment 11. The ADC of embodiment 1, wherein the antibody further comprises a cysteine or a non-canonical amino acid amino acid substitution at one or more position(s) selected from the group consisting of: positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

Embodiment 12. The ADC of embodiment 11, wherein the antibody comprises a cysteine or a non-canonical amino acid amino acid substitution at position 375 of the constant domain of the heavy chain.

Embodiment 13. The ADC of embodiment 12, wherein the antibody comprises a heavy chain comprising the amino acid sequence of SEQ ID NO: 19, 20, 61, or 62.

Embodiment 14. The ADC of any one of embodiments 11 to 13, wherein the linker-MMAE payload is conjugated to the cysteine or noncanonical amino acid.

Embodiment 15. The ADC of embodiment 1, wherein the antibody comprises a cysteine residue in which the thiol (SH) group thereof is conjugated to a linker-MMAE payload comprising the formula:

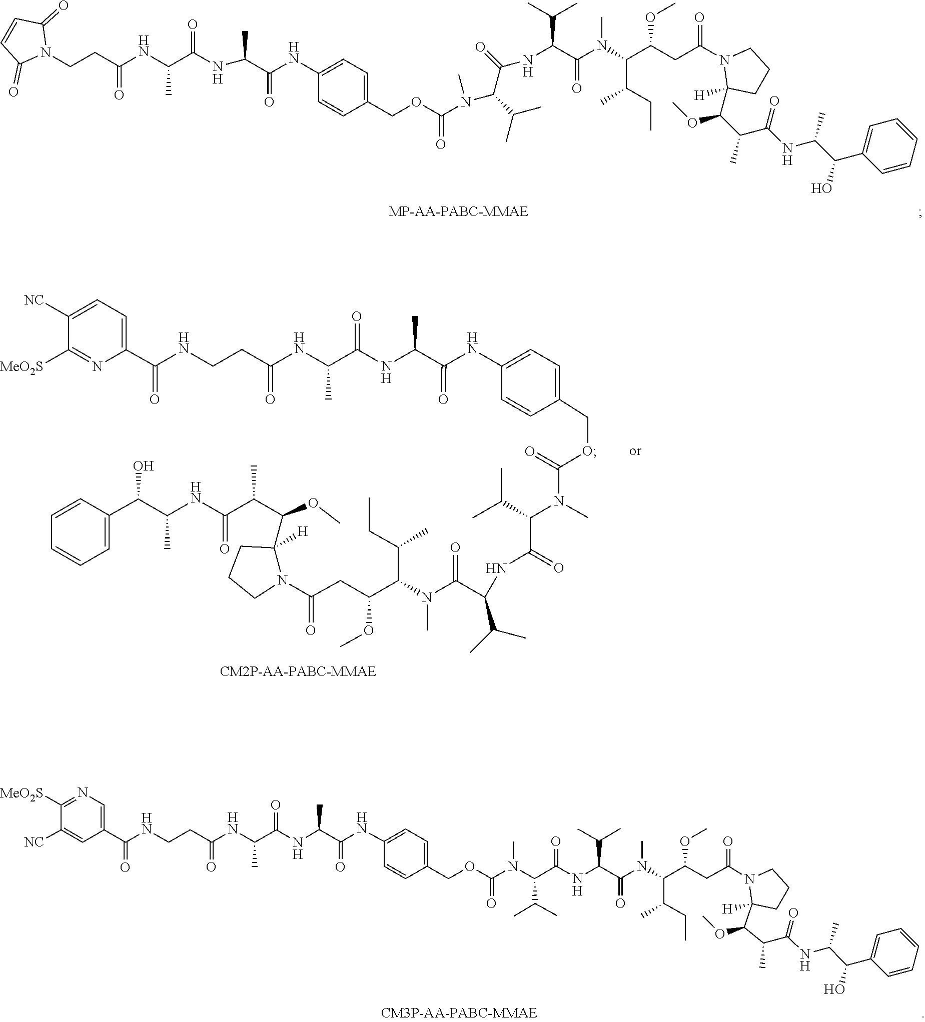

Embodiment 16. The ADC of embodiment 1, wherein the ADC comprises the formula:

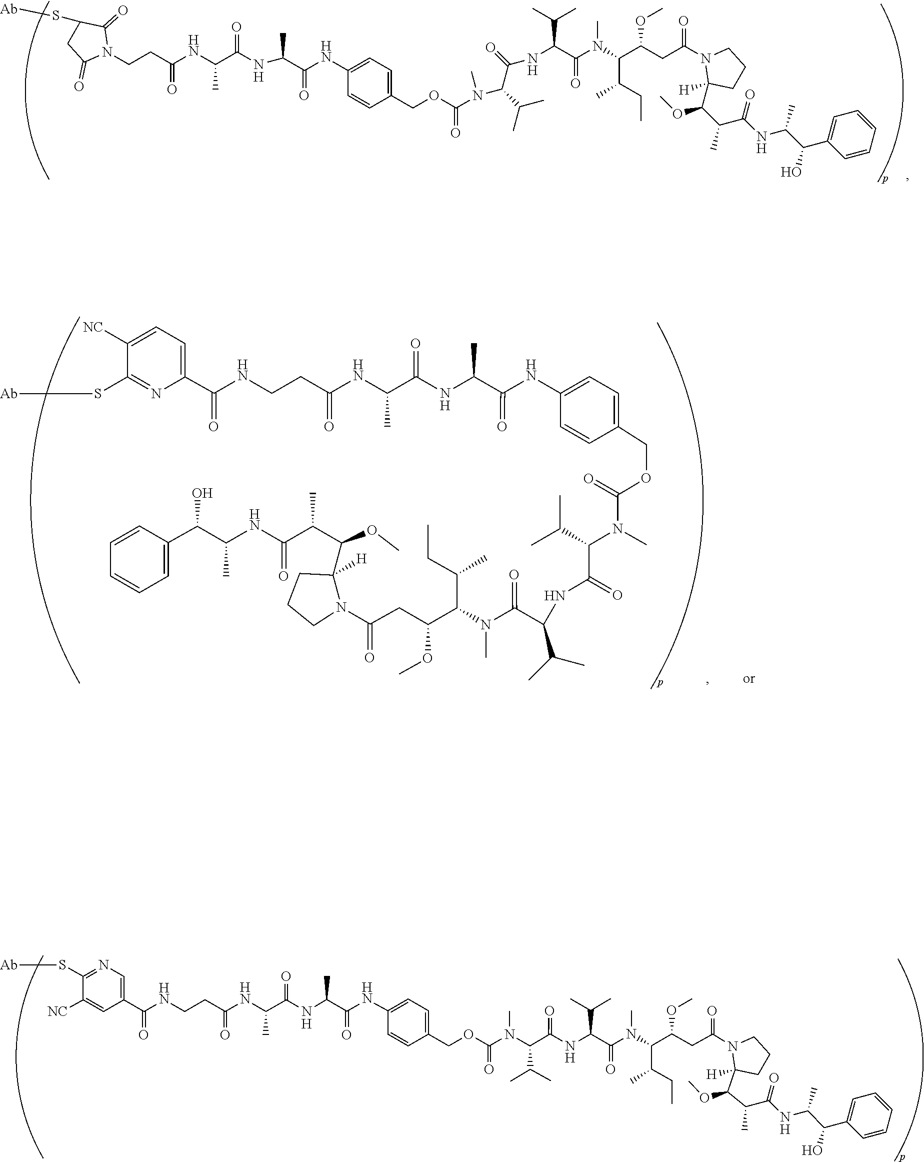

wherein Ab is an anti-TROP2 antibody; and p, is an integer from 1 to 8, wherein S is from the side chain of a cysteine residue of the antibody.

Embodiment 17. The ADC of embodiment 1, wherein the ADC comprises the formula:

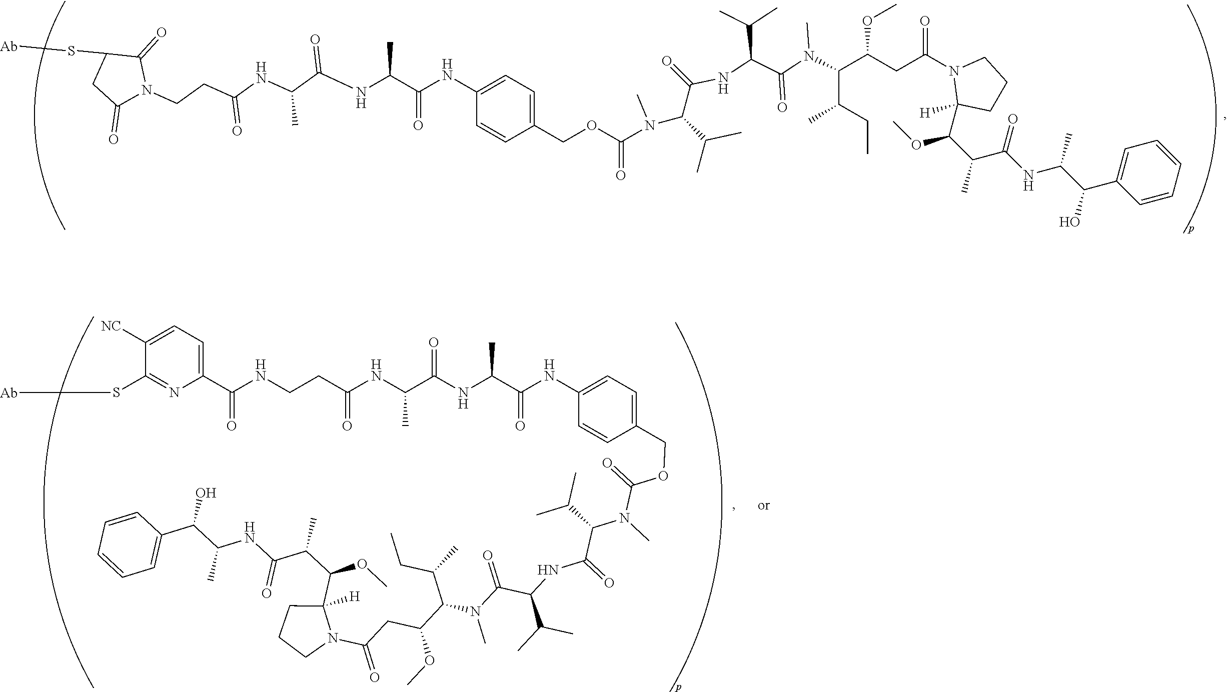

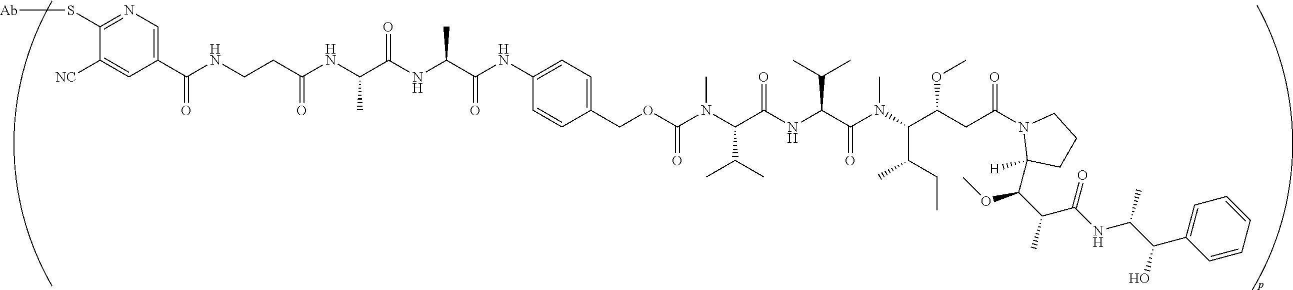

wherein Ab is an anti-TROP2 antibody comprising heavy chain engineered cysteine residues or light chain engineered cysteine residues, wherein the anti-TROP2 antibody comprising the engineered cysteine residues is (A) selected from the group consisting of:
(a) αTROP2 (HC:Sac-E152C) (LC:Sac-Y53D) comprising two heavy chains having the amino acid sequence set forth in SEQ ID No: 64 and two light chains having the amino acid sequence set forth in SEQ ID NO: 13;
(b) αTROP2 (HC:Sac-E153C) (LC:Sac-Y53D) two heavy chains having the amino acid sequence set forth in SEQ ID No: 65 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 66 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 67 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 68 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 69 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 71 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 72 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 73 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 74 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 75 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 76 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 58 and two light chains having the amino acid sequence set forth in SEQ ID NO: 50;
(n) αTROP2 (HC:Sac) (LC:Sac-Y53D-E168C) comprising two heavy chains having the amino acid sequence set forth in SEQ ID No: 58 and two light chains having the amino acid sequence set forth in SEQ ID NO: 51;
(o) αTROP2 (HC:Sac-YTE) (LC:Sac-Y53D-E165C) comprising two heavy chains having the amino acid sequence set forth in SEQ ID No: 63 and two light chains having the amino acid sequence set forth in SEQ ID NO: 50;
(p) αTROP2 (HC:Sac-YTE) (LC:Sac-Y53D-E168C) comprising two heavy chains having the amino acid sequence set forth in SEQ ID No: 63 and two light chains having the amino acid sequence set forth in SEQ ID NO: 51;
(q) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 21;
(r) αTROP2 (HC:BSM-E152C) (LC:BSM-Y53D) comprising two heavy chains having the amino acid sequence set forth in SEQ ID No: 78 and two light chains having the amino acid sequence set forth in SEQ ID NO: 22;
(s) αTROP2 (HC:BSM-E153C) (LC:BSM-Y53D) comprising two heavy chains having the amino acid sequence set forth in SEQ ID No: 79 and two light chains having the amino acid sequence set forth in SEQ ID NO: 22;
(t) αTROP2 (HC:BSM-E171C) (LC:BSM-Y53D) comprising two heavy chains having the amino acid sequence set forth in SEQ ID No: 80 and two light chains having the amino acid sequence set forth in SEQ ID NO: 22;
(u) αTROP2 (HC:BSM-E172C) (LC:BSM-Y53D) comprising two heavy chains having the amino acid sequence set forth in SEQ ID No: 81 and two light chains having the amino acid sequence set forth in SEQ ID NO: 22;
(v) αTROP2 (HC:BSM-E173C) (LC:BSM-Y53D) comprising two heavy chains having the amino acid sequence set forth in SEQ ID No: 82 and two light chains having the amino acid sequence set forth in SEQ ID NO: 22;
(w) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 22;
(x) αTROP2 (HC:BSM) (LC:BSM-Y53D-E165C) comprising two heavy chains having the amino acid sequence set forth in SEQ ID No: 59 and two light chains having the amino acid sequence set forth in SEQ ID NO: 54;
(y) αTROP2 (HC:BSM) (LC:BSM-Y53D-E168C) comprising two heavy chains having the amino acid sequence set forth in SEQ ID No: 59 and two light chains having the amino acid sequence set forth in SEQ ID NO: 55;
(z) αTROP2 (HC:BSM-YTE) (LC:BSM-Y53D-E165C) comprising two heavy chains having the amino acid sequence set forth in SEQ ID No: 60 and two light chains having the amino acid sequence set forth in SEQ ID NO: 54;
(aa) αTROP2 (HC:BSM-YTE) (LC:BSM-Y53D-E168C) comprising two heavy chains having the amino acid sequence set forth in SEQ ID No: 58 and two light chains having the amino acid sequence set forth in SEQ ID NO: 57;
(bb) αTROP2 (HC:BSM-YTE-E152C) (LC:BSM-Y53D) comprising two heavy chains having the amino acid sequence set forth in SEQ ID No: 85 and two light chains having the amino acid sequence set forth in SEQ ID NO: 22;
(cc) αTROP2 (HC:BSM-YTE-E153C) (LC:BSM-Y53D) comprising two heavy chains having the amino acid sequence set forth in SEQ ID No: 86 and two light chains having the amino acid sequence set forth in SEQ ID NO: 22;
(dd) αTROP2 (HC:BSM-YTE-E171C) (LC:BSM-Y53D) comprising two heavy chains having the amino acid sequence set forth in SEQ ID No: 87 and two light chains having the amino acid sequence set forth in SEQ ID NO: 22;
(ee) αTROP2 (HC:BSM-YTE-E172C) (LC:BSM-Y53D) comprising two heavy chains having the amino acid sequence set forth in SEQ ID No: 88 and two light chains having the amino acid sequence set forth in SEQ ID NO: 22;
(ff) αTROP2 (HC:BSM-YTE-E173C) (LC:BSM-Y53D) comprising two heavy chains having the amino acid sequence set forth in SEQ ID No: 89 and two light chains having the amino acid sequence set forth in SEQ ID NO: 22; and
(gg) αTROP2 (HC:BSM-YTE-S375C) (LC:BSM-Y53D) comprising two heavy chains having the amino acid sequence set forth in SEQ ID No: 90 and two light chains having the amino acid sequence set forth in SEQ ID NO: 22;
wherein S is from the side chain of the engineered cysteine residue; and, wherein p is an integer selected from 1 or 2; or (B) selected from the group consisting of:
(a) αTROP2 (HC:Sac-E152C-S375C) (LC:Sac-Y53D) comprising two heavy chains having the amino acid sequence set forth in SEQ ID No: 70 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 77 and two light chains having the amino acid sequence set forth in SEQ ID NO: 13;
(c) αTROP2 (HC:BSM-E152C-S375C) (LC:BSM-Y53D) comprising two heavy chains having the amino acid sequence set forth in SEQ ID No: 84 and two light chains having the amino acid sequence set forth in SEQ ID NO: 22; and
(d) αTROP2 (HC:BSM-YTE-E152C-S375C) (LC:BSM-Y53D) comprising two heavy chains having the amino acid sequence set forth in SEQ ID No: 91 and two light chains having the amino acid sequence set forth in SEQ ID NO: 22;
wherein S is from the side chain of the engineered cysteine residue; and, wherein p is an integer selected from 1, 2, 3, or 4.

Embodiment 18. The ADC of embodiment 1, wherein the ADC comprises the formula:

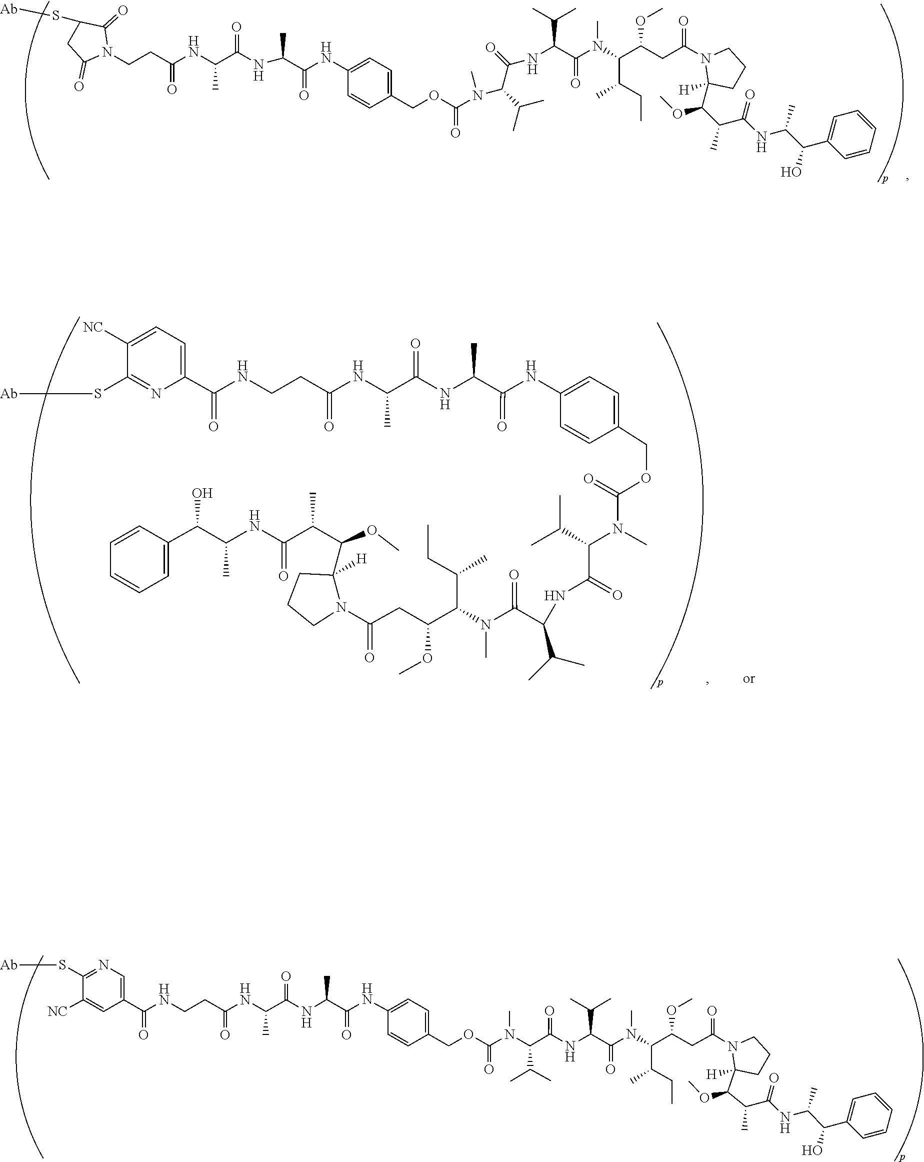

wherein Ab is an anti-TROP2 antibody comprising heavy chain engineered cysteine residues or light chain engineered cysteine residues, wherein the anti-TROP2 antibody comprising the engineered cysteine residues is (A) selected from the group consisting of:
(a) αTROP2 (HC:Sac-E152C) (LC:Sac-Y53D) comprising two heavy chains having the amino acid sequence set forth in SEQ ID No: 24 and two light chains having the amino acid sequence set forth in SEQ ID NO: 13;
(b) αTROP2 (HC:Sac-E153C) (LC:Sac-Y53D) comprising two heavy chains having the amino acid sequence set forth in SEQ ID No: 25 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 26 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 27 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 28 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 29 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 31 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 32 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 33 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 34 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 35 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 36 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 11 and two light chains having the amino acid sequence set forth in SEQ ID NO: 52;
(n) αTROP2 (HC:Sac) (LC:Sac-Y53D-E168C) comprising two heavy chains having the amino acid sequence set forth in SEQ ID No: 11 and two light chains having the amino acid sequence set forth in SEQ ID NO: 53;
(o) αTROP2 (HC:Sac-YTE) (LC:Sac-Y53D-E165C) comprising two heavy chains having the amino acid sequence set forth in SEQ ID No: 23 and two light chains having the amino acid sequence set forth in SEQ ID NO: 52;
(p) αTROP2 (HC:Sac-YTE) (LC:Sac-Y53D-E168C) comprising two heavy chains having the amino acid sequence set forth in SEQ ID No: 23 and two light chains having the amino acid sequence set forth in SEQ ID NO: 53;
(q) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 21;
(r) αTROP2 (HC:BSM-E152C) (LC:BSM-Y53D) comprising two heavy chains having the amino acid sequence set forth in SEQ ID No: 38 and two light chains having the amino acid sequence set forth in SEQ ID NO: 22;
(s) αTROP2 (HC:BSM-E153C) (LC:BSM-Y53D) comprising two heavy chains having the amino acid sequence set forth in SEQ ID No: 39 and two light chains having the amino acid sequence set forth in SEQ ID NO: 22;
(t) αTROP2 (HC:BSM-E171C) (LC:BSM-Y53D) comprising two heavy chains having the amino acid sequence set forth in SEQ ID No: 40 and two light chains having the amino acid sequence set forth in SEQ ID NO: 22;
(u) αTROP2 (HC:BSM-E172C) (LC:BSM-Y53D) comprising two heavy chains having the amino acid sequence set forth in SEQ ID No: 41 and two light chains having the amino acid sequence set forth in SEQ ID NO: 22;
(v) αTROP2 (HC:BSM-E173C) (LC:BSM-Y53D) comprising two heavy chains having the amino acid sequence set forth in SEQ ID No: 42 and two light chains having the amino acid sequence set forth in SEQ ID NO: 22;
(w) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 22;
(x) αTROP2 (HC:BSM) (LC:BSM-Y53D-E168C) comprising two heavy chains having the amino acid sequence set forth in SEQ ID No: 17 and two light chains having the amino acid sequence set forth in SEQ ID NO: 55;
(y) αTROP2 (HC:BSM-YTE) (LC:BSM-Y53D-E165C) comprising two heavy chains having the amino acid sequence set forth in SEQ ID No: 18 and two light chains having the amino acid sequence set forth in SEQ ID NO: 54;
(z) αTROP2 (HC:BSM-YTE) (LC:BSM-Y53D-E168C) comprising two heavy chains having the amino acid sequence set forth in SEQ ID No: 18 and two light chains having the amino acid sequence set forth in SEQ ID NO: 55;
(aa) αTROP2 (HC:BSM-YTE-E152C) (LC:BSM-Y53D) comprising two heavy chains having the amino acid sequence set forth in SEQ ID No: 44 and two light chains having the amino acid sequence set forth in SEQ ID NO: 22;
(bb) αTROP2 (HC:BSM-YTE-E153C) (LC:BSM-Y53D) comprising two heavy chains having the amino acid sequence set forth in SEQ ID No: 45 and two light chains having the amino acid sequence set forth in SEQ ID NO: 22;
(cc) αTROP2 (HC:BSM-YTE-E171C) (LC:BSM-Y53D) comprising two heavy chains having the amino acid sequence set forth in SEQ ID No: 46 and two light chains having the amino acid sequence set forth in SEQ ID NO: 22;
(dd) αTROP2 (HC:BSM-YTE-E172C) (LC:BSM-Y53D) comprising two heavy chains having the amino acid sequence set forth in SEQ ID No: 47 and two light chains having the amino acid sequence set forth in SEQ ID NO: 22;
(ee) αTROP2 (HC:BSM-YTE-E173C) (LC:BSM-Y53D) comprising two heavy chains having the amino acid sequence set forth in SEQ ID No: 48 and two light chains having the amino acid sequence set forth in SEQ ID NO: 22; and
(ff) αTROP2 (HC:BSM-YTE-S375C) (LC:BSM-Y53D) comprising two heavy chains having the amino acid sequence set forth in SEQ ID No: 20 and two light chains having the amino acid sequence set forth in SEQ ID NO: 22;
wherein S is from the side chain of the engineered cysteine residue; and, wherein p is an integer selected from 1 or 2; or (B) selected from the group consisting of:
(a) αTROP2 (HC:Sac-E152C-S375C) (LC:Sac-Y53D) comprising two heavy chains having the amino acid sequence set forth in SEQ ID No: 30 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 37 and two light chains having the amino acid sequence set forth in SEQ ID NO: 13;
(c) αTROP2 (HC:BSM-E152C-S375C) (LC:BSM-Y53D) comprising two heavy chains having the amino acid sequence set forth in SEQ ID No: 43 and two light chains having the amino acid sequence set forth in SEQ ID NO: 22; and
(d) αTROP2 (HC:BSM-YTE-E152C-S375C) (LC:BSM-Y53D) comprising two heavy chains having the amino acid sequence set forth in SEQ ID No: 49 and two light chains having the amino acid sequence set forth in SEQ ID NO: 22;
wherein S is from the side chain of the engineered cysteine residue; and, wherein p is an integer selected from 1, 2, 3, or 4.

Embodiment 19. The ADC of embodiment 1, wherein the ADC comprises the

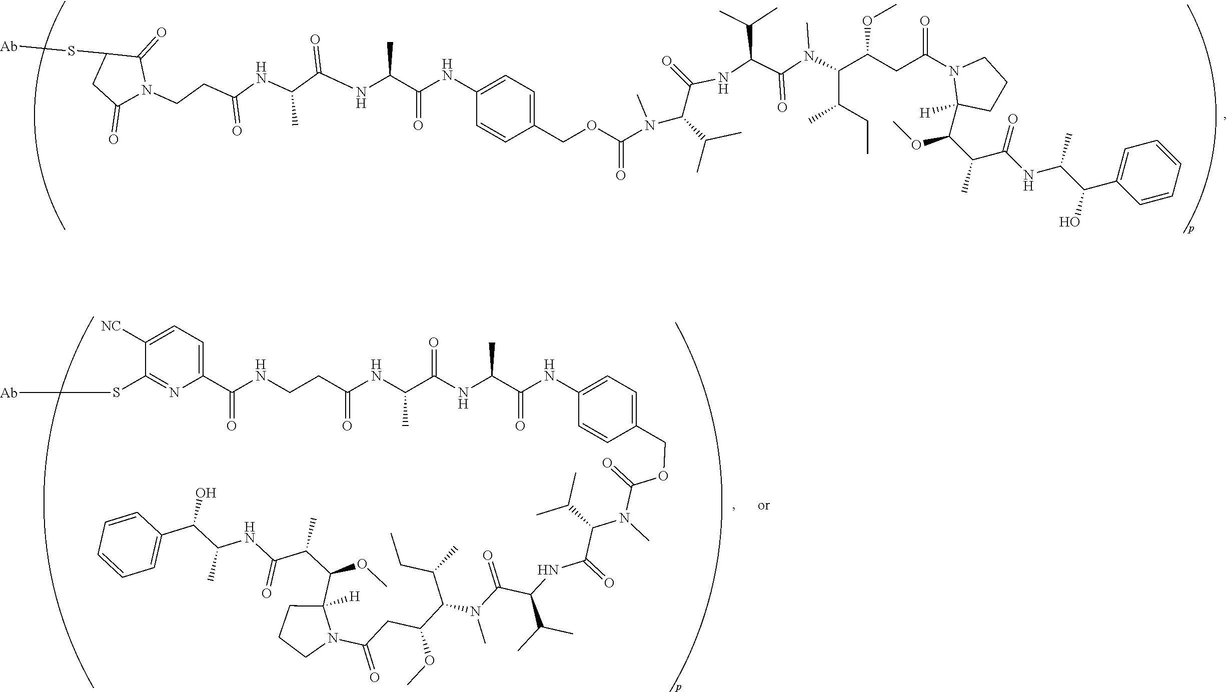

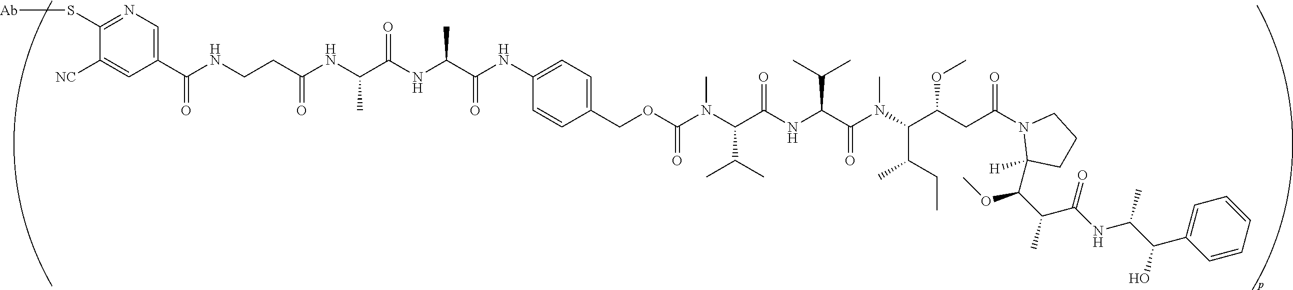

wherein Ab is an anti-Trop2 antibody comprising two heavy chains having the amino acid sequence set forth in SEQ ID NO: 20 and two light chains having the amino acid sequence set forth in SEQ ID NO: 22; wherein p is 1 or 2; and wherein S is from the side chain of an engineered cysteine residue at position 375 of the constant domain of the heavy chain, the position as defined according to Eu numbering.

Embodiment 20. The ADC of embodiment 1, wherein the ADC comprises the formula:

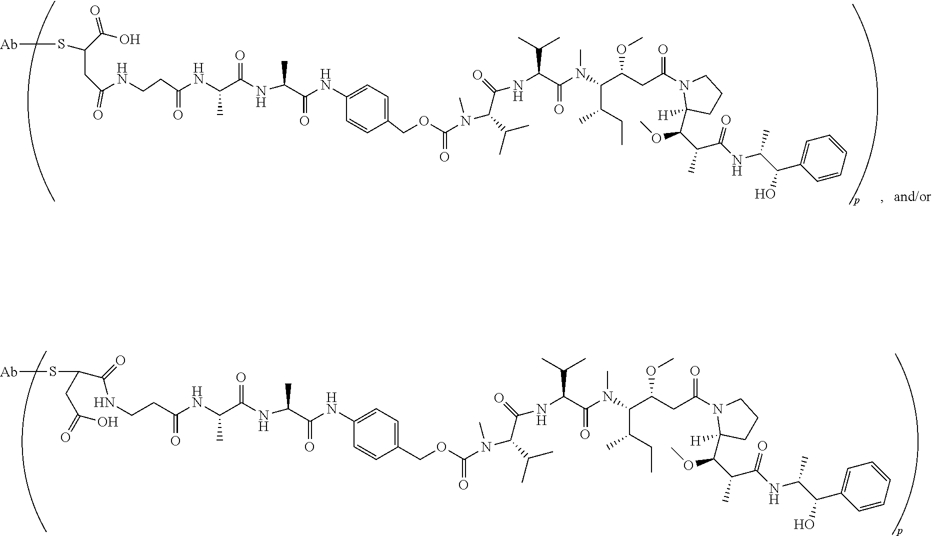

wherein Ab is an anti-TROP2 antibody; wherein p is 1 or 2; and wherein S is from the side chain of an engineered cysteine residue at position 375 of the constant domain of the heavy chain, the position as defined according to Eu numbering.

Embodiment 21. A composition comprising the ADC of any one of embodiments 1 to 20 and a pharmaceutically acceptable carrier.

Embodiment 22. The composition of embodiment 21, wherein the predominant ADC species in the composition comprises (i) antibodies in which the heavy chain C-terminus lacks a lysine residue; (ii) antibodies in which the heavy chain N-terminus is glutamine, glutamic acid, or pyroglutamate; or, (iii) antibodies in which the heavy chain C-terminus lacks a lysine residue and the heavy chain N-terminus is glutamine, glutamic acid, or pyroglutamate.

Embodiment 23. A method for treating a cancer in an individual in need thereof comprising administering to the individual a therapeutically effective amount of the ADC of any one of embodiments 1 to 20 or the composition of embodiment 21 or embodiment 22 to treat the cancer, wherein the cancer is a cancer that overexpresses TROP2.

Embodiment 24. The method of embodiment 23, wherein the cancer is selected from the group consisting of breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 25. Use of an ADC of any one of embodiments 1 to 20 or the composition of embodiment 21 or 22 for the manufacture of a medicament for treatment of a cancer that overexpresses TROP2.

Embodiment 26. The use of embodiment 25, wherein the cancer is selected from the group consisting of breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 27. The ADC of any one of embodiments 1 to 20 or the composition of embodiment 21 or embodiment 22 for treatment of a cancer that overexpresses TROP2.

Embodiment 28. The ADC of embodiment 27, wherein the cancer is selected from the group consisting of breast cancer (e.g., triple negative breast cancer), cervix cancer, lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 29. A combination therapy for treating cancer comprising the ADC of any one of embodiments 1 to 20 or the composition of embodiment 21 or embodiment 22 and a therapeutic agent, wherein the cancer is a cancer that overexpresses TROP2.

Embodiment 30. The combination therapy of embodiment 29, wherein the therapeutic agent is a chemotherapy agent or a therapeutic antibody.

Embodiment 31. The combination therapy of embodiment 30, wherein the therapeutic antibody is a checkpoint inhibitor.

Embodiment 32. The combination therapy of embodiment 31, wherein the therapeutic antibody is an anti-PD1 antibody or an anti-PD-L1 antibody.

Embodiment 33. The combination therapy of embodiment 29, wherein the cancer is selected from the group consisting of breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 34. An antibody-drug conjugate (ADC) comprising an antibody that specifically binds to human TROP2 conjugated to a linker-monomethylauristatin E (linker-MMAE) payload, wherein the antibody comprises two heavy chains, each heavy chain comprising a variable domain and a constant domain, the variable domain comprising the amino acid sequence of SEQ ID NO: 14, and two light chains, each light chain comprising a variable domain and a constant domain, the variable domain comprising the amino acid sequence of SEQ ID NO: 15.

Embodiment 35. The ADC of embodiment 34, wherein the antibody displays reduced hydrophobicity compared to Sacituzumab as determined by hydrophobic interaction chromatography (HIC).

Embodiment 36. The ADC of embodiment 34, wherein the antibody further comprises a cysteine or a non-canonical amino acid amino acid substitution at one or more position(s) selected from the group consisting of: positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

Embodiment 37. The ADC of embodiment 36, wherein the antibody comprises a cysteine or a non-canonical amino acid amino acid substitution at position 375 of the constant domain of the heavy chain.

Embodiment 38. The ADC of embodiment 34, wherein the antibody comprises a heavy chain comprising the amino acid sequence of SEQ ID NO: 17, 18, 19, 20, 59, 60, or 61 and a light chain comprising the amino acid sequence of SEQ ID NO: 21.

Embodiment 39. The ADC of embodiment 36, wherein the linker-MMAE payload is conjugated to the cysteine or noncanonical amino acid.

Embodiment 40. The ADC of embodiment 34, wherein the antibody comprises a cysteine residue in which the thiol (SH) group thereof is conjugated to a linker-MMAE payload comprising the formula:

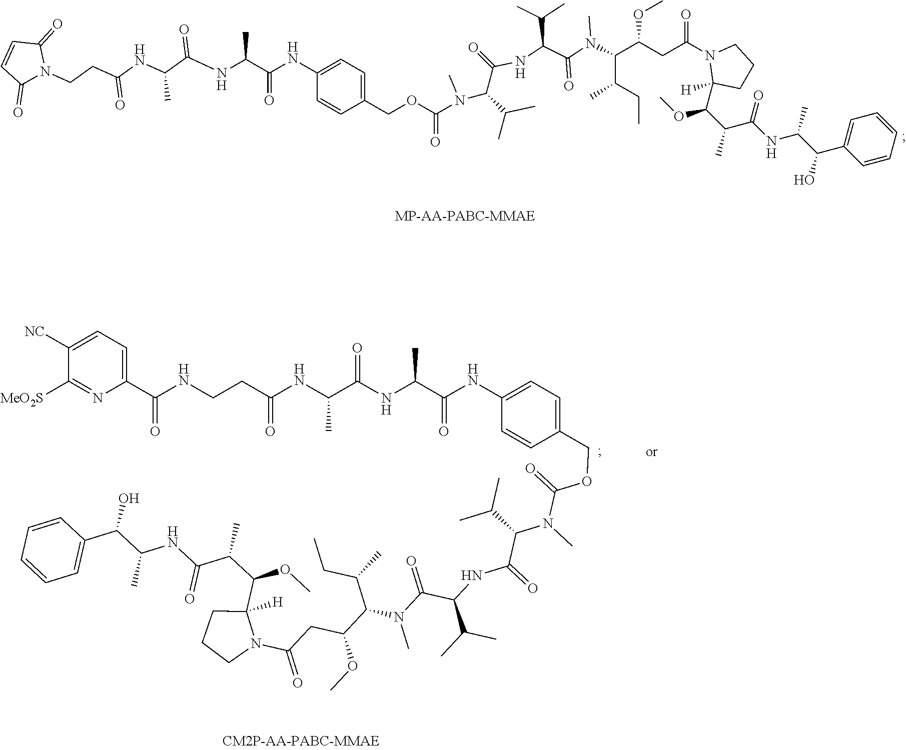 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2ccc(C#N)c(S(C)(=O)=O)n2)cc1)C(C)C]

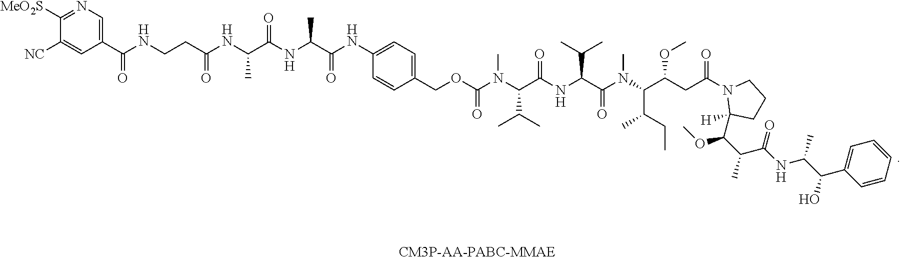 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2cnc(S(C)(=O)=O)c(C#N)c2)cc1)C(C)C]

Embodiment 41. The ADC of embodiment 34, wherein the ADC comprises the

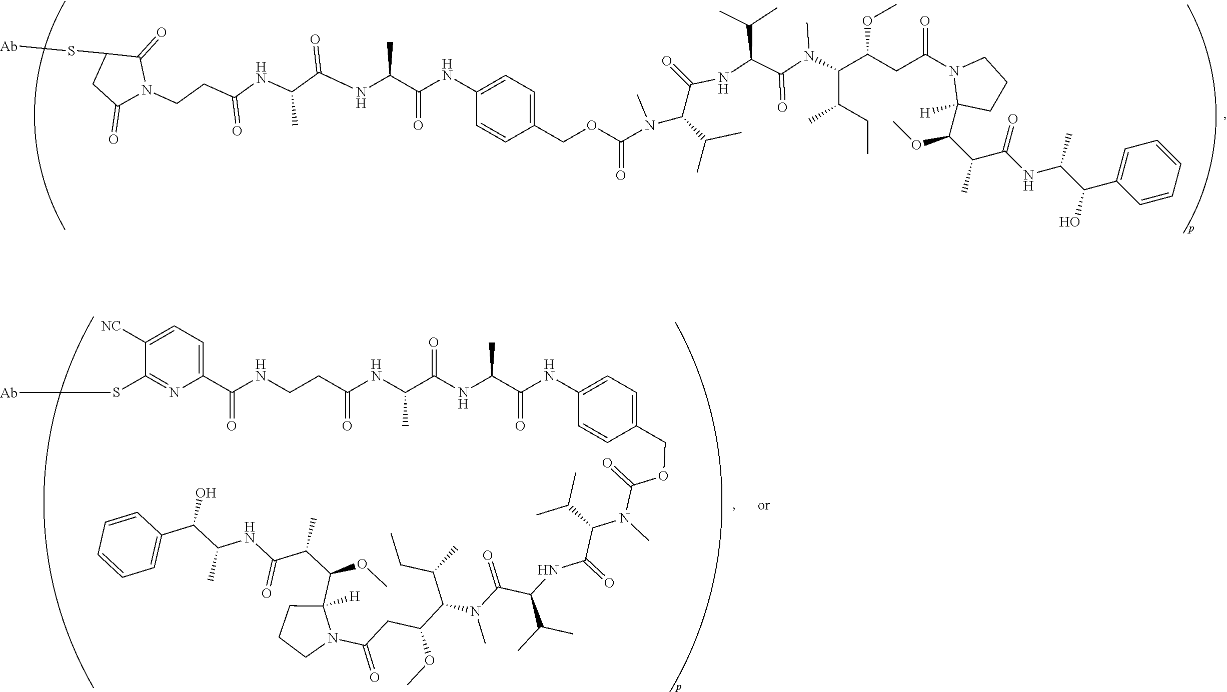

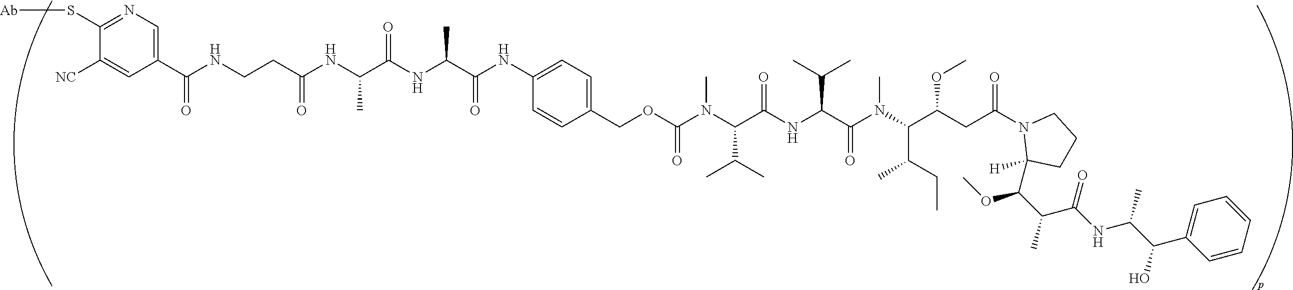

wherein Ab is the antibody; and p, is an integer from 1 to 8, wherein S is from the side chain of a cysteine residue of the antibody.

Embodiment 42. The ADC of embodiment 34, wherein the ADC comprises the formula:

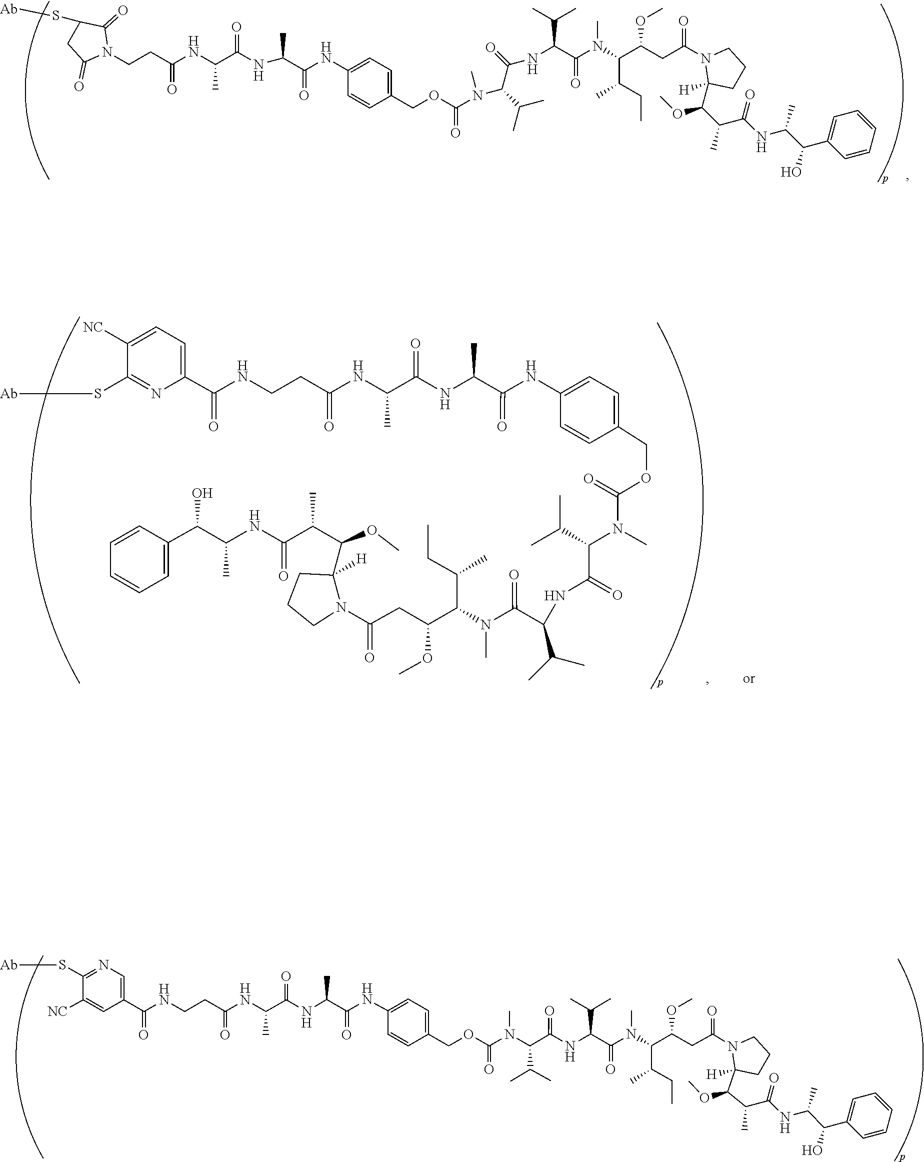

wherein Ab is the antibody, wherein the antibody comprises a heavy chain engineered cysteine residues or light chain engineered cysteine residues, wherein the antibody comprising the engineered cysteine residues is: (A) selected from the group consisting of:
(a) αTROP2 (HC:BSM-E152C) (LC:BSM) comprising two heavy chains having the amino acid sequence set forth in SEQ ID No: 78 and two light chains having the amino acid sequence set forth in SEQ ID NO: 21;
(b) αTROP2 (HC:BSM-E153C) (LC:BSM) comprising two heavy chains having the amino acid sequence set forth in SEQ ID No: 79 and two light chains having the amino acid sequence set forth in SEQ ID NO: 21;
(c) αTROP2 (HC:BSM-E171C) (LC:BSM) comprising two heavy chains having the amino acid sequence set forth in SEQ ID No: 80 and two light chains having the amino acid sequence set forth in SEQ ID NO: 21;
(d) αTROP2 (HC:BSM-E172C) (LC:BSM) comprising two heavy chains having the amino acid sequence set forth in SEQ ID No: 81 and two light chains having the amino acid sequence set forth in SEQ ID NO: 21;
(e) αTROP2 (HC:BSM-E173C) (LC:BSM) comprising two heavy chains having the amino acid sequence set forth in SEQ ID No: 82 and two light chains having the amino acid sequence set forth in SEQ ID NO: 21;
(f) αTROP2 (HC:BSM) (LC:BSM-E165C) comprising two heavy chains having the amino acid sequence set forth in SEQ ID No: 59 and two light chains having the amino acid sequence set forth in SEQ ID NO: 52;
(g) αTROP2 (HC:BSM) (LC:BSM-E168C) comprising two heavy chains having the amino acid sequence set forth in SEQ ID No: 59 and two light chains having the amino acid sequence set forth in SEQ ID NO: 53;
(h) αTROP2 (HC:BSM-YTE) (LC:BSM-E165C) comprising two heavy chains having the amino acid sequence set forth in SEQ ID No: 60 and two light chains having the amino acid sequence set forth in SEQ ID NO: 52;
(i) αTROP2 (HC:BSM-YTE) (LC:BSM-E168C) comprising two heavy chains having the amino acid sequence set forth in SEQ ID No: 60 and two light chains having the amino acid sequence set forth in SEQ ID NO: 53;
(j) αTROP2 (HC:BSM-YTE-E152C) (LC:BSM) comprising two heavy chains having the amino acid sequence set forth in SEQ ID No: 85 and two light chains having the amino acid sequence set forth in SEQ ID NO: 21;
(k) αTROP2 (HC:BSM-YTE-E153C) (LC:BSM) comprising two heavy chains having the amino acid sequence set forth in SEQ ID No: 86 and two light chains having the amino acid sequence set forth in SEQ ID NO: 21;
(l) αTROP2 (HC:BSM-YTE-E171C) (LC:BSM) comprising two heavy chains having the amino acid sequence set forth in SEQ ID No: 87 and two light chains having the amino acid sequence set forth in SEQ ID NO: 21;
(m) αTROP2 (HC:BSM-YTE-E172C) (LC:BSM) comprising two heavy chains having the amino acid sequence set forth in SEQ ID No: 88 and two light chains having the amino acid sequence set forth in SEQ ID NO: 21;
(n) αTROP2 (HC:BSM-YTE-E173C) (LC:BSM) comprising two heavy chains having the amino acid sequence set forth in SEQ ID No: 89 and two light chains having the amino acid sequence set forth in SEQ ID NO: 21; and
(o) αTROP2 (HC:BSM-YTE-S375C) (LC:BSM) comprising two heavy chains having the amino acid sequence set forth in SEQ ID No: 90 and two light chains having the amino acid sequence set forth in SEQ ID NO: 21;
wherein S is from the side chain of the engineered cysteine residue; and, wherein p is an integer selected from 1, 2, 3, or 4; or (B) selected from the group consisting of:
(a) αTROP2 (HC:BSM-E152C-S375C) (LC:BSM) comprising two heavy chains having the amino acid sequence set forth in SEQ ID No: 84 and two light chains having the amino acid sequence set forth in SEQ ID NO: 21; and
(b) αTROP2 (HC:BSM-YTE-E152C-S375C) (LC:BSM) comprising two heavy chains having the amino acid sequence set forth in SEQ ID No: 91 and two light chains having the amino acid sequence set forth in SEQ ID NO: 21; and
wherein S is from the side chain of the engineered cysteine residue; and, wherein p is an integer selected from 1, 2, 3, or 4.

Embodiment 43. The ADC of embodiment 34, wherein the ADC comprises the formula:

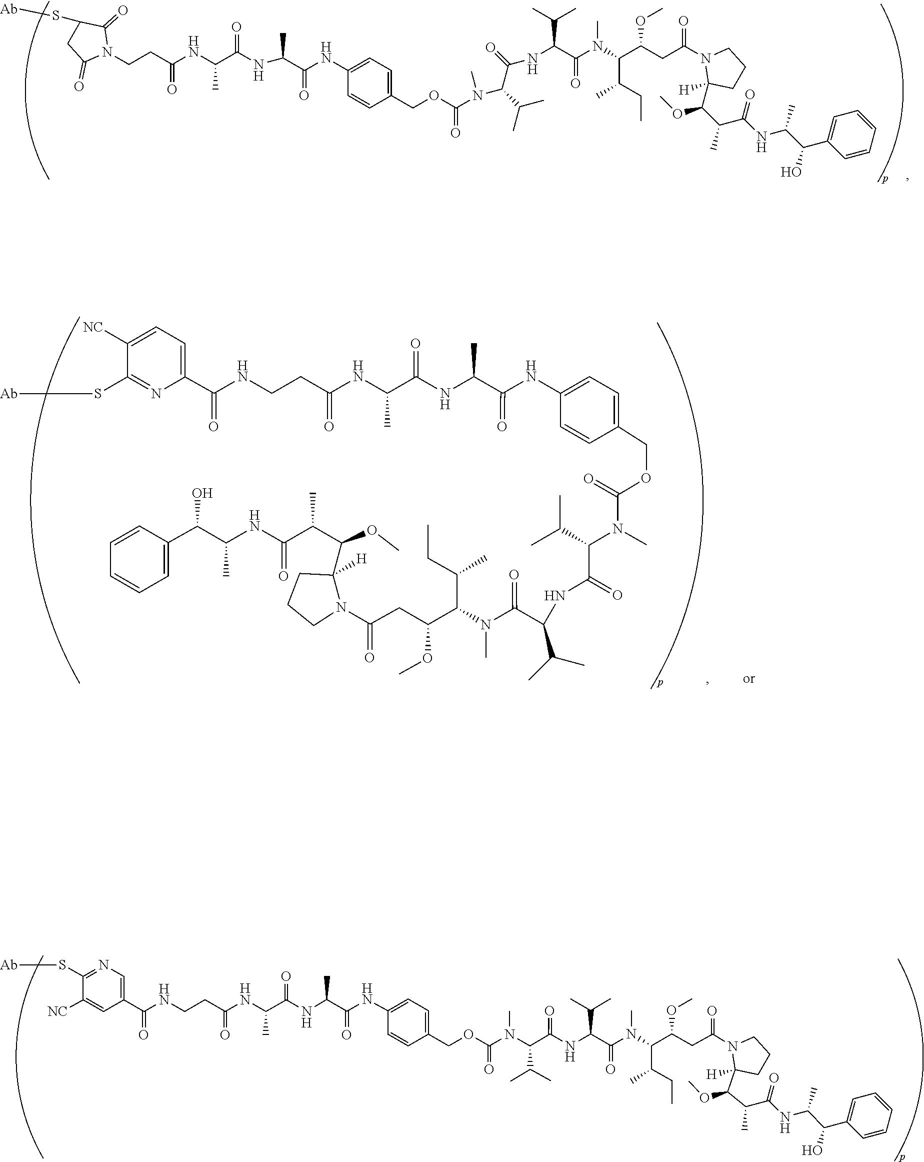

wherein Ab is the antibody, wherein the antibody comprises a heavy chain engineered cysteine residues or light chain engineered cysteine residues, wherein the antibody comprising the engineered cysteine residues is: (A) selected from the group consisting of:
(a) αTROP2 (HC:BSM-E152C) (LC:BSM) comprising two heavy chains having the amino acid sequence set forth in SEQ ID No: 38 and two light chains having the amino acid sequence set forth in SEQ ID NO: 21;
(b) αTROP2 (HC:BSM-E153C) (LC:BSM) comprising two heavy chains having the amino acid sequence set forth in SEQ ID No: 39 and two light chains having the amino acid sequence set forth in SEQ ID NO: 21;
(c) αTROP2 (HC:BSM-E171C) (LC:BSM) comprising two heavy chains having the amino acid sequence set forth in SEQ ID No: 40 and two light chains having the amino acid sequence set forth in SEQ ID NO: 21;
(d) αTROP2 (HC:BSM-E172C) (LC:BSM) comprising two heavy chains having the amino acid sequence set forth in SEQ ID No: 41 and two light chains having the amino acid sequence set forth in SEQ ID NO: 21;
(e) αTROP2 (HC:BSM-E173C) (LC:BSM) comprising two heavy chains having the amino acid sequence set forth in SEQ ID No: 42 and two light chains having the amino acid sequence set forth in SEQ ID NO: 21;
(f) αTROP2 (HC:BSM) (LC:BSM-E165C) comprising two heavy chains having the amino acid sequence set forth in SEQ ID No: 17 and two light chains having the amino acid sequence set forth in SEQ ID NO: 52;
(g) αTROP2 (HC:BSM) (LC:BSM-E168C) comprising two heavy chains having the amino acid sequence set forth in SEQ ID No: 17 and two light chains having the amino acid sequence set forth in SEQ ID NO: 53;
(h) αTROP2 (HC:BSM-YTE) (LC:BSM-E165C) comprising two heavy chains having the amino acid sequence set forth in SEQ ID No: 18 and two light chains having the amino acid sequence set forth in SEQ ID NO: 52;
(i) αTROP2 (HC:BSM-YTE) (LC:BSM-E168C) comprising two heavy chains having the amino acid sequence set forth in SEQ ID No: 18 and two light chains having the amino acid sequence set forth in SEQ ID NO: 53;
(j) αTROP2 (HC:BSM-YTE-E152C) (LC:BSM) comprising two heavy chains having the amino acid sequence set forth in SEQ ID No: 44 and two light chains having the amino acid sequence set forth in SEQ ID NO: 21;
(k) αTROP2 (HC:BSM-YTE-E153C) (LC:BSM) comprising two heavy chains having the amino acid sequence set forth in SEQ ID No: 45 and two light chains having the amino acid sequence set forth in SEQ ID NO: 21;
(l) αTROP2 (HC:BSM-YTE-E171C) (LC:BSM) comprising two heavy chains having the amino acid sequence set forth in SEQ ID No: 46 and two light chains having the amino acid sequence set forth in SEQ ID NO: 21;
(m) αTROP2 (HC:BSM-YTE-E172C) (LC:BSM) comprising two heavy chains having the amino acid sequence set forth in SEQ ID No: 47 and two light chains having the amino acid sequence set forth in SEQ ID NO: 21;
(n) αTROP2 (HC:BSM-YTE-E173C) (LC:BSM) comprising two heavy chains having the amino acid sequence set forth in SEQ ID No: 48 and two light chains having the amino acid sequence set forth in SEQ ID NO: 21; and
(o) αTROP2 (HC:BSM-YTE-S375C) (LC:BSM) comprising two heavy chains having the amino acid sequence set forth in SEQ ID No: 20 and two light chains having the amino acid sequence set forth in SEQ ID NO: 21;
wherein S is from the side chain of the engineered cysteine residue; and, wherein p is an integer selected from 1 or 2; or (B) selected from the group consisting of:
(a) αTROP2 (HC:BSM-E152C-S375C) (LC:BSM) comprising two heavy chains having the amino acid sequence set forth in SEQ ID No: 43 and two light chains having the amino acid sequence set forth in SEQ ID NO: 21; and
(b) αTROP2 (HC:BSM-YTE-E152C-S375C) (LC:BSM) comprising two heavy chains having the amino acid sequence set forth in SEQ ID No: 49 and two light chains having the amino acid sequence set forth in SEQ ID NO: 21;
wherein S is from the side chain of the engineered cysteine residue; and, wherein p is an integer selected from 1, 2, 3, or 4.

Embodiment 44. The ADC of embodiment 34, wherein the ADC comprises the formula

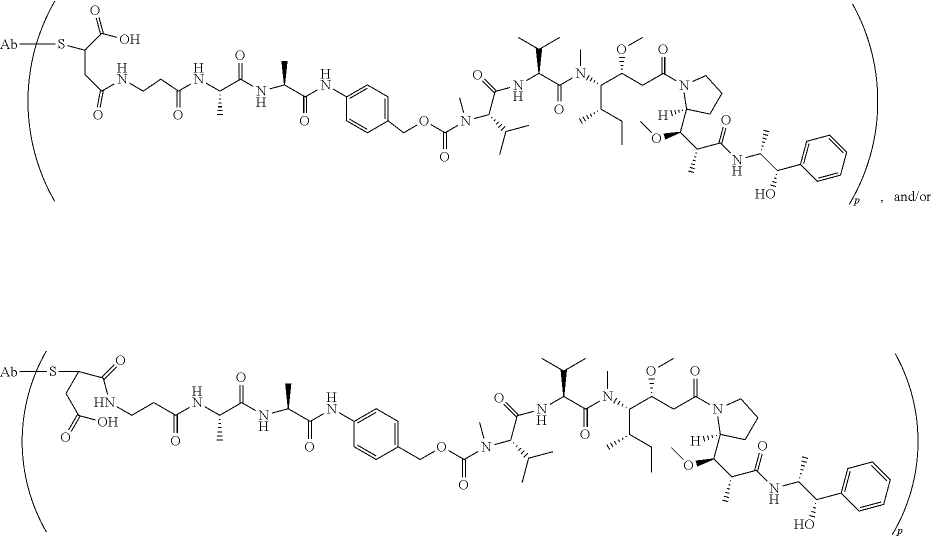

wherein Ab is the antibody; wherein p is 1 or 2; and wherein S is from the side chain of a cysteine residue of the antibody.

Embodiment 45. A composition comprising the ADC of any one of embodiments 34 to 44 and a pharmaceutically acceptable carrier.

Embodiment 46. The composition of embodiment 45, wherein the predominant ADC species in the composition comprises (i) antibodies in which the heavy chain C-terminus lacks a lysine residue; (ii) antibodies in which the heavy chain N-terminus is glutamine, glutamic acid, or pyroglutamate; or, (iii) antibodies in which the heavy chain C-terminus lacks a lysine residue and the heavy chain N-terminus is glutamine, glutamic acid, or pyroglutamate.

Embodiment 47. A method for treating a cancer in an individual in need thereof comprising administering to the individual a therapeutically effective amount of the ADC of any one of embodiments 34 to 44 or the composition of embodiment 45 or embodiment 46 to treat the cancer, wherein the cancer is a cancer that overexpresses TROP2.

Embodiment 48. The method of embodiment 47, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 49. Use of an ADC of any one of embodiments 34 to 44 or the composition of embodiment 45 or embodiment 46 for the manufacture of a medicament for the treatment of a cancer that overexpresses TROP2.

Embodiment 50. The use of embodiment 49, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 51. The ADC of any one of embodiments 34 to44 or the composition of embodiment 45 or embodiment 46 for the treatment of a cancer that overexpresses TROP2.

Embodiment 52. The ADC of embodiment 51, wherein the cancer is selected from the group consisting of: breast cancer (e.g., triple negative breast cancer), cervix cancer, lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 53. A combination therapy for treating cancer comprising the ADC of any one of embodiments 34 to 44 or the composition of embodiment 45 or embodiment 46 and a therapeutic agent, wherein the cancer is a cancer that overexpresses TROP2.

Embodiment 54. The combination therapy of embodiment 53 wherein the therapeutic agent is a chemotherapy agent or a therapeutic antibody.

Embodiment 55. The combination therapy of embodiment 54, wherein the therapeutic antibody is a checkpoint inhibitor.

Embodiment 56. The combination therapy of embodiment 55, wherein the therapeutic antibody is an anti-PD1 antibody or an anti-PD-L1 antibody.

Embodiment 57. The combination therapy of embodiment 53, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 58. An antibody or antigen binding fragment thereof that specifically binds to human TROP2, comprising: a heavy chain variable domain comprising a CDRH1, a CDRH2, and a CDRH3, and a light chain variable domain comprising a CDRL1, a CDRL2, and a CDRL3, wherein the CDRH1, CDRH2, CDRH3, CDRL1, CDRL2, and CDRL3, respectively, comprise the amino acid sequence NYGMN (SEQ ID NO: 4), WINTYTGEPTYTDDFKG (SEQ ID NO: 5), GGFGSSYWYFDV (SEQ ID NO: 6), KASQDVSIAVA (SEQ ID NO: 7), SASDRYT (SEQ ID NO: 10), and QQHYITPLT (SEQ ID NO: 9).

Embodiment 59. The antibody or antigen binding fragment thereof of embodiment 58, wherein the antibody or antigen binding fragment thereof displays reduced binding to low TROP2-expressing cells compared to high TROP2-expressing cells and has reduced hydrophobicity compared to Sacituzumab as determined by hydrophobic interaction chromatography (HIC).

Embodiment 60. The antibody or antigen binding fragment thereof of embodiment 58 or embodiment 59, wherein the antibody or antigen-binding fragment thereof is humanized.

Embodiment 61. The antibody or antigen binding fragment thereof of any one of embodiments 58 to 60, wherein the antigen binding fragment is a Fab fragment, a Fab′ fragment, or a F(ab′)2 fragment.

Embodiment 62. The antibody or antigen binding fragment thereof of any one of embodiments 58 to 61, wherein the heavy chain variable domain comprises the amino acid sequence of SEQ ID NO: 1 or 14 and the light chain variable domain comprises the amino acid sequence of SEQ ID NO: 3 or 16.

Embodiment 63. The antibody or antigen binding fragment thereof of embodiment 62, wherein the heavy chain variable domain comprises the amino acid sequence of SEQ ID NO: 1 and the light chain variable domain comprises the amino acid sequence of SEQ ID NO: 3 or the heavy chain variable domain comprises the amino acid sequence of SEQ ID NO: 14 and the light chain variable domain comprises the amino acid sequence of SEQ ID NO: 16.

Embodiment 64. The antibody or antigen binding fragment thereof of embodiment 58, wherein the antibody comprises a light chain comprising the amino acid sequence of SEQ ID NO: 13 or 22 and the heavy chain comprises the amino acid sequence of SEQ ID NO: 11, 17, or 18.

Embodiment 65. The antibody or antigen binding fragment thereof of embodiment 64, wherein the antibody comprises: (a) a light chain comprising the amino acid sequence of SEQ ID NO: 13 and a heavy chain comprising the amino acid sequence of SEQ ID NO: 11; (b) a light chain comprising the amino acid sequence of SEQ ID NO: 22 and a heavy chain comprising the amino acid sequence of SEQ ID NO: 17; or (c) a light chain comprising the amino acid sequence of SEQ ID NO: 22 and a heavy chain comprising the amino acid sequence of SEQ ID NO: 18.

Embodiment 66. The antibody or antigen binding fragment thereof of embodiment 58, wherein the antibody comprises a light chain comprising the amino acid sequence of SEQ ID NO: 13 or 22 and a heavy chain comprising the amino acid sequence of SEQ ID NO: 58, 59, or 60.

Embodiment 67. The antibody or antigen binding fragment thereof of embodiment 66, wherein the antibody comprises: (a) a light chain comprising the amino acid sequence of SEQ ID NO: 13 and a heavy chain comprising the amino acid sequence of SEQ ID NO: 58; (b) a light chain comprising the amino acid sequence of SEQ ID NO: 22 and a heavy chain comprising the amino acid sequence of SEQ ID NO: 59; or (c) a light chain comprising the amino acid sequence of SEQ ID NO: 22 and a heavy chain comprising the amino acid sequence of SEQ ID NO: 60.

Embodiment 68. The antibody or antigen binding fragment thereof of embodiment 58, wherein the antibody further comprises a cysteine or a non-canonical amino acid amino acid substitution at one or more position(s) selected from the group consisting of positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain according to sequential numbering of the whole light chain sequence.

Embodiment 69. The antibody or antigen binding fragment thereof of embodiment 68, wherein the antibody comprises a cysteine or a non-canonical amino acid amino acid substitution at position 375 of the constant domain of the heavy chain.

Embodiment 70. The antibody or antigen binding fragment thereof of embodiment 69, wherein the antibody comprises a heavy chain comprising the amino acid sequence of SEQ ID NO: 19, 20, 61, or 62.

Embodiment 71. The antibody or antigen binding fragment thereof of embodiment 58, wherein the antibody is conjugated to a payload.

Embodiment 72. The antibody or antigen binding fragment thereof of embodiment 68, wherein the cysteine or noncanonical amino acid is conjugated to a payload.

Embodiment 73. The antibody or antigen binding fragment thereof of embodiment 71 or 72, wherein the payload is a therapeutic moiety, a detectable label, a radionuclide, or a protecting group.

Embodiment 74. The antibody or antigen binding fragment thereof of embodiment 73, wherein the therapeutic moiety is a cytotoxic moiety, an anti-inflammatory moiety, a peptide, a nucleic acid molecule, or a nucleic acid analog.

Embodiment 75. The antibody or antigen binding fragment thereof of embodiment 74, wherein the cytotoxic moiety is taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoid such as DM1, DM2, DM3, DM4), auristatins including monomethylauristatin E (MMAE), monomethylauristatin F (MMAF), monomethylauristatin D (MMAD), 7-ethyl-10-hydroxy-camptothecin (SN-38), anthracycline, alkylcycline, and derivatives thereof.

Embodiment 76. The antibody or antigen binding fragment thereof of embodiment 74, wherein the cytotoxic moiety is an inhibitor of topoisomerase I, topoisomerase II, or microtubule polymerization.

Embodiment 77. A method for treating a cancer in an individual in need thereof comprising administering to the individual a therapeutically effective amount of the antibody or antigen binding fragment thereof of embodiment 58 to treat the cancer, wherein the cancer is a cancer that overexpresses TROP2.

Embodiment 78. The method of embodiment 77, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 79. Use of the antibody or antigen binding fragment thereof of embodiment 58 for the manufacture of a medicament for the treatment of a cancer that overexpresses TROP2.

Embodiment 80. The use of embodiment 79, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 81. The antibody or antigen binding fragment thereof of embodiment 58 for the treatment of a cancer that overexpresses TROP2.

Embodiment 82. The antibody or antigen binding fragment thereof of embodiment 81, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 83. A combination therapy for treating cancer comprising the antibody or antigen binding fragment thereof of embodiment 58 and a therapeutic agent, wherein the cancer is a cancer that overexpresses TROP2.

Embodiment 84. The combination therapy of embodiment 83, wherein the therapeutic agent is a chemotherapy agent or a therapeutic antibody.

Embodiment 85. The combination therapy of embodiment 84, wherein the therapeutic antibody is a checkpoint inhibitor.

Embodiment 86. The combination therapy of embodiment 84, wherein the therapeutic antibody is an anti-PD1 antibody or an anti-PD-L1 antibody.

Embodiment 87. The combination therapy of embodiment 83, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 88. An antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15.

Embodiment 89. The antibody or antigen binding fragment thereof of embodiment 88, wherein the antibody or antigen-binding fragment thereof displays reduced hydrophobicity compared to Sacituzumab as determined by hydrophobic interaction chromatography (HIC).

Embodiment 90. The antibody or antigen binding fragment thereof of embodiment 88 or embodiment 89, wherein the antigen binding fragment is a Fab fragment, a Fab′ fragment, or a F(ab′)2 fragment.

Embodiment 91. The antibody or antigen binding fragment thereof of any one of embodiments 88 to 90, wherein the light chain comprises the amino acid sequence of SEQ ID NO: 21 and the heavy chain comprises the amino acid sequence of SEQ ID NO: 17 or 18.

Embodiment 92. The antibody or antigen binding fragment thereof of any one of embodiments 88 to 90, wherein the antibody comprises a light chain comprising the amino acid sequence of SEQ ID NO: 21 and a heavy chain comprising the amino acid sequence of SEQ ID NO: 59 or 60.

Embodiment 93. The antibody or antigen binding fragment thereof of any one of embodiments 88 to 92, wherein the antibody further comprises a cysteine or a non-canonical amino acid amino acid substitution at one or more position(s) selected from the group consisting of positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

Embodiment 94. The antibody or antigen binding fragment thereof of embodiment 93, wherein the antibody comprises a cysteine or a non-canonical amino acid amino acid substitution at position 375 of the constant domain of the heavy chain.

Embodiment 95. The antibody or antigen binding fragment thereof of embodiment 94, wherein the antibody comprises a heavy chain comprising the amino acid sequence of SEQ ID NO: 19, 20, 61, or 62.

Embodiment 96. The antibody or antigen binding fragment thereof of embodiment 88, wherein the antibody is conjugated to a payload.

Embodiment 97. The antibody or antigen binding fragment thereof of embodiment 93, wherein the cysteine or noncanonical amino acid is conjugated to a payload.

Embodiment 98. The antibody or antigen binding fragment thereof of embodiment 96 or 97, wherein the payload is a therapeutic moiety, a detectable label, a radionuclide, or a protecting group.

Embodiment 99. The antibody or antigen binding fragment thereof of embodiment 98, wherein the therapeutic moiety is a cytotoxic moiety, an anti-inflammatory moiety, a peptide, a nucleic acid molecule, or a nucleic acid analog.

Embodiment 100. The antibody or antigen binding fragment thereof of embodiment 99, wherein the cytotoxic moiety is taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin, podophyllotoxin derivatives such as etoposide or etoposide phosphate, vinblastine, vincristine, vindesine, taxanes such as taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoid such as DM1, DM2, DM3, DM4), auristatins including monomethylauristatin E (MMAE), monomethylauristatin F (MMAF), monomethylauristatin D (MMAD), 7-ethyl-10-hydroxy-camptothecin (SN-38), anthracycline, alkylcycline, and derivatives thereof.

Embodiment 101. The antibody or antigen binding fragment thereof of embodiment 99, wherein the cytotoxic moiety is an inhibitor of topoisomerase I, topoisomerase II, or microtubule polymerization inhibitor.

Embodiment 102. A method for treating a cancer in an individual in need thereof comprising administering to the individual a therapeutically effective amount of the antibody or antigen binding fragment thereof of embodiment 88 to treat the cancer, wherein the cancer is a cancer that overexpresses TROP2.

Embodiment 103. The method of embodiment 102, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 104. Use of the antibody or antigen binding fragment thereof of embodiment 88 for the manufacture of a medicament for the treatment of a cancer that overexpresses TROP2.

Embodiment 105. The use of embodiment 104, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 106. The antibody or antigen binding fragment thereof of embodiment 88 for the treatment of a cancer that overexpresses TROP2.

Embodiment 107. The antibody or antigen binding fragment thereof of embodiment 106, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

Embodiment 108. A combination therapy for treating cancer comprising the antibody or antigen binding fragment thereof of embodiment 88 and a therapeutic agent, wherein the cancer is a cancer that overexpresses TROP2.

Embodiment 109. The combination therapy of embodiment 108, wherein the therapeutic agent is a chemotherapy agent or a therapeutic antibody.

Embodiment 110. The combination therapy of embodiment 109, wherein the therapeutic antibody is a checkpoint inhibitor.

Embodiment 111. The combination therapy of embodiment 109, wherein the therapeutic antibody is an anti-PD1 antibody or an anti-PD-L1 antibody.

Embodiment 112. The combination therapy of embodiment 108, wherein the cancer is selected from the group consisting of: breast cancer (e.g., triple negative breast cancer), cervix cancer, colorectal cancer, esophagus cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

The examples describe the discovery of a new class of TROP2 binders of the present invention and ADCs of the present invention comprising the new class of TROP2 binders.

### General Methods

### Ultra-High Performance Size-Exclusion Chromatography (UP-SEC)

To determine aggregation by UP-SEC, 5 μg of purified antibody is injected onto an Acquity BEH200 SEC, 1.7 μm, 4.6×150 mm size exclusion column (Waters Corporation, Milford, MA) that is equilibrated with 100 mM sodium phosphate, 200 mM sodium chloride, 0.02% sodium azide pH 7, at 0.5 mL/minute using a Waters H-Class ultra-high performance chromatography (UPLC) column. Chromatograms are collected at both 215 and 280 nm wavelengths, and integration of the absorption at 280 nm (wavelength) trace was performed using EMPOWER 2 (Waters).

### Hydrophobic Interaction Chromatography (HIC)

To determine the hydrophobicity of a given antibody or ADC using HIC, 50 μg of sample at about 0.5-1 mg/mL is mixed 1/1 (v/v) with a 1.5 M ammonium sulfate, 50 mM sodium phosphate, pH 7.0/isopropanol (95:5 v/v) solution. Prepared samples are subsequently filtered through a 0.22 um polyvinylidene difluoride (PVDF) membrane prior to loading on a Thermofisher Scientific, Inc. (Waltham, MA USA) MAbPac™ HIC-Butyl high performance liquid chromatography (HPLC) according the Manufacturer's manual equilibrated in 1.5 M ammonium sulfate, 50 mM sodium phosphate, pH 7.0/isopropanol (95:5 v/v) (mobile phase A). The samples are eluted using an inverted gradient from mobile phase A to 50 mM sodium phosphate, pH 7.0/isopropanol (80:20 v/v) (mobile phase B). The elution is followed by recording the A280 nm as a function of time, the data is then exported and analyzed using the Empower software. The retention time of each sample is compared to a reference and is characteristic of the antibody's hydrophobicity, with longer elution times correlating with higher degree of hydrophobicity.

### Reverse Phase High-Performance Liquid Chromatography (RP-HPLC)

To determine purity using RP-HPLC, 15 μg of purified antibody is loaded onto a POROS R2/10 2.1×30 mm column (Applied Biosystems, Thermo Fisher Corporation) and equilibrated with 30% acetonitrile, 0.2% trifluoroacetic acid at 70° C. and 2 mL/min. Samples are eluted with a linear gradient from 30-58% acetonitrile in 0.2% trifluoroacetic acid over 5 minutes. Chromatograms are collected at both 215 and 280 nm wavelengths, and integration of the A280 nm trace was performed using Chemstation Rev. B.04.01 (Agilent Technologies, Inc., Santa Clara, CA).

### Tonset/Tm/Tagg Measurements by Nano-Differential Scanning Fluorimetry (Nano-DSF)

Nano-DSF is a method for measuring ultra-high-resolution protein stability using intrinsic tryptophan or tyrosine fluorescence. All nano-DSF studies are performed using the NanoTemper Prometheus NT.48 instrument (NanoTemper Technologies, Inc., South San Francisco, CA). Samples (˜10 μL at 0.5-1 mg/mL) are loaded by capillarity into standard grade nano-DSF capillaries, placed on the Prometheus capillary holder and subjected to a temperature ramping of 1° C./minute from 20° C. to 94.8° C. Up to 48 samples may be analyzed in parallel and assessed for stability in 3 seconds at the respective temperature.

The melting point (Tm) onset (C) and Tm (° C.) values indicate the structural stability of the samples and are obtained by monitoring the intrinsic tryptophan and tyrosine fluorescence at the emission wavelengths of 330 nm and 350 nm. To generate an unfolding curve, the ratio of the fluorescence intensities (F350 nm/F330 nm) is plotted vs. temperature or time. The thermal stability of a sample is described by the thermal unfolding transition midpoint Tm (° C.), at which half of the protein population is unfolded. The Tm corresponds to the inflection point of the unfolding curve and is determined via the derivative of the curve.

The aggregation point Tagg (° C.) is representative of the colloidal stability of the samples and is obtained by monitoring the back-reflection of near ultraviolet (UV) light using back reflection optics. The back-reflection optics uses near UV light scattering by protein aggregates, and thus only non-scattered light reaches the detector. The reduction of back reflected light is therefore a direct measure for aggregation in the sample.

### Non-Reducing Sodium Dodecyl Sulfate Capillary Electrophoresis (CE-SDS NR)

Five μL of each sample at 1 mg/mL are mixed in a 96-well plate with 35 μL of loading buffer (HT Protein Express Sample Buffer) (Perkin Elmer, Waltham, MA) containing either 50 mM iodoacetamide or 50 mM dithiothreitol. The plate is incubated at 70° C. for 20 minutes and 75 μL of water is added to each well. Each sample was analyzed on a LabChip GXII (Perkin Elmer) using an HT Protein Express Chip (Perkin Elmer). Electropherograms are collected by measuring the fluorescence of the sample over time and integrated using the LabChip GX software V4.1.1619.0 SP1 (Perkin Elmer).

### Affinity-Capture Self-Interaction Nanoparticle Spectroscopy (AC-SINS)

The AC-SINS assay measures protein self-interaction by capturing the antibody on the surface of a gold colloid that displays surface resonance oscillations in frequency with visible light. As the immobilized antibodies self-interact, the colloids aggregate, changing the oscillation frequency to absorb at a longer wavelength. Gold nanoparticles are incubated overnight with an 80/20 (v/v) capture antibody/non-capture antibody mixture. The coated gold nanoparticles are then spun down and resuspended into the conjugation buffers (20 mM sodium acetate pH 5.5 and PBS 1× pH 7.4) to a final volume of 50 μL. The samples are diluted to 0.05 mg/mL into the conjugation buffers and 45 μL of each dilution is loaded onto a 384-well plate. Five μL of previously prepared gold nanoparticles are then added to each well of the plate including antibodies and buffer controls. The plate is then covered with an aluminum lid, incubated at room temperature for 2 hours, and quickly spun down at 3000 rpm prior to reading the absorbance spectra of each well from 450 to 650 nm using a plate reader. Each sample spectra is recorded and analyzed for red-shifting of the maximum of absorption peak compared to buffers and antibody controls. The red-shifting and its strength is indicative of the self-interaction propensity of the tested antibody sample.

### Assessment of Aggregation and Self-Interaction by Dynamic Light Scattering (DLS)

All DLS studies are performed at 25° C. on the undiluted samples in glass bottom 96-well plates using a DynaPro Plate Reader II (Wyatt Technology Corporation, Santa Barbara, CA). Twenty (20) acquisitions (5 seconds each) per well are averaged and the hydrodynamic radius (Rh), % polydispersity, and % mass was modeled using Dynamics version 7.1.9.3 (Wyatt Technology Corporation) to assess aggregation.

For the determination of self-interaction, the diffusion interaction parameter (k_{D}) is determined by DLS. High-concentration samples are diluted with the buffers of interest (20 mM sodium acetate pH 5.5 and 10 mM histidine hydrogen chloride pH 6.5) to obtain a concentration of 20 mg/mL, filtered through 0.22 μm filters, and diluted in filtered buffers (with desired pH and ionic strength) to obtain lower concentration samples (2, 5, 10, 15, and 20 mg/mL), which are then added to the microplate. The k_{D} is determined by a linear fit of the measured (mutual) diffusion coefficients as a function of concentration.

### PEG 6000 Solubility Measurement

To determine the PEG 6000 solubility of a given protein, samples are dialyzed and diluted to 2 mg/mL with the filtered buffers of interest (20 mM sodium acetate pH 5.5 and 10 mM histidine hydrogen chloride pH 6.5). The PEG 6000 concentration screens (0 to 40% w/v) are generated using the Andrew robot (Alliance, Geneva, Switzerland) by diluting a 40% w/v PEG 6000 stock solution in buffers of interest with stock solutions of the corresponding buffers. Ten (10) μL of the 2 mg/mL samples were loaded into a half-well UV microplate (Corning, Corning, NY) pre-loaded with 90 μL of the previously prepared PEG 6000 concentration screen solutions, mixed, and incubated 1 hour at room temperature prior to reading the plate using an EPOCH/2 Microplate reader from BioTek Instruments, Inc. (Winooski, VT) measuring the optical densitometry at 320 nm wavelength. The optical density at 280 nm (OD280) can also be used to analyze the filtered samples. The value reported is the PEG 6000 concentration at the midpoint at which half of the protein population was precipitated and is determined via the derivative of the precipitation curve.

### Viscosity Assessment

This method accurately measures dynamic viscosities of antibody and protein formulations for a range of concentrations and viscosities (1-80 centipoises (cP)). Samples are evaluated for viscosity in 3 different formulations (10 mM sodium acetate pH 5.5, 10 mm histidine-HCl pH 6.5, and 1×PBS pH 7.4) and within a 10-200 mg/ml concentration range. The prepared samples are then filtered using a 0.2 um PVDF membrane prior to loading 60 μL into glass vials. The vials are quickly spun down and placed into the VROC initium (Rheosense Inc., San Ramon, CA) sample vial tray. Forty-eight (48) μL of the sample are then injected into the instrument cell where the viscosity of the solution was measured between 1 and 80 cP at 25° C. Viscosity values are then plotted as a function of protein concentration. Each reported viscosity value is the average of 10 measurements.

Assessment of Aggregation Formation after Low pH Hold

In this method, the samples from small-scale purification are quickly buffer exchanged using a 96-well ZEBA Spin desalting plate (Thermo Fisher Scientific Corporation) prior to lowering the pH to 3.5 using 2 M acetic acid. The plate is then covered using a Roche light cycler foil and incubated for 30 minutes at room temperature prior to adjusting the pH of the solution to 5 using 1 M TRIS base. The samples are then spun down quickly at 3000 rpm prior to injecting 5-10 μg on a Waters BEH200 size-exclusion chromatography (SEC) column equilibrated in 100 mM sodium phosphate, 200 mM sodium chloride, 0.02% sodium azide pH 7 using a Waters UPLC system to assess the sample purity by UP-SEC.

### Oxidation by Azobis(2-Amidinopropane) Dihydrochloride (AAPH)

Antibody at 1-2 mg/mL is incubated in 1 mM AAPH at 40° C. for 6 hours protected from light, buffer exchanged into 20 mM sodium acetate, pH 5.5 and stored at −80° C. until analysis.

### Light Stress

Antibody at 1-2 mg/mL is placed in a reusable quartz cuvette, exposed to 1× light (200 W-h/m2 ultraviolet and 1200 k-lux visible light) at 25° C. and stored at −80° C. until analysis.

Assessment of Isoelectric Point and Charge Variants by Capillary Isoelectric Focusing (cIEF)

To determine the isoelectric point (pI) by cIEF, samples are diluted to 0.2 mg/mL in buffer containing 0.35% methyl cellulose, 3 M urea, 1% Pharmalyte 3-10 (GE Healthcare), 0.5% Pharmalyte 5-8, 0.5% Pharmalyte 8-10, 0.5% pI marker 5.85 (ProteinSimple Inc., San Jose, CA) and 0.5% pI marker 9.77. Samples are run on an iCE3 (ProteinSimple) using an FC-coated capillary focusing for 1 minute at 1500 V followed by 8 minutes at 3000 V. Data is exported into and integrated using Empower 2 (Waters Corporation).

### Analysis of Antibodies by LC-MS MS Peptide Mapping

For peptide mapping by mass spectrometry, 100 μg of each sample is denatured with 30 μL of 8 M guanidine/1 M tris hydrochloride solution (15:1), reduced with 2 μL of 1 M dithiothreitol for 30 minutes at 60° C. and alkylated with 5 μL of 1 M iodoacetamide for 45 minutes in dark. Before digestion, samples are buffer exchanged into 50 mM ammonium bicarbonate using 7 kDa molecular weight cut off ZEBA cartridges. Samples are digested with 2 μg of trypsin and chymotrypsin for 2 hours at 37° C. Digestion is quenched by the addition of 3 μL of 5 M hydrochloride to each sample. Data is acquired in a Dionex/QE plus MS using a linear gradient over 50 min from 2-36% acetonitrile in 0.1% formic acid. Samples are analyzed using PEAKS DB (Bioinformatics Solutions Inc., Waterloo, Ontario, Canada) for database searching as well as PepFinder (Thermo Fisher Scientific Corporation) and manual verification for the percent change assessment.

### Analysis by Surface Plasmon Resonance (SPR)

Binding kinetics of the antibodies to the target is determined by SPR on a BIAcore T200 or BIAcore 4000 (GE Healthcare). The running buffer, 10 mM HEPES, 150 mM NaCl, 0.05% v/v Surfactant P20, 3 mM Ethylenediaminetetraacetic acid (EDTA), pH 7.4 (HBS-EP+, GE Healthcare) is used for immobilization and reagent dilutions. All binding kinetics are measured at 25° C.

For each injection cycle, antibodies are first captured in different flow cells with an anti-human Fc antibody (Human Antibody Capture Kit, GE Healthcare) immobilized to the sensor chip (Series S CM5, GE Healthcare). Reference flow cell with no captured antibody is also used. Serial dilutions of the target protein, ranging in concentration from 0.16 nM to 80 nM, and buffer blanks are injected in multiple cycles over the captured antibodies and reference surfaces for a 3-minute association followed by a 10-minute dissociation. The surfaces are regenerated with a 30 second injection of 3 M MgCl₂ after each cycle.

Double-referenced titration data is globally fit to a 1:1 Langmuir binding model to determine the association rate constant, kₐ (M−1 s−1), and the dissociation rate constant, k_{d} (s−1), using the BIAcore T200 Evaluation Software version 2.0 or BIAcore 4000 Evaluation Software version 1.1 (GE Healthcare). The equilibrium dissociation constant was calculated as K_{D}(M)=k_{d}/kₐ.

### Subvisible Particle Assessment Following 10 Days Incubation at 50° C. by Flow Cytometry

A Guava EasyCyte 5HT™ flow cytometer (GFC) used in this study is purchased from EMD Millipore Corp (Billerica, MA). Briefly, the plate carrying the protein samples (160 μL of 1 mg/mL protein solution) is left undisturbed at 5° C. overnight for degassing and minimizing potential interference from micro air bubbles that might be trapped in the solution during the sample preparation. The data are collected for 250 seconds to allow an analyzed sample volume of 60 μL, unless the particle counts hit the 200,000 count limit of this instrument before 250 seconds and the instrument automatically ended the data collection and moved onto the next sample. The number of measured particles is limited by the analyzed sample volume (60 μL) or 200,000 counts. The number of reported particles in particles/mL is proportional to the volume multiplication factor and measured volume. The instrument performance is confirmed with Easy Check Kit (EMD Millipore Corporation, St. Louis, MO) before sample analysis. The sample plate is gently hand-mixed prior to being loaded on the GFC instrument and is assayed without using the GFC mixer in order to avoid potentially altering protein aggregate populations and generating air bubbles.

### Example 1

### Sacituzumab CDR and Framework Saturation Mutagenesis.

57 V_{H} and 43 V_{L} positions were selected in the CDR and Framework region of the Sacituzumab (hRS7) sequence. For each position, each of 17 amino acids were tested (excluding M, C and W) for a total sequence variation of 976 V_{H}+731 V_{L}=1707 point mutant variants. Yeast cells expressing the Sacituzumab variants (mAbs) were cell sorted with increasing amounts of labelled TROP2 protein (10, 30, and 100 nM) and lower affinity cells were selected by gating to the left of the wild-type sequence. Selected mutant sequences were accurately measured using 40 single point (20 V_{L}+20 V_{H}). Mutant variants were identified with decreased affinity and assessed for binding using surface plasmon resonance single cycle kinetics. The results are shown in Table 9. Unless indicated otherwise in the table, all the light chain muteins (Lm) are paired with a Sacituzumab heavy chain comprising an S375C substitution (Hm_S375C having the amino acid sequence shown in SEQ ID NO: 162) and all the Hm muteins comprise the Sacituzumab light chain (LC having the amino acid sequence shown in SEQ ID NO:12). The amino acid sequences comprising the antibodies disclosed in Table 9 may be found in Table 27.

TABLE 9

| | | | pH 7.4 | Fold |
|---|---|---|---|---|
| | kₐ | k_{d} | K_{D} | De- |
| Antibody | (1/M*s) | (1/s) | (M) | crease |
| mAb_LC_Hm_S375C | 4.40E+05 | 2.40E−04 | 5.47E−10 | 1x |
| mAb_Lm_T94F_Hm_S375C | 2.87E+05 | 9.68E−04 | 3.37E−09 | 6x |
| mAb_Lm_I93G_Hm_S375C | 4.10E+05 | 1.39E−03 | 3.38E−09 | 6x |
| mAb_Lm_V29R_Hm_S375C | 4.13E+05 | 2.36E−03 | 5.72E−09 | 10x |
| mAb_LC_Hm_F108Q_S375C | 1.92E+05 | 1.21E−03 | 6.32E−09 | 12x |
| mAb_Lm_D28H_Hm_S375C | 3.75E+05 | 2.93E−03 | 7.83E−09 | 14x |
| mAb_LC_Hm_F29G_S375C | 1.58E+05 | 1.30E−03 | 8.25E−09 | 15x |
| mAb_LC_Hm_F108S_S375C | 2.52E+05 | 2.64E−03 | 1.05E−08 | 19x |
| mAb_Lm_Y53V_Hm_S375C | 3.33E+05 | 5.62E−03 | 1.69E−08 | 31x |
| mAb_LC_Hm_Y107N_ | 4.06E+04 | 1.10E−03 | 2.70E−08 | 49x |
| S375C | | | | |
| mAb_LC_Hm_F101T_S375C | 8.94E+04 | 3.16E−03 | 3.53E−08 | 65x |
| mAb_Lm_Y53S_Hm_S375C | 1.90E+05 | 6.79E−03 | 3.57E−08 | 65x |
| mAb_LC_Hm_N31P_S375C | 2.31E+05 | 8.83E−03 | 3.83E−08 | 70x |
| mAb_LC_Hm_Y32E_S375C | 6.47E+04 | 2.54E−03 | 3.92E−08 | 72x |
| mAb_Lm_Y53P_Hm_S375C | 7.65E+05 | 3.30E−02 | 4.31E−08 | 79x |
| mAb_LC_Hm_G33I_S375C | 6.07E+05 | 2.77E−02 | 4.56E−08 | 83x |
| mAb_LC_Hm_S104D_S375C | 2.47E+09 | 1.16E+02 | 4.69E−08 | 86x |
| mAb_Lm_Y53R_Hm_S375C | 2.28E+05 | 1.08E−02 | 4.74E−08 | 87x |
| mAb_Lm_Y53N_Hm_S375C | 2.51E+05 | 1.35E−02 | 5.36E−08 | 98x |
| mAb_Lm_S50T_Hm_S375C | 4.16E+05 | 2.39E−02 | 5.74E−08 | 105x |
| mAb_Lm_Q89V_Hm_S375C | 1.31E+04 | 1.19E−03 | 9.03E−08 | 165x |
| mAb_Lm_Y53D_Hm_S375C | 1.87E+05 | 1.83E−02 | 9.79E−08 | 179x |
| mAb_LC_Hm_F101E_S375C | 5.04E+04 | 4.94E−03 | 9.80E−08 | 179x |
| mAb_LC_Hm_Y107I_S375C | 3.15E+03 | 3.12E−04 | 9.92E−08 | 181x |
| mAb_Lm_S30R_Hm_S375C | 3.79E+04 | 4.78E−03 | 1.26E−07 | 230x |
| mAb_Lm_H91K_Hm_S375C | 2.67E+04 | 3.93E−03 | 1.47E−07 | 269x |
| mAb_Lm_Y92E_Hm_S375C | 8.95E+08 | 1.40E+02 | 1.57E−07 | 287x |
| mAb_LC_Hm_G33S_S375C | 1.50E+04 | 2.39E−03 | 1.60E−07 | 293x |
| mAb_LC_Hm_W106L_ | 1.34E+05 | 2.59E−02 | 1.93E−07 | 353x |
| S375C | | | | |
| mAb_LC_Hm_W106H_ | 2.80E+05 | 5.57E−02 | 1.99E−07 | 364x |
| S375C | | | | |
| mAb_LC_Hm_Y105S_S375C | 1.65E+05 | 3.42E−02 | 2.07E−07 | 378x |
| mAb_Lm_Y92N_Hm_S375C | 4.68E+05 | 1.60E−01 | 3.41E−07 | 623x |
| mAb_Lm_A32P_Hm_S375C | 1.01E+05 | 3.86E−02 | 3.83E−07 | 700x |
| mAb_LC_Hm_W106A_ | 6.12E+04 | 2.60E−02 | 4.24E−07 | 775x |
| S375C | | | | |
| mAb_Lm_A32Q_Hm_S375C | 1.59E+05 | 8.20E−02 | 5.15E−07 | 941x |
| mAb_Lm_H91Y_Hm_S375C | 1.81E+05 | 1.65E−01 | 9.15E−07 | 1673x |
| mAb_LC_Hm_F108T_S375C | 1.79E+04 | 1.65E−02 | 9.20E−07 | 1682x |
| mAb_LC_Hm_W106E_ | 3.63E+04 | 5.02E−02 | 1.38E−06 | 2523x |
| S375C | | | | |
| mAb_LC_Hm_G33P_S375C | 3.25E+04 | 5.55E−02 | 1.71E−06 | 3126x |
| mAb_LC_Hm_S103T_S375C | 3.12E+04 | 1.20E−01 | 3.84E−06 | 7020x |
| mAb_Lm_Y92A_Hm_S375C | 5.09E+04 | 2.84E−01 | 5.57E−06 | 10183x |
| mAb_Lm_S50L_Hm_S375C | 1.92E+05 | 1.41E+00 | 7.35E−06 | 13437x |

mAb = Sacituzumab
LC = light chain of Sacituzumab
Lm = light chain mutation (amino acid substitution in V_{L} domain); positions are defined according to sequential numbering
Hm = heavy chain mutation (amino acid substitution in V_{H} domain); positions are defined according to sequential numbering

### Example 2

On-Cell ELISA binding affinities of Sacituzumab mutants, comparison of BxPC3 and MDA-MB-231 cell lines.

Cell lines expressing TROP2. Quantitative fluorescence-activated cell sorter (qFAC) analysis of BxPC3 and MDA-MB-231-KWL cells lines for TROP2 copy number expression (Table 10).

TABLE 10

| | | Trop-2 | Category Based |
|---|---|---|---|
| | | Copy | on TROP2 |
| Cell Line | Indication | Number | Expression |
| BxPC-3 | Pancreatic | 456K | High |
| MDA-MB- | Breast | 42.7K | Low |
| 231-KWL | | | |
| EMT6 | Breast (Mouse) | 0 | Negative Control |

On-Cell ELISA binding affinities of Sacituzumab mutants, comparison of BxPC3 and MDA-MB-231 cell lines. Cells were plated in duplicate onto 96-well plates, and primary antibody was diluted in cell culture medium with a starting concentration of 30 μg/mL (200 nM) with a dilution factor of 1:5. After 3× wash, secondary antibody was added for 1 hour at 1 ug/ml, washed three times and absorbance was measured at 450 nm. Data was plotted and analyzed. EC50 (kD) was measured along with area under the curve (AUC) for each cell line. A large differential between AUC of BxPC3 binding and MDA-MB-231-KWL (ΔΔUC/AUC (BxPC-3)) was used to select mutants for further investigation. The results are shown in Table 11. Unless indicated otherwise in the table, all the light chain muteins (Lm) are paired with a Sacituzumab heavy chain (HC having the amino acid sequence shown in SEQ ID NO: 11) and all the Hm muteins comprise the Sacituzumab light chain (LC having the amino acid sequence shown in SEQ ID NO:12). The amino acid sequences comprising the antibodies disclosed in Table 11 may be found in Table 27.

TABLE 11

| | | MDA- | | AUC | | ΔAUC/ |
|---|---|---|---|---|---|---|
| | BxPC-3 | MB-231-KWL | AUC | (MDA-MB- | | AUC |
| Antibody | EC₅₀ | EC₅₀ | (BxPC-3) | 231-KWL) | ΔAUC | (BxPC-3) |
| mAb (parental) | 0.22 | 0.261 | 318.2 | 248.1 | 70.1 | 0.22 |
| mAb_Lm_Y53R_HC | 0.357 | 0.546 | 322.9 | 179.7 | 143.2 | 0.456 |
| mAb_Lm_Q89V_HC | 1.675 | 8.458 | 299.1 | 201 | 98.1 | 0.312 |
| mAb_Lm_T94F_HC | 0.233 | 0.374 | 317.6 | 240.2 | 77.4 | 0.246 |
| mAb_Lm_Y53V_HC | 0.225 | 0.343 | 317.4 | 212.6 | 104.8 | 0.334 |
| mAb_Lm_Y92A_HC | 9.202 | | 159.7 | 13.09 | 146.61 | 0.467 |
| mAb_Lm_Y53P_HC | 0.218 | 0.629 | 303.5 | 169.6 | 133.9 | 0.426 |
| mAb_Lm_A32Q_HC | 18.45 | | 142.2 | 17.84 | 124.36 | 0.396 |
| mAb_Lm_Y53D_HC | 0.238 | 4.89 | 288 | 95.09 | 192.91 | 0.614 |
| mAb_Lm_D28H_HC | 0.159 | 0.136 | 300.3 | 213.7 | 86.6 | 0.276 |
| mAb_Lm_H91K_HC | 0.899 | 1.956 | 293.7 | 162 | 131.7 | 0.419 |
| mAb_Lm_V29R_HC | 0.285 | 0.3 | 291 | 210.5 | 80.5 | 0.256 |
| mAb_Lm_S50T_HC | 0.222 | 0.568 | 305.3 | 157.4 | 147.9 | 0.471 |
| mAb_Lm_A32P_HC | 0.922 | | 256.1 | 64.29 | 191.81 | 0.611 |
| mAb_Lm_H91Y_HC | 3.562 | | 184.5 | 16.92 | 167.58 | 0.534 |
| mAb_Lm_Y92N_HC | 0.839 | | 225.9 | 45.05 | 180.85 | 0.576 |
| mAb_Lm_I93G_HC | 0.182 | 0.221 | 305.3 | 220 | 85.3 | 0.272 |
| mAb_Lm_Y92E_HC | 6.945 | | 164.6 | 16.12 | 148.48 | 0.473 |
| mAb_Lm_S50L_HC | 23.76 | | 133.1 | 4.218 | 128.88 | 0.41 |
| mAb_Lm_S30R_HC | 0.697 | | 215.8 | 47.06 | 168.74 | 0.537 |
| mAb_Lm_Y53N_HC | 0.225 | 0.843 | 309.2 | 140.3 | 168.9 | 0.538 |
| mAb_LC_Hm_F101E | 0.482 | 2.689 | 299.5 | 157.6 | 141.9 | 0.452 |
| mAb_ LC_Hm_Y32E | 0.416 | 1.581 | 297.5 | 193 | 104.5 | 0.333 |
| mAb_ LC_Hm_S104D | 0.214 | | 285 | 106.9 | 178.1 | 0.56 |
| mAb_ LC_Hm_W106E | | | 139.6 | 22.22 | 117.38 | 0.369 |
| mAb_ LC_Hm_G33P | | | 141.9 | 7.698 | 134.2 | 0.422 |
| mAb_ LC_Hm_Y107I | | | 264.6 | 163.3 | 101.3 | 0.318 |
| mAb_ LC_Hm_F108Q | 0.316 | 0.365 | 324.4 | 229.5 | 94.9 | 0.298 |
| mAb_ LC_Hm_G33S | 2.236 | | 281.2 | 137 | 144.2 | 0.453 |
| mAb_ LC_Hm_Y105S | 0.312 | | 268 | 79.95 | 188.05 | 0.591 |
| mAb_ LC_Hm_Y107N | 0.715 | 1.879 | 316.9 | 230.4 | 86.5 | 0.272 |
| mAb_ LC_Hm_F101T | 0.336 | 0.871 | 313.8 | 220.7 | 93.1 | 0.293 |
| mAb_ LC_Hm_W106A | 1.676 | | 214.6 | 28.69 | 185.91 | 0.584 |
| mAb_ LC_Hm_F108S | 0.321 | 0.651 | 302.9 | 226.8 | 76.1 | 0.239 |
| mAb_ LC_Hm_N31P | 0.369 | 1.155 | 289 | 173.2 | 115.8 | 0.364 |
| mAb_ LC_Hm_F29G | 0.32 | 0.448 | 290.9 | 219 | 71.9 | 0.226 |
| mAb_ LC_Hm_W106H | 1.163 | | 215.2 | 22.73 | 192.47 | 0.605 |
| mAb_ LC_Hm_W106L | 0.326 | | 251.4 | 60.85 | 190.55 | 0.599 |
| mAb_ LC_Hm_G33I | 0.217 | 0.768 | 285.5 | 129.3 | 156.2 | 0.491 |
| mAb_ LC_Hm_F108T | 0.681 | 1.197 | 278.3 | 206.9 | 71.4 | 0.224 |
| mAb_Lm_Y53S_HC | 0.28 | 0.304 | 307.2 | 205.4 | 101.8 | 0.32 |
| mAb_ LC_Hm_S103T | | | 204.7 | 33.66 | 171.04 | 0.538 |

mAb = Sacituzumab
LC = light chain of Sacituzumab
HC = heavy chain of Sacituzumab
Lm = light chain mutation (amino acid substitution in V_{L} domain); positions are defined according to sequential numbering
Hm = heavy chain mutation (amino acid substitution in V_{H} domain); positions are defined according to sequential numbering
AUC = area under curve

Surface Plasmon Resonance (SPR) analysis of the Y53D substitution in CDR2 of the light chain showed about a 10-fold reduction in affinity when the antibody binds in a bivalent mode while about 100-fold affinity difference when the Fab of the antibody binds (monovalent mode). See Table 12.

TABLE 12

| | Binding to Human Trop2 at pH 7.4 (TROP2 capture) | | | | |
|---|---|---|---|---|---|
| | | | | | Affinity |
| SPR | kₐ | k_{d} | K_{Dapp} | Binding | differences +/− |
| Antibody or Fab | (1/Ms) | (1/s) | (M) | mode | Y53D |
| αTROP2 (HC: Sac-S375C) | 1.7E+05 | 1.3E−04 | 7.3E−10 | bivalent | About 10-fold |
| (LC: Sac) | | | | | affinity difference |
| αTROP2 (HC: Sac-S375C) | 6.6E+05 | 4.6E−03 | 6.9E−09 | bivalent | |
| (LC: Sac-Y53D) | | | | | |
| αTROP2 Fab | 3.1E+05 | 3.2E−04 | 1.1E−09 | monovalent | About 100-fold |
| αTROP2 Fab (Y53D) | 8.0E+04 | 1.1E−02\| | 1.3E−07 | monovalent | affinity difference |

HC = heavy chain
LC = light chain
Sac = Sacituzumab
SPR = surface plasmon resonance

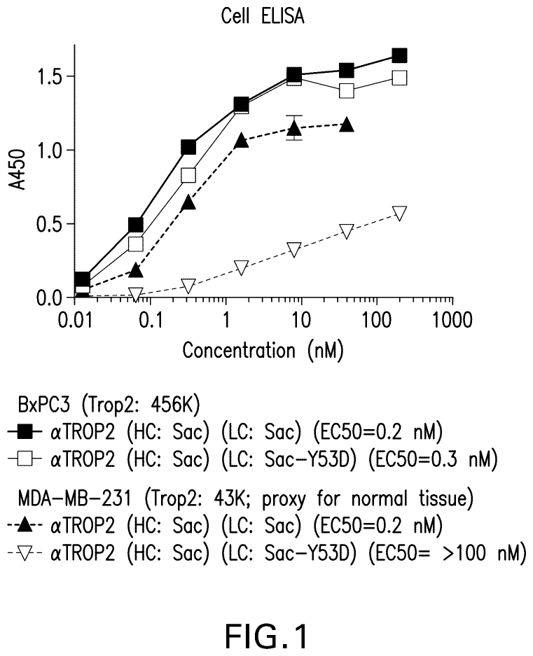

FIG. 1 shows that the Y53D substitution in CDR2 of the Sacituzumab light chain results in an avidity-tuned anti-TROP2 antibody that preferentially binds high TROP2-expressing BxPC3 cells (TROP2^{high} cells) over low TROP2-expressing MDA-MB-231-KWL cells (TROP2^{low} cells).

### Example 3

### Developability Characteristics of Various Mutants.

Developability characteristics for various mutants were performed as in Bailly Et al., Predicting Antibody Developability Profiles Through Early Stage Discovery Screening, MABS 12 (1): e1743053 (2020) (doi: 10.1080/19420862.2020.17430530), which is incorporated herein by reference in its entirety. Various methods used may be found herein under General Methods.

The results of the various assays are presented in Table 13, Table 14, and Table 15. Unless indicated otherwise, in Table 13, Table 14, and Table 15, all the light chain muteins (Lm) are paired with a Sacituzumab heavy chain (HC having the amino acid sequence shown in SEQ ID NO: 11) and all the Hm muteins comprise the Sacituzumab light chain (LC having the amino acid sequence shown in SEQ ID NO:12). The amino acid sequences comprising the antibodies disclosed in Table 13, Table 14, and Table 15 may be found in Table 27.

TABLE 13
Part A

| | | UP- | CE-SDS | CE-SDS | RP-HPLC | RP-HPLC | RP-HPLC |
|---|---|---|---|---|---|---|---|
| | UP-SEC | SEC | NR | NR | 70° C. | 70° C. | 80° C. |
| | % | % | % | % | % pre- | % | % pre- |
| Antibody | HMW | Mono | LMW | Main | Peak | Main | Peak |
| mAb_Hm_S103T | 5.2 | 94.8 | 0 | 100 | 5.7 | 94.3 | 1.9 |
| mAb_Lm_Y53S | 1.9 | 98.1 | 0 | 100 | 4.9 | 95.1 | 2.5 |
| mAb_Hm_F108T | 88.2 | 11.8 | 3.7 | 96.3 | 15.7 | 84.3 | 9.2 |
| mAb_Hm_G33I | 1.3 | 98.7 | 0 | 100 | 4.1 | 95.9 | 2.2 |
| mAb_Hm_W106L | 2.8 | 97.2 | 0.6 | 99.4 | 3.2 | 96.8 | 1.2 |
| mAb_Hm_W106H | 2.1 | 97.9 | 0 | 100 | 1.4 | 98.7 | 0.8 |
| mAb_Hm_F29G | 1.3 | 98.7 | 0 | 100 | 4.1 | 95.9 | 1.8 |
| mAb_Hm_N31P | 0.4 | 99.6 | 0 | 100 | 4.2 | 95.8 | 2 |
| mAb_Hm_F108S | 2.1 | 97.9 | 0 | 100 | 2 | 98 | 1.2 |
| mAb_Hm_W106A | 3.1 | 96.9 | 0.7 | 99.3 | 2.5 | 97.5 | 1.4 |
| mAb_Hm_F101T | 3.2 | 96.8 | 0.3 | 99.7 | 3.3 | 96.7 | 1.6 |
| mAb_Hm_Y107N | 2.6 | 97.4 | 0.3 | 99.7 | 3.1 | 97 | 1.6 |
| mAb_Hm_Y105S | 5.5 | 94.5 | 0.7 | 99.3 | 3.7 | 96.3 | 1.6 |
| mAb_Hm_G33S | 5.2 | 94.8 | 0.4 | 99.6 | 5.2 | 94.8 | 2.1 |
| mAb_Hm_F108Q | 2.7 | 97.3 | 0.6 | 99.4 | 0.9 | 99.1 | 0.5 |
| mAb_Hm_Y107I | 3.3 | 96.7 | 0.6 | 99.4 | 4.9 | 95.1 | 1.9 |
| mAb_Hm_G33P | 1.7 | 98.3 | 0 | 100 | 4.4 | 95.6 | 1.9 |
| mAb_Hm_W106E | 2.8 | 97.2 | 0.2 | 99.8 | 2.2 | 97.8 | 1.1 |
| mAb_Hm_S104D | 3.4 | 96.6 | 0.5 | 99.5 | 6.7 | 93.3 | 2.5 |
| mAb_Hm_Y32E | 2.1 | 97.9 | | 100 | 5.2 | 94.8 | 2 |
| mAb_Hm_F101E | 3.5 | 96.5 | 0.8 | 99.2 | 7.2 | 96.6 | 1.5 |
| mAb_Lm_Y53N | 1.4 | 98.6 | 0 | 100 | 5.5 | 94.5 | 2.1 |
| mAb_Lm_S30R | 1.2 | 98.8 | 0 | 100 | 4.9 | 95.1 | 2.2 |
| mAb_Lm_S50L | 1.2 | 98.8 | 0 | 100 | 5.1 | 94.9 | 2 |
| mAb_Lm_Y92E | 1.3 | 98.7 | 0 | 100 | 5.1 | 94.9 | 2.2 |
| mAb_Lm_I93G | 4.2 | 95.8 | 0.9 | 99.1 | 4.8 | 95.2 | 1.9 |
| mAb_Lm_Y92N | 3.7 | 96.3 | 0.9 | 99.1 | 5.8 | 94.2 | 2 |
| mAb_Lm_H91Y | 2.6 | 97.4 | 0.4 | 99.6 | 9.7 | 90.3 | 2.3 |
| mAb_Lm_A32P | 3.1 | 96.9 | 0 | 100 | 6 | 94 | 2.4 |
| mAb_Lm_S50T | 1.6 | 98.4 | 0 | 100 | 5.5 | 94.5 | 2.3 |
| mAb_Lm_V29R | 6 | 94 | 0 | 100 | 5.2 | 94.8 | 2.3 |
| mAb_Lm_H91K | 1.5 | 98.5 | 0.6 | 99.4 | 6.3 | 93.7 | 1.5 |
| mAb_Lm_D28H | 1.1 | 98.9 | | 100 | 5.8 | 94.2 | 2.2 |
| mAb_Lm_Y53D | 3.7 | 96.3 | 0.7 | 99.3 | 4.9 | 95.1 | 1.9 |
| mAb_Lm_A32Q | 1.2 | 98.8 | 0 | 100 | 7.7 | 92.3 | 2.8 |
| mAb_Lm_Y53P | 1 | 99 | 0 | 100 | 6 | 94 | 2.5 |
| mAb_Lm_Y92A | 0.8 | 99.2 | 0 | 100 | 6 | 94 | 2.2 |
| mAb_Lm_Y53V | 2.9 | 97.1 | 0.7 | 99.3 | 5 | 95 | 1.9 |
| mAb_Lm_T94F | 1.9 | 98.1 | 0.7 | 99.3 | 4.5 | 95.5 | 1.7 |
| mAb_Lm_Q89V | 1.4 | 98.6 | 0.7 | 99.3 | 5.1 | 94.9 | 1.7 |
| mAb_Lm_Y53R | 2.2 | 97.8 | 0 | 100 | 5.4 | 94.6 | 2.2 |
| mAb (parental) | 6.2 | 93.8 | 0.6 | 99.4 | 4 | 96.1 | 1.7 |

mAb = Sacituzumab
Lm = light chain mutation (amino acid substitution in V_{L} domain); positions are defined according to
sequential numbering
Hm = heavy chain mutation (amino acid substitution in V_{H} domain); positions are defined according to
sequential numbering
UP-SEC = ultra-high performance size-exclusion chromatography
RP-HPLC = high performance reverse phase chromatography
HMW = high molecular weight
LMW = low molecular weight
CE-SDS NR = non-reducing sodium dodecyl sulfate capillary electrophoresis
Part B

| | RP- | RP- | | | | | |
|---|---|---|---|---|---|---|---|
| | HPLC | HPLC | Nano- | | | | |
| | 80° C. | 80° C. | DSF | Nano- | Nano- | HIC | HIC- |
| | % | % Post | Tm | DSF | DSF | % | RT |
| Antibody | Main | Peak | onset | Tm1 | Tagg | area | (mins) |
| mAb_Hm_S103T | 98.1 | 0 | 63.5 | 69.9 | 82.7 | 100 | 32.3 |
| mAb_Lm_ Y53S | 97.5 | 0 | 63.5 | 70.1 | 83.3 | 100 | 21.1 |
| mAb_Hm_F108T | 82.8 | 8 | 47 | 55.1 | 75.2 | ND | ND |
| mAb_Hm_G33I | 97.9 | 0 | 55.1 | 61 | 83.3 | 100 | 31.7 |
| mAb_Hm_W106L | 98.8 | 0 | 63.6 | 69.7 | 80.7 | 100 | 30.8 |
| mAb_Hm_W106H | 96 | 3.2 | 64.9 | 70.1 | 77.4 | 100 | 27.9 |
| mAb_Hm_F29G | 98.2 | 0 | 65.3 | 70.1 | 81.7 | 100 | 30.9 |
| mAb_Hm_N31P | 98 | 0 | 63.4 | 70.5 | 77.8 | ND | ND |
| mAb_Hm_F108S | 98.8 | 0 | 58.7 | 69.5 | 74.6 | 100 | 33.7 |
| mAb_Hm_W106A | 98.7 | 0 | 62.5 | 70.1 | 81.5 | 100 | 28.8 |
| mAb_Hm_F101T | 98.4 | 0 | 62.9 | 69.7 | 82.3 | 100 | 21.9 |
| mAb_Hm_Y107N | 98.4 | 0 | 63.4 | 70.2 | 76.1 | 100 | 36.1 |
| mAb_Hm_Y105S | 98.4 | 0 | 64.1 | 70 | 83.2 | 100 | 21.1 |
| mAb_Hm_G33S | 97.9 | 0 | 64 | 69.7 | 84.4 | ND | ND |
| mAb_Hm_F108Q | 98.1 | 1.4 | 62.2 | 69.9 | 77.1 | 100 | 35.6 |
| mAb_Hm_Y107I | 98.1 | 0 | 63.6 | 69.9 | 81.1 | 100 | 30.4 |
| mAb_Hm_G33P | 98.1 | 0 | 64.6 | 70 | 82.7 | 100 | 38.2 |
| mAb_Hm_W106E | 98.9 | 0 | 65.2 | 70 | 77.9 | 100 | 27.4 |
| mAb_Hm_S104D | 97.6 | 0 | 64 | 69.9 | 82.4 | 100 | 25.4 |
| mAb_Hm_Y32E | 98 | 0 | 63.3 | 69.8 | 77.2 | 100 | 23.7 |
| mAb_Hm_F101E | 98.5 | 0 | 64.2 | 69.9 | 80.7 | 100 | 21.7 |
| mAb_Lm_Y53N | 97.9 | 0 | 63.2 | 70.1 | 83.5 | 100 | 21.4 |
| mAb_Lm_S30R | 97.8 | 0 | 63.3 | 70 | 83.3 | 100 | 25.9 |
| mAb_Lm_S50L | 98 | 0 | 62.3 | 69.9 | 83.7 | ND | ND |
| mAb_Lm_Y92E | 97.8 | 0 | 64 | 70.1 | 82.7 | 100 | 26.8 |
| mAb_Lm_I93G | 98.2 | 0 | 64.5 | 70.1 | 81.8 | 100 | 31.1 |
| mAb_Lm_Y92N | 98 | 0 | 64 | 69.9 | 82.4 | 100 | 28.2 |
| mAb_Lm_H91Y | 97.7 | 0 | 63.7 | 70.1 | 82.5 | 100 | 33 |
| mAb_Lm_A32P | 97.6 | 0 | 62.9 | 70 | 82.2 | 64.29 | 32 |
| mAb_Lm_S50T | 97.7 | 0 | 64.1 | 69.9 | 82.8 | 100 | 32 |
| mAb_Lm_V29R | 97.7 | 0 | 63.2 | 70.3 | 79.3 | 98.45 | 31.6 |
| mAb_Lm_H91K | 98.5 | 0 | 65.7 | 70.1 | 78.1 | ND | ND |
| mAb_Lm_D28H | 97.8 | 0 | 62.7 | 70 | 82.4 | 100 | 36.3 |
| mAb_Lm_Y53D | 98.1 | 0 | 63.3 | 69.9 | 81.4 | 100 | 22.5 |
| mAb_Lm_A32Q | 97.2 | 0 | 63.4 | 70 | 82.4 | 100 | 31.9 |
| mAb_Lm_Y53P | 97.5 | 0 | 62.4 | 70.1 | 80.9 | 100 | 28.1 |
| mAb_Lm_Y92A | 97.8 | 0 | 61.9 | 70.2 | 82.7 | 100 | 30.2 |
| mAb_Lm_Y53V | 98.1 | 0 | 63.8 | 70.1 | 81.8 | 93.32 | 27.9 |
| mAb_Lm_T94F | 98.4 | 0 | 62 | 69.8 | 82.4 | ND | ND |
| mAb_Lm_Q89V | 98.3 | 0 | 62.9 | 69.9 | 78 | ND | ND |
| mAb_Lm_Y53R | 97.9 | 0 | 63.8 | 69.9 | 82.1 | 100 | 22.8 |
| mAb (parental) | 98.3 | 0 | 64.1 | 69.9 | 82 | 100 | 31.8 |

mAb = Sacituzumab
Lm = light chain mutation (amino acid substitution in V_{L} domain); positions are defined according to sequential numbering
Hm = heavy chain mutation (amino acid substitution in V_{H} domain); positions are defined according to sequential numbering
ND = not determined
RP-HPLC = high performance reverse phase chromatography
HIC = hydrophobic interaction chromatography
HIC-RT = HIC retention time
DSF = differential scanning fluorimetry
Tagg = temperature of aggregation onset
Tm = thermal unfolding transition midpoint
Tm onset = onset of melting temperature

TABLE 14
SPR binding of Selected Sacituzumab Variants
(average of 8 and 28.8 RU)
TROP2 immobilized

| | Human Trop2 pH 7.4 | | | | |
|---|---|---|---|---|---|
| | | | | | % |
| | kₐ | k_{d} | K_{Dapp} | K_{D}/ | Ref |
| Antibody | (1/Ms) | (1/s) | (M) | K_{Dref} | BC |

Part A

| | | | | | |
|---|---|---|---|---|---|
| mAb (parental sequence) | 6.2E+04 | 1.7E−04 | 2.8E−09 | 0.0 | 100% |
| mAb_Lm_Q89V | 1.8E+04 | 5.8E−04 | 3.3E−08 | 0.4 | 37% |
| mAb_Lm_Y92A | | | weak | NA | |
| mAb_Lm_Y53D | 3.6E+08 | 6.1E+00 | 2.1E−08 | 0.2 | 39% |
| mAb_Lm_A32P | 1.7E+05 | 4.5E−03 | 2.7E−08 | 0.3 | 36% |
| mAb_Lm_Y92N | 1.8E+05 | 1.7E−02 | 9.5E−08 | 1.0 | 30% |
| mAb_Lm_S30R | 1.6E+05 | 1.4E−02 | 9.0E−08 | 1.0 | 31% |
| mAb_Lm_Y53N | 1.5E+07 | 3.2E−01 | 1.7E−08 | 0.2 | 51% |
| mAb_Hm_S104D | 1.8E+05 | 6.6E−03 | 3.7E−08 | 0.4 | 33% |
| mAb_Hm_W106E | | | weak | NA | |
| mAb_Hm_G33S | 1.4E+05 | 1.0E−02 | 7.2E−08 | 0.8 | 28% |
| mAb_Hm_Y105S | | | weak | NA | |
| mAb_Hm_W106A | 1.6E+05 | 1.2E−02 | 7.1E−08 | 0.8 | 34% |
| mAb_Hm_W106L | 5.2E+04 | 1.7E−03 | 3.3E−08 | 0.4 | 52% |

Part B

| | | | | | |
|---|---|---|---|---|---|
| mAb (parental sequences) | 6.5E+04 | 5.1E−04 | 8.2E−09 | 0.0 | 100% |
| mAb_Lm_Q89V | 1.4E+04 | 8.0E−04 | 6.1E−08 | 0.3 | 36% |
| mAb_Lm_Y92A | | | weak | NA | |
| mAb_Lm_Y53D | 3.7E+08 | 7.0E+00 | 3.5E−08 | 0.2 | 37% |
| mAb_Lm_A32P | 1.7E+05 | 7.7E−03 | 4.5E−08 | 0.2 | 34% |
| mAb_Lm_Y92N | 2.5E+05 | 4.4E−02 | 1.7E−07 | 0.7 | 29% |
| mAb_Lm_S30R | 2.2E+05 | 4.9E−02 | 2.3E−07 | 1.0 | 30% |
| mAb_Lm_Y53N | 1.8E+05 | 4.9E−03 | 2.8E−08 | 0.1 | 40% |
| mAb_Hm_S104D | 2.5E+05 | 3.3E−03 | 1.4E−08 | 0.1 | 51% |
| mAb_Hm_W106E | 1.1E+05 | 1.7E−02 | 1.5E−07 | 0.7 | 26% |
| mAb_Hm_G33S | 1.7E+05 | 2.5E−02 | 1.4E−07 | 0.6 | 29% |
| mAb_Hm_Y105S | | | weak | NA | |
| mAb_Hm_W106A | 2.3E+05 | 2.9E−02 | 1.3E−07 | 0.6 | 32% |
| mAb_Hm_W106L | 5.9E+04 | 1.9E−03 | 3.3E−08 | 0.1 | 43% |

mAb = Sacituzumab
Lm = light chain mutation (amino acid substitution in V_{L} domain); positions are defined according to sequential numbering
Hm = heavy chain mutation (amino acid substitution in V_{H} domain); positions are defined according to sequential numbering
NA = not applicable
SPR = surface plasmon resonance

TABLE 15
SPR binding of Selected Sacituzumab Variants
(average of 28, 38, 49 RU)
TROP2 immobilized

| | Human Trop2 pH 7.4 | | | | |
|---|---|---|---|---|---|
| | | | | | % |
| | kₐ | k_{d} | K_{Dapp} | K_{D}/ | Ref |
| Antibody Mutein | (1/Ms) | (1/s) | (M) | K_{Dref} | BC |

Part A

| | | | | | |
|---|---|---|---|---|---|
| mAb (parental | 5.5E+04 | 2.0E−04 | 3.7E−09 | 1.0 | 100% |
| sequence) | | | | | |
| mAb_Lm_Y92A | 1.4E+05 | 5.1E−02 | 3.7E−07 | 100.0 | 41% |
| mAb_Lm_S30R | 1.5E+08 | 2.2E+01 | 1.8E−07 | 48.2 | 48% |
| mAb_Hm_S104D | 3.8E+08 | 1.3E+01 | 3.9E−08 | 10.7 | 53% |
| mAb_Hm_W106E | | | weak | NA | |
| mAb_Hm_Y105S | 1.0E+05 | 9.8E−03 | 9.7E−08 | 26.3 | 49% |
| mAb_Hm_W106A | 8.9E+04 | 2.6E−02 | 2.9E−07 | 80.1 | 46% |

Part B

| | | | | | |
|---|---|---|---|---|---|
| mAb (parental | 6.6E+04 | 1.8E−04 | 2.8E−09 | 1.0 | 100% |
| sequence) | | | | | |
| mAb_Lm_Y92A | 2.1E+05 | 9.9E−02 | 4.7E−07 | 167.7 | 53% |
| mAb_Lm_S30R | 1.5E+05 | 3.2E−02 | 2.1E−07 | 75.4 | 56% |
| mAb_Hm_S104D | 3.5E+05 | 2.1E−03 | 5.9E−09 | 2.1 | 79% |
| mAb_Hm_W106E | 1.0E+08 | 9.2E+00 | 9.4E−08 | 34.0 | 48% |
| mAb_Hm_Y105S | 1.5E+05 | 1.5E−02 | 1.0E−07 | 37.6 | 57% |
| mAb_Hm_W106A | 1.4E+05 | 3.9E−02 | 2.8E−07 | 100.0 | 54% |

mAb = Sacituzumab
Lm = light chain mutation (amino acid substitution in V_{L} domain); positions are defined according to sequential numbering
Hm = heavy chain mutation (amino acid substitution in V_{H} domain); positions are defined according to sequential numbering
NA = not applicable
SPR = surface plasmon resonance

In the tables, K_{DApp} is “apparent” K_{D} as it reflects an average of measurements of different chip loading. The RU (Response Units) refer to the amount of loading on the chip (8 and 28.8 RU means the measurements were performed with lower antigen density (8RU) and higher antigen density (28.8 RU). BC (Binding Capacity) is a measure of number of molecules that stay on the chip with regards to the reference, (lower k_{D} normally equals lower BC because of off-rate) it is a qualitative measure.

### Example 4

### Sacituzumab Re-Humanization and Best Single Mutation (BSM) Identification.

The amino acid sequence for Sacituzumab was evaluated using the software package BIOVIA Discovery Studio using the ‘Predict Humanizing Mutations’ tool. The configuration was set to exclude substitutions in the Vernier Zone, Kabat and IMGT CDR Residues. Calculation Mutation Energy was set to True. The germline gene match for the light chain was IGKJ4 and IGKV1_39_01 and heavy chain IGHJ4 and IGHV7_4_1. The BSM was determined by summation of each individual point mutant in the framework region predicted to be stabilizing. Overall, the BSM mutations are calculated to provide-9.03 kcal/mol stabilization using Chemistry at Harvard Macromolecular Mechanics (CHARMM) energetics over the clinical Sacituzumab sequence. The resulting Sacituzumab BSM sequence has higher identity to human germline with 83% identity to IGHV7-4-1*02 and 84% identity to IGKV1-13*02 versus the clinical sequence (81% identity for both chains).

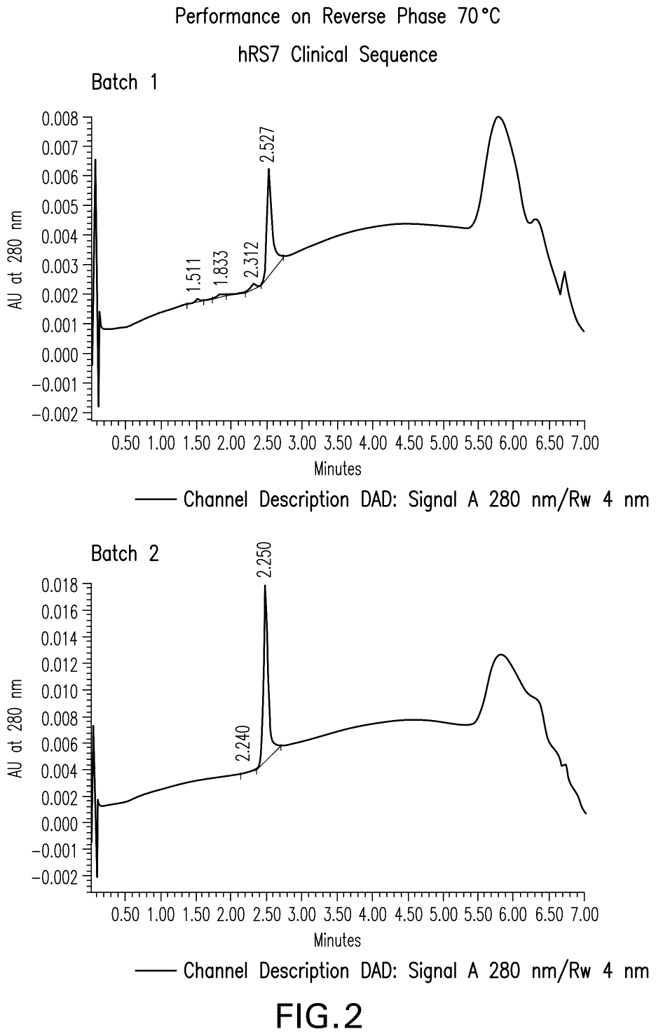

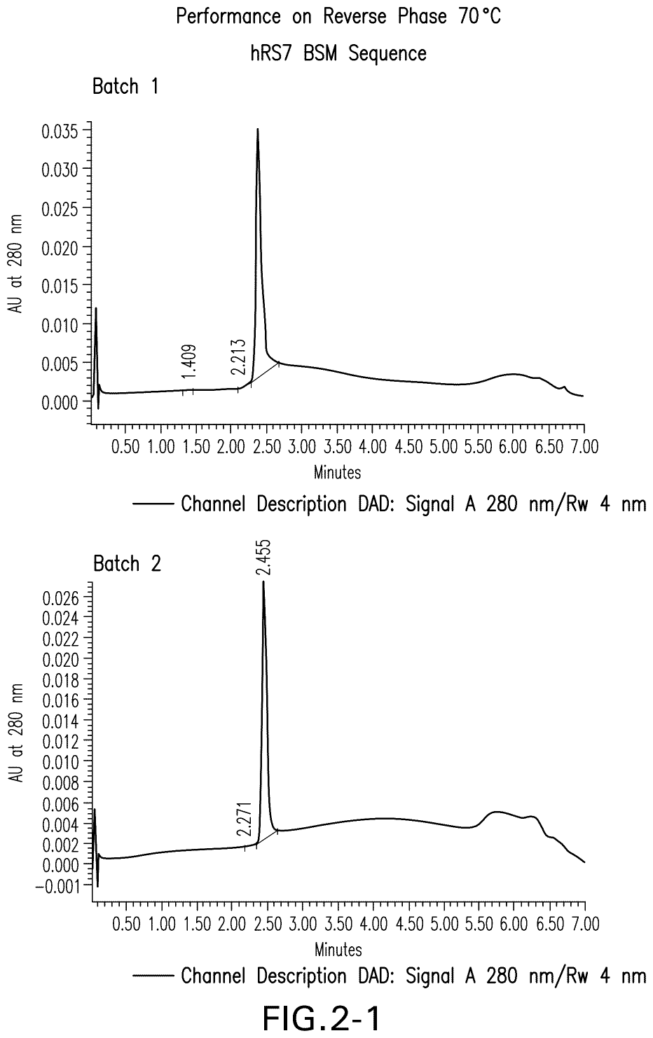

A comparison of the performance of Sacituzumab to Sacituzumab BSM on reverse phase high performance liquid chromatography (RP-HPLC) were comparable as shown in FIGS. 2 and 2-1. The RP-HPLC was performed as described in the General Methods on POROS™ resin at 70° C.

Hydrophobicity determinations were made for various Sacituzumab variants using HIC that was performed using as described in the General Methods on butyl HIC columns.

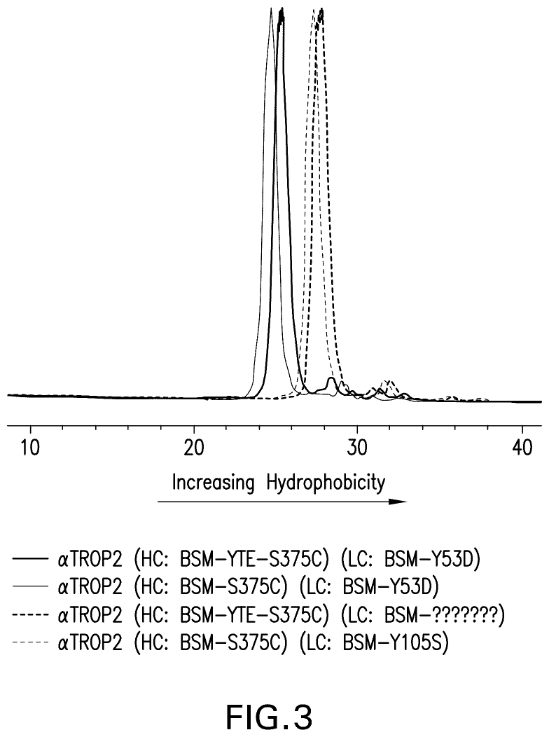

FIG. 3 shows a comparison of the hydrophobicity of the αTROP2 (HC:BSM-YTE-S375C) (LC:BSM-Y53D) antibody to the αTROP2 (HC:BSM-S375C) (LC:BSM-Y53D) antibody and the αTROP2 (HC:BSM-YTE-S375C) (LC:BSM-Y105S) antibody to the αTROP2 (HC:BSM-S375C) (LC:BSM-Y105S) antibody. The YTE substitution in the heavy chain effected little increase in hydrophobicity.

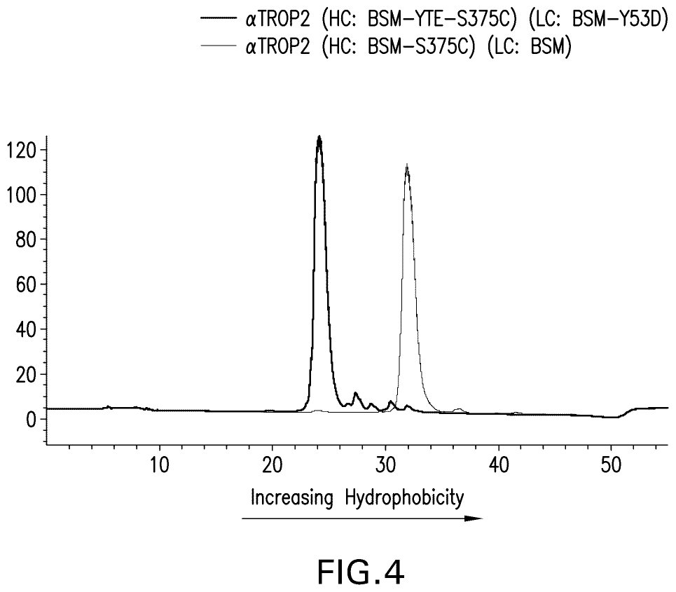

FIG. 4 shows a comparison of the hydrophobicity of the αTROP2 (HC:BSM-YTE-S375C) (LC:BSM-Y53D) antibody to the αTROP2 (HC:BSM-S375C) (LC:BSM) antibody. The αTROP2 (HC:BSM-YTE-S375C) (LC:BSM-Y53D) antibody peak elution was at fraction 24.14 whereas the αTROP2 (HC:BSM-S375C) (LC:BSM) antibody eluted at fraction 31.89. The Y53D substitution in the light chain effected a substantial decrease in hydrophobicity.

### Example 5

αTROP2 mAb variants were intravenously administered to biologic naïve male Rhesus monkeys to evaluate their pharmacokinetics (PK). The study design is listed in Table 16. Blood samples were collected from a peripheral vessel at indicated time points and serum was separated from blood cells for PK analysis with a ligand-binding assay using anti-human IgG framework antibodies. The PK parameters were estimated by noncompartmental analysis with Phoenix WinNonlin (Ver. 6.3, Certara).

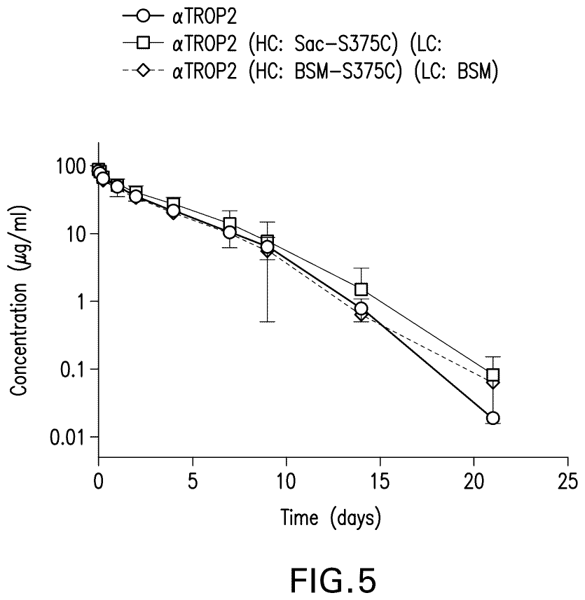

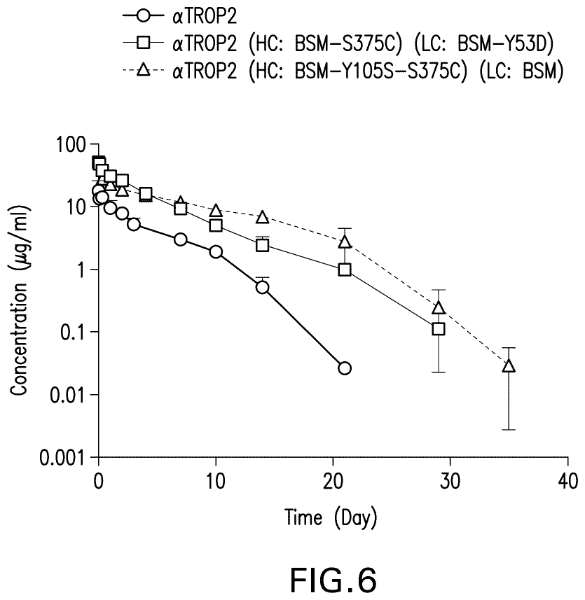

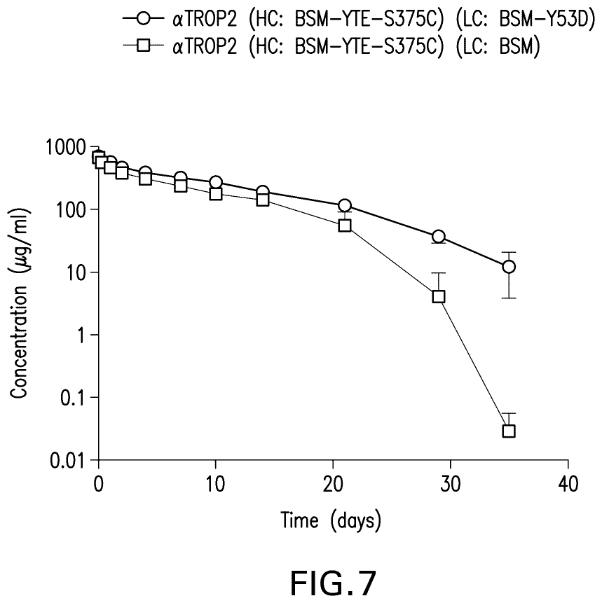

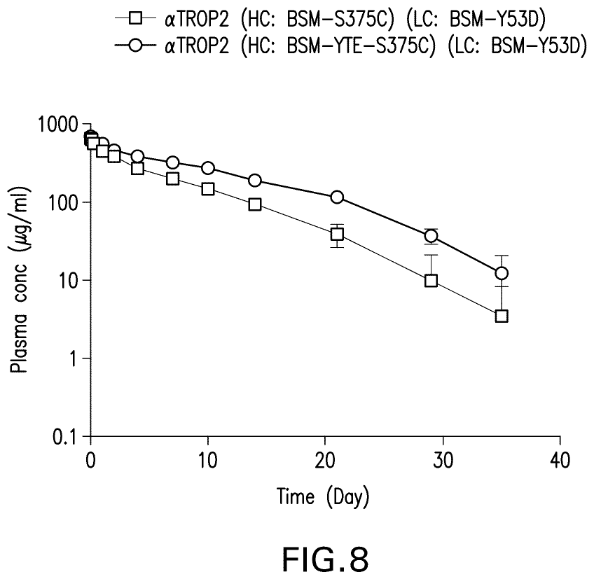

Following a single 3 mpk IV administration in Rhesus monkey, BSM, S375C mutations did not significantly affect parental αTROP2 PK as shown in FIG. 5 (Framework variants Rhesus PK after 3 mpk IV bolus administration) with clearance values between 11-13 mL/day/kg as shown by the data in Table 16. Avidity variants Y53D (light chain) and Y105S (heavy chain) on the BSM backbone had significantly lower plasma clearance (˜5 mL/day/kg) compared to parental αTROP2 mAb (˜18 mL/day/kg) after a single 1 mpk IV administration as shown in FIG. 6 (Affinity variants Rhesus PK after 1 mpk IV bolus administration) and the data in Table 17. Y53D mutation on the BSM-YTE backbone also improved monkey PK by reducing clearance from 4.5 mL/day/kg for BSM-YTE to 3 mL/day/kg for BSM-YTE-Y53D, and the terminal half-life increased from 1.3 days to 4.3 days as shown in FIG. 7 (BSM-YTE and BSM-YTE-Y53D Rhesus PK after a single 20 mpk IV administration) and the data in Table 18). The data shows that reducing the avidity and increasing the hydrophilicity can improve the PK of the αTROP2 mAb. Introducing YTE mutations to improve the FcRn based antibody recycling indeed also improved the PK of BSM-Y53D by increasing its AUC from 3930 μg/mL*day to 6560 μg/mL*day and extends half-life from 3.4 to 4.3 days as shown in FIG. 8 and the data in Table 19).

TABLE 16
NHP study design

| | No. of | | Dose Level | | |
|---|---|---|---|---|---|
| Group | Animal | Antibody | (mg/kg) | Route | PK Time point |
| 1 | 2 | αTROP2 | 1 | IV | 0, 0.083, 0.5, 1, 8 |
| 2 | 2 | αTROP2 | 3 | IV | hr, Day 1, Day 2, |
| 3 | 2 | αTROP2 (HC: S375C) | 3 | IV | Day 4, Day 5, Day |
| 4 | 2 | αTROP2 (HC: YTE) | 3 | IV | 7, Day 14, Day 21 |
| 5 | 2 | αTROP2 (HC: BSM)(LC: BSM) | 3 | IV | |
| 6 | 2 | αTROP2 (HC: BSM-Y105S- | 1 | IV | 0, 0.25, 2, 6 hr, Day |
| | | S375C)(LC: BSM) | | | 1, Day 2, Day 4, |
| 7 | 2 | αTROP2 (HC: BSM-S375C)(LC: | 1 | IV | Day 7, Day 10, Day |
| | | BSM-Y53D) | | | 14, Day 21, Day 29, |
| 8 | 2 | αTROP2 (HC: BSM-S375C)(LC: | 20 | IV | Day 35 |
| | | BSM-Y53D) | | | |
| 9 | 2 | αTROP2 (HC: BSM-YTE- | 20 | IV | |
| | | S375C)(LC: BSM-Y53D) | | | |
| 10 | 2 | αTROP2 (HC: BSM-YTE- | 20 | IV | |
| | | S375C)(LC: BSM) | | | |

HC = heavy chain;
LC = light chain;
BSM = Best Single Mutations;
HC and LC substitutions are defined by sequential numbering except as follows:
YTE = M252Y, S254T, T256E substitutions, positions defined according to Eu numbering;
HC constant domain S375C substitution, position defined according to Eu numbering

TABLE 17
BSM-Y53D, BSM-YTE, BSM-YTE-Y53D PK parameters

| | | AUC₍₀₋ₜ₎ | CL | Vₛₛ | |
|---|---|---|---|---|---|
| | Dose | (mg/ | (mL/ | (mL/ | t_{1/2} |
| Antibody | (mg/kg) | mL*day) | day/kg) | kg) | (day) |
| αTROP2 | 3 | 247 | 12 | 41 | 1.4 |
| αTROP2 (HC: S375C) | 3 | 289 | 11 | 37 | 1.9 |
| αTROP2 (HC: BSM) | | | | | |
| (LC: BSM) | 3 | 233 | 13 | 42 | 1.8 |

HC = heavy chain;
LC = light chain;
BSM = Best Single Mutations;
AUC = area under curve HC and LC substitutions are defined by sequential numbering except as follows:
HC constant domain S375C substitution, position defined according to Eu numbering
Vₛₛ = volume in steady state
CL = clearance
t_{1/2} = terminal half-life

TABLE 18
BSM-Y53D, BSM-YTE, BSM-YTE−Y53D PK parameters

| | | AUC₍₀₋ₜ₎ | CL | Vₛₛ | |
|---|---|---|---|---|---|
| | Dose | (mg/ | (mL/ | (mL/ | t_{1/2} |
| Antibody | (mg/kg) | mL*day) | day/kg) | kg) | (day) |
| αTROP2 | 1 | 56.0 | 18.4 | 80.6 | 2.00 |
| αTROP2 (HC: BSM- | 1 | 225 | 4.50 | 34.3 | 2.08 |
| Y105S-S375C)(LC: BSM) | | | | | |
| αTROP2 (HC: BSM- | 1 | 189 | 5.18 | 25.6 | 3.22 |
| S375C)(LC: BSM-Y53D) | | | | | |

HC = heavy chain;
LC = light chain;
BSM = Best Single Mutations;
HC and LC substitutions are defined by sequential numbering except as follows:
HC constant domain S375C substitution, position defined according to Eu numbering
AUC = area under curve
Vₛₛ = volume in steady state
CL = clearance
t_{1/2} = terminal half-life

TABLE 19
BSM-Y53D, BSM-YTE, BSM-YTE-Y53D PK parameters

| | | AUC₍₀₋ₜ₎ | CL | Vₛₛ | |
|---|---|---|---|---|---|
| | Dose | (mg/ | (mL/ | (mL/ | t_{1/2} |
| Antibody | (mg/kg) | mL*day) | day/kg) | kg) | (day) |
| αTROP2 (HC: BSM-YTE- | 20 | 6560 | 3.03 | 30.3 | 4.25 |
| S375C)(LC: BSM-Y53D) | | | | | |
| αTROP2 (HC: BSM-YTE- | 20 | 4480 | 4.54 | 33.9 | 1.27 |
| S375C)(LC: BSM) | | | | | |
| αTROP2 (HC: BSM- | 20 | 3930 | 5.12 | 38.2 | 3.38 |
| S375C)(LC: BSM-Y53D) | | | | | |

HC = heavy chain;
LC = light chain;
BSM = Best Single Mutations;
HC and LC substitutions are defined by sequential numbering except as follows:
YTE = M252Y, S254T, T256E substitutions, positions defined according to Eu numbering;
HC constant domain S375C substitution, position defined according to Eu numbering
AUC = area under curve
Vₛₛ = volume in steady state
CL = clearance
t_{1/2} = terminal half-life

### Example 6

In silico immunogenicity analysis (Protein immunogenicity scores).

Immunogenicity risk profiles for the amino acid sequence of various anti-TROP2 variants, including αTROP2 (HC:BSM-S375C) (LC:BSM-Y53D), were performed using the Interactive Screening and Protein Reengineering Interface (ISPRI) from EpiVax (Providence, RI). Within the ISPRI software suite, epitope prediction and self-homology assessments were performed using the EpiMatrix, ClusterMatrix, Antibody Analysis, and Janus Matrix Homology tools (Moise et al., Clin. Immunol. 142:320-331 (2012)).

First, the variant amino acid sequences were screened for MHC Class II epitope content using the EpiMatrix tool. The input amino acid sequences were evaluated for predicted binding to a panel of nine class II HLA-DRB1 alleles (*0101, *0301, *0401, *0701, *0801, *0901, *1101, *1301 and *1501). Regions of high epitope density were then identified in the protein sequences, using the ClustiMer algorithm. ClustiMer searches for contiguous segments of 15-30 amino acids with elevated binding across common HLA-DR alleles. Epitope clusters identified in ClustiMer were subsequently evaluated with the Janus Matrix algorithm to determine epitope selfness to assess the potential for immune tolerance for a given epitope. Janus Matrix Algorithm (Moise et al., Hum. Vaccin. Immunother. 9:1577-1586 (2013)) was used to identify epitopes that share T cell receptor (TCR)-face conservation (positions 2, 3, 5, 7 and 8) with epitopes restricted by the same alleles found in the human proteome. Epitopes with identical TCR-facing residues, which are also predicted to bind to the same MHC allele, are more likely to induce cross-reactive T cells. Additionally, the Janus-Matrix algorithm identifies epitopes known to correspond to an immunosuppressive regulatory T cell response (termed Tregitopes), which are thought to reduce the immunogenic potential of the construct. A Janus Matrix homology score threshold of two (cross conserved HLA allele-specific epitopes averaged over the length of the sequence) for cross-conservation with human (self) proteins was applied to identify epitopes with elevated potential to be tolerated or actively regulatory.

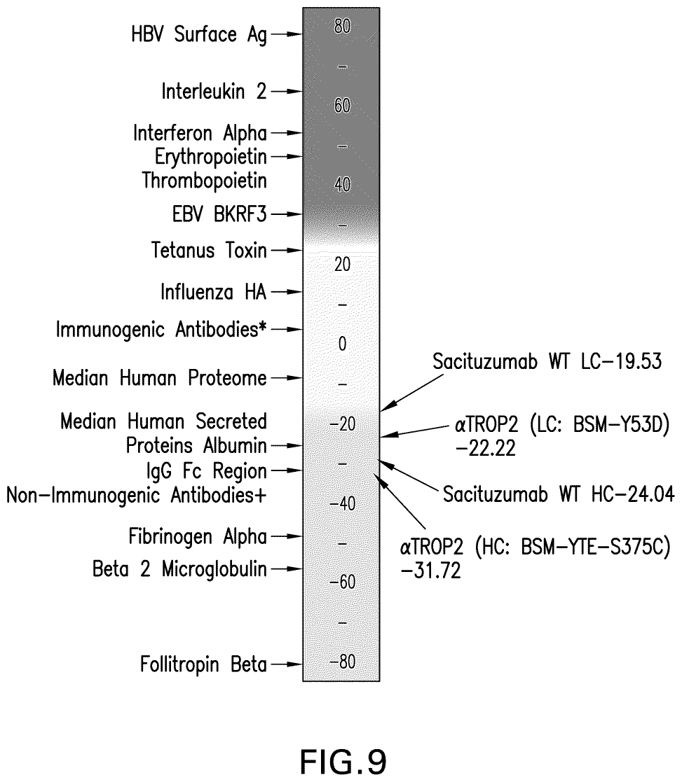

Of the TROP2 variant sequences analyzed, αTROP2 (HC:BSM-S375C) (LC:BSM-Y53D) had the lowest predicted immunogenicity, demonstrating an overall Tregitope-adjusted Protein Immunogenicity Score of (−31.72), which is comparable to the immunogenic profile of known, non-immunogenic antibodies tested in the clinic. The Antibody analysis tool predicted a 1.85% ADA response in clinical populations and bucketed αTROP2 (HC:BSM-S375C) (LC:BSM-Y53D) as an “optimal antibody” construct due to its relatively low predicted effector epitope content and high predicted Tregitope content. Overall, αTROP2 (HC:BSM-S375C) (LC:BSM-Y53D) was found to have less predicted epitope content than the other candidate sequences, including wild-type Sacituzumab (See FIG. 9). The K38R mutation in the heavy chain of αTROP2 (HC:BSM-S375C) (LC:BSM-Y53D) disrupts two, promiscuous, non-self-epitopes found in wild-type Sacituzumab and introduces two Tregitopes with high probability of HLA binding, reducing the overall epitope content and predicted immunogenic risk for this molecule.

### Example 7

Synthesis of 4-((S)-2-((S)-2-(3-(2,5-dioxo-2,5-dihydro-1H-pyrrol-1-yl)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1).

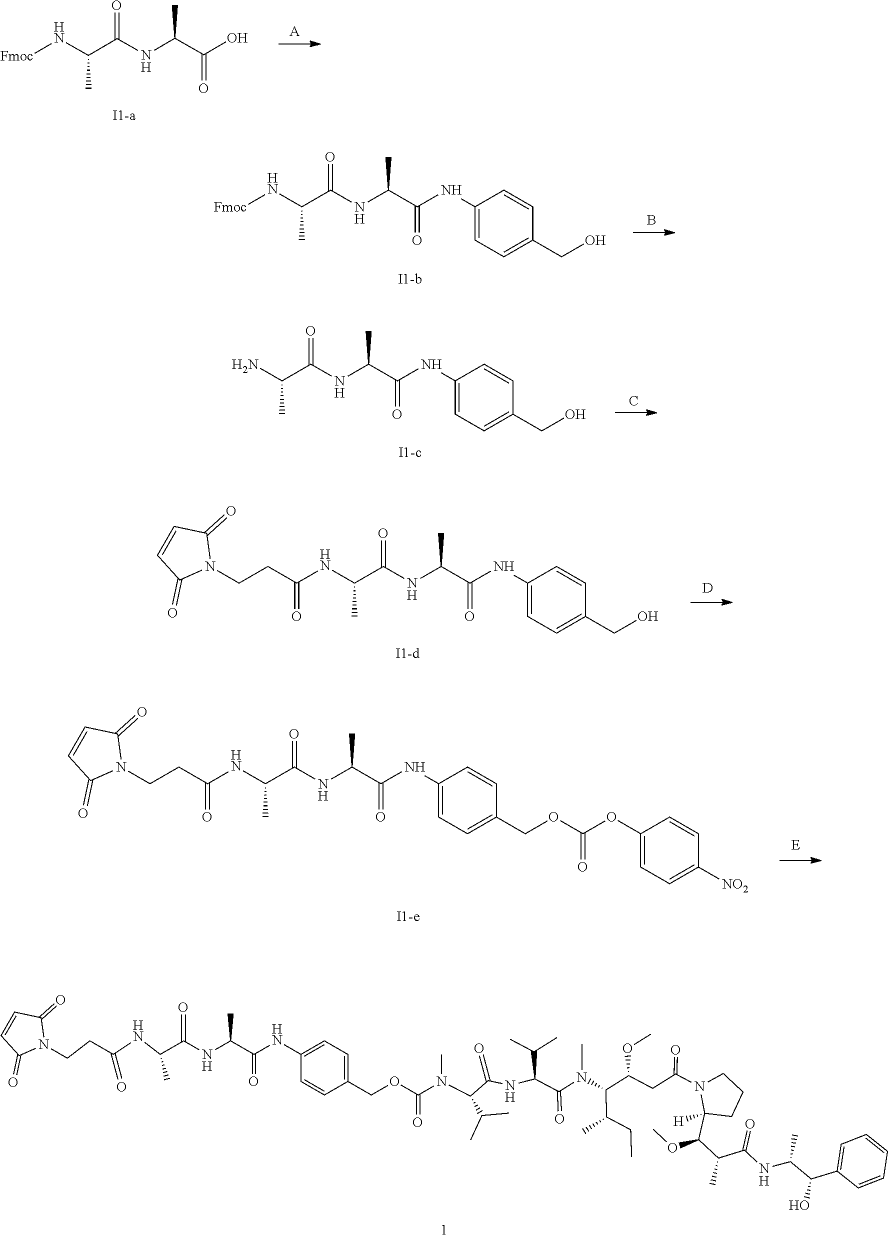

### Step A—Synthesis of Compound 11-b

P-aminobenzylalcohol (154 g, 125 mmol) was added to a stirred mixture of ethyl 2-ethoxy-2H-quinoline-1-carboxylate (103 g, 418 mmol) and (2S)-2-[(2S)-2-{[(9H-fluoren-9-ylmethoxy)carbonyl]amino}propanamido]propanoic acid I1-a (100 g, 261 mmol) in dichloromethane (DCM): MeOH (2:1) (3000 mL) at 0° C. The reaction mixture was stirred at room temperature for 18 hours and monitored by LCMS. The solvent was evaporated in vacuo, and the residue was diluted with methyl tertiary butyl ether (TBME) (2000 mL) and stirred for 30 minutes. The solids were collected by filtration, washed with TBME (1000 mL) and dried in vacuo to provide 9H-fluoren-9-ylmethyl N-[(1S)-1-{[(1S)-1-{[4-(hydroxymethyl)phenyl]carbamoyl}ethyl]-carbamoyl}ethyl]carbamate (11-b). LCMS: (ES, m/z): [M+H]⁺=488.

### Step B—Synthesis of Compound 11-c

Diethylamine (756 g, 10.3 mol) was added to a stirred mixture of 9H-fluoren-9-ylmethyl N-[(1S)-1-{[(1S)-1-{[4-(hydroxymethyl)phenyl]carbamoyl}ethyl]-carbamoyl}ethyl]carbamate (I1-b) (140 g, 287 mmol) in DMF (1.4 L) at room temperature. The mixture was stirred at room temperature for 18 hours. The reaction was monitored by LCMS. The solvent was evaporated, and the residue was diluted with ethyl acetate (EtOAc) (500 mL) and stirred for 30 minutes. The solids were collected by filtration, washed with EtOAc (300 mL), and dried in vacuo to provide (2S)-2-[(2S)-2-aminopropanamido]-N-[4-(hydroxymethyl)phenyl]propanamide (I1-c). LCMS: (ES, m/z): [M+H]⁺=266.

### Step C—Synthesis of Compound 11-d

A mixture of 2,5-dioxopyrrolidin-1-yl 3-(2,5-dioxopyrrol-1-yl) propanoate (55.2 g, 207 mmol), I1-c (55.0 g, 207 mmol) and DIPEA (40.2 g, 311 mmol) in DMF (550 mL) at room temperature was stirred for 16 hours. The reaction mixture was added to H₂O (600 mL) with stirring. The solids were collected by filtration, washed with water (500 mL), and dried in vacuo to provide (2S)-2-[3-(2,5-dioxopyrrol-1-yl) propanamido]-N-[(1S)-1-{[4-(hydroxymethyl)phenyl]carbamoyl}ethyl]propanamide (11-d) that was used directly in the next step.

### Step D—Synthesis of Compound 11-e

N,N-Diisopropylethylamine (DIPEA) (30.7 g, 237 mmol) was added to a stirred mixture of bis(4-nitrophenyl) carbonate (60.3 g, 198 mmol) and (2S)-2-[3-(2,5-dioxopyrrol-1-yl) propanamido]-N-[(1S)-1-{[4-(hydroxymethyl)phenyl]carbamoyl}ethyl]propanamide (I1-d) (55 g, 32 mmol) in dimethylformamide (DMF) (550 mL) at room temperature. The mixture was stirred for 16 hours and monitored by liquid chromatography-mass spectrometry (LCMS). The reaction solution was added to H₂O (400 mL) with stirring. The mixture was filtered, and the filter cake was washed with water (1×100 mL). The solids were collected by filtration and purified using reverse phase chromatography (Dynamic axial chromatographic column C-18, eluting with 15% to 65% ACN/Water (with 0.1% ammonium acetate (NH₄OAc) as modifier)). The resulting mixture was concentrated in vacuo, and the solids were dried in vacuo at 45° C. to provide {4-[(2S)-2-[(2S)-2-[3-(2,5-dioxopyrrol-1-yl) propanamido]propanamido]propanamido]phenyl}methyl-4-nitrophenyl carbonate (I1-e). LC-MS: (ES, m/z): [M+H]⁺=582.

### Step E—Synthesis of 1

To a 60 mL round-bottomed flask under N₂ was added {4-[(2S)-2-[(2S)-2-[3-(2,5-dioxopyrrol-1-yl)propanamido]propanamido]propanamido]phenyl}methyl-4-nitrophenyl carbonate (11-e) (3.00 g, 5.15 mmol) and DMF (45 mL), followed by hydroxybenzotriazole (HOBt) (140 mg, 1.03 mmol). The reaction mixture was stirred at 25° C. for 10 minutes, after which time MMAE (4.00 g. 5.57 mmol) was added at 25° C. The reaction mixture was stirred at 25° C. for 16 hours. The reaction was then purified using reverse phase column chromatography (AQ C18 30% to 60% acetonitrile (MeCN)/water with 0.1% ammonium acetate as modifier). The MeCN was concentrated in vacuo, the remaining aqueous mixture extracted with ethyl acetate (300 ml×3), and the combined organics were concentrated in vacuo. Four batches were run in parallel, combined, and dissolved in 200 mL of MeCN. 400 mL water was added, and the solution was frozen and then lyophilized to afford 4-((S)-2-((S)-2-(3-(2,5-dioxo-2,5-dihydro-1H-pyrrol-1-yl)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1). LC-MS: (ES, m/z): [M+H]⁺=1160. ¹H NMR (400 MHZ, CD₃OD) δ 7.53 (br d, J=8.19 Hz, 2H), 7.03-7.32 (m, 7H), 6.67 (s, 2H), 5.21-5.29 (m, 2H), 4.90-5.14 (m, 2H), 4.28-4.66 (m, 4H), 4.04-4.22 (m, 4H), 3.52-3.81 (m, 4H), 3.24-3.46 (m, 6H), 3.17 (s, 2H), 2.97-3.09 (m, 2H), 2.73-2.90 (m, 4H), 2.28-2.47 (m, 4H), 1.43-2.20 (m, 8H), 1.16-1.38 (m, 10H), 1.00-1.12 (m, 6H), 0.61-0.94 (m, 18H). Not all exchangeable protons are reported.

### Example 8

### Synthesis of 4-((S)-2-((S)-2-(3-(5-cyano-6-(methylsulfonyl)picolinamido)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2)

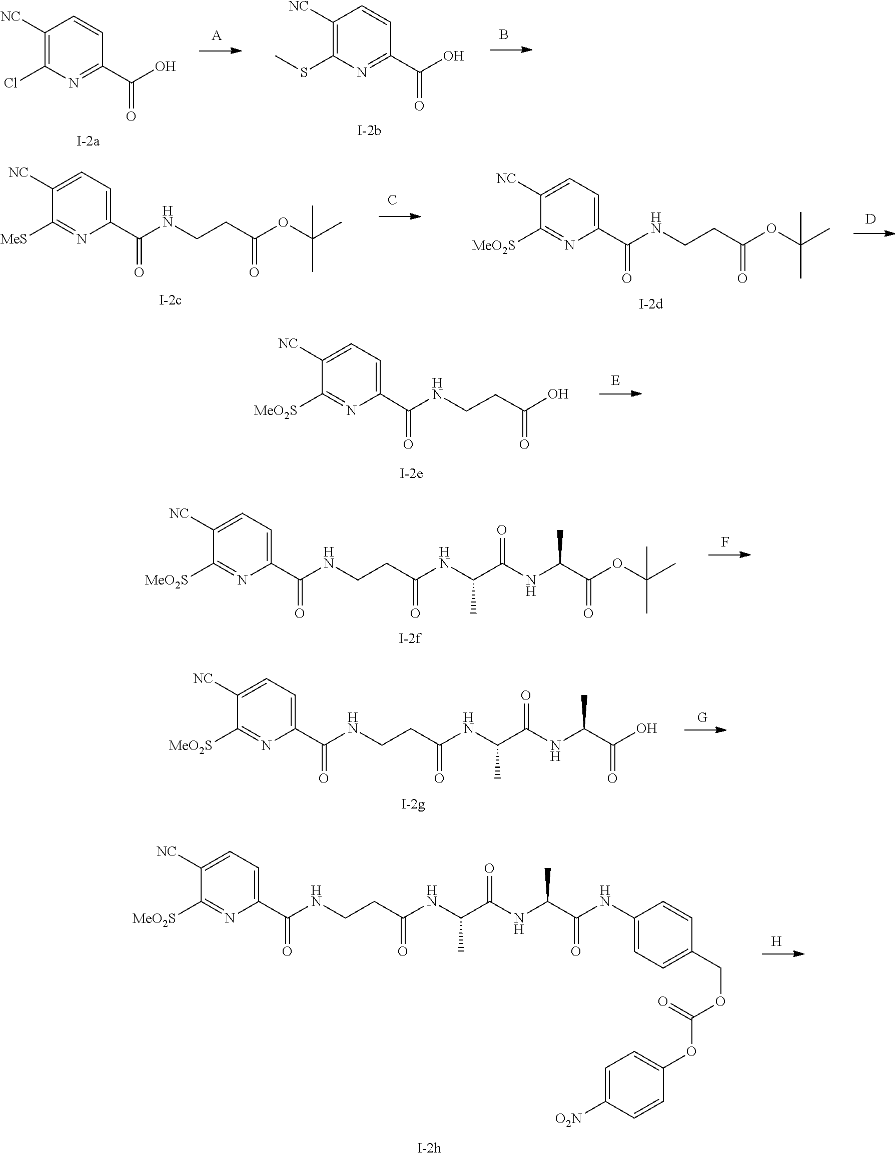

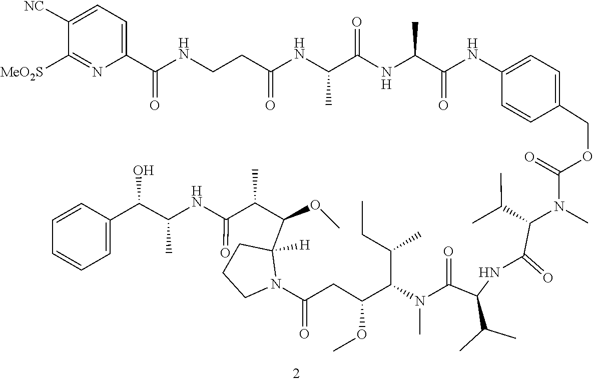 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2ccc(C#N)c(S(C)(=O)=O)n2)cc1)C(C)C]

### Step A—Synthesis of Compound I-2b

6-Chloro-5-cyanopicolinic acid (I-2a) (80 g, 0.44 mol) was dissolved in DMF (5000 mL), and sodium methane thiolate (77 g, 1.1 mol) was added into the mixture in batches. The reaction mixture was stirred at 25° C. for 16 hours. The reaction mixture was then diluted with ethyl acetate and added to water. The mixture was extracted with ethyl acetate and the aqueous phase was adjusted to pH 5 with 10% citric acid. The mixture was extracted with ethyl acetate 3 times and the combined organics were concentrated in vacuo to provide 5-cyano-6-(methylthio) picolinic acid (I-2b). LC-MS: (ES, m/z): [M+H]⁺=195.

### Step B—Synthesis of Compound I-2c

To 5-cyano-6-(methylthio) picolinic acid (I-2b) (66 g, 0.34 mol) dissolved in tetrahydrofuran (THF) (3000 mL) was added (1-[Bis(dimethylamino)methylene]-1H-1,2,3-triazolo[4,5-b]pyridinium 3-oxide hexafluorophosphate (HATU) (155 g, 0.41 mol). The resulting mixture was stirred for 30 minutes at 25° C. tert-Butyl 3-aminopropanoate HCl salt (67.7 g, 0.37 mol) was added into the mixture and then it was cooled to 10° C. N,N-Diisopropylethylamine (DIEA) (175 g, 1.35 mol) was added dropwise over 1 hour at 10° C. The reaction mixture was stirred at 25° C. for 16 hours, concentrated in vacuo and diluted with ethyl acetate. The resulting solution was washed with water 3 times and the combined organics were concentrated in vacuo. The residue was purified using silica gel column chromatography (ethyl acetate:petroleum ether 1:2) to provide tert-butyl 3-(5-cyano-6-(methylthio) picolinamido) propanoate (I-2c). LC-MS: (ES, m/z): [M+Na]⁺=344

### Step C-Synthesis of Compound I-2d

Tert-Butyl 3-(5-cyano-6-(methylthio) picolinamido) propanoate (I-2c). (96 g, 0.30 mol) was dissolved in DCM (1500 mL) and a solution of meta-chloroperoxybenzoic acid (m-CPBA) (206 g, 1.19 mol) in DCM (1500 mL) was added dropwise at 0° C. The reaction mixture was stirred at room temperature for 16 hours. The resulting reaction mixture was diluted with DCM and poured into ice water. The organic phase was washed with 10% sodium bicarbonate in water 4 times and the combined organics were dried and concentrated in vacuo to provide tert-butyl 3-(5-cyano-6-(methylsulfonyl) picolinamido) propanoate (I-2d). LC-MS: (ES, m/z): [M+Na]⁺=376

### Step D-Synthesis of Compound I-2e

To tert-butyl 3-(5-cyano-6-(methylsulfonyl) picolinamido) propanoate (I-2d) (90 g, 0.25 mol) dissolved in 1,4-dioxane (1000 mL) was added 4 M HCl in 1,4-dioxane (2500 mL). The resulting mixture was stirred at 25° C. for 16 hours and then filtered. The solid was washed with n-heptane and dried under nitrogen to provide 3-(5-cyano-6-(methylsulfonyl) picolinamido) propanoic acid (I-2e). LC-MS: (ES, m/z): [M+Na]⁺=320

### Step E Synthesis of Compound I-2f

Tert-Butyl L-alanyl-L-alaninate (iii) (50 g, 0.23 mol; Example 9) was dissolved in THF (2300 mL). To this mixture was added 3-(5-cyano-6-(methylsulfonyl) picolinamido) propanoic acid (I-2e) (69 g, 0.23 mol) and HATU (106 g, 0.28 mol). The reaction mixture was stirred for 30 minutes and then cooled to 10° C. DIEA (90 g) was then added dropwise over 30 minutes. The reaction mixture was stirred overnight at 20° C. The reaction mixture was then concentrated in vacuo, and the resulting residue was diluted with ethyl acetate. The combined organics were washed with water 3 times, dried, and concentrated in vacuo. The resulting residue was purified using silica gel chromatography with DCM:MeOH (2:1) to provide tert-butyl (3-(5-cyano-6-(methylsulfonyl) picolinamido) propanoyl)-L-alanyl-L-alaninate (I-2f). LC-MS: (ES, m/z): [M+Na]⁺=518

### Step F Synthesis of Compound I-2g

4 M HCl in 1,4-dioxane (1800 mL) was added to tert-butyl (3-(5-cyano-6-(methylsulfonyl) picolinamido) propanoyl)-L-alanyl-L-alaninate (I-2f) (90 g, 0.18 mol) dissolved in MeCN (2000 mL). The reaction mixture was stirred at 25° C. for 16 hours, then concentrated in vacuo. The resulting solid was washed with MTBE and filtered. The solid was air-dried overnight to provide (3-(5-cyano-6-(methylsulfonyl) picolinamido) propanoyl)-L-alanyl-L-alanine (I-2g). LC-MS: (ES, m/z): [M+Na]⁺=462

### Step G Synthesis of Compound I-2h

Into a 500 mL 4-necked round-bottom flask was purged and maintained with an inert atmosphere of nitrogen, was added (3-(5-cyano-6-(methylsulfonyl) picolinamido) propanoyl)-L-alanyl-L-alanine (I-2g) (30 g, 0.65 mol), DMF (300 mL), and HATU (31.2 g, 0.780 mol). The reaction mixture was stirred for 30 minutes at room temperature. Then (4-aminophenyl) methanol (9.0 g, 0.068 mmol) was added at 20° C., and the reaction mixture was cooled to 10° C. DIEA (90 g, 0.19 mmol) was added dropwise into the reaction mixture over 30 minutes at 10° C., and the resulting mixture was stirred at 25° C. for 5 hours. Then bis(4-nitrophenyl) carbonate (42 g, 0.13 mmol) was added into reaction mixture at 25° C., and stirred at 25° C. for 1 hour. The reaction mixture was purified using C-18 flash column chromatography (30% to 60% ACN/water (with 0.05% TFA as modifier)) to provide 4-((S)-2-((S)-2-(3-(5-cyano-6-(methylsulfonyl)picolinamido)propanamido)propanamido)propanamido)benzyl (4-nitrophenyl) carbonate (I-2h). LC-MS: (ES, m/z): [M+Na]⁺=732

### Step H-Synthesis of Compound 2

To 4-((S)-2-((S)-2-(3-(5-cyano-6-(methylsulfonyl)picolinamido)propanamido)propanamido)propanamido)benzyl (4-nitrophenyl) carbonate (I-2h) (5.6 g, 7.9 mmol) and 1H-benzo[d][1,2,3]triazol-1-ol (0.213 g, 1.58 mmol) was added DMF (56.0 mL). MMAE (5.67 g, 7.89 mmol) was added to the reaction at 20° C. and the reaction mixture was stirred at 40° C. 16 hours. The resulting mixture was purified using Prep-HPLC (10-95% MeCN/water, 0.05% TFA) to provide 4-((S)-2-((S)-2-(3-(5-cyano-6-(methylsulfonyl)picolinamido)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2). 1H NMR (400 MHZ, DMSO-d6) δ 9.86 (s, 1H), 9.14 (t, J=6.0 Hz, 1H), 8.79 (d, J=8.1 Hz, 1H), 8.38 (d, J=8.1 Hz, 1H), 8.33-7.97 (m, 3H), 7.96-7.51 (m, 3H), 7.41-6.87 (m, 7H), 6.09 (s, 1H), 5.37 (dd, J=26.2, 5.0 Hz, 1H), 5.04 (tt, J=25.6, 12.6 Hz, 2H), 4.69 (d, J=44.2 Hz, 1H), 4.59-4.16 (m, 5H), 4.15-3.90 (m, 2H), 3.80-3.43 (m, 8H), 3.36-2.95 (m, 9H), 2.94-2.58 (m, 2H), 2.50-2.20 (m, 3H), 2.19-1.79 (m, 3H), 1.65-1.37 (m, 3H), 1.32-1.18 (m, 9H), 1.06-0.96 (m, 7H), 0.89-0.59 (m, 20H).

### Example 9

### Preparation of Intermediate tert-butyl L-alanyl-L-alaninate (iii)

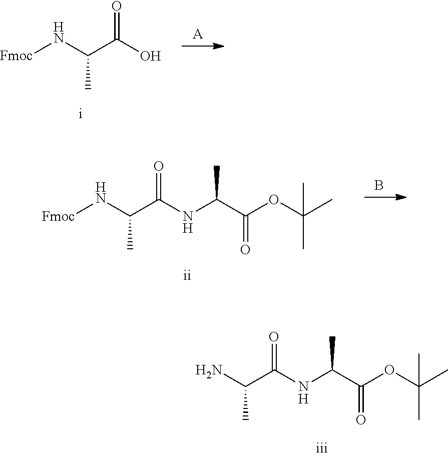

### Step A—Synthesis of Compound ii

To (((9H-fluoren-9-yl) methoxy) carbonyl)-L-alanine (i) (140 g, 450 mmol) dissolved with DCM (4500 mL) was added PyBOP (281 g, 540 mmol, 1.2 eq) at room temperature. The reaction mixture was stirred for 20 minutes. tert-Butyl L-alaninate HCl (85.6 g, 471 mmol) was added, and the solution was cooled to 0° C. DIEA (175 g, 1.35 mol) was then added dropwise into the reaction over 1 hour, and it was then stirred overnight at 20° C. The reaction mixture was concentrated in vacuo, diluted with ethyl acetate and washed with aqueous sodium carbonate (1 M), potassium bisulfate (1 M), water, and brine. The organic phase was then concentrated in vacuo and evaporated to 25% of the original solvent volume. MTBE (1400 mL) was added dropwise, and the mixture was stirred for 5 hours. The suspension was filtered, and the solids were washed with MTBE to provide tert-butyl (((9H-fluoren-9-yl) methoxy) carbonyl)-L-alanyl-L-alaninate (ii). LCMS: (ESI, m/z): [M+H]⁺=439.2.

### Step B—Synthesis of Compound iii

To tert-butyl (((9H-fluoren-9-yl) methoxy) carbonyl)-L-alanyl-L-alaninate (ii) (172 g, 392 mmol) dissolved in DCM (2000 mL) was added triethanolamine (TEA) (2000 mL). The reaction mixture was warmed to 40° C. and stirred for 16 hours. The reaction mixture was then concentrated in vacuo and purified using silica gel chromatography (DCM: 4 M NH₃ in MeOH 5:1) to provide tert-butyl L-alanyl-L-alaninate (iii). LCMS: (ESI, m/z): [M+H]⁺=217.1.

### Example 10

### Preparation of Intermediate Compound 3-(5-cyano-6-methylsulfonyl)nicotinamido)propanoic acid (x)

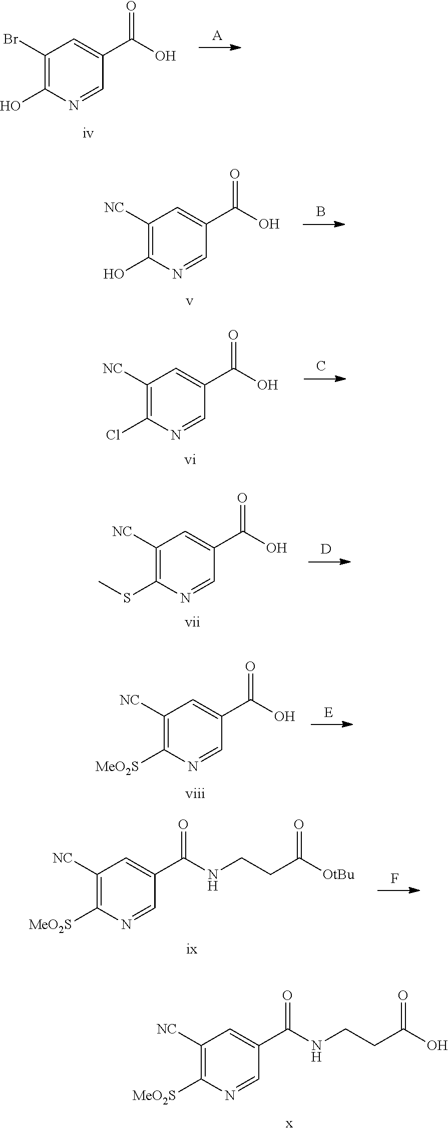

### Step A—Synthesis of Compound v

A solution of 5-bromo-6-hydroxypyridine-3-carboxylic acid (iv, 42.8 g, 196 mmol) and cuprous cyanide (35.2 g, 393 mmol) in N-Methyl-2-pyrrolidone (NMP) (430 mL) was stirred for 2 hours at 165° C. under a N₂ atmosphere. The mixture was concentrated in vacuo, and the crude product was purified using reverse phase flash chromatography (AQ C18 silica gel, ACN in water 0% to 20% gradient (with 0.5% NH₃·H₂O as modifier)) to provide crude product. The resulting mixture was filtered, and the filter cake was washed with H₂O. The filtrate was concentrated in vacuo to provide 5-cyano-6-hydroxypyridine-3-carboxylic acid (v). LCMS: (ESI, m/z): [M−H]⁻=163.

### Step B—Synthesis of Compound vi

A solution of 5-cyano-6-hydroxypyridine-3-carboxylic acid (v) (25.7 g, 157 mmol) and phosphorus oxychloride (130 mL) was stirred for 2 hours at 110° C. The reaction was monitored by LCMS and then concentrated in vacuo. Water and EtOAc cooled to 10° C. were added, and the solid was filtered out. The mixture was extracted with ethyl acetate, dried and concentrated in vacuo. The residue was purified using reverse phase flash chromatography (column AQ silica gel, 0% to 15% acetonitrile/sodium bicarbonate (aq.)). The pH value of the aqueous layer was adjusted to 2-3 with 1 M HCl and then the aqueous layer was extracted with ethyl acetate (3×), dried, and concentrated in vacuo to provide 6-chloro-5-cyanopyridine-3-carboxylic acid (vi). LCMS: (ESI, m/z): [M−H]⁻=181.

### Step C—Synthesis of Compound vii

Into a 500 mL three-necked bottle under a nitrogen atmosphere was added dimethylformamide (115 mL) and 6-chloro-5-cyanopyridine-3-carboxylic acid (vi) (7.7 g, 42 mmol) at 25° C. To the above mixture was added (methylsulfanyl)sodium (7.39 g, 105 mmol) in portions at 0° C. The resulting mixture was stirred for 8 hours at 25° C. The reaction was monitored by LCMS. This solution was slowly transferred to H₂O (1200 mL), then extracted with ethyl acetate (1×700 mL). The pH value of the aqueous layer was adjusted to 2-3 with 1 M HCl. The solid was collected by filtration to provide 5-cyano-6-(methylsulfanyl)pyridine-3-carboxylic acid (vii). LCMS: (ESI, m/z): [M−H]⁻=193.

### Step D—Synthesis of Compound viii

A solution of 5-cyano-6-(methylsulfanyl)pyridine-3-carboxylic acid (vii) (7.4 g, 38 mmol) in DCM (185 mL) was treated with m-CPBA (26.3 g, 152 mmol) for 24 hours at 45° C. under nitrogen atmosphere. The reaction was monitored by LCMS and then quenched with saturated sodium bisulfite at 0° C. and concentrated in vacuo. To the residue was added 2-methyltetrahydrofuran, and then the mixture was filtered and concentrated in vacuo to provide the crude product. The residue was purified using silica gel column chromatography eluting with DCM/MeOH to afford 5-cyano-6-(methylsulfonyl) nicotinic acid (viii). LCMS: (ESI, m/z): [M+H]⁺=227.05.

### Step E—Synthesis of Compound ix

5-Cyano-6-(methylsulfonyl) nicotinic acid (viii) (0.57 g, 2.5 mmol) and HATU (1.0 g, 2.7 mmol) were dissolved in 10 mL DMF and stirred for 30 minutes at 25° C. Then tert-butyl 3-aminopropanoate (0.42 g, 2.8 mmol) was added, and the mixture was cooled to 10° C. DIPEA (0.873 mL, 5.00 mmol) was added dropwise into the reaction mixture at 10° C. The reaction mixture was stirred at 25° C. for 2 hours and then diluted with water and extracted with EtOAc (3×). The organic phase was concentrated in vacuo, and the residue was purified using silica gel column chromatography eluting with 2:1 to 1:1 hexanes:EtOAc to provide tert-butyl 3-(5-cyano-6-(methylsulfonyl) nicotinamido) propanoate (ix). 1H NMR (500 MHZ, CD₃OD) δ 8.49 (d, J=2.0 Hz, 1H), 8.07 (d, J=2.0 Hz, 1H), 4.12 (s, 3H), 2.92 (t, J=6.9 Hz, 2H), 1.88 (t, J=6.9 Hz, 2H), 0.74 (s, 9H).

### Step F—Synthesis of Compound x

Tert-Butyl 3-(5-cyano-6-(methylsulfonyl) nicotinamido) propanoate (ix) (0.10 g, 0.28 mmol) was dissolved in 10 mL 1,4-dioxane. Then 4 M HCl in 1,4-dioxane (10 mL) was added into the mixture. The resulting mixture was stirred at 25° C. for 16 hours, and the reaction mixture was filtered. The solid was washed with n-heptane and dried under nitrogen for 5 hours to provide 3-(5-cyano-6-(methylsulfonyl) nicotinamido) propanoic acid (x). 1H NMR (500 MHZ, CD₃OD) δ 9.22 (d, J=1.9 Hz, 1H), 8.80 (d, J=1.9 Hz, 1H), 3.66 (t, J=5.8 Hz, 2H), 3.44 (s, 3H), 2.67 (t, J=6.8 Hz, 2H).

### Example 11

### Preparation of Intermediate Compound 4-((S)-2-((S)-2-aminopropanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiv)

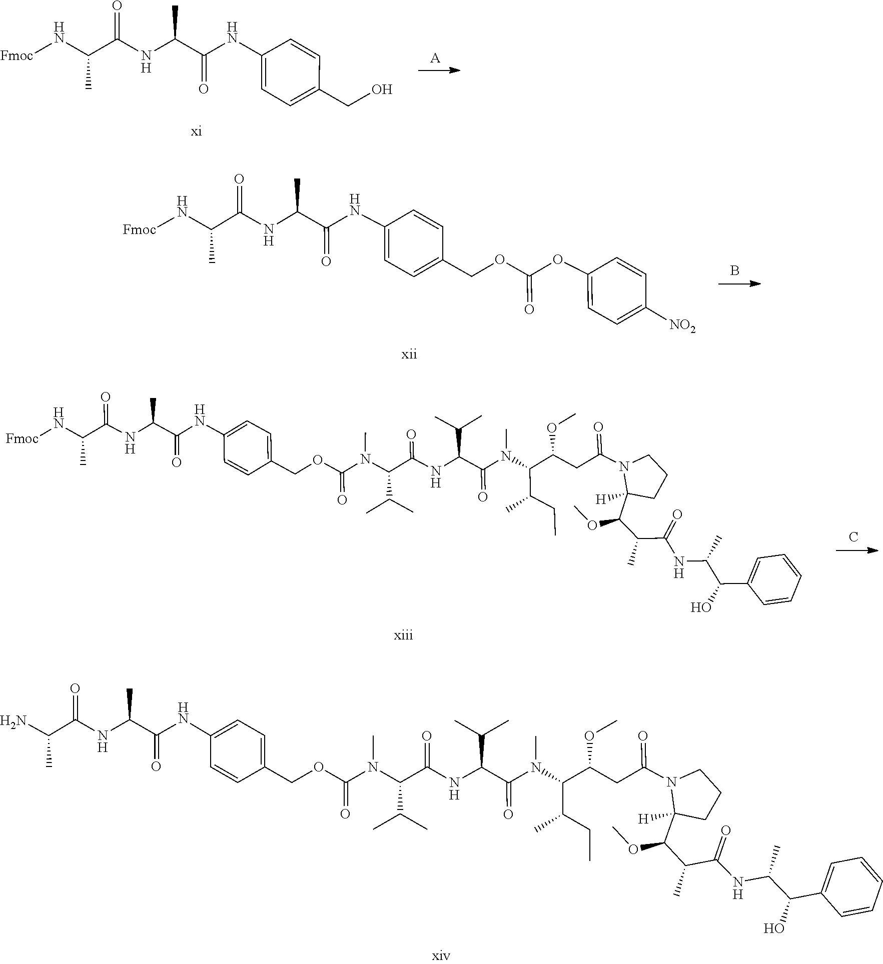

### Step A—Synthesis of Compound xii

A solution of (9H-fluoren-9-yl)methyl ((S)-1-(((S)-1-((4-(hydroxymethyl)phenyl)amino)-1-oxopropan-2-yl)amino)-1-oxopropan-2-yl) carbamate (xi) (Fmoc-Ala-Ala-PAB) (13 g, 27 mmol, 1 eq) in DMF (130 mL,) was added into a 500 mL 4-necked round-bottom flask purged and maintained with an inert atmosphere of nitrogen. Then 2 mol % DIEA (0.534 mmol) and bis(4-nitrophenyl) carbonate (16.3 g, 53.6 mmol, 2 eq) were added into the reaction mixture at 20° C. The reaction mixture was warmed to 45° C. and stirred for 16 hours. The reaction mixture was cooled to room temperature and purified using reverse phase flash column chromatography (30% to 60%, MeCN/water with 0.05% TFA as modifier) to provide (9H-fluoren-9-yl)methyl ((S)-1-(((S)-1-((4-((((4-nitrophenoxy) carbonyl)oxy)methyl)phenyl)amino)-1-oxopropan-2-yl)amino)-1-oxopropan-2-yl) carbamate (xii). LCMS: (ESI, m/z): [M+H]⁺=653.2.

### Step B—Synthesis of Compound xiii

A solution of (9H-fluoren-9-yl)methyl ((S)-1-(((S)-1-((4-((((4-nitrophenoxy) carbonyl)oxy)methyl)phenyl)amino)-1-oxopropan-2-yl)amino)-1-oxopropan-2-yl) carbamate (xii) (8.30 g, 12.7 mmol) in DMF (83 mL) was added into a 250 mL 4-necked round-bottom flask purged and maintained with an inert atmosphere of nitrogen. Then HOBt (340 mg, 2.5 mmol) was added, and the reaction mixture was stirred for 10 minutes at room temperature. MMAE (9 g, 12.7 mmol) was then added to the reaction mixture, and the resulting mixture was stirred for 16 hours at room temperature. The reaction mixture was purified using reverse phase flash column chromatography (20% to 50% MeCN/water with 0.05% TFA as modifier) to provide 4-((S)-2-((S)-2-((((9H-fluoren-9-yl) methoxy) carbonyl)amino) propanamido) propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiii). LCMS: (ESI, m/z): [M+H]⁺=1231.7.

### Step C—Synthesis of Compound xiv

4-((S)-2-((S)-2-((((9H-Fluoren-9-yl) methoxy) carbonyl)amino) propanamido) propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiii) (0.092 g, 0.075 mmol) was dissolved in DCM (0.5 mL) and TEA (0.5 mL) was added into the reaction mixture. The reaction mixture was stirred for 16 hours at 40° C. The reaction mixture which contained 4-((S)-2-((S)-2-aminopropanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiv) was used directly without further purification or concentration. LC-MS: (ESI, m/z): [M+H]⁺=1009.8.

### Example 12

Synthesis of 4-((S)-2-((S)-2-(3-(5-cyano-6-(methylsulfonyl) nicotinamido) propanamido) propanamido) propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (3).

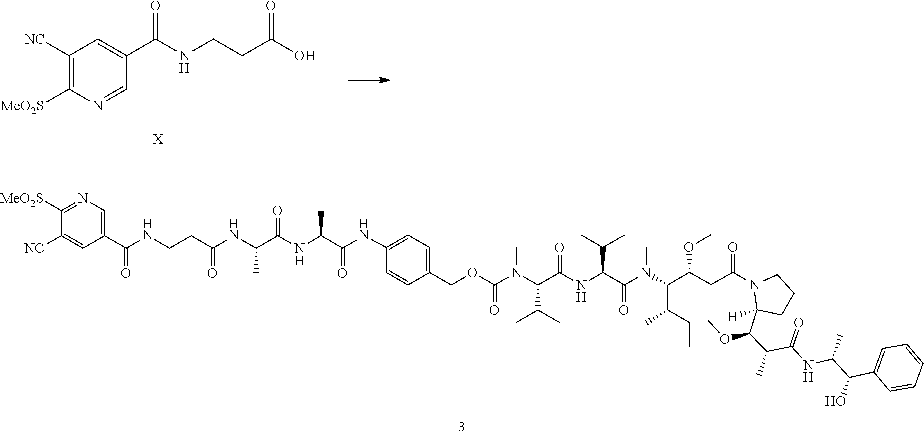

3-(5-Cyano-6-(methylsulfonyl) nicotinamido) propanoic acid (x; Example 10) (0.027 g, 0.090 mmol) and HATU (0.037 g, 0.097 mmol) were dissolved in 0.4 mL of DMF and stirred for 10 minutes. Then xiv was added dropwise followed by DIEA (0.034 ml, 0.19 mmol). The reaction was stirred for 1.5 hours and then purified using reverse phase chromatography (Waters CSH—C18 Column, 19×250 mm×5 μm, 35-70% acetonitrile in water (with 0.1% formic acid as modifier) to provide 4-((S)-2-((S)-2-(3-(5-cyano-6-(methylsulfonyl) nicotinamido) propanamido) propanamido) propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (3). LC-MS: (ESI, m/z): [M+H]⁺=1288.665. ¹H NMR (600 MHZ, CD₃CN) δ 9.12 (s, 1H), 8.68 (s, 1H), 8.63 (s, 1H), 7.66 (d, J=8.4 Hz, 2H), 7.4-7.06 (m, 9H) 6.66 (d, J=8.5 Hz, 1H), 6.55 (d, J=7.75 Hz, 1H), 5.18 (d, J=12.18 Hz, 1H), 5.04 (d, J=12.18 Hz, 1H), 4.71 (d, J=4.16 Hz, 1H), 4.63 (m, 1H), 4.45 (q, J=7.3 Hz, 1H), 4.32-4.00 (m, 4H), 3.9-3.73 (m, 3H), 3.67 (m, 2H), 3.58-3.38 (m, 3H), 3.36 (s, 3H), 3.35 (s, 3H), 3.28 (3, 3H), 3.18 (m, 1H), 3.01 (s, 3H) 2.86 (b, 3H), 2.63 (m, 1H), 2.52 (m, 1H), 2.46 (b, 2H), 2.17 (b, 2H) 1.99 (m, 1H) 1.9-1.6 (m, 4H), 1.59 (b, 1H), 1.48 (d, J=7.38 Hz, 3H) 1.36 (m, 4H), 1.12 (d, J=6.81 Hz, 3H), 1.02 (d, J=6.95 Hz, 3H), 0.98 (m, 4H) 0.92-0.67 (m, 15H).

### Example 13

Conjugation protocol: Antibody with two engineered Cys residues (S375C) was decapped and the interchain disulfides reduced using tris(2-carboxyethyl) phosphine (TCEP) (20 equiv., 0.5M in water pH adjusted to 7.0 with ammonium hydroxide) for 2 hours at 37° C., and monitored with HPLC-MS. The reduced antibody was then buffer exchanged via AKTA™ (desalting column, monitoring at 280 nm) into pH 7.4 PBS buffer or 30 mM pH 7.0 ACES (N-(2-Acetamido)-2-aminoethanesulfonic acid, N-(Carbamoylmethyl)-2-aminoethanesulfonic acid, N-(Carbamoylmethyl) taurine) buffer and was diluted to 10 mg/mL for subsequent steps using the same buffer. A 100 mM solution of dehydroascorbic acid (8.0 equiv.) in water was added slowly and the solution was mixed at room temperature for 4-10 hours or until completion (monitored by reverse phase liquid chromatography (RP-LC) and sodium dodecyl sulfate capillary electrophoresis (CE-SDS)). A 20 mg/mL solution of linker-payload (3.0 equiv.) in DMSO was added and the conjugation was incubated at room temperature for 2 hours (monitored by Quadrupole time-of-flight (QTOF) mass spectrometry (MS)) before addition of L-cysteine (5 equiv., 50 mM in water) to quench residual linker-payload. The ADC was purified via AKTA™ (desalting column, 10 mM histidine buffer pH 6.0, monitoring at 280 nm) and was characterized by LCMS (Agilent polymeric reverse phase (PLRP)—S column, 1000 Å, 5 μm, 15-90% MeCN/H₂O with 0.1% formic acid, 80° C. column temperature) and size-exclusion chromatography (SEC) (Acquity UPLC Protein BEH SEC, 200 Å, 1.7 μm, 100 mM sodium phosphate, 200 mM NaCl, 0.02% azide, 5% isopropyl alcohol (IPA) added to mobile phase for hydrophobic ADCs). The ADC was brought to the final desired concentration using Sartorius Vivacell 70 Centrifugal Concentrators and Amicon® Ultra Centrifugal Filters.

Exemplary conjugates were made according to the above protocol to make the following ADCs.
αTROP2 (HC:BSM-YTE-S375C) (LC:BSM-Y53D) conjugated to MP-AA-PABC-MMAE (ADC 1)
αTROP2 (HC:BSM-YTE-S375C-MMAE) (LC:BSM) conjugated to MP-AA-PABC-MMAE (ADC 2)
αTROP2 (HC:Sac-S375C-MMAE) (LC:Sac) conjugated to MP-AA-PABC-MMAE (ADC 3)

The ADCs were formulated in solution comprising 10 mM histidine, 7.5% sucrose, and 0.02% polysorbate 80 (PS80) at a pH of 6.0.

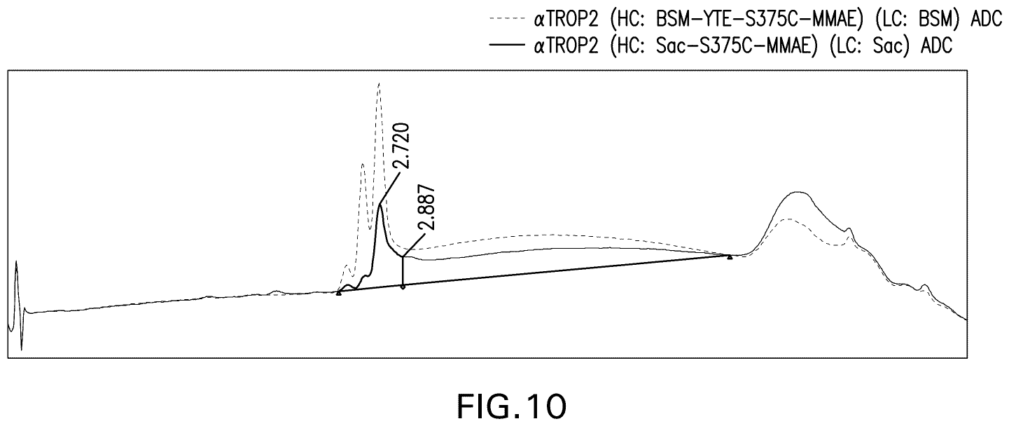

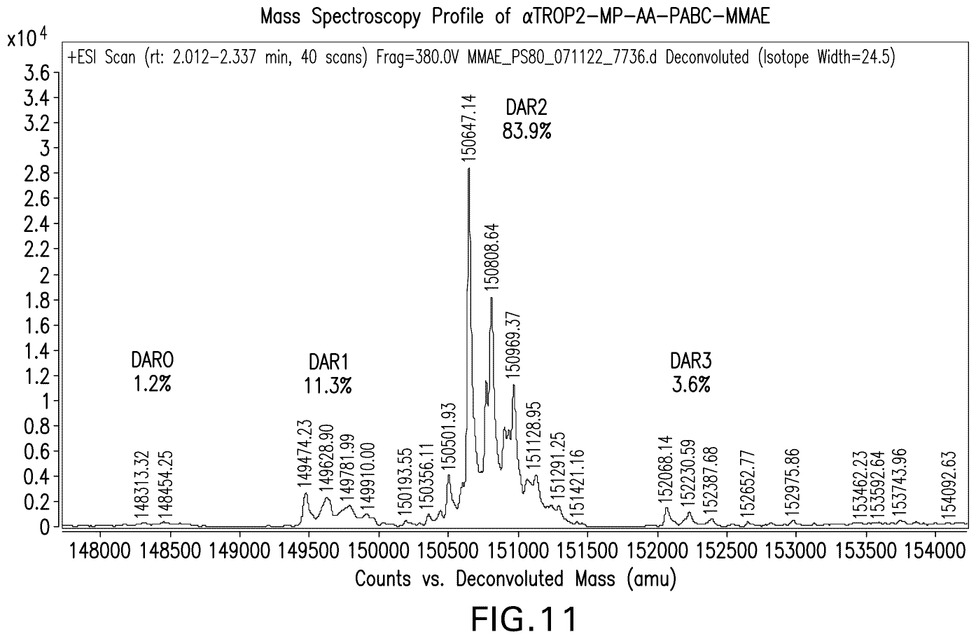

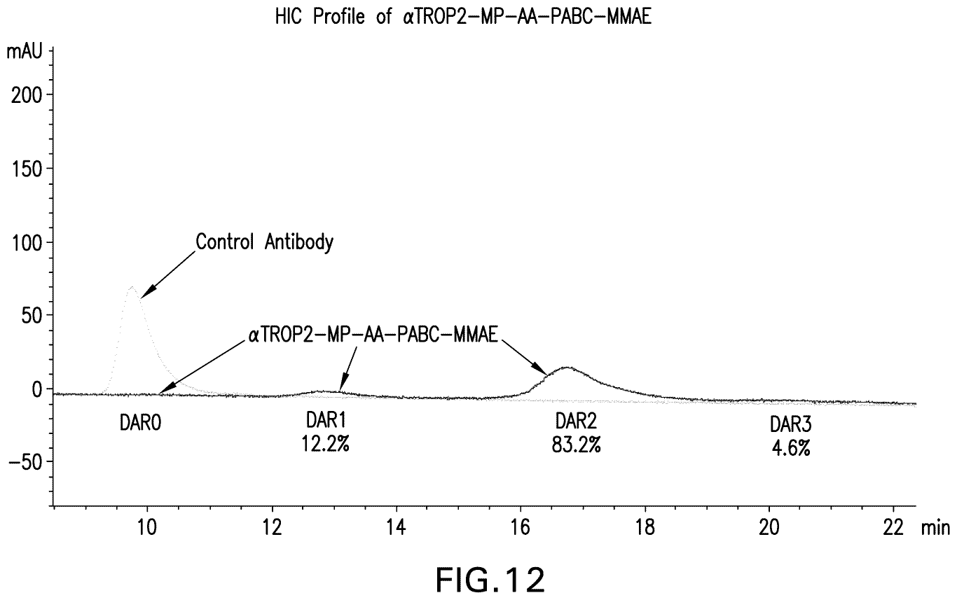

FIG. 10 shows a comparison of RP-HPLC performance of ADC 2 to ADC 3. ADC 2 showed better recovery and intensity compared to ADC 3. FIG. 11 shows a mass spectroscopy (MS) profile of a composition comprising ADC 1 with the positions for DAR0, DAR1, DAR2, and DAR3 indicated. FIG. 12 shows a HIC profile of a composition comprising ADC 1 with the positions for DAR0, DAR1, DAR2, and DAR3 indicated. The αTROP2 antibody comprises HC:BSM-YTE-S375C-MMAE and LC:BSM-Y53D. The control antibody profile is the unconjugated antibody.

Table 20 summarizes the results and shows that the average DAR for ADC 1 based on MS was about 1.9 to 2.0.

TABLE 20

| Assays | ADC 1 |
|---|---|
| Protein concentration | 30.6 mg/mL |
| Binding to target by SPR | Comparable affinities and % binding |
| | capacities to control antibody |
| Purity by UP-SEC | 96.4% |
| Purity by CE-SDS NR | 94.7% |
| Drug-antibody ratio (MS) | DAR: 1.9; |
| | unconjugated antibody (1.2%) |

UP-SEC = ultra-high performance size exclusion chromatography
CE-SDS NR = non-reducing sodium dodecyl sulfate capillary electrophoresis
MS = mass spectroscopy
SPR = surface plasmon resonance

### Example 14

In vitro cytotoxicity of ADCs comprising avidity tuned Sacituzumab variants comprising an S375C substitution in which the cysteine is conjugated to MP-AA-PABC-MMAE.

The cytotoxicity data values are the average IC₅₀ for each test article and may include data from different lots of the same construct. All data in this example comes from a 3-day assay. A control ADC (Control mAb (HC: S375C-MMAE)) consisting of a non-anti-TROP2 antibody conjugated at the cysteine at position 375 to MP-AA-PABC-MMAE was included.

TROP2+ cells with different TROP2 surface density (quantified TROP2 density values are in the parentheses). Certain bioconjugates were run on other cells. Human tumor (BxPC3, Calu-3, HCC1806, HCC78, JIMT-1 and NCI-N87) and primary cells (PCS-301 (primary small airway) and PCS-200 (primary keratinocytes)) from ATCC were seeded in growth media (Table 21) on white tissue culture (TC)-treated 384-well microplates (Corning, Cat #3570) at 1500 or 300 cells, respectively on day 0. On day one, 10× Intermediate assay plates (Waters plate, Cat #186002632) were prepared using a BRAVO liquid handler. ADC formulation buffer (10 mM pH 6.5 histidine 9% sucrose) was used to make serial dilutions. Media (no cells) was used as a Max_E and media with cells used as a Min_E. Then added 5 μL of 10×ADC from intermediate plate to assay plate using a Bravo liquid handler using a very slow speed so the cell monolayer wasn't disturbed. Afterwards, plates were incubated in 37° C. for four days. On day five, 20 μL of CellTiter-Glo 2.0 Reagent (Promega Corporation, Madison, WI, Cat #G9242) was added to 50 μL of medium containing cells using Standard Cassette Combi. The contents were mixed for 2-3 minutes on an orbital shaker to induce cell lysis. The plates were allowed to incubate at room temperature (RT) for 5 minutes to stabilize the luminescent signal. The luminescence was recorded to calculate an EC50 value, using an integration time of 0.25-1 second per well as a guideline. Activity data was normalized as percent effect according to the equation: % E=((Response−Min_E)/(Max_E−Min_E)*100. Cytotoxicity results are shown in Table 22.

TABLE 21
Cell growth media

| Cell lines | Growth media |
|---|---|
| BxPC3, Calu-3, | RPMI 1640 + 10% HI-FBS + 1% P/S |
| HCC1806, HCC78 | |
| and NCI-N87 | |
| JIMT-1 | DMEM + 10% HI-FBS + 1% P/S |
| Primary small airway | Airway Epithelial Cell Basal Medium + |
| (PCS-301) | Bronchial Epithelial Cell Growth Kit |
| | (Small Airway Epithelial Cells, Bronchial/ |
| | Tracheal Epithelial Cells) |
| Primary keratinocytes | Dermal Cell Basal Medium + Keratinocyte |
| (PCS-200) | Growth Kit (Epidermal Keratinocytes) |

TABLE 22
Part A

| ADC | | | Cell Line | | |
|---|---|---|---|---|---|
| | | % HMW | JIMT-1 | BxPC3 | HCC78 |
| | DAR | (SEC) | (605K) | (456K) | (407K) |
| Control mAb (HC: S375C-MMAE) | 1.8 | 1.46 | 144.0 | 149.0 | 195.0 |
| αTROP2 (HC: Sac-S375C-MMAE) | 1.8 | 5.17 | 0.2 | 0.2 | 0.1 |
| αTROP2 (HC: BSM-YTE-S375C- | 1.9 | 1.01 | 0.5 | 0.1 | 0.1 |
| MMAE)(LC: BSM) | | | | | |
| αTROP2 (HC: BSM-YTE-S375C- | 1.6 | 3.54 | 22.9 | 22.5 | 26.8 |
| MMAE)(LC: BSM-S30R) | | | | | |
| αTROP2 (HC: BSM-YTE-S375C- | 1.6 | 3.01 | 35.9 | 7.7 | 11.1 |
| MMAE)(LC: BSM-A32P) | | | | | |
| αTROP2 (HC: BSM-YTE-S375C- | 1.8 | 1.14 | 40.4 | 19.1 | 22.7 |
| MMAE)(LC: BSM-I39K) | | | | | |
| αTROP2 (HC: BSM-YTE-S375C- | 1.7 | 3.26 | 2.4 | 1.3 | 0.4 |
| MMAE)(LC: BSM-I39S) | | | | | |
| αTROP2 (HC: BSM-YTE-S375C- | 1.7 | 4.33 | 25.2 | 6.7 | 5.4 |
| MMAE)(LC: BSM-Y53D) | | | | | |
| αTROP2 (HC: BSM-YTE-S375C- | 1.6 | 4.26 | 9.3 | 0.5 | 2.2 |
| MMAE)(LC: BSM-Y53N) | | | | | |
| αTROP2 (HC: BSM-YTE-S375C- | 1.6 | 2.71 | 11.1 | 5.7 | 16.7 |
| MMAE)(LC: BSM-H91K) | | | | | |
| αTROP2 (HC: BSM-YTE-S375C- | 1.7 | 3.82 | 66.0 | 17.4 | 28.9 |
| MMAE)(LC: BSM-Y92A) | | | | | |
| αTROP2 (HC: BSM-YTE-S375C- | 1.6 | 5.51 | 31.8 | 5.4 | 6.7 |
| MMAE)(LC: BSM-Y92N) | | | | | |
| αTROP2 (HC: BSM-YTE-F101E- | 1.8 | 2.21 | 17.4 | 4.4 | 3.6 |
| S375C-MMAE)(LC: BSM) | | | | | |
| αTROP2 (HC: BSM-YTE-S104D- | 1.6 | 1.59 | 20.0 | 2.2 | 3.0 |
| S375C-MMAE)(LC: BSM) | | | | | |
| αTROP2 (HC: BSM-YTE-Y105S- | 1.7 | 2.16 | 28.1 | 3.9 | 5.0 |
| S375C-MMAE)(LC: BSM) | | | | | |
| αTROP2 (HC: BSM-YTE-W106A- | 1.7 | 2.76 | 46.3 | 10.1 | 12.7 |
| S375C-MMAE)(LC: BSM) | | | | | |
| αTROP2 (HC: BSM-YTE-W106H- | 1.7 | 3.86 | 22.5 | 4.9 | 9.3 |
| S375C-MMAE)(LC: BSM) | | | | | |
| αTROP2 (HC: BSM-YTE-W106L- | 1.7 | 3.62 | 18.7 | 2.6 | 3.7 |
| S375C-MMAE)(LC: BSM) | | | | | |
| αTROP2 (HC: BSM-YTE-R109S- | 1.7 | 2.76 | 0.9 | 0.4 | 0.2 |
| S375C-MMAE)(LC: BSM) | | | | | |

Part B

| ADC | | | Cell Line | | |
|---|---|---|---|---|---|
| | | % HMW | NCI-N87 | HCC 1806 | Calu-3 |
| | DAR | (SEC) | (249K) | (133K) | (106K) |
| Control mAb (HC: S375C-MMAE) | 1.8 | 1.46 | 91.8 | 54.4 | 493.4 |
| αTROP2 (HC: S375C-MMAE) | 1.8 | 5.17 | 0.2 | 15.7 | 51.2 |
| αTROP2 (HC: BSM-YTE-S375C- | 1.9 | 1.01 | 1.2 | 26.2 | 348.7 |
| MMAE)(LC: BSM) | | | | | |
| αTROP2 (HC: BSM-YTE-S375C- | 1.6 | 3.54 | 21.7 | 53.8 | 194.9 |
| MMAE)(LC: BSM-S30R) | | | | | |
| αTROP2 (HC: BSM-YTE-S375C- | 1.6 | 3.01 | 25.2 | 31.4 | 442.7 |
| MMAE)(LC: BSM-A32P) | | | | | |
| αTROP2 (HC: BSM-YTE-S375C- | 1.8 | 1.14 | 35.7 | 201.5 | 143.2 |
| MMAE)(LC: BSM-I39K) | | | | | |
| αTROP2 (HC: BSM-YTE-S375C- | 1.7 | 3.26 | 30.3 | 138.8 | 127.8 |
| MMAE)(LC: BSM-I39S) | | | | | |
| αTROP2 (HC: BSM-YTE-S375C- | 1.7 | 4.33 | 8.8 | 21.5 | 217.3 |
| MMAE)(LC: BSM-Y53D) | | | | | |
| αTROP2 (HC: BSM-YTE-S375C- | 1.6 | 4.26 | 1.7 | 45.8 | 1178.0 |
| MMAE)(LC: BSM-Y53N) | | | | | |
| αTROP2 (HC: BSM-YTE-S375C- | 1.6 | 2.71 | 10.0 | 44.0 | 53.3 |
| MMAE)(LC: BSM-H91K) | | | | | |
| αTROP2 (HC: BSM-YTE-S375C- | 1.7 | 3.82 | 39.0 | 80.2 | 485.6 |
| MMAE)(LC: BSM-Y92A) | | | | | |
| αTROP2 (HC: BSM-YTE-S375C- | 1.6 | 5.51 | 16.7 | 36.9 | 429.5 |
| MMAE)(LC: BSM-Y92N) | | | | | |
| αTROP2 (HC: BSM-YTE-F101E- | 1.8 | 2.21 | 20.1 | 294.0 | 44.4 |
| S375C-MMAE)(LC: BSM) | | | | | |
| αTROP2 (HC: BSM-YTE-S104D- | 1.6 | 1.59 | 7.2 | 269.1 | 44.3 |
| S375C-MMAE)(LC: BSM) | | | | | |
| αTROP2 (HC: BSM-YTE-Y105S- | 1.7 | 2.16 | 17.7 | 43.4 | 365.2 |
| S375C-MMAE)(LC: BSM) | | | | | |
| αTROP2 (HC: BSM-YTE-W106A- | 1.7 | 2.76 | 20.7 | 84.5 | 505.4 |
| S375C-MMAE)(LC: BSM) | | | | | |
| αTROP2 (HC: BSM-YTE-W106H- | 1.7 | 3.86 | 9.4 | 20.5 | 332.4 |
| S375C-MMAE)(LC: BSM) | | | | | |
| αTROP2 (HC: BSM-YTE-W106L- | 1.7 | 3.62 | 2.6 | 17.2 | 254.5 |
| S375C-MMAE)(LC: BSM) | | | | | |
| αTROP2 (HC: BSM-YTE-R109S- | 1.7 | 2.76 | 9.5 | 94.4 | 165.8 |
| S375C-MMAE)(LC: BSM) | | | | | |

HC = heavy chain;
LC = light chain;
BSM = Best Single Mutations;
MMAE = MP-AA-PABC-MMAE
HC and LC substitutions are defined by sequential numbering except as follows:
YTE = M252Y, S254T, T256E substitutions, positions defined according to Eu numbering;
HC constant domain S375C substitution, position defined according to Eu numbering

### Example 15

In vitro cytotoxicity of wild-type Sacituzumab and Y53D variants thereof on different framework or backbone constructs comprising an S375C substitution in which the cysteine is conjugated to MP-AA-PABC-MMAE were evaluated on cells having different TROP2 surface densities. A control ADC (Control mAb (HC: S375C-MMAE)) consisting of a non-anti-TROP2 antibody conjugated at the cysteine at position 375 to MP-AA-PABC-MMAE was included.

The cytotoxicity data is presented in Table 23. The reported values are the average IC₅₀ for each test article and may include data from different lots of the same construct. All data in this table comes from a 4-day assay using various TROP2+ cells with different TROP2 surface density. Certain ADCs have been tested on other cells.

TABLE 23
Part A

| ADC | | | Cell Line | | | |
|---|---|---|---|---|---|---|
| | | | | | | NCI- |
| | | % HMW | JIMT-1 | BxPC3 | HCC78 | N87 |
| | DAR | (SEC) | (605K) | (456K) | (407K) | (249K) |
| Control mAb (HC: S375C- | 1.8 | 1.46 | 322.7 | 373.0 | 398.0 | 233.6 |
| MMAE) | | | | | | |
| αTROP2 (HC: Sac-S375C- | 1.8 | 5.17 | 0.4 | 0.4 | 0.3 | 0.3 |
| MMAE)(LC: Sac) | | | | | | |
| αTROP2 (HC: BSM-S375C- | 1.7 | 1.53 | 0.6 | 0.9 | 0.5 | 0.5 |
| MMAE)(LC: BSM) | | | | | | |
| αTROP2 (HC: YTE-S375C- | 1.7 | 3.63 | 0.6 | 0.4 | 0.3 | 0.4 |
| MMAE) | | | | | | |
| αTROP2 (HC: BSM-YTE- | 1.9 | 1.01 | 0.4 | 0.5 | 0.2 | 0.2 |
| S375C-MMAE)(LC: BSM) | | | | | | |
| αTROP2 (HC: BSM-YTE- | 1.6 | 0.26 | 0.6 | 0.5 | 0.3 | 0.4 |
| LALADS-S375C-MMAE)(LC: | | | | | | |
| BSM) | | | | | | |
| αTROP2 (HC: S375C- | 1.6 | 7.25 | 5.7 | 1.8 | 1.9 | 2.9 |
| MMAE)(LC: Y53D) | | | | | | |
| αTROP2 (HC: BSM-S375C- | 1.8 | 1.46 | 6.8 | 1.5 | 1.7 | 5.5 |
| MMAE)(LC: BSM-Y53D) | | | | | | |
| αTROP2 (HC: BSM-YTE- | 1.7 | 4.33 | 3.1 | 2.4 | 1.1 | 2.2 |
| S375C-MMAE)(LC: BSM- | | | | | | |
| Y53D) | | | | | | |

Part B

| ADC | | | Cell Line | | | |
|---|---|---|---|---|---|---|
| | | | | | PCS-301 (36K; | PCS-200 (56K; |
| | | % HMW | HCC1806 | Calu-3 | primary small | primary keratin- |
| | DAR | (SEC) | (133K) | (106K) | airway) | ocytes) |
| Control mAb (HC: S375C- | 1.8 | 1.46 | 143.0 | 531.0 | 139.8 | 444.0 |
| MMAE) | | | | | | |
| αTROP2 (HC: Sac-S375C- | 1.8 | 5.17 | 21.6 | 15.1 | 22.8 | 64.3 |
| MMAE)(LC: Sac) | | | | | | |
| αTROP2 (HC: BSM-S375C- | 1.7 | 1.53 | 85.1 | 33.2 | 68.1 | 119.4 |
| MMAE)(LC: BSM) | | | | | | |
| αTROP2 (HC: YTE-S375C- | 1.7 | 3.63 | 42.4 | 51.1 | 31.0 | 37.0 |
| MMAE) | | | | | | |
| αTROP2 (HC: BSM-YTE- | 1.9 | 1.01 | 29.6 | 2.6 | 12.4 | 12.4 |
| S375C-MMAE)(LC: BSM) | | | | | | |
| αTROP2 (HC: BSM-YTE- | 1.6 | 0.26 | 43.4 | 30.1 | 50.0 | 47.1 |
| LALADS-S375C-MMAE)(LC: | | | | | | |
| BSM) | | | | | | |
| αTROP2 (HC: S375C- | 1.6 | 7.25 | 110.4 | 24.7 | 163.9 | 219.2 |
| MMAE)(LC: Y53D) | | | | | | |
| αTROP2 (HC: BSM-S375C- | 1.8 | 1.46 | 58.9 | 31.4 | 40.6 | 68.6 |
| MMAE)(LC: BSM-Y53D) | | | | | | |
| αTROP2 (HC: BSM-YTE- | 1.7 | 4.33 | 57.3 | 4.6 | 28.6 | 33.1 |
| S375C-MMAE)(LC: BSM- | | | | | | |
| Y53D) | | | | | | |

### Example 16

In vivo anti-tumor efficacy of several avidity-tuned Sacituzumab variants conjugated to MMAE linker payload MP-AA-PABC-MMAE in a BxPC3 mouse model.

Experimental protocol: 7-8 week old female BALB/c nude mice were purchased from Gempharmatech Co., Ltd. (La Jolla, CA). Mice were anesthetized with isoflurane inhalant and inoculated subcutaneously into the right lower flank with a single-cell suspension of 10×106 BxPC3 pancreatic cancer cells (>95% viability) in 0.1 mL volume of PBS:matrigel (50:50). Mouse body weights and tumors were measured twice weekly, and tumor volumes calculated using the formula V=0.5 [a*b2]; where a and b are the long and short diameters of the tumor, respectively. 110 mice were assigned to 11 groups of 10 mice each using a computer-generated randomization procedure to minimize tumor volume variance across groups. Treatments were started when tumors reached an average size of 200 mm³. PBS and the MMAE conjugates were dosed intravenously (IV) at 2 mg/kg according to Table 25.

TABLE 25

| | | Dose |
|---|---|---|
| Group | Treatment | (mg/kg) |
| 1 | PBS | — |
| 2 | αTROP2 (HC: Sac-S375C-MMAE)(LC: Sac) | 2 |
| 3 | αTROP2 (HC: BSM-YTE-S375C-MMAE)(LC: | 2 |
| | BSM) | |
| 4 | αTROP2 (HC: BSM-YTE-S375C-MMAE)(LC: | 2 |
| | BSM-Y53D) | |
| 5 | αTROP2 (HC: BSM-YTE-S375C-MMAE)(LC: | 2 |
| | BSM-Y92N) | |
| 6 | αTROP2 (HC: BSM-YTE-S375C-MMAE)(LC: | 2 |
| | BSM-S30R) | |
| 7 | αTROP2 (HC: BSM-YTE-S375C-MMAE)(LC: | 2 |
| | BSM-Y53N) | |
| 8 | αTROP2 (HC: BSM-YTE-Y105S-S375C- | 2 |
| | MMAE)(LC: BSM) | |
| 9 | αTROP2 (HC: BSM-YTE-F101E-S375C- | 2 |
| | MMAE)(LC: BSM) | |
| 10 | αTROP2 (HC: BSM-YTE-S104D-S375C- | 2 |
| | MMAE)(LC: BSM) | |
| 11 | αTROP2 (HC: BSM-YTE-S375C-MMAE) | 2 |
| | (LC: BSM-I39K) | |

HC = heavy chain;
LC = light chain;
BSM = Best Single Mutations;
MMAE = MP-AA-PABC-MMAE
PBS = phosphate buffered saline
HC and LC substitutions are defined by sequential numbering except as follows:
YTE = M252Y, S254T, T256E substitutions, positions defined according to Eu numbering;
HC constant domain S375C substitution, position defined according to Eu numbering

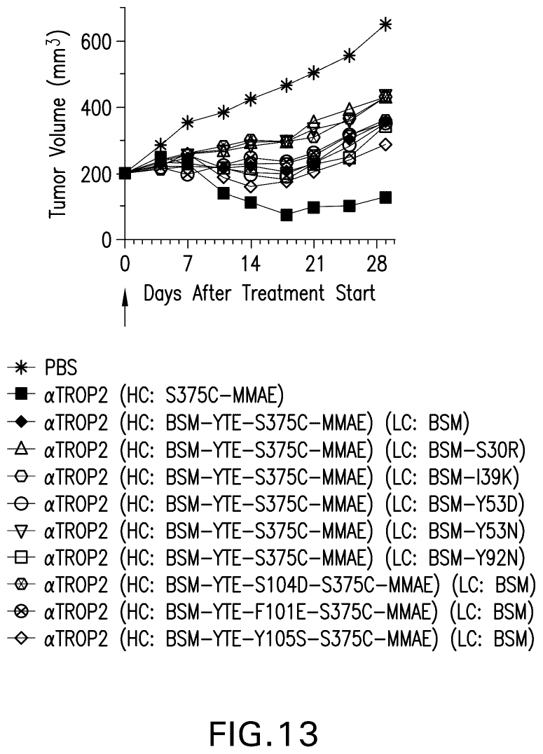

FIG. 13 shows that avidity-tuned Sacituzumab variants conjugated to MP-AA-PABC-MMAE were effective at reducing tumor volume follow treatment dosing.

### Example 17

Dose response study with αTROP2 (HC:BSM-YTE-S375C-MMAE) (LC:BSM-Y53D) in a BxPC3 mouse model. A control ADC (Control mAb (HC: S375C-MMAE)) consisting of a non-anti-TROP2 antibody conjugated at the cysteine at position 375 to MP-AA-PABC-MMAE was included.

Experimental protocol: 7-8 week old female BALB/c nude mice were purchased from Gempharmatech Co., Ltd. Mice were anesthetized with isoflurane inhalant and inoculated subcutaneously into the right lower flank with a single-cell suspension of 10×106 BxPC3 pancreatic cancer cells (>95% viability) in 0.1 mL volume of PBS:matrigel (50:50). Mouse body weights and tumors were measured twice weekly, and tumor volumes calculated using the formula V=0.5 [a*b2]; where a and b are the long and short diameters of the tumor, respectively. 70 mice were assigned to 7 groups of 10 mice each using a computer-generated randomization procedure to minimize tumor volume variance across groups. Treatments were started when tumors reached an average size of 200 mm³. PBS, a control mAb, and bioconjugate were dosed intravenously (IV) according to Table 26.

TABLE 26

| | Dose | Dosing |
|---|---|---|
| Treatment | (mg/kg) | Schedule |
| Control mAb (HC: S375C-MMAE) | 10 | Single dose IV |
| αTROP2 (HC: BSM-YTE-S375C-MMAE) | 10 | Single dose IV |
| (LC: BSM-Y53D) | | |
| αTROP2 (HC: BSM-YTE-S375C-MMAE) | 5 | Single dose IV |
| (LC: BSM-Y53D) | | |
| αTROP2 (HC: BSM-YTE-S375C-MMAE) | 2.5 | Single dose IV |
| (LC: BSM-Y53D) | | |
| αTROP2 (HC: BSM-YTE-S375C-MMAE) | 1.25 | Single dose IV |
| (LC: BSM-Y53D) | | |
| αTROP2 (HC: BSM-YTE-S375C) | 10 | Single dose IV |
| (LC: BSM-Y53D) | | |
| PBS | — | Single dose IV |

HC = heavy chain;
LC = light chain;
BSM = Best Single Mutations;
MMAE = MP-AA-PABC-MMAE
PBS = phosphate buffered saline
HC and LC substitutions are defined by sequential numbering except as follows:
YTE = M252Y, S254T, T256E substitutions, positions defined according to Eu numbering; HC constant domain S375C substitution, position defined according to Eu numbering

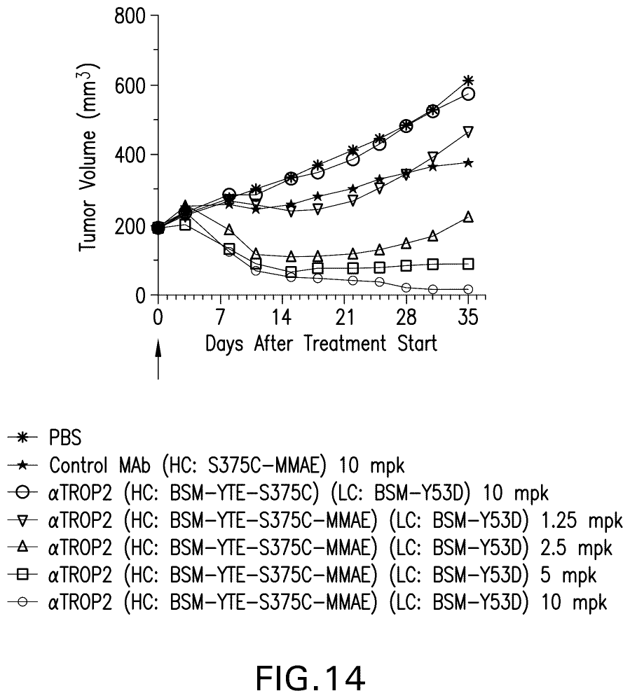

FIG. 14 shows that αTROP2 (HC:BSM-YTE-S375C-MMAE) (LC:BSM-Y53D) was effective at reducing tumor volume follow treatment dosing.

### Example 18

αTROP2 (HC:BSM-YTE-S375C-MMAE) (LC:BSM-Y53D) antibodies in rat PK assays showed linker stability with little payload bleeding off the antibody. In these experiments, αTROP2 (HC:BSM-YTE-S375C) (LC:BSM-Y53D) was conjugated at the cysteine at position 375 to MP-AA-PABC-MMAE.

αTROP2 MMAE ADCs were intravenously administered at 5 mg/kg to male Wistar Hannover Rats. Plasma concentrations of payload conjugated anti-TROP2 antibody (cAb), total antibody (tAb), and released payload were measured up to 14 days post dose. The PK parameters were estimated by noncompartmental analysis with Phoenix WinNonlin (Ver. 6.3, Certara. Released MMAE concentrations were determined using LC/MS-MS methods with lower limit of quantitation (LLOQ) of 0.055 ng/ml for MMAE. Concentrations of cAb and tAb in plasma were determined with a ligand-binding assay method using anti-TROP2 MMAE antibody conjugates and anti-human IgG framework antibodies.

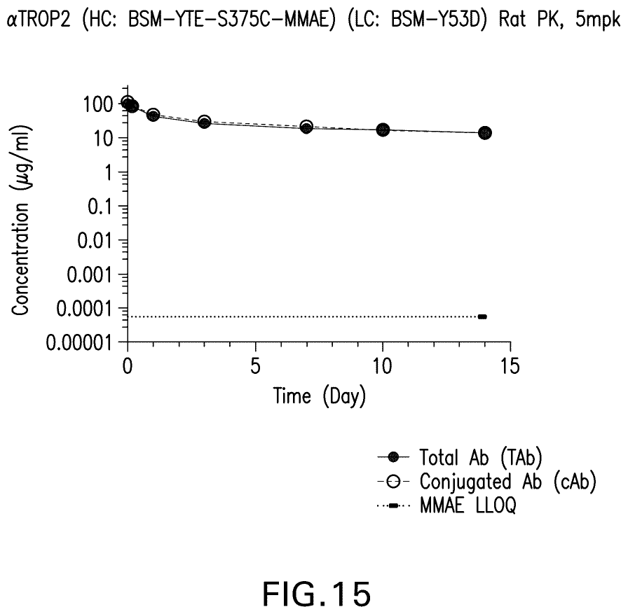

The plasma tAb, cAb, and released payload concentrations along the time course for αTROP2 (HC:BSM-YTE-S375C-MMAE) (LC:BSM-Y53D) was plotted in FIG. 15. For the ADC, the measured tAb and cAb PK curves overlapped with each other and there was no detectable unconjugated MMAE in the plasma, indicating the stability of the linkers in vivo. The terminal half-life was 9.9 days.

Table 27 provides sequences referred to in the instant disclosure.

TABLE 27
Sequences

| SEQ | | |
|---|---|---|
| ID | | |
| NO: | Description | Sequence |
| 1 | Alpha-TROP2 VH | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSS |
| 2 | Alpha-TROP2 VL | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHYITPLTFGAGTKVEIK |
| 3 | Alpha-TROP2 VL | DIQLTQSPSSLSASVGDRVSITCKASQDVSIAVAWYQQKPG |
| | (Y53D) | KAPKLLIYSASDRYTGVPDRFSGSGSGTDFTLTISSLQPEDF |
| | | AVYYCQQHYITPLTFGAGTKVEIK |
| 4 | Alpha-TROP2 | NYGMN |
| | HCDR1 | |
| 5 | Alpha-TROP2 | WINTYTGEPTYTDDFKG |
| | HCDR2 | |
| 6 | Alpha-TROP2 | GGFGSSYWYFDV |
| | HCDR3 | |
| 7 | Alpha-TROP2 | KASQDVSIAVA |
| | LCDR1 | |
| 8 | Alpha-TROP2 | SASYRYT |
| | LCDR2 | |
| 9 | Alpha-TROP2 | QQHYITPLT |
| | LCDR3 | |
| 10 | Alpha-TROP2 | SASDRYT |
| | LCDR2 (Y53D) | |
| 11 | Alpha-TROP2 HC | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 12 | Alpha-TROP2 LC | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 13 | Alpha-TROP2 LC | DIQLTQSPSSLSASVGDRVSITCKASQDVSIAVAWYQQKPG |
| | (Y53D) | KAPKLLIYSASDRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 14 | Alpha-TROP2 VH | QVQLLQSGSELKKPGASVKVSCKASGYTFTNYGMNWVR |
| | (BSM) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSS |
| 15 | Alpha-TROP2 VL | DIQLTQSPSSLSASVGDRVTITCKASQDVSIAVAWYQQKPG |
| | (BSM) | KAPKLLIYSASYRYTGVPSRFSGSGSGTDFTLTISSLQPEDF |
| | | ATYYCQQHYITPLTFGPGTKVEIK |
| 16 | Alpha-TROP2 VL | DIQLTQSPSSLSASVGDRVTITCKASQDVSIAVAWYQQKPG |
| | (BSM-Y53D) | KAPKLLIYSASDRYTGVPSRFSGSGSGTDFTLTISSLQPEDF |
| | | ATYYCQQHYITPLTFGPGTKVEIK |
| 17 | Alpha-TROP2 HC | QVQLLQSGSELKKPGASVKVSCKASGYTFTNYGMNWVR |
| | (BSM) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 18 | Alpha-TROP2 HC | QVQLLQSGSELKKPGASVKVSCKASGYTFTNYGMNWVR |
| | (BSM-YTE) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 19 | Alpha-TROP2 HC | QVQLLQSGSELKKPGASVKVSCKASGYTFTNYGMNWVR |
| | (BSM-S375C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 20 | Alpha-TROP2 HC | QVQLLQSGSELKKPGASVKVSCKASGYTFTNYGMNWVR |
| | (BSM-YTE-S375C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 21 | Alpha-TROP2 LC | DIQLTQSPSSLSASVGDRVTITCKASQDVSIAVAWYQQKPG |
| | (BSM) | KAPKLLIYSASYRYTGVPSRFSGSGSGTDFTLTISSLQPEDF |
| | | ATYYCQQHYITPLTFGPGTKVEIKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSFNR |
| | | GEC |
| 22 | Alpha-TROP2 LC | DIQLTQSPSSLSASVGDRVTITCKASQDVSIAVAWYQQKPG |
| | (BSM-Y53D) | KAPKLLIYSASDRYTGVPSRFSGSGSGTDFTLTISSLQPEDF |
| | | ATYYCQQHYITPLTFGPGTKVEIKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSFNR |
| | | GEC |
| 23 | Alpha-TROP2 HC | QVQLQQSGSELKKPGASVKVSCKASGYTFTNYGMNWVK |
| | (YTE) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 24 | Alpha-TROP2 | QVQLQQSGSELKKPGASVKVSCKASGYTFTNYGMNWVK |
| | (HC:Sac-E152C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 25 | Alpha-TROP2 | QVQLQQSGSELKKPGASVKVSCKASGYTFTNYGMNWVK |
| | (HC:Sac-P153C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPECVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 26 | Alpha-TROP2 | QVQLQQSGSELKKPGASVKVSCKASGYTFTNYGMNWVK |
| | (HC:Sac-P171C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFCAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 27 | Alpha-TROP2 | QVQLQQSGSELKKPGASVKVSCKASGYTFTNYGMNWVK |
| | (HC:Sac-A172C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPCVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 28 | Alpha-TROP2 | QVQLQQSGSELKKPGASVKVSCKASGYTFTNYGMNWVK |
| | (HC:Sac-V173C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPACLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 29 | Alpha-TROP2 | QVQLQQSGSELKKPGASVKVSCKASGYTFTNYGMNWVK |
| | (HC:Sac-S375C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 30 | Alpha-TROP2 | QVQLQQSGSELKKPGASVKVSCKASGYTFTNYGMNWVK |
| | (HC:Sac- | QAPGQGLKWMGWINTYTGEPTYTDDFKGRFAFSLDTSVS |
| | E152C/S375C) | TAYLQISSLKADDTAVYFCARGGFGSSYWYFDVWGQGSL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 31 | Alpha-TROP2 | QVQLQQSGSELKKPGASVKVSCKASGYTFTNYGM |
| | (HC:Sac-YTE- | NWVKQAPGQGLKWMGWINTYTGEPTYTDDFKGRFAFSL |
| | E152C) | DTSVSTAYLQISSLKADDTAVYFCARGGFGSSYWYFDVW |
| | | GQGSLVTVSSASTKGPSVFPLAPSSKSTSGGTAALGCLVKDYF |
| | | PCPVTVSWNSGALTSGVHTFPAVLQSSGLYSLSSVVTVPSSSLG |
| | | TQTYICNVNHKPSNTKVDKRVEPKSCDKTHTCPPCPAPELLG |
| | | GPSVFLFPPKPKDTLYITREPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGK |
| 32 | Alpha-TROP2 | QVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | P153C) | TAYLQISSLKADDTAVYFCARGGFGSSYWYFDVWGQGSL |
| | | VTVSSASTKGPSVFPLAPSSKSTSGGTAALGCLVKDYFPECVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 33 | Alpha-TROP2 | QVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | P171C) | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFCAVLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 34 | Alpha-TROP2 | QVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | A172C) | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPCVLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 35 | Alpha-TROP2 | QVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | V173C) | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPACLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 36 | Alpha-TROP2 | QVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | S375C) | TAYLQISSLKADDTAVYFCARGGFGSSYWYFDVWGQGSL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 37 | Alpha-TROP2 | QVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | E152C/S375C) | TAYLQISSLKADDTAVYFCARGGFGSSYWYFDVWGQGSL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 38 | Alpha-TROP2 | QVQLLQSGSELKKPGASVKVSCKASGYTFTNYGMNWVR |
| | (HC:BSM-E152C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 39 | Alpha-TROP2 | QVQLLQSGSELKKPGASVKVSCKASGYTFTNYGMNWVR |
| | (HC:BSM-P153C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPECVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 40 | Alpha-TROP2 | QVQLLQSGSELKKPGASVKVSCKASGYTFTNYGMNWVR |
| | (HC:BSM-P171C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFCAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 41 | Alpha-TROP2 | QVQLLQSGSELKKPGASVKVSCKASGYTFTNYGMNWVR |
| | (HC:BSM-A172C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPCVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 42 | Alpha-TROP2 | QVQLLQSGSELKKPGASVKVSCKASGYTFTNYGMNWVR |
| | (HC:BSM-V173C) | QAPGQGLKWMGWINTYTGEPTYTDDFKGRFSFSLDTSVS |
| | | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPACLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 43 | Alpha-TROP2 | QVQLLQSGSELKKPGASVKVSCKASGYTFTNYGMNWVR |
| | (HC:BSM- | QAPGQGLKWMGWINTYTGEPTYTDDFKGRFSFSLDTSVS |
| | E152C/S375C) | QAYLQISSLKAEDTAVYYCARGGFGSSYWYFDVWGQGTL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 44 | Alpha-TROP2 | QVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | E152C) | QAYLQISSLKAEDTAVYYCARGGFGSSYWYFDVWGQGTL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 45 | Alpha-TROP2 | QVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | P153C) | QAYLQISSLKAEDTAVYYCARGGFGSSYWYFDVWGQGTL |
| | | VTVSSASTKGPSVFPLAPSSKSTSGGTAALGCLVKDYFPECVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 46 | Alpha-TROP2 | QVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | P171C) | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFCAVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 47 | Alpha-TROP2 | QVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | A172C) | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPCVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 48 | Alpha-TROP2 | QVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | V173C) | QAYLQISSLKAEDTAVYYCARGGFGSSYWYFDVWGQGTL |
| | | VTVSSASTKGPSVFPLAPSSKSTSGGTAALGCLVKDYFPEPVT |
| | | VSWNSGALTSGVHTFPACLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 49 | Alpha-TROP2 | QVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | E152C/S375C) | QAYLQISSLKAEDTAVYYCARGGFGSSYWYFDVWGQGTL |
| | | VTVSSASTKGPSVFPLAPSSKSTSGGTAALGCLVKDYFPCPVT |
| | | VSWNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPGK |
| 50 | Alpha-TROP2 | DIQLTQSPSSLSASVGDRVSITCKASQDVSIAVAWYQQKPG |
| | (LC:Sac-Y53D- | KAPKLLIYSASDRYTGVPDRFSGSGSGTDFTLTISSLQPEDF |
| | E165C) | AVYYCQQHYITPLTFGAGTKVEIKRTVAAPSVFIFPPSDEQL |
| | | KSGTASVVCLLNNFYPREAKVQWKVDNALQSGNSQESVTCQD |
| | | SKDSTYSLSSTLTLSKADYEKHKVYACEVTHQGLSSPVTKSFNR |
| | | GEC |
| 51 | Alpha-TROP2 | DIQLTQSPSSLSASVGDRVSITCKASQDVSIAVAWYQQKPG |
| | (LC:Sac-Y53D- | KAPKLLIYSASDRYTGVPDRFSGSGSGTDFTLTISSLQPEDF |
| | S168C) | AVYYCQQHYITPLTFGAGTKVEIKRTVAAPSVFIFPPSDEQL |
| | | KSGTASVVCLLNNFYPREAKVQWKVDNALQSGNSQESVTEQD |
| | | CKDSTYSLSSTLTLSKADYEKHKVYACEVTHQGLSSPVTKSFNR |
| | | GEC |
| 52 | Alpha-TROP2 | DIQLTQSPSSLSASVGDRVTITCKASQDVSIAVAWYQQKPG |
| | (LC:BSM-E165C) | KAPKLLIYSASYRYTGVPSRFSGSGSGTDFTLTISSLQPEDF |
| | | ATYYCQQHYITPLTFGPGTKVEIKRTVAAPSVFIFPPSDEQL |
| | | KSGTASVVCLLNNFYPREAKVQWKVDNALQSGNSQESVTCQD |
| | | SKDSTYSLSSTLTLSKADYEKHKVYACEVTHQGLSSPVTKSFNR |
| | | GEC |
| 53 | Alpha-TROP2 | DIQLTQSPSSLSASVGDRVTITCKASQDVSIAVAWYQQKPG |
| | (LC:BSM-S168C) | KAPKLLIYSASYRYTGVPSRFSGSGSGTDFTLTISSLQPEDF |
| | | ATYYCQQHYITPLTFGPGTKVEIKRTVAAPSVFIFPPSDEQL |
| | | KSGTASVVCLLNNFYPREAKVQWKVDNALQSGNSQESVTEQD |
| | | CKDSTYSLSSTLTLSKADYEKHKVYACEVTHQGLSSPVTKSFNR |
| | | GEC |
| 54 | Alpha-TROP2 | DIQLTQSPSSLSASVGDRVTITCKASQDVSIAVAWYQQKPG |
| | (LC:BSM-Y53D- | KAPKLLIYSASDRYTGVPSRFSGSGSGTDFTLTISSLQPEDF |
| | E165C) | ATYYCQQHYITPLTFGPGTKVEIKRTVAAPSVFIFPPSDEQL |
| | | KSGTASVVCLLNNFYPREAKVQWKVDNALQSGNSQESVTCQD |
| | | SKDSTYSLSSTLTLSKADYEKHKVYACEVTHQGLSSPVTKSFNR |
| | | GEC |
| 55 | Alpha-TROP2 | DIQLTQSPSSLSASVGDRVTITCKASQDVSIAVAWYQQKPG |
| | (LC:BSM-Y53D- | KAPKLLIYSASDRYTGVPSRFSGSGSGTDFTLTISSLQPEDF |
| | S168C) | ATYYCQQHYITPLTFGPGTKVEIKRTVAAPSVFIFPPSDEQL |
| | | KSGTASVVCLLNNFYPREAKVQWKVDNALQSGNSQESVTEQD |
| | | CKDSTYSLSSTLTLSKADYEKHKVYACEVTHQGLSSPVTKSFNR |
| | | GEC |
| 56 | Leader peptide A | MSVPTQVLGLLLLWLTDARC |
| 57 | Leader peptide B | MEWSWVFLFFLSVTTGVHS |
| 58 | Alpha-TROP2 HC | XVQLQQSGSELKKPGASVKVSCKASGYTFTNYGMNWVK |
| | X1 is Q, E, or | QAPGQGLKWMGWINTYTGEPTYTDDFKGRFAFSLDTSVS |
| | pyroglutamate | TAYLQISSLKADDTAVYFCARGGFGSSYWYFDVWGQGSL |
| | X450 is G, GK, or | VTVSSASTKGPSVFPLAPSSKSTSGGTAALGCLVKDYFPEPVT |
| | absent | VSWNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 59 | Alpha-TROP2 HC | XVQLLQSGSELKKPGASVKVSCKASGYTFTNYGMNWVR |
| | (BSM) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 60 | Alpha-TROP2 HC | XVQLLQSGSELKKPGASVKVSCKASGYTFTNYGMNWVR |
| | (BSM-YTE) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 61 | Alpha-TROP2 HC | XVQLLQSGSELKKPGASVKVSCKASGYTFTNYGMNWVR |
| | (BSM-S375C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 62 | Alpha-TROP2 HC | XVQLLQSGSELKKPGASVKVSCKASGYTFTNYGMNWVR |
| | (BSM-YTE-S375C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 63 | Alpha-TROP2 HC | XVQLQQSGSELKKPGASVKVSCKASGYTFTNYGMNWVK |
| | (YTE) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKRVEPKSCDKTHTCPPCPAPELLGGPSVFL |
| | | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 64 | Alpha-TROP2 | XVQLQQSGSELKKPGASVKVSCKASGYTFTNYGMNWVK |
| | (HC:Sac-E152C) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPCPVT |
| | X450 is G, GK, or | VSWNSGALTSGVHTFPAVLQSSGLYSLSSVVTVPSSSLGTQTYIC |
| | absent | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 65 | Alpha-TROP2 | XVQLQQSGSELKKPGASVKVSCKASGYTFTNYGMNWVK |
| | (HC:Sac-P153C) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPECVT |
| | X450 is G, GK, or | VSWNSGALTSGVHTFPAVLQSSGLYSLSSVVTVPSSSLGTQTYIC |
| | absent | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 66 | Alpha-TROP2 | XVQLQQSGSELKKPGASVKVSCKASGYTFTNYGMNWVK |
| | (HC:Sac-P171C) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPEPVT |
| | X450 is G, GK, or | VSWNSGALTSGVHTFCAVLQSSGLYSLSSVVTVPSSSLGTQTYIC |
| | absent | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 67 | Alpha-TROP2 | XVQLQQSGSELKKPGASVKVSCKASGYTFTNYGMNWVK |
| | (HC:Sac-A172C) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPEPVT |
| | X450 is G, GK, or | VSWNSGALTSGVHTFPCVLQSSGLYSLSSVVTVPSSSLGTQTYIC |
| | absent | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 68 | Alpha-TROP2 | XVQLQQSGSELKKPGASVKVSCKASGYTFTNYGMNWVK |
| | (HC:Sac-V173C) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPEPVT |
| | X450 is G, GK, or | VSWNSGALTSGVHTFPACLQSSGLYSLSSVVTVPSSSLGTQTYIC |
| | absent | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 69 | Alpha-TROP2 | XVQLQQSGSELKKPGASVKVSCKASGYTFTNYGMNWVK |
| | (HC:Sac-S375C) | QAPGQGLKWMGWINTYTGEPTYTDDFKGRFAFSLDTSVS |
| | X1 is Q, E, or | TAYLQISSLKADDTAVYFCARGGFGSSYWYFDVWGQGSL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 70 | Alpha-TROP2 | XVQLQQSGSELKKPGASVKVSCKASGYTFTNYGMNWVK |
| | (HC:Sac- | QAPGQGLKWMGWINTYTGEPTYTDDFKGRFAFSLDTSVS |
| | E152C/S375C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPCPVT |
| | pyroglutamate | VSWNSGALTSGVHTFPAVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 71 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | E152C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPCPVT |
| | pyroglutamate | VSWNSGALTSGVHTFPAVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 72 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | P153C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPECVT |
| | pyroglutamate | VSWNSGALTSGVHTFPAVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 73 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | P171C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPEPVT |
| | pyroglutamate | VSWNSGALTSGVHTFCAVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 74 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | A172C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPEPVT |
| | pyroglutamate | VSWNSGALTSGVHTFPCVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 75 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | V173C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPEPVT |
| | pyroglutamate | VSWNSGALTSGVHTFPACLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 76 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | S375C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPEPVT |
| | pyroglutamate | VSWNSGALTSGVHTFPAVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 77 | Alpha-TROP2 | XVQLQQSGSELKKPGASVKVSCKASGYTFTNYGMNWVK |
| | (HC:Sac-YTE- | QAPGQGLKWMGWINTYTGEPTYTDDFKGRFAFSLDTSVS |
| | E152C/S375C) | TAYLQISSLKADDTAVYFCARGGFGSSYWYFDVWGQGSL |
| | X1 is Q, E, or | VTVSSASTKGPSVFPLAPSSKSTSGGTAALGCLVKDYFPCPVT |
| | pyroglutamate | VSWNSGALTSGVHTFPAVLQSSGLYSLSSVVTVPSSSLGTQTYIC |
| | X450 is G, GK, or | NVNHKPSNTKVDKRVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 78 | Alpha-TROP2 | XVQLLQSGSELKKPGASVKVSCKASGYTFTNYGMNWVR |
| | (HC:BSM-E152C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPCPVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 79 | Alpha-TROP2 | XVQLLQSGSELKKPGASVKVSCKASGYTFTNYGMNWVR |
| | (HC:BSM-P153C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPECVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 80 | Alpha-TROP2 | XVQLLQSGSELKKPGASVKVSCKASGYTFTNYGMNWVR |
| | (HC:BSM-P171C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFCAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 81 | Alpha-TROP2 | XVQLLQSGSELKKPGASVKVSCKASGYTFTNYGMNWVR |
| | (HC:BSM-A172C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPCVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 82 | Alpha-TROP2 | XVQLLQSGSELKKPGASVKVSCKASGYTFTNYGMNWVR |
| | (HC:BSM-V173C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPACLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 83 | Alpha-TROP2 | XVQLLQSGSELKKPGASVKVSCKASGYTFTNYGMNWVR |
| | (HC:BSM-S375C) | QAPGQGLKWMGWINTYTGEPTYTDDFKGRFSFSLDTSVS |
| | X1 is Q, E, or | QAYLQISSLKAEDTAVYYCARGGFGSSYWYFDVWGQGTL |
| | pyroglutamate | VTVSSASTKGPSVFPLAPSSKSTSGGTAALGCLVKDYFPEPVT |
| | X450 is G, GK, or | VSWNSGALTSGVHTFPAVLQSSGLYSLSSVVTVPSSSLGTQTYIC |
| | absent | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 84 | Alpha-TROP2 | XVQLLQSGSELKKPGASVKVSCKASGYTFTNYGMNWVR |
| | (HC:BSM- | QAPGQGLKWMGWINTYTGEPTYTDDFKGRFSFSLDTSVS |
| | E152C/S375C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPCPVT |
| | pyroglutamate | VSWNSGALTSGVHTFPAVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 85 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | E152C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPCPVT |
| | pyroglutamate | VSWNSGALTSGVHTFPAVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 86 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | P153C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPECVT |
| | pyroglutamate | VSWNSGALTSGVHTFPAVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 87 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | P171C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPEPVT |
| | pyroglutamate | VSWNSGALTSGVHTFCAVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 88 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | A172C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPEPVT |
| | pyroglutamate | VSWNSGALTSGVHTFPCVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 89 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | V173C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPEPVT |
| | pyroglutamate | VSWNSGALTSGVHTFPACLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPX |
| 90 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | S375C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPEPVT |
| | pyroglutamate | VSWNSGALTSGVHTFPAVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 91 | Alpha-TROP2 | XVQLLQSGSELKKPGASVKVSCKASGYTFTNYGMNWVR |
| | (HC:BSM-YTE- | QAPGQGLKWMGWINTYTGEPTYTDDFKGRFSFSLDTSVS |
| | E152C/S375C) | QAYLQISSLKAEDTAVYYCARGGFGSSYWYFDVWGQGTL |
| | X1 is Q, E, or | VTVSSASTKGPSVFPLAPSSKSTSGGTAALGCLVKDYFPCPVT |
| | pyroglutamate | VSWNSGALTSGVHTFPAVLQSSGLYSLSSVVTVPSSSLGTQTYIC |
| | X450 is G, GK, or | NVNHKPSNTKVDKKVEPKSCDKTHTCPPCPAPELLGGPSVFL |
| | absent | FPPKPKDTLYITREPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPCDIAVEWESNGQPENNYKTTPPVLDSDGSFFLYSKLTVD |
| | | KSRWQQGNVFSCSVMHEALHNHYTQKSLSLSPX |
| 92 | Human IgG1 HC | ASTKGPSVFPLAPSSKSTSGGTAALGCLVKDYFPEPVTVSW |
| | constant domain | NSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 93 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (YTE) | CNVNHKPSNTKVDKKVEPKSCDKTHTCPPCPAPELLGGPS |
| | | VFLFPPKPKDTLYITREPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 94 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (E233A/L235A) | CNVNHKPSNTKVDKKVEPKSCDKTHTCPPCPAPALAGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 95 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (L234A L235A | CNVNHKPSNTKVDKKVEPKSCDKTHTCPPCPAPEAAGGPS |
| | D265S) | VFLFPPKPKDTLMISRTPEVTCVVVSVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 96 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (L234A L235A | CNVNHKPSNTKVDKKVEPKSCDKTHTCPPCPAPEAAGGPS |
| | P329G) | VFLFPPKPKDTLMISRTPEVTCVVVSVSHEDPEVKFNWYV |
| | | DGVEVHNAKTKPREEQYNSTYRVVSVLTVLHQDWLNGK |
| | | EYKCKVSNKALGAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 97 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (L235E) | CNVNHKPSNTKVDKKVEPKSCDKTHTCPPCPAPELEGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 98 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (D265A) | CNVNHKPSNTKVDKKVEPKSCDKTHTCPPCPAPELLGGPS |
| | | VFLFPPKPKDTLMISRTPEVTCVVVAVSHEDPEVKENWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 99 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (D265A N297G) | CNVNHKPSNTKVDKKVEPKSCDKTHTCPPCPAPELLGGPS |
| | | VFLFPPKPKDTLMISRTPEVTCVVVAVSHEDPEVKFNWYV |
| | | DGVEVHNAKTKPREEQYGSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 100 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (N297X, wherein X | CNVNHKPSNTKVDKKVEPKSCDKTHTCPPCPAPELLGGPS |
| | is any amino acid | VFLFPPKPKDTLMISRTPEVTCVVVDVSHEDPEVKFNWYV |
| | other than N) | DGVEVHNAKTKPREEQYXSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 101 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (N297A/D356E/ | CNVNHKPSNTKVDKKVEPKSCDKTHTCPPCPAPELLGGPS |
| | L358M) | VFLFPPKPKDTLMISRTPEVTCVVVDVSHEDPEVKFNWYV |
| | | DGVEVHNAKTKPREEQYASTYRVVSVLTVLHQDWLNGK |
| | | EYKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 102 | Human LC Kappa | RTVAAPSVFIFPPSDEQLKSGTASVVCLLNNFYPREAKVQ |
| | constant domain | WKVDNALQSGNSQESVTEQDSKDSTYSLSSTLTLSKADYE |
| | | KHKVYACEVTHQGLSSPVTKSFNRGEC |
| 103 | Human IgG1 HC | ASTKGPSVFPLAPSSKSTSGGTAALGCLVKDYFPEPVTVSW |
| | constant domain | NSGALTSGVHTFPAVLQSSGLYSLSSVVTVPSSSLGTQTYI |
| | (S375C) | CNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 104 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (YTE S375C) | CNVNHKPSNTKVDKKVEPKSCDKTHTCPPCPAPELLGGPS |
| | | VFLFPPKPKDTLYITREPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 105 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (E233A/L235A | CNVNHKPSNTKVDKKVEPKSCDKTHTCPPCPAPALAGGPS |
| | S375C) | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 106 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (L234A L235A | CNVNHKPSNTKVDKKVEPKSCDKTHTCPPCPAPEAAGGPS |
| | D265S S375C) | VFLFPPKPKDTLMISRTPEVTCVVVSVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 107 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (L234A L235A | CNVNHKPSNTKVDKKVEPKSCDKTHTCPPCPAPEAAGGPS |
| | P329G S375C) | VFLFPPKPKDTLMISRTPEVTCVVVSVSHEDPEVKFNWYV |
| | | DGVEVHNAKTKPREEQYNSTYRVVSVLTVLHQDWLNGK |
| | | EYKCKVSNKALGAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 108 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (L235E S375C) | CNVNHKPSNTKVDKKVEPKSCDKTHTCPPCPAPELEGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 109 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (D265A S375C) | CNVNHKPSNTKVDKKVEPKSCDKTHTCPPCPAPELLGGPS |
| | | VFLFPPKPKDTLMISRTPEVTCVVVAVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 110 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (D265A N297G | CNVNHKPSNTKVDKKVEPKSCDKTHTCPPCPAPELLGGPS |
| | S375C) | VFLFPPKPKDTLMISRTPEVTCVVVAVSHEDPEVKFNWYV |
| | | DGVEVHNAKTKPREEQYGSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 111 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (N297X, wherein X | CNVNHKPSNTKVDKKVEPKSCDKTHTCPPCPAPELLGGPS |
| | is any amino acid | VFLFPPKPKDTLMISRTPEVTCVVVDVSHEDPEVKFNWYV |
| | other than N) | DGVEVHNAKTKPREEQYXSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 112 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYI |
| | (N297A/D356E/ | CNVNHKPSNTKVDKKVEPKSCDKTHTCPPCPAPELLGGPS |
| | L358M S375C) | VFLFPPKPKDTLMISRTPEVTCVVVDVSHEDPEVKFNWYV |
| | | DGVEVHNAKTKPREEQYASTYRVVSVLTVLHQDWLNGK |
| | | EYKCKVSNKALPAPIEKTISKAKGQPREPQVYTLPPSREEM |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 113 | Lm_T94F | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHYIFPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 114 | Lm_I93G | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHYGTPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 115 | Lm_V29R | DIQLTQSPSSLSASVGDRVSITCKASQDRSIAVAWYQQKPG |
| | | KAPKLLIYSASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 116 | Hm_F108Q_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYQDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 117 | Lm_D28H | DIQLTQSPSSLSASVGDRVSITCKASQHVSIAVAWYQQKPG |
| | | KAPKLLIYSASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 118 | Hm_F29G_S375C | QVQLQQSGSELKKPGASVKVSCKASGYTGTNYGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 119 | Hm_F108S_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYSDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 120 | Lm_Y53V | DIQLTQSPSSLSASVGDRVSITCKASQDVSIAVAWYQQKPG |
| | | KAPKLLIYSASVRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 121 | Hm_Y107N_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWNFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 122 | Hm_F101T_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGTGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 123 | Lm_Y53S | DIQLTQSPSSLSASVGDRVSITCKASQDVSIAVAWYQQKPG |
| | | KAPKLLIYSASSRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 124 | Hm_N31P_S375C | QVQLQQSGSELKKPGASVKVSCKASGYTFTPYGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 125 | Hm_Y32E_S375C | QVQLQQSGSELKKPGASVKVSCKASGYTFTNEGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 126 | Hm_G33I_S375C | QVQLQQSGSELKKPGASVKVSCKASGYTFTNYIMNWVKQ |
| | | APGQGLKWMGWINTYTGEPTYTDDFKGRFAFSLDTSVST |
| | | AYLQISSLKADDTAVYFCARGGFGSSYWYFDVWGQGSLV |
| | | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKRVEPKSCDKTHTCPPCPAPELL |
| | | GGPSVFLFPPKPKDTLMISRTPEVTCVVVDVSHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPCDIAVEWESNGQPENNYKTT |
| | | PPVLDSDGSFFLYSKLTVDKSRWQQGNVFSCSVMHEALH |
| | | NHYTQKSLSLSPGK |
| 127 | Hm_S104D_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSDYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 128 | Lm_Y53R | DIQLTQSPSSLSASVGDRVSITCKASQDVSIAVAWYQQKPG |
| | | KAPKLLIYSASRRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 129 | Lm_Y53N | DIQLTQSPSSLSASVGDRVSITCKASQDVSIAVAWYQQKPG |
| | | KAPKLLIYSASNRYTGVPDRESGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 130 | Lm_S50T | DIQLTQSPSSLSASVGDRVSITCKASQDVSIAVAWYQQKPG |
| | | KAPKLLIYTASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 131 | Lm_Q89V | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCVQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 132 | Hm_F101E_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGEGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 133 | Hm_Y107I_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWIFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 134 | Lm_S30R | DIQLTQSPSSLSASVGDRVSITCKASQDVRIAVAWYQQKP |
| | | GKAPKLLIYSASYRYTGVPDRFSGSGSGTDFTLTISSLQPED |
| | | FAVYYCQQHYITPLTFGAGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| 135 | Lm_H91K | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQKYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 136 | Lm_Y92E | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHEITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 137 | Hm_G33S_S375C | QVQLQQSGSELKKPGASVKVSCKASGYTFTNYSMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 138 | Hm_W106L_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYLYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 139 | Hm_W106H_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYHYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 140 | Hm_Y105S_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSSWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 141 | Lm_Y92N | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHNITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 142 | Lm_A32P | DIQLTQSPSSLSASVGDRVSITCKASQDVSIPVAWYQQKPG |
| | | KAPKLLIYSASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 143 | Hm_W106A_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYAYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 144 | Lm_A32Q | DIQLTQSPSSLSASVGDRVSITCKASQDVSIQVAWYQQKPG |
| | | KAPKLLIYSASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 145 | Lm_H91Y | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQYYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 146 | Hm_F108T_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYTDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 147 | Hm_W106E_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYEYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 148 | Hm_G33P_S375C | QVQLQQSGSELKKPGASVKVSCKASGYTFTNYPMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 149 | Hm_S103T_S375C | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGTSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 150 | Lm_Y92A | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHAITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 151 | Lm_S50L | DIQLTQSPSSLSASVGDRVSITCKASQDVSIAVAWYQQKPG |
| | | KAPKLLIYLASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 152 | Lm_Y92A | DIQLTQSPSSLSASVGDRVSITCKASQDVSIAVAWYQQKPG |
| | | KAPKLLIYSASYRYTGVPDRFSGSGSGTDFTLTISSLQPEDF |
| | | AVYYCQQHAITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 153 | Lm_A32P | DIQLTQSPSSLSASVGDRVSITCKASQDVSIPVAWYQQKPG |
| | | KAPKLLIYSASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 154 | Lm_S50L | DIQLTQSPSSLSASVGDRVSITCKASQDVSIAVAWYQQKPG |
| | | KAPKLLIYLASYRYTGVPDRFSGSGSGTDFTLTISSLQPEDF |
| | | AVYYCQQHYITPLTFGAGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 155 | Hm_S104D | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSDYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 156 | Hm_W106E | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYEYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 157 | Hm_G33S | QVQLQQSGSELKKPGASVKVSCKASGYTFTNYSMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 158 | Hm_Y105S | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSSWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 159 | Hm_W106A | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYAYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 160 | Hm_W106L | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYLYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 161 | Hm_S103T | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGTSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 162 | Hm_F108T | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYTDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 163 | Hm_G33I | QVQLQQSGSELKKPGASVKVSCKASGYTFTNYIMNWVKQ |
| | | APGQGLKWMGWINTYTGEPTYTDDFKGRFAFSLDTSVST |
| | | AYLQISSLKADDTAVYFCARGGFGSSYWYFDVWGQGSLV |
| | | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKRVEPKSCDKTHTCPPCPAPELL |
| | | GGPSVFLFPPKPKDTLMISRTPEVTCVVVDVSHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 164 | Hm_W106H | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYFYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 165 | Hm_F29G | QVQLQQSGSELKKPGASVKVSCKASGYTGTNYGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 166 | Hm_N31P | QVQLQQSGSELKKPGASVKVSCKASGYTFTPYGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 167 | Hm_F108S | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYSDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 168 | Hm_F101T | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGTGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 169 | Hm_Y107N | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWNFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 170 | Hm_F108Q | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWYQDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 171 | Hm_Y107I | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGFGSSYWIFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 172 | Hm_G33P | QVQLQQSGSELKKPGASVKVSCKASGYTFTNYPMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 173 | Hm_Y32E | QVQLQQSGSELKKPGASVKVSCKASGYTFTNEGMNWVK |
| | | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 174 | Hm_F101E | QVQLQQSGSELKKPGASVKVSCKASGYTFTNYGMNWVK |
| | | QAPGQGLKWMGWINTYTGEPTYTDDFKGRFAFSLDTSVS |
| | | TAYLQISSLKADDTAVYFCARGGEGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| 175 | Alpha-TROP2 HC | ASTKGPSVFPLAPSSKSTSGGTAALGCLVKDYFPEPVTVSW |
| | constant domain | NSGALTSGVHTFPAVLQSSGLYSLSSVVTVPSSSLGTQTYI |
| | | CNVNHKPSNTKVDKRVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 176 | Alpha-TROP2 HC | ASTKGPSVFPLAPSSKSTSGGTAALGCLVKDYFPEPVTVSW |
| | constant domain | NSGALTSGVHTFPAVLQSSGLYSLSSVVTVPSSSLGTQTYI |
| | (S375C) | CNVNHKPSNTKVDKRVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSREEM |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 177 | Alpha-TROP2 LC | RTVAAPSVFIFPPSDEQLKSGTASVVCLLNNFYPREAKVQ |
| | constant domain | WKVDNALQSGNSQESVTEQDSKDSTYSLSSTLTLSKADYE |
| | | KHKVYACEVTHQGLSSPVTKSFNRGEC |
| 178 | Alpha-TROP2 HC | ASTKGPSVFPLAPSSKSTSGGTAALGCLVKDYFPEPVTVSW |
| | (BSM) | NSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 179 | Alpha-TROP2 HC | ASTKGPSVFPLAPSSKSTSGGTAALGCLVKDYFPEPVTVSW |
| | (BSM-YTE) | NSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLYITREPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 180 | Alpha-TROP2 HC | ASTKGPSVFPLAPSSKSTSGGTAALGCLVKDYFPEPVTVSW |
| | (BSM-S375C) | NSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 181 | Alpha-TROP2 HC | ASTKGPSVFPLAPSSKSTSGGTAALGCLVKDYFPEPVTVSW |
| | (BSM-YTE-S375C) | NSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLYITREPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSRDEL |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 182 | Alpha-TROP2 HC | ASTKGPSVFPLAPSSKSTSGGTAALGCLVKDYFPEPVTVSW |
| | (YTE) | NSGALTSGVHTFPAVLQSSGLYSLSSVVTVPSSSLGTQTYI |
| | | CNVNHKPSNTKVDKRVEPKSCDKTHTCPPCPAPELLGGPS |
| | | VFLFPPKPKDTLYITREPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 183 | Alpha-TROP2 HC | ASTKGPSVFPLAPSSKSTSGGTAALGCLVKDYFPEPVTVSW |
| | (YTE S375C) | NSGALTSGVHTFPAVLQSSGLYSLSSVVTVPSSSLGTQTYI |
| | | CNVNHKPSNTKVDKRVEPKSCDKTHTCPPCPAPELLGGPS |
| | | VFLFPPKPKDTLYITREPEVTCVVVDVSHEDPEVKFNWYV |
| | | DGVEVHNAKTKPREEQYNSTYRVVSVLTVLHQDWLNGK |
| | | EYKCKVSNKALPAPIEKTISKAKGQPREPQVYTLPPSREEM |
| | | TKNQVSLTCLVKGFYPCDIAVEWESNGQPENNYKTTPPVL |
| | | DSDGSFFLYSKLTVDKSRWQQGNVFSCSVMHEALHNHYT |
| | | QKSLSLSPGK |
| 184 | Alpha-TROP2 HC | QVQLQQSGSELKKPGASVKVSCKASGYTFTNYGMNWVK |
| | (S375C) | QAPGQGLKWMGWINTYTGEPTYTDDFKGRFAFSLDTSVS |
| | | TAYLQISSLKADDTAVYFCARGGFGSSYWYFDVWGQGSL |
| | | VTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REEMTKNQVSLTCLVKGFYPCDIAVEWESNGQPENNYKT |
| | | TPPVLDSDGSFFLYSKLTVDKSRWQQGNVFSCSVMHEALH |
| | | NHYTQKSLSLSPGK |
| 185 | GGFG peptide | GGFG |
| 186 | GGGG peptide | GGGG |
| 187 | G4S | G4S |
| 188 | (G4S)n, n = 1-5 | GGGGSGGGGSGGGGSGGGGSGGGGS |
| 189 | (G4S)3 | GGGGSGGGGSGGGGS |
| 190 | (G4S)1-10 | GGGGSGGGGSGGGGSGGGGSGGGGSGGGGSGGGGSGGG |
| | | GSGGGGSGGGGS |

--

While the present invention is described herein with reference to illustrated embodiments, it should be understood that the invention is not limited hereto. Those having ordinary skill in the art and access to the teachings herein will recognize additional modifications and embodiments within the scope thereof. Various modifications of the invention in addition to those described herein will become apparent to those skilled in the art from the foregoing description. Such modifications are intended to fall within the scope of the claims. Therefore, the present invention is limited only by the language of the specification and the claims attached herein.

Patents, patent applications, publications, product descriptions, and protocols are cited throughout this application, the disclosures of which are incorporated herein by reference in their entireties for all purposes.

What is claimed:

1. An antibody drug conjugate (ADC) comprising an antibody or antigen-binding fragment thereof that specifically binds to human TROP2 conjugated to a linker-monomethylauristatin E (linker-MMAE) payload, wherein the antibody comprises two heavy chains, each heavy chain comprising a variable domain and a constant domain, the variable domain comprising a complementarity determining region (CDR) H₁, a CDRH2, and a CDRH3, and two light chains, each light chain comprising a variable domain and a constant domain, the variable domain comprising a CDRL1, a CDRL2, and a CDRL3, wherein the CDRH1, CDRH2, CDRH3, CDRL1, CDRL2, and CDRL3, respectively, comprise the amino acid sequence of NYGMN (SEQ ID NO: 4), WINTYTGEPTYTDDFKG (SEQ ID NO: 5), GGFGSSYWYFDV (SEQ ID NO: 6), KASQDVSIAVA (SEQ ID NO: 7), SASDRYT (SEQ ID NO: 10), and QQHYITPLT (SEQ ID NO: 9).

2. The ADC of claim 1, wherein the antibody or antigen-binding fragment thereof displays reduced binding to low TROP2-expressing cells compared to high TROP2-expressing cells and has reduced hydrophobicity compared to Sacituzumab as determined by hydrophobic interaction chromatography (HIC).

3. The ADC of claim 1, wherein the antibody comprises a light chain comprising the amino acid sequence of SEQ ID NO: 13 or 22 and a heavy chain comprising the amino acid sequence of SEQ ID NO: 11, 17, or 18.

4. The ADC of claim 1, wherein the antibody comprises a light chain comprising the amino acid sequence of SEQ ID NO: 13 or 22 and a heavy chain comprising the amino acid sequence of SEQ ID NO: 58, 59, or 60.

5. The ADC of claim 1, wherein the antibody further comprises a cysteine or a non-canonical amino acid amino acid substitution at one or more position(s) selected from the group consisting of: positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

6. The ADC of claim 5, wherein the antibody comprises a cysteine or a non-canonical amino acid amino acid substitution at position 375 of the constant domain of the heavy chain.

7. The ADC of claim 6, wherein the antibody comprises a heavy chain comprising the amino acid sequence of SEQ ID NO: 19, 20, 61, or 62.

8. The ADC of claim 6, wherein the linker-MMAE payload is conjugated to the cysteine or noncanonical amino acid.

9. The ADC of claim 1, wherein the antibody comprises a cysteine residue in which the thiol (SH) group thereof is conjugated to a linker-MMAE payload comprising the

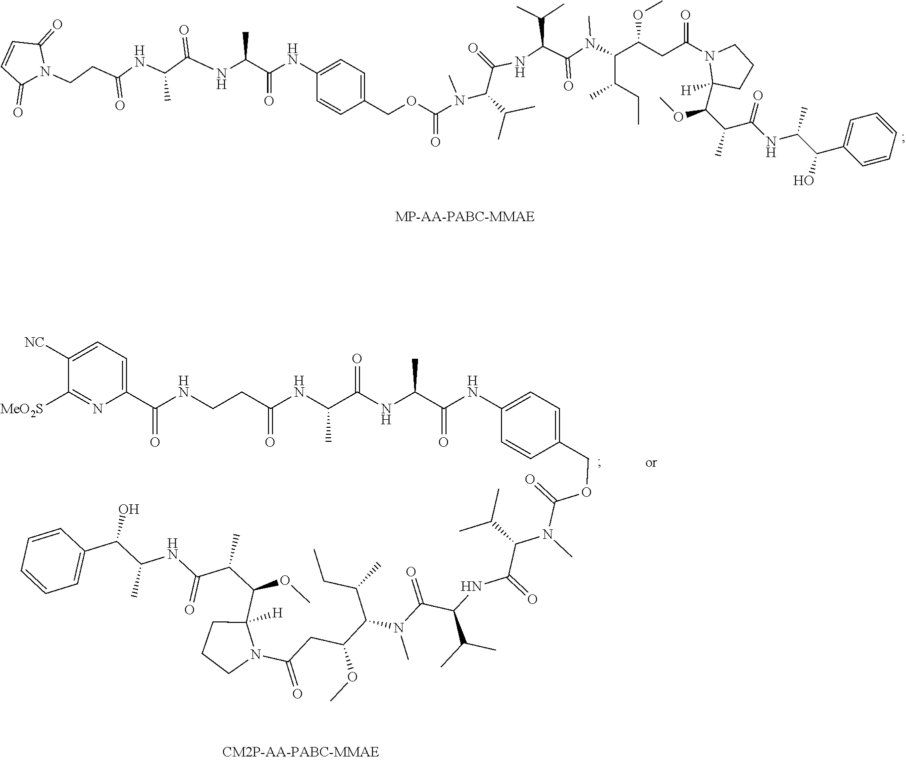 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2ccc(C#N)c(S(C)(=O)=O)n2)cc1)C(C)C]

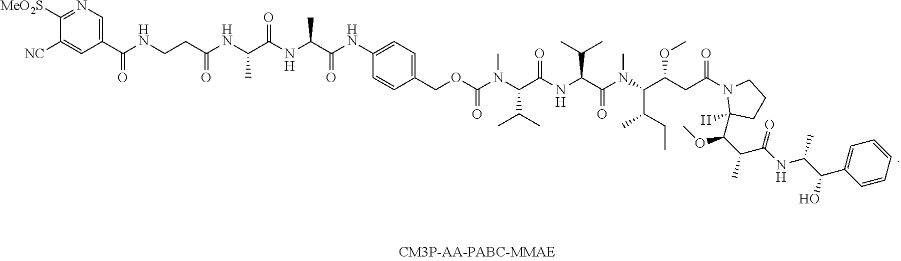 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2cnc(S(C)(=O)=O)c(C#N)c2)cc1)C(C)C]

10. The ADC of claim 1, wherein the ADC comprises the formula: 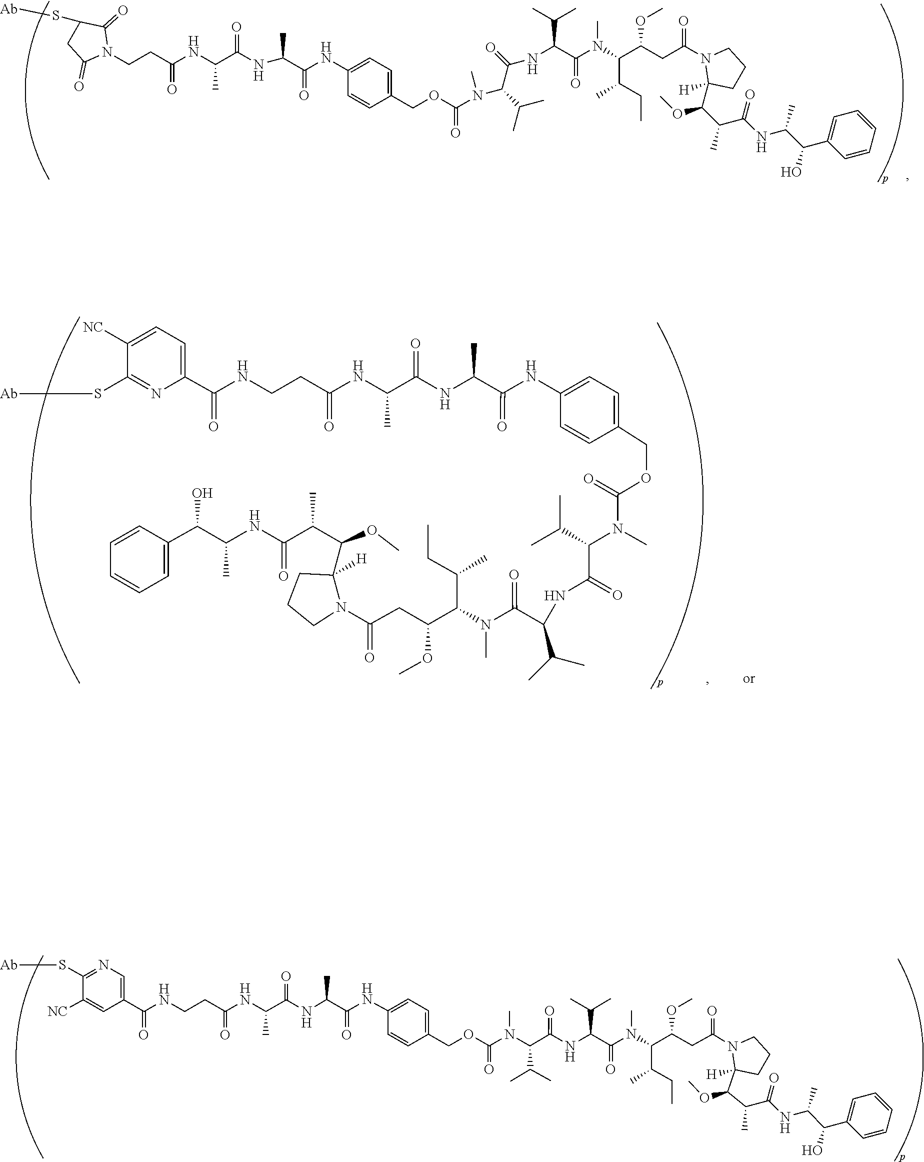
wherein Ab is an anti-TROP2 antibody comprising heavy chain engineered cysteine residues or light chain engineered cysteine residues, wherein the anti-TROP2 antibody comprising the engineered cysteine residues is (A) selected from the group consisting of:
(a) αTROP2 (HC:Sac-E152C) (LC:Sac-Y53D) comprising two heavy chains having the amino acid sequence set forth in SEQ ID No: 64 and two light chains having the amino acid sequence set forth in SEQ ID NO: 13;
(b) αTROP2 (HC:Sac-E153C) (LC:Sac-Y53D) two heavy chains having the amino acid sequence set forth in SEQ ID No: 65 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 66 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 67 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 68 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 69 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 71 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 72 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 73 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 74 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 75 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 76 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 58 and two light chains having the amino acid sequence set forth in SEQ ID NO: 50;
(n) αTROP2 (HC:Sac) (LC:Sac-Y53D-E168C) comprising two heavy chains having the amino acid sequence set forth in SEQ ID No: 58 and two light chains having the amino acid sequence set forth in SEQ ID NO: 51;
(o) αTROP2 (HC:Sac-YTE) (LC:Sac-Y53D-E165C) comprising two heavy chains having the amino acid sequence set forth in SEQ ID No: 63 and two light chains having the amino acid sequence set forth in SEQ ID NO: 50;
(p) αTROP2 (HC:Sac-YTE) (LC:Sac-Y53D-E168C) comprising two heavy chains having the amino acid sequence set forth in SEQ ID No: 63 and two light chains having the amino acid sequence set forth in SEQ ID NO: 51;
(q) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 21;
(r) αTROP2 (HC:BSM-E152C) (LC:BSM-Y53D) comprising two heavy chains having the amino acid sequence set forth in SEQ ID No: 78 and two light chains having the amino acid sequence set forth in SEQ ID NO: 22;
(s) αTROP2 (HC:BSM-E153C) (LC:BSM-Y53D) comprising two heavy chains having the amino acid sequence set forth in SEQ ID No: 79 and two light chains having the amino acid sequence set forth in SEQ ID NO: 22;
(t) αTROP2 (HC:BSM-E171C) (LC:BSM-Y53D) comprising two heavy chains having the amino acid sequence set forth in SEQ ID No: 80 and two light chains having the amino acid sequence set forth in SEQ ID NO: 22;
(u) αTROP2 (HC:BSM-E172C) (LC:BSM-Y53D) comprising two heavy chains having the amino acid sequence set forth in SEQ ID No: 81 and two light chains having the amino acid sequence set forth in SEQ ID NO: 22;
(v) αTROP2 (HC:BSM-E173C) (LC:BSM-Y53D) comprising two heavy chains having the amino acid sequence set forth in SEQ ID No: 82 and two light chains having the amino acid sequence set forth in SEQ ID NO: 22;
(w) αTROP2 (HC:BSM-S375C) (LC:BSM-Y53D) comprising two heavy chains having the amino acid sequence set forth in SEQ ID No: 83 and two light chains having the amino acid sequence set forth in SEQ ID NO: 22;
(x) αTROP2 (HC:BSM) (LC:BSM-Y53D-E165C) comprising two heavy chains having the amino acid sequence set forth in SEQ ID No: 59 and two light chains having the amino acid sequence set forth in SEQ ID NO: 54;
(y) αTROP2 (HC:BSM) (LC:BSM-Y53D-E168C) comprising two heavy chains having the amino acid sequence set forth in SEQ ID No: 59 and two light chains having the amino acid sequence set forth in SEQ ID NO: 55;
(z) αTROP2 (HC:BSM-YTE) (LC:BSM-Y53D-E165C) comprising two heavy chains having the amino acid sequence set forth in SEQ ID No: 60 and two light chains having the amino acid sequence set forth in SEQ ID NO: 54;
(aa) αTROP2 (HC:BSM-YTE) (LC:BSM-Y53D-E168C) comprising two heavy chains having the amino acid sequence set forth in SEQ ID No: 58 and two light chains having the amino acid sequence set forth in SEQ ID NO: 57;
(bb) αTROP2 (HC:BSM-YTE-E152C) (LC:BSM-Y53D) comprising two heavy chains having the amino acid sequence set forth in SEQ ID No: 85 and two light chains having the amino acid sequence set forth in SEQ ID NO: 22;
(cc) αTROP2 (HC:BSM-YTE-E153C) (LC:BSM-Y53D) comprising two heavy chains having the amino acid sequence set forth in SEQ ID No: 86 and two light chains having the amino acid sequence set forth in SEQ ID NO: 22;
(dd) αTROP2 (HC:BSM-YTE-E171C) (LC:BSM-Y53D) comprising two heavy chains having the amino acid sequence set forth in SEQ ID No: 87 and two light chains having the amino acid sequence set forth in SEQ ID NO: 22;
(ee) αTROP2 (HC:BSM-YTE-E172C) (LC:BSM-Y53D) comprising two heavy chains having the amino acid sequence set forth in SEQ ID No: 88 and two light chains having the amino acid sequence set forth in SEQ ID NO: 22;
(ff) αTROP2 (HC:BSM-YTE-E173C) (LC:BSM-Y53D) comprising two heavy chains having the amino acid sequence set forth in SEQ ID No: 89 and two light chains having the amino acid sequence set forth in SEQ ID NO: 22; and
(gg) αTROP2 (HC:BSM-YTE-S375C) (LC:BSM-Y53D) comprising two heavy chains having the amino acid sequence set forth in SEQ ID No: 90 and two light chains having the amino acid sequence set forth in SEQ ID NO: 22;
wherein S is from the side chain of the engineered cysteine residue; and, wherein p is an integer selected from 1 or 2; or (B) selected from the group consisting of:
(a) αTROP2 (HC:Sac-E152C-S375C) (LC:Sac-Y53D) comprising two heavy chains having the amino acid sequence set forth in SEQ ID No: 70 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 77 and two light chains having the amino acid sequence set forth in SEQ ID NO: 13;
(c) αTROP2 (HC:BSM-E152C-S375C) (LC:BSM-Y53D) comprising two heavy chains having the amino acid sequence set forth in SEQ ID No: 84 and two light chains having the amino acid sequence set forth in SEQ ID NO: 22; and
(d) αTROP2 (HC:BSM-YTE-E152C-S375C) (LC:BSM-Y53D) comprising two heavy chains having the amino acid sequence set forth in SEQ ID No: 91 and two light chains having the amino acid sequence set forth in SEQ ID NO: 22;
wherein S is from the side chain of the engineered cysteine residue; and, wherein p is an integer selected from 1, 2, 3, or 4.

11. The ADC of claim 1, wherein the ADC comprises the formula: 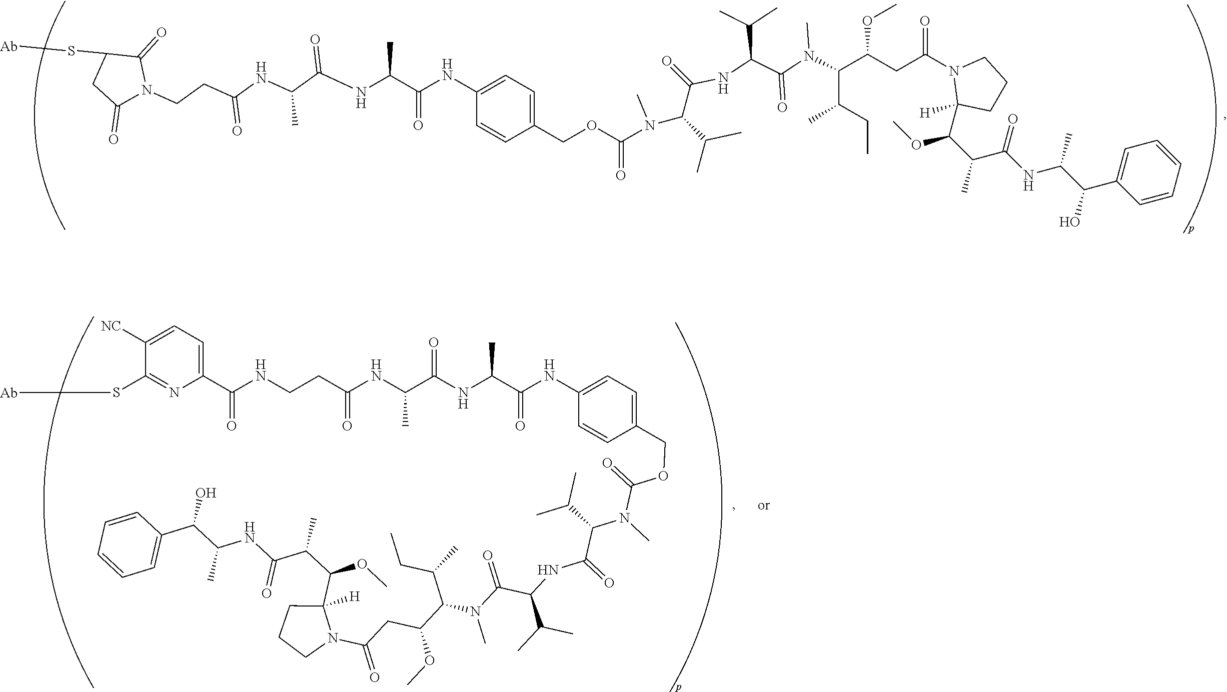 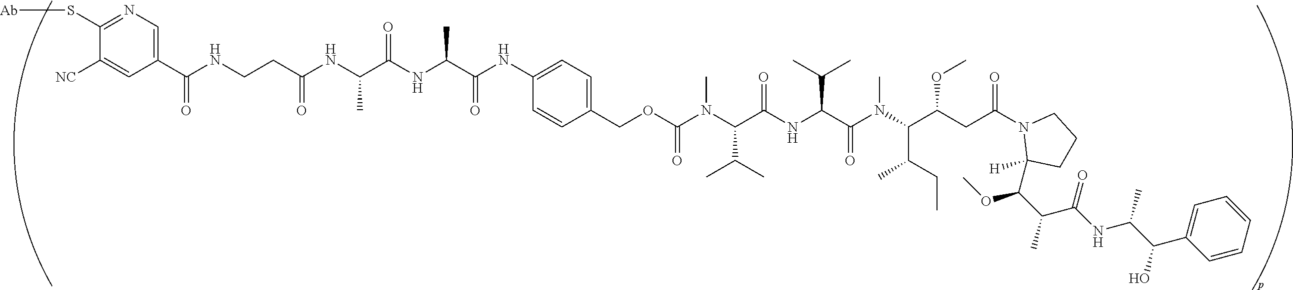
wherein Ab is an anti-TROP2 antibody comprising heavy chain engineered cysteine residues or light chain engineered cysteine residues, wherein the anti-TROP2 antibody comprising the engineered cysteine residues is (A) selected from the group consisting of:
(a) αTROP2 (HC:Sac-E152C) (LC:Sac-Y53D) comprising two heavy chains having the amino acid sequence set forth in SEQ ID No: 24 and two light chains having the amino acid sequence set forth in SEQ ID NO: 13;
(b) αTROP2 (HC:Sac-E153C) (LC:Sac-Y53D) comprising two heavy chains having the amino acid sequence set forth in SEQ ID No: 25 and two light chains having the amino acid sequence set forth in SEQ ID NO: 13;
(c) αTROP2 (HC:Sac-E171C) (LC:Sac-Y53D) comprising two heavy chains having the amino acid sequence set forth in SEQ ID No: 26 and two light chains having the amino acid sequence set forth in SEQ ID NO: 13;
(d) αTROP2 (HC:Sac-E172C) (LC:Sac-Y53D) comprising two heavy chains having the amino acid sequence set forth in SEQ ID No: 27 and two light chains having the amino acid sequence set forth in SEQ ID NO: 13;
(e) αTROP2 (HC:Sac-E173C) (LC:Sac-Y53D) comprising two heavy chains having the amino acid sequence set forth in SEQ ID No: 28 and two light chains having the amino acid sequence set forth in SEQ ID NO: 13;
(f) αTROP2 (HC:Sac-S375C) (LC:Sac-Y53D) comprising two heavy chains having the amino acid sequence set forth in SEQ ID No: 29 and two light chains having the amino acid sequence set forth in SEQ ID NO: 13;
(g) αTROP2 (HC:Sac-YTE-E152C) (LC:Sac-Y53D) comprising two heavy chains having the amino acid sequence set forth in SEQ ID No: 31 and two light chains having the amino acid sequence set forth in SEQ ID NO: 13;
(h) αTROP2 (HC:Sac-YTE-E153C) (LC:Sac-Y53D) comprising two heavy chains having the amino acid sequence set forth in SEQ ID No: 32 and two light chains having the amino acid sequence set forth in SEQ ID NO: 13;
(i) αTROP2 (HC:Sac-YTE-E171C) (LC:Sac-Y53D) comprising two heavy chains having the amino acid sequence set forth in SEQ ID No: 33 and two light chains having the amino acid sequence set forth in SEQ ID NO: 13;
(j) αTROP2 (HC:Sac-YTE-E172C) (LC:Sac-Y53D) comprising two heavy chains having the amino acid sequence set forth in SEQ ID No: 34 and two light chains having the amino acid sequence set forth in SEQ ID NO: 13;
(k) αTROP2 (HC:Sac-YTE-E173C) (LC:Sac-Y53D) comprising two heavy chains having the amino acid sequence set forth in SEQ ID No: 35 and two light chains having the amino acid sequence set forth in SEQ ID NO: 13;
(l) αTROP2 (HC:Sac-YTE-S375C) (LC:Sac-Y53D) comprising two heavy chains having the amino acid sequence set forth in SEQ ID No: 36 and two light chains having the amino acid sequence set forth in SEQ ID NO: 13;
(m) αTROP2 (HC:Sac) (LC:Sac-Y53D-E165C) comprising two heavy chains having the amino acid sequence set forth in SEQ ID No: 11 and two light chains having the amino acid sequence set forth in SEQ ID NO: 52;
(n) αTROP2 (HC:Sac) (LC:Sac-Y53D-E168C) comprising two heavy chains having the amino acid sequence set forth in SEQ ID No: 11 and two light chains having the amino acid sequence set forth in SEQ ID NO: 53;
(o) αTROP2 (HC:Sac-YTE) (LC:Sac-Y53D-E165C) comprising two heavy chains having the amino acid sequence set forth in SEQ ID No: 23 and two light chains having the amino acid sequence set forth in SEQ ID NO: 52;
(p) αTROP2 (HC:Sac-YTE) (LC:Sac-Y53D-E168C) comprising two heavy chains having the amino acid sequence set forth in SEQ ID No: 23 and two light chains having the amino acid sequence set forth in SEQ ID NO: 53;
(q) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 21;
(r) αTROP2 (HC:BSM-E152C) (LC:BSM-Y53D) comprising two heavy chains having the amino acid sequence set forth in SEQ ID No: 38 and two light chains having the amino acid sequence set forth in SEQ ID NO: 22;
(s) αTROP2 (HC:BSM-E153C) (LC:BSM-Y53D) comprising two heavy chains having the amino acid sequence set forth in SEQ ID No: 39 and two light chains having the amino acid sequence set forth in SEQ ID NO: 22;
(t) αTROP2 (HC:BSM-E171C) (LC:BSM-Y53D) comprising two heavy chains having the amino acid sequence set forth in SEQ ID No: 40 and two light chains having the amino acid sequence set forth in SEQ ID NO: 22;
(u) αTROP2 (HC:BSM-E172C) (LC:BSM-Y53D) comprising two heavy chains having the amino acid sequence set forth in SEQ ID No: 41 and two light chains having the amino acid sequence set forth in SEQ ID NO: 22;
(v) αTROP2 (HC:BSM-E173C) (LC:BSM-Y53D) comprising two heavy chains having the amino acid sequence set forth in SEQ ID No: 42 and two light chains having the amino acid sequence set forth in SEQ ID NO: 22;
(w) αTROP2 (HC:BSM-S375C) (LC:BSM-Y53D) comprising two heavy chains having the amino acid sequence set forth in SEQ ID No: 19 and two light chains having the amino acid sequence set forth in SEQ ID NO: 22;
(x) αTROP2 (HC:BSM) (LC:BSM-Y53D-E168C) comprising two heavy chains having the amino acid sequence set forth in SEQ ID No: 17 and two light chains having the amino acid sequence set forth in SEQ ID NO: 55;
(y) αTROP2 (HC:BSM-YTE) (LC:BSM-Y53D-E165C) comprising two heavy chains having the amino acid sequence set forth in SEQ ID No: 18 and two light chains having the amino acid sequence set forth in SEQ ID NO: 54;
(z) αTROP2 (HC:BSM-YTE) (LC:BSM-Y53D-E168C) comprising two heavy chains having the amino acid sequence set forth in SEQ ID No: 18 and two light chains having the amino acid sequence set forth in SEQ ID NO: 55;
(aa) αTROP2 (HC:BSM-YTE-E152C) (LC:BSM-Y53D) comprising two heavy chains having the amino acid sequence set forth in SEQ ID No: 44 and two light chains having the amino acid sequence set forth in SEQ ID NO: 22;
(bb) αTROP2 (HC:BSM-YTE-E153C) (LC:BSM-Y53D) comprising two heavy chains having the amino acid sequence set forth in SEQ ID No: 45 and two light chains having the amino acid sequence set forth in SEQ ID NO: 22;
(cc) αTROP2 (HC:BSM-YTE-E171C) (LC:BSM-Y53D) comprising two heavy chains having the amino acid sequence set forth in SEQ ID No: 46 and two light chains having the amino acid sequence set forth in SEQ ID NO: 22;
(dd) αTROP2 (HC:BSM-YTE-E172C) (LC:BSM-Y53D) comprising two heavy chains having the amino acid sequence set forth in SEQ ID No: 47 and two light chains having the amino acid sequence set forth in SEQ ID NO: 22;
(ee) αTROP2 (HC:BSM-YTE-E173C) (LC:BSM-Y53D) comprising two heavy chains having the amino acid sequence set forth in SEQ ID No: 48 and two light chains having the amino acid sequence set forth in SEQ ID NO: 22; and
(ff) αTROP2 (HC:BSM-YTE-S375C) (LC:BSM-Y53D) comprising two heavy chains having the amino acid sequence set forth in SEQ ID No: 20 and two light chains having the amino acid sequence set forth in SEQ ID NO: 22;
wherein S is from the side chain of the engineered cysteine residue; and, wherein p is an integer selected from 1 or 2; or (B) selected from the group consisting of:
(a) αTROP2 (HC:Sac-E152C-S375C) (LC:Sac-Y53D) comprising two heavy chains having the amino acid sequence set forth in SEQ ID No: 30 and two light chains having the amino acid sequence set forth in SEQ ID NO: 13;
(b) αTROP2 (HC:Sac-YTE-E152C-S375C) (LC:Sac-Y53D) comprising two heavy chains having the amino acid sequence set forth in SEQ ID No: 37 and two light chains having the amino acid sequence set forth in SEQ ID NO: 13;
(c) αTROP2 (HC:BSM-E152C-S375C) (LC:BSM-Y53D) comprising two heavy chains having the amino acid sequence set forth in SEQ ID No: 43 and two light chains having the amino acid sequence set forth in SEQ ID NO: 22; and
(d) αTROP2 (HC:BSM-YTE-E152C-S375C) (LC:BSM-Y53D) comprising two heavy chains having the amino acid sequence set forth in SEQ ID No: 49 and two light chains having the amino acid sequence set forth in SEQ ID NO: 22;
wherein S is from the side chain of the engineered cysteine residue; and, wherein p is an integer selected from 1, 2, 3, or 4.

12. The ADC of claim 1, wherein the ADC comprises the formula: 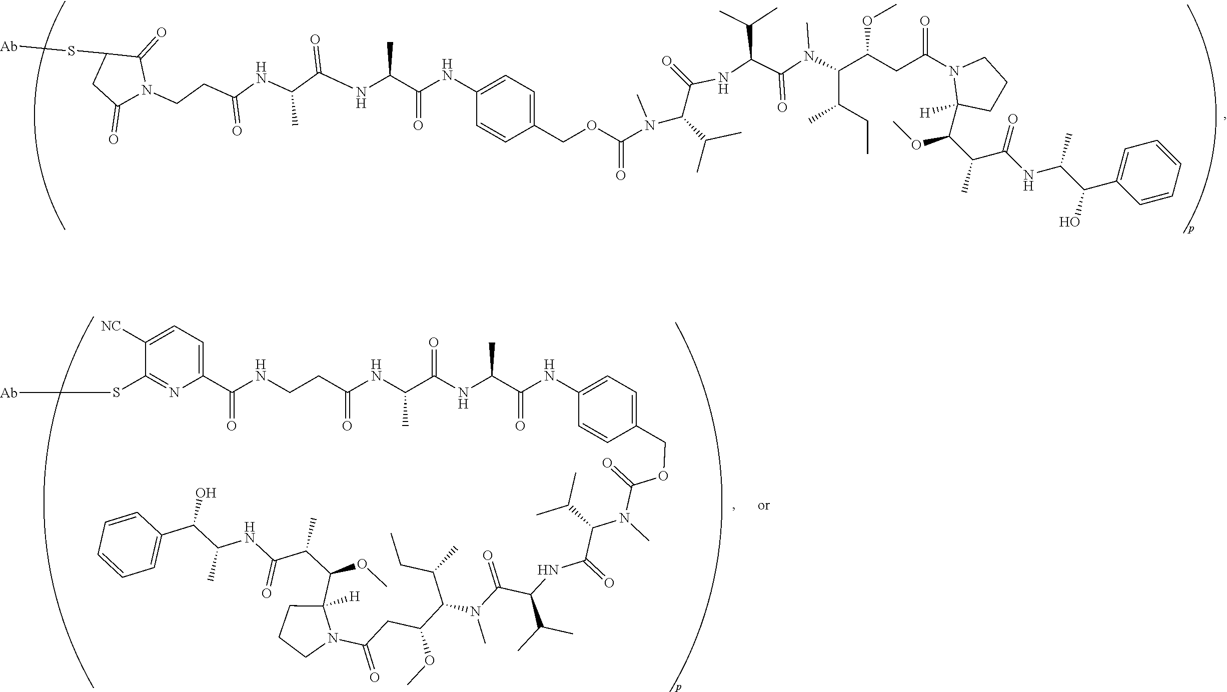 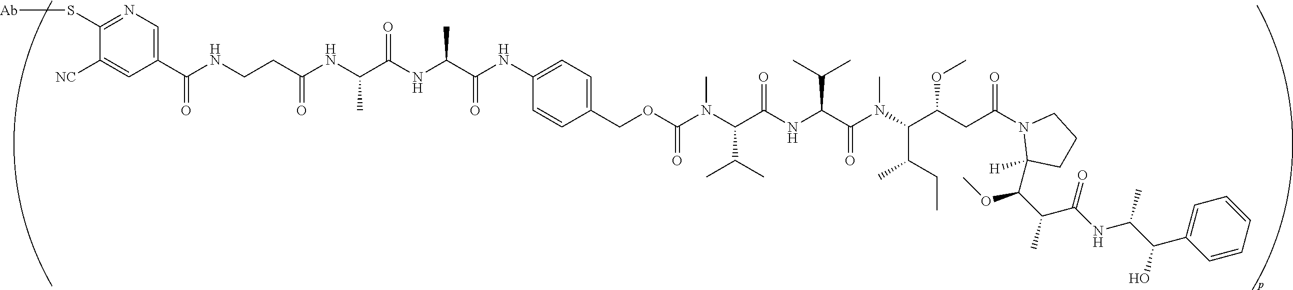
wherein Ab is an anti-Trop2 antibody comprising two heavy chains having the amino acid sequence set forth in SEQ ID NO: 20 and two light chains having the amino acid sequence set forth in SEQ ID NO: 22; wherein p is 1 or 2; and wherein S is from the side chain of an engineered cysteine residue at position 375 of the constant domain of the heavy chain, the position as defined according to Eu numbering.

13. A composition comprising the ADC of claim 1 and a pharmaceutically acceptable carrier.

14. A method for treating a cancer in an individual in need thereof comprising administering to the individual a therapeutically effective amount of the ADC of a claim 1 or the composition of claim 13 to treat the cancer, wherein the cancer is a cancer that overexpresses TROP2.

15. A combination therapy for treating cancer comprising the ADC of claim 1 or the composition of claim 13 and a therapeutic agent, wherein the cancer is a cancer that overexpresses TROP2.

16. An antibody-drug conjugate (ADC) comprising an antibody that specifically binds to human TROP2 conjugated to a linker-monomethylauristatin E (linker-MMAE) payload, wherein the antibody comprises two heavy chains, each heavy chain comprising a variable domain and a constant domain, the variable domain comprising the amino acid sequence of SEQ ID NO: 14, and two light chains, each light chain comprising a variable domain and a constant domain, the variable domain comprising the amino acid sequence of SEQ ID NO: 15.

17. The ADC of claim 16, wherein the antibody comprises a cysteine residue in which the thiol (SH) group thereof is conjugated to a linker-MMAE payload comprising the formula:

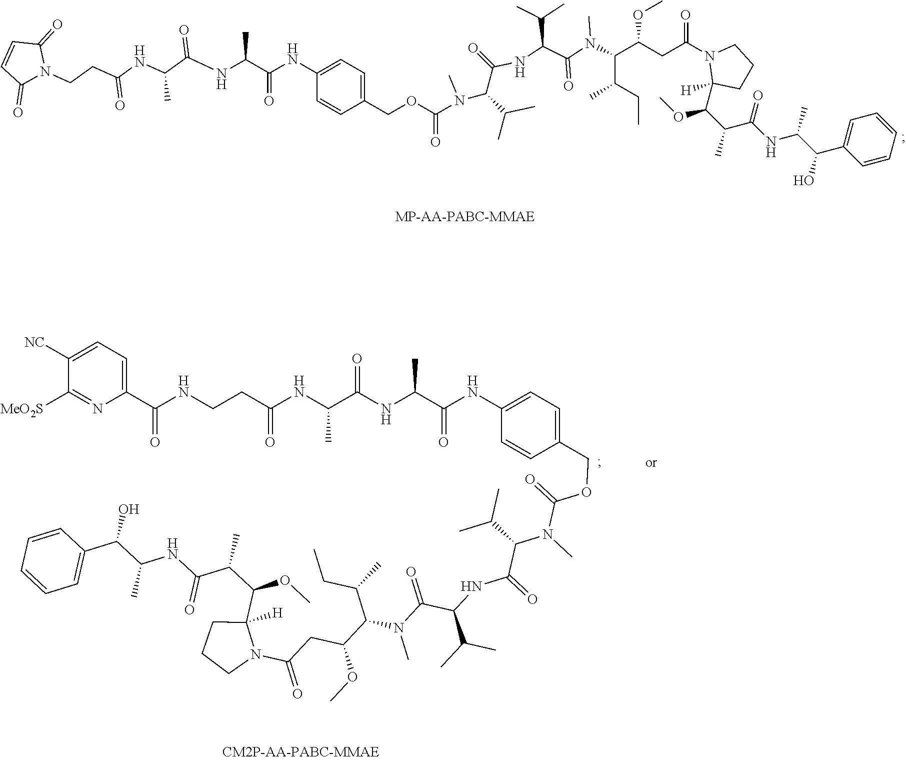 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2ccc(C#N)c(S(C)(=O)=O)n2)cc1)C(C)C]

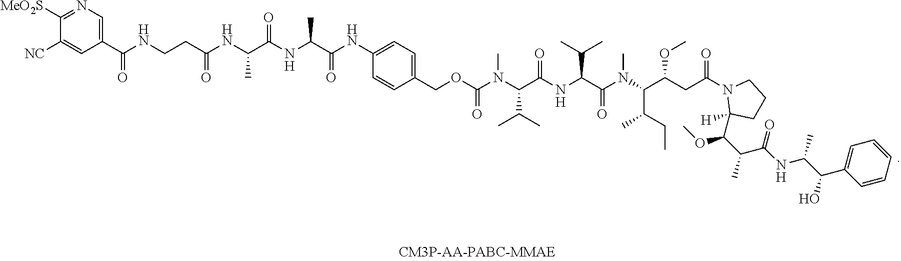 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCNC(=O)c2cnc(S(C)(=O)=O)c(C#N)c2)cc1)C(C)C]

18. The ADC of claim 16, wherein the ADC comprises the formula: 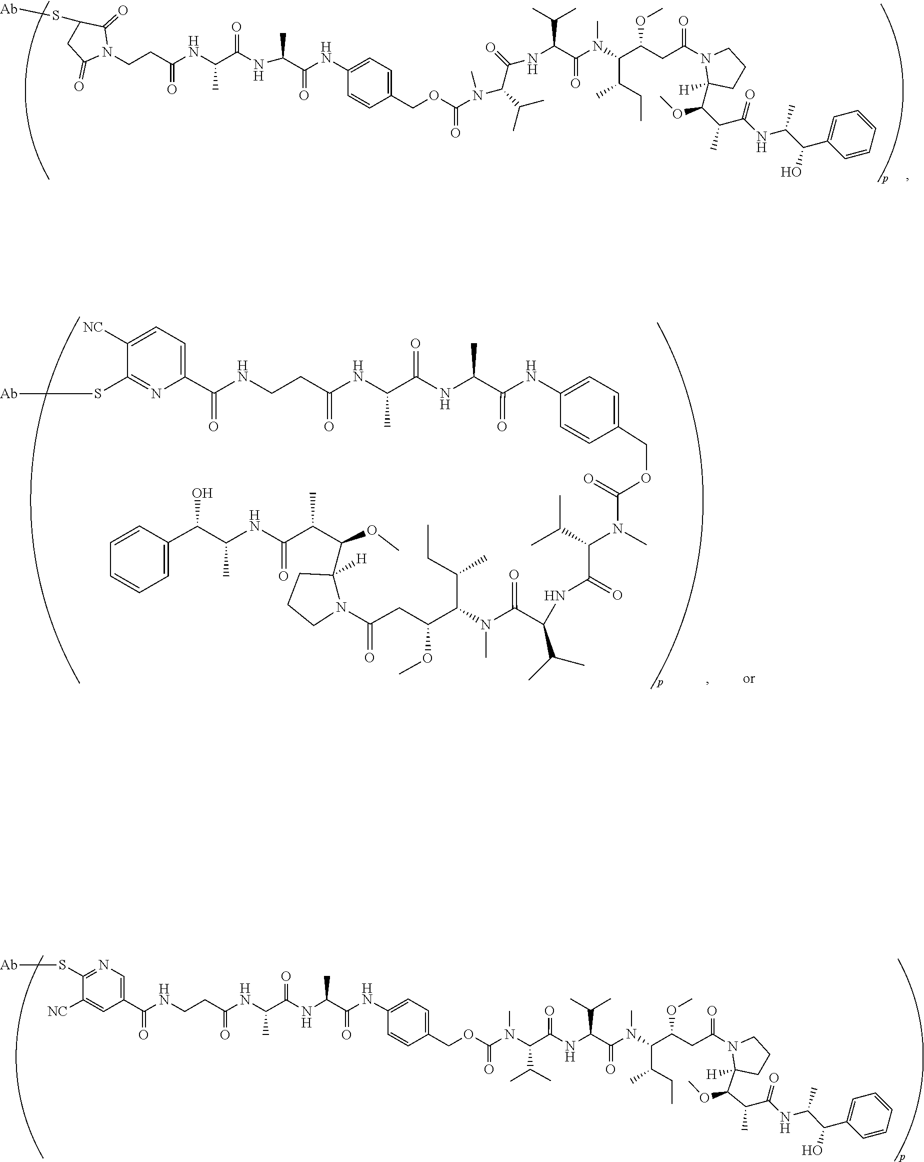
wherein Ab is the antibody, wherein the antibody comprises a heavy chain engineered cysteine residues or light chain engineered cysteine residues, wherein the antibody comprising the engineered cysteine residues is: (A) selected from the group consisting of:
(a) αTROP2 (HC:BSM-E152C) (LC:BSM) comprising two heavy chains having the amino acid sequence set forth in SEQ ID No: 78 and two light chains having the amino acid sequence set forth in SEQ ID NO: 21;
(b) αTROP2 (HC:BSM-E153C) (LC:BSM) comprising two heavy chains having the amino acid sequence set forth in SEQ ID No: 79 and two light chains having the amino acid sequence set forth in SEQ ID NO: 21;
(c) αTROP2 (HC:BSM-E171C) (LC:BSM) comprising two heavy chains having the amino acid sequence set forth in SEQ ID No: 80 and two light chains having the amino acid sequence set forth in SEQ ID NO: 21;
(d) αTROP2 (HC:BSM-E172C) (LC:BSM) comprising two heavy chains having the amino acid sequence set forth in SEQ ID No: 81 and two light chains having the amino acid sequence set forth in SEQ ID NO: 21;
(e) αTROP2 (HC:BSM-E173C) (LC:BSM) comprising two heavy chains having the amino acid sequence set forth in SEQ ID No: 82 and two light chains having the amino acid sequence set forth in SEQ ID NO: 21;
(f) αTROP2 (HC:BSM) (LC:BSM-E165C) comprising two heavy chains having the amino acid sequence set forth in SEQ ID No: 59 and two light chains having the amino acid sequence set forth in SEQ ID NO: 52;
(g) αTROP2 (HC:BSM) (LC:BSM-E168C) comprising two heavy chains having the amino acid sequence set forth in SEQ ID No: 59 and two light chains having the amino acid sequence set forth in SEQ ID NO: 53;
(h) αTROP2 (HC:BSM-YTE) (LC:BSM-E165C) comprising two heavy chains having the amino acid sequence set forth in SEQ ID No: 60 and two light chains having the amino acid sequence set forth in SEQ ID NO: 52;
(i) αTROP2 (HC:BSM-YTE) (LC:BSM-E168C) comprising two heavy chains having the amino acid sequence set forth in SEQ ID No: 60 and two light chains having the amino acid sequence set forth in SEQ ID NO: 53;
(j) αTROP2 (HC:BSM-YTE-E152C) (LC:BSM) comprising two heavy chains having the amino acid sequence set forth in SEQ ID No: 85 and two light chains having the amino acid sequence set forth in SEQ ID NO: 21;
(k) αTROP2 (HC:BSM-YTE-E153C) (LC:BSM) comprising two heavy chains having the amino acid sequence set forth in SEQ ID No: 86 and two light chains having the amino acid sequence set forth in SEQ ID NO: 21;
(l) αTROP2 (HC:BSM-YTE-E171C) (LC:BSM) comprising two heavy chains having the amino acid sequence set forth in SEQ ID No: 87 and two light chains having the amino acid sequence set forth in SEQ ID NO: 21;
(m) αTROP2 (HC:BSM-YTE-E172C) (LC:BSM) comprising two heavy chains having the amino acid sequence set forth in SEQ ID No: 88 and two light chains having the amino acid sequence set forth in SEQ ID NO: 21;
(n) αTROP2 (HC:BSM-YTE-E173C) (LC:BSM) comprising two heavy chains having the amino acid sequence set forth in SEQ ID No: 89 and two light chains having the amino acid sequence set forth in SEQ ID NO: 21; and
(o) αTROP2 (HC:BSM-YTE-S375C) (LC:BSM) comprising two heavy chains having the amino acid sequence set forth in SEQ ID No: 90 and two light chains having the amino acid sequence set forth in SEQ ID NO: 21;
wherein S is from the side chain of the engineered cysteine residue; and, wherein p is an integer selected from 1, 2, 3, or 4; or (B) selected from the group consisting of:
(a) αTROP2 (HC:BSM-E152C-S375C) (LC:BSM) comprising two heavy chains having the amino acid sequence set forth in SEQ ID No: 84 and two light chains having the amino acid sequence set forth in SEQ ID NO: 21; and
(b) αTROP2 (HC:BSM-YTE-E152C-S375C) (LC:BSM) comprising two heavy chains having the amino acid sequence set forth in SEQ ID No: 91 and two light chains having the amino acid sequence set forth in SEQ ID NO: 21; and
wherein S is from the side chain of the engineered cysteine residue; and, wherein p is an integer selected from 1, 2, 3, or 4.

19. The ADC of claim 16, wherein the ADC comprises the formula: 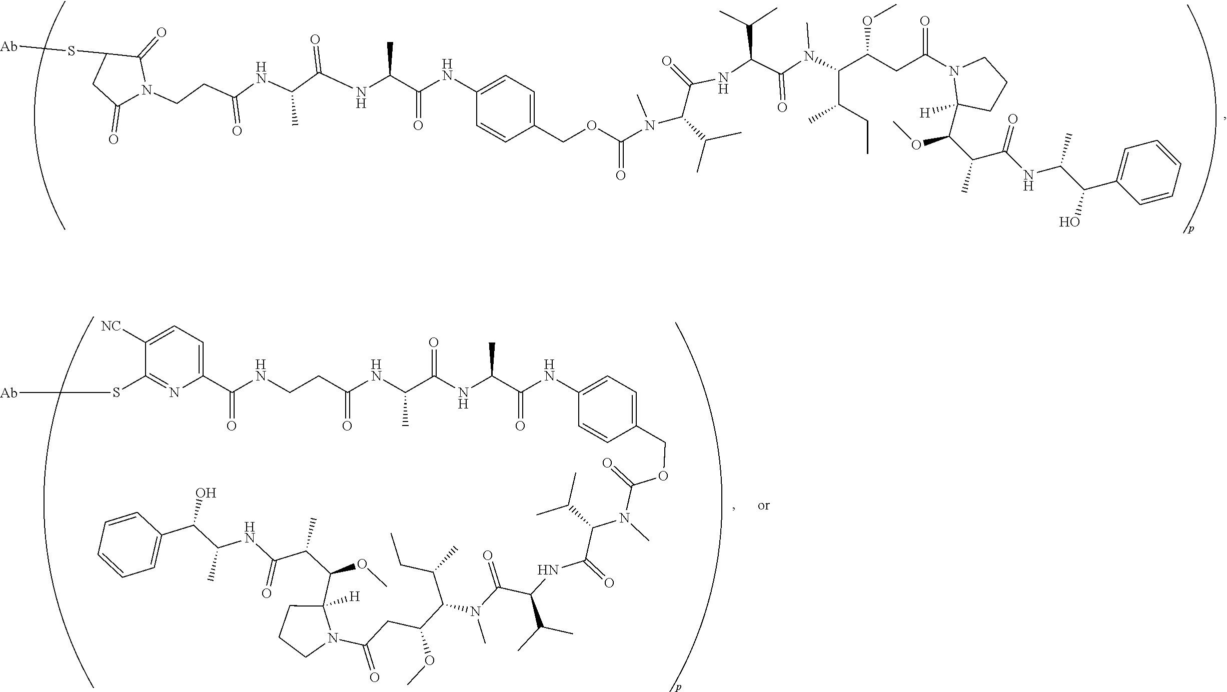 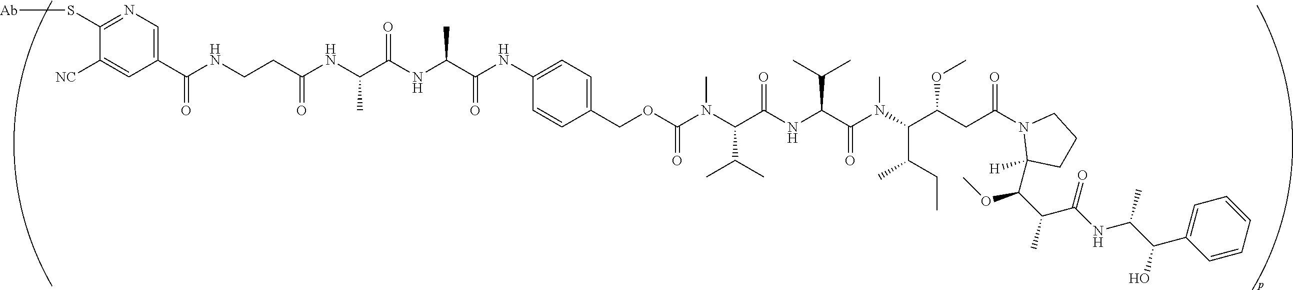
wherein Ab is the antibody, wherein the antibody comprises a heavy chain engineered cysteine residues or light chain engineered cysteine residues, wherein the antibody comprising the engineered cysteine residues is: (A) selected from the group consisting of:
(a) αTROP2 (HC:BSM-E152C) (LC:BSM) comprising two heavy chains having the amino acid sequence set forth in SEQ ID No: 38 and two light chains having the amino acid sequence set forth in SEQ ID NO: 21;
(b) αTROP2 (HC:BSM-E153C) (LC:BSM) comprising two heavy chains having the amino acid sequence set forth in SEQ ID No: 39 and two light chains having the amino acid sequence set forth in SEQ ID NO: 21;
(c) αTROP2 (HC:BSM-E171C) (LC:BSM) comprising two heavy chains having the amino acid sequence set forth in SEQ ID No: 40 and two light chains having the amino acid sequence set forth in SEQ ID NO: 21;
(d) αTROP2 (HC:BSM-E172C) (LC:BSM) comprising two heavy chains having the amino acid sequence set forth in SEQ ID No: 41 and two light chains having the amino acid sequence set forth in SEQ ID NO: 21;
(e) αTROP2 (HC:BSM-E173C) (LC:BSM) comprising two heavy chains having the amino acid sequence set forth in SEQ ID No: 42 and two light chains having the amino acid sequence set forth in SEQ ID NO: 21;
(f) αTROP2 (HC:BSM) (LC:BSM-E165C) comprising two heavy chains having the amino acid sequence set forth in SEQ ID No: 17 and two light chains having the amino acid sequence set forth in SEQ ID NO: 52;
(g) αTROP2 (HC:BSM) (LC:BSM-E168C) comprising two heavy chains having the amino acid sequence set forth in SEQ ID No: 17 and two light chains having the amino acid sequence set forth in SEQ ID NO: 53;
(h) αTROP2 (HC:BSM-YTE) (LC:BSM-E165C) comprising two heavy chains having the amino acid sequence set forth in SEQ ID No: 18 and two light chains having the amino acid sequence set forth in SEQ ID NO: 52;
(i) αTROP2 (HC:BSM-YTE) (LC:BSM-E168C) comprising two heavy chains having the amino acid sequence set forth in SEQ ID No: 18 and two light chains having the amino acid sequence set forth in SEQ ID NO: 53;
(j) αTROP2 (HC:BSM-YTE-E152C) (LC:BSM) comprising two heavy chains having the amino acid sequence set forth in SEQ ID No: 44 and two light chains having the amino acid sequence set forth in SEQ ID NO: 21;
(k) αTROP2 (HC:BSM-YTE-E153C) (LC:BSM) comprising two heavy chains having the amino acid sequence set forth in SEQ ID No: 45 and two light chains having the amino acid sequence set forth in SEQ ID NO: 21;
(l) αTROP2 (HC:BSM-YTE-E171C) (LC:BSM) comprising two heavy chains having the amino acid sequence set forth in SEQ ID No: 46 and two light chains having the amino acid sequence set forth in SEQ ID NO: 21;
(m) αTROP2 (HC:BSM-YTE-E172C) (LC:BSM) comprising two heavy chains having the amino acid sequence set forth in SEQ ID No: 47 and two light chains having the amino acid sequence set forth in SEQ ID NO: 21;
(n) αTROP2 (HC:BSM-YTE-E173C) (LC:BSM) comprising two heavy chains having the amino acid sequence set forth in SEQ ID No: 48 and two light chains having the amino acid sequence set forth in SEQ ID NO: 21; and
(o) αTROP2 (HC:BSM-YTE-S375C) (LC:BSM) comprising two heavy chains having the amino acid sequence set forth in SEQ ID No: 20 and two light chains having the amino acid sequence set forth in SEQ ID NO: 21;
wherein S is from the side chain of the engineered cysteine residue; and, wherein p is an integer selected from 1 or 2; or (B) selected from the group consisting of:
(a) αTROP2 (HC:BSM-E152C-S375C) (LC:BSM) comprising two heavy chains having the amino acid sequence set forth in SEQ ID No: 43 and two light chains having the amino acid sequence set forth in SEQ ID NO: 21; and
(b) αTROP2 (HC:BSM-YTE-E152C-S375C) (LC:BSM) comprising two heavy chains having the amino acid sequence set forth in SEQ ID No: 49 and two light chains having the amino acid sequence set forth in SEQ ID NO: 21;
wherein S is from the side chain of the engineered cysteine residue; and, wherein p is an integer selected from 1, 2, 3, or 4.

20. The ADC of claim 16, wherein the ADC comprises the formula 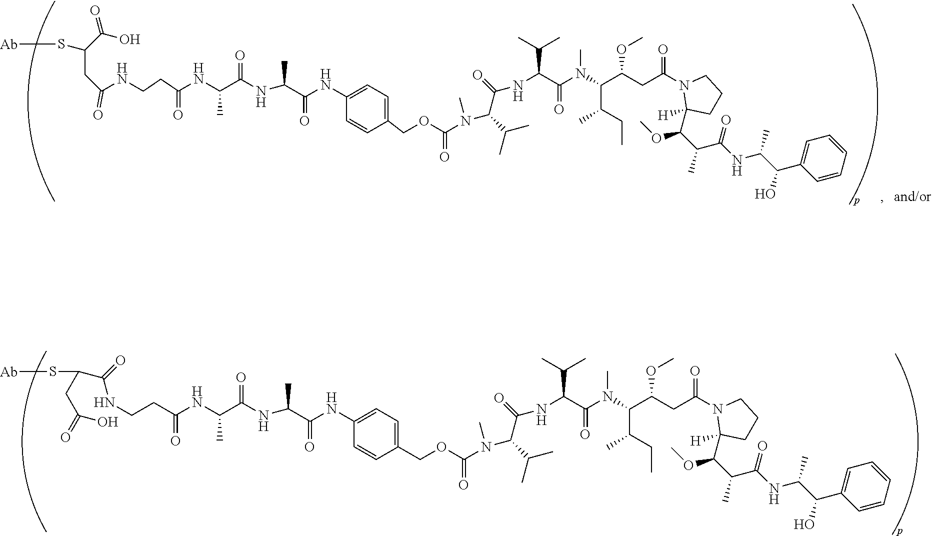
wherein Ab is the antibody; wherein p is 1 or 2; and wherein S is from the side chain of a cysteine residue of the antibody.

21. A composition comprising the ADC of claim 16 and a pharmaceutically acceptable carrier.

22. A method for treating a cancer in an individual in need thereof comprising administering to the individual a therapeutically effective amount of the ADC of claim 16 or the composition of claim 21 to treat the cancer, wherein the cancer is a cancer that overexpresses TROP2.

23. A combination therapy for treating cancer comprising the ADC of claim 16 or the composition of claim 21 and a therapeutic agent, wherein the cancer is a cancer that overexpresses TROP2.

24. An antibody or antigen binding fragment thereof that specifically binds to human TROP2, comprising:
a heavy chain variable domain comprising a CDRH1, a CDRH2, and a CDRH3, and a light chain variable domain comprising a CDRL1, a CDRL2, and a CDRL3, wherein the CDRH1, CDRH2, CDRH3, CDRL1, CDRL2, and CDRL3, respectively, comprise the amino acid sequence NYGMN (SEQ ID NO: 4), WINTYTGEPTYTDDFKG (SEQ ID NO: 5), GGFGSSYWYFDV (SEQ ID NO: 6), KASQDVSIAVA (SEQ ID NO: 7), SASDRYT (SEQ ID NO: 10), and QQHYITPLT (SEQ ID NO: 9).

25. The antibody or antigen binding fragment thereof of claim 24, wherein the antibody or antigen binding fragment thereof displays reduced binding to low TROP2-expressing cells compared to high TROP2-expressing cells and has reduced hydrophobicity compared to Sacituzumab as determined by hydrophobic interaction chromatography (HIC).

26. The antibody or antigen binding fragment thereof of claim 24, wherein the heavy chain variable domain comprises the amino acid sequence of SEQ ID NO: 1 or 14 and the light chain variable domain comprises the amino acid sequence of SEQ ID NO: 3 or 16.

27. The antibody or antigen binding fragment thereof of claim 26, wherein the heavy chain variable domain comprises the amino acid sequence of SEQ ID NO: 1 and the light chain variable domain comprises the amino acid sequence of SEQ ID NO: 3 or the heavy chain variable domain comprises the amino acid sequence of SEQ ID NO: 14 and the light chain variable domain comprises the amino acid sequence of SEQ ID NO: 16.

28. The antibody or antigen binding fragment thereof of claim 24, wherein the antibody comprises a light chain comprising the amino acid sequence of SEQ ID NO: 13 or 22 and the heavy chain comprises the amino acid sequence of SEQ ID NO: 11, 17, or 18.

29. The antibody or antigen binding fragment thereof of claim 24, wherein the antibody comprises a light chain comprising the amino acid sequence of SEQ ID NO: 13 or 22 and a heavy chain comprising the amino acid sequence of SEQ ID NO: 58, 59, or 60.

30. The antibody or antigen binding fragment thereof of claim 24, wherein the antibody further comprises a cysteine or a non-canonical amino acid amino acid substitution at one or more position(s) selected from the group consisting of positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain according to sequential numbering of the whole light chain sequence.

31. A method for treating a cancer in an individual in need thereof comprising administering to the individual a therapeutically effective amount of the antibody or antigen binding fragment thereof of claim 24 or composition comprising the antibody or antigen binding fragment thereof a pharmaceutically acceptable carrier to treat the cancer, wherein the cancer is a cancer that overexpresses TROP2.

32. A combination therapy for treating cancer comprising the antibody or antigen binding fragment thereof of claim 24 or composition comprising the antibody or antigen binding fragment thereof a pharmaceutically acceptable carrier and a therapeutic agent, wherein the cancer is a cancer that overexpresses TROP2.

33. An antibody or antigen binding fragment thereof that specifically binds to human TROP2 comprising a heavy chain variable domain comprising the amino acid sequence of SEQ ID NO: 14 and a light chain variable domain comprising the amino acid sequence of SEQ ID NO: 15.

34. The antibody or antigen binding fragment thereof of claim 33, wherein the antibody or antigen-binding fragment thereof displays reduced hydrophobicity compared to Sacituzumab as determined by hydrophobic interaction chromatography (HIC).

35. The antibody or antigen binding fragment thereof of claim 33, wherein the light chain comprises the amino acid sequence of SEQ ID NO: 21 and the heavy chain comprises the amino acid sequence of SEQ ID NO: 17 or 18.

36. The antibody or antigen binding fragment thereof of claim 33, wherein the antibody comprises a light chain comprising the amino acid sequence of SEQ ID NO: 21 and a heavy chain comprising the amino acid sequence of SEQ ID NO: 59 or 60.

37. The antibody or antigen binding fragment thereof of claim 33, wherein the antibody further comprises a cysteine or a non-canonical amino acid amino acid substitution at one or more position(s) selected from the group consisting of positions 152, 153, 171, 172, 173, and 375 of the constant domain of the heavy chain and positions 165 and 168 of the constant domain of the light chain, wherein the position numbering of the heavy chain constant domain is according to Eu numbering and the position numbering of the light chain constant domain is according to sequential numbering of the whole light chain sequence.

38. A method for treating a cancer in an individual in need thereof comprising administering to the individual a therapeutically effective amount of the antibody or antigen binding fragment thereof of claim 33 or composition comprising the antibody or antigen binding fragment thereof and a pharmaceutically acceptable carrier to treat the cancer, wherein the cancer is a cancer that overexpresses TROP2.

39. A combination therapy for treating cancer comprising the antibody or antigen binding fragment thereof of claim 33 or composition comprising the antibody or antigen binding fragment thereof and a pharmaceutically acceptable carrier and a therapeutic agent, wherein the cancer is a cancer that overexpresses TROP2.
